SEQUENCE LISTING (323 sequences)

SEQ ID NO: 1 - protein, 20 aa, Homo sapiens
MQSGTHWRVLGLCLLSVGVW

SEQ ID NO: 2 - protein, 104 aa, Homo sapiens
DGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMD

SEQ ID NO: 3 - protein, 26 aa, Homo sapiens
VMSVATIVIVDICITGGLLLLVYYWS

SEQ ID NO: 4 - protein, 55 aa, Homo sapiens
KNRKAKAKPVTRGAGAGGRQRGQNKERPPPVPNPDYEPIRKGQRDLYSGLNQRRI

SEQ ID NO: 5 - DNA, 624 bp, Homo sapiens
  source 1..624: mol_type = genomic DNA
atgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctga

SEQ ID NO: 6 - protein, 20 aa, Homo sapiens
MEHSTFLSGLVLATLLSQVS

SEQ ID NO: 7 - protein, 84 aa, Homo sapiens [sequence omitted]

SEQ ID NO: 8 - protein, 21 aa, Homo sapiens
GIIVTDVIATLLLALGVFCFA

SEQ ID NO: 9 - protein, 45 aa, Homo sapiens
GHETGRLSGAADTQALLRNDQVYQPLRDRDDAQYSHLGGNWARNK

SEQ ID NO: 10 - DNA, 516 bp, Homo sapiens
  source 1..516: mol_type = genomic DNA
atggaacatagcacgtttctctctggcctggtactggctacccttctctcgcaagtgagccccttcaagatacctatagaggaacttgaggacagagtgtttgtgaattgcaataccagcatcacatgggtagagggaacggtgggaacactgctctcagacattacaagactggacctgggaaaacgcatcctggacccacgaggaatatataggtgtaatgggacagatatatacaaggacaaagaatctaccgtgcaagttcattatcgaatgtgccagagctgtgtggagctggatccagccaccgtggctggcatcattgtcactgatgtcattgccactctgctccttgctttgggagtcttctgctttgctggacatgagactggaaggctgtctggggctgccgacacacaagctctgttgaggaatgaccaggtctatcagcccctccgagatcgagatgatgctcagtacagccaccttggaggaaactgggctcggaacaagtga

SEQ ID NO: 11 - protein, 22 aa, Homo sapiens
MEQGKGLAVLILAIILLQGTLA

SEQ ID NO: 12 - protein, 94 aa, Homo sapiens
QSIKGNHLVKVYDYQEDGSVLLTCDAEAKNITWFKDGKMIGFLTEDKKKWNLGSNAKDPRGMYQCKGSQNKSKPLQVYYRMCQNCIELNAATIS

SEQ ID NO: 13 - protein, 21 aa, Homo sapiens
GFLFAEIVSIFVLAVGVYFIA

SEQ ID NO: 14 - protein, 45 aa, Homo sapiens
GQDGVRQSRASDKQTLLPNDQLYQPLKDREDDQYSHLQGNQLRRN

SEQ ID NO: 15 - DNA, 549 bp, Homo sapiens
  source 1..549: mol_type = genomic DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgagggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaattga

SEQ ID NO: 16 - protein, 21 aa, Homo sapiens
MKWKALFTAAILQAQLPITEA

SEQ ID NO: 17 - protein, 9 aa, Homo sapiens
QSFGLLDPK

SEQ ID NO: 18 - protein, 21 aa, Homo sapiens
LCYLLDGILFIYGVILTALFL

SEQ ID NO: 19 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 20 - DNA, 495 bp, Homo sapiens
  source 1..495: mol_type = genomic DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 21 - protein, 21 aa, synthetic construct
GSGEGRGSLLTCGDVEENPGP

SEQ ID NO: 22 - protein, 22 aa, synthetic construct
GSGATNFSLLKQAGDVEENPGP

SEQ ID NO: 23 - protein, 23 aa, synthetic construct
GSGQCTNYALLKLAGDVESNPGP

SEQ ID NO: 24 - protein, 25 aa, synthetic construct
GSGVKQTLNFDLLKLAGDVESNPGP

SEQ ID NO: 25 - protein, 255 aa, synthetic construct
XQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 26 - protein, 290 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 27 - DNA, 822 bp, synthetic construct
  source 1..822: mol_type = other DNA
atggactcctggaccttctgctgtgtgtccctttgcatcctggtagcaaagcacacagatgctggacaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacccagtgatgtaggcatctacttctgtgctggacccatgaaaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 28 - protein, 274 aa, synthetic construct
MDSWTFCCVSLCILVAKHTDAGQQLNQSPQSMFIQEGEDVSMNCTSSSIFNTWLWYKQDPGEGPVLLIALYKAGELTSNGRLTAQFGITRKDSFLNISASIPSDVGIYFCAGPMKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 29 - DNA, 924 bp, synthetic construct
  source 1..924: mol_type = other DNA
atggactcctggaccttctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttcggcaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 30 - protein, 308 aa, synthetic construct [sequence omitted]

SEQ ID NO: 31 - DNA, 492 bp, Homo sapiens
  source 1..492: mol_type = genomic DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 32 - protein, 164 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - DNA, 546 bp, Homo sapiens
  source 1..546: mol_type = genomic DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaat

SEQ ID NO: 34 - protein, 182 aa, Homo sapiens
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 35 - DNA, 513 bp, Homo sapiens
  source 1..513: mol_type = genomic DNA
atggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaag

SEQ ID NO: 36 - protein, 171 aa, Homo sapiens
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 37 - DNA, 621 bp, synthetic construct
  source 1..621: mol_type = other DNA
atgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatc

SEQ ID NO: 38 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 39 - protein, 1499 aa, Homo sapiens
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 40 - protein, 245 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRAD

SEQ ID NO: 41 - DNA, 735 bp, synthetic construct
  source 1..735: mol_type = other DNA
atgctcgagggagtgacccagacccccaagttccaggtgctgaagaccggacagagcatgaccctgcagtgcgcccaggacatgaaccacgagtacatgagctggtaccggcaggaccccggaatgggactgcggctgatccactacagcgtgggagccggaatcaccgaccagggagaggtgcccaacggatacaacgtgagccggagcaccaccgaggacttccccctgcggctgctgagcgccgcccccagccagaccagcgtgtacttctgcgccagcagccccgtgaccggaggaatctacggatacaccttcggaagcggaacccggctgaccgtggtggaggacctgaacaaggtgttcccccccgaggtggccgtgttcgagcccagcgaggccgagatcagccacacccagaaggccaccctggtgtgcctggccaccggattcttccccgaccacgtggagctgagctggtgggtgaacggaaaggaggtgcacagcggagtgagcaccgacccccagcccctgaaggagcagcccgccctgaacgacagccggtactgcctgagcagccggctgcgggtgagcgccaccttctggcagaacccccggaaccacttccggtgccaggtgcagttctacggactgagcgagaacgacgagtggacccaggaccgggccaagcccgtgacccagatcgtgagcgccgaggcctggggacgggccgac

SEQ ID NO: 42 - protein, 132 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRATNFSLLKQAGDVEENPGP

SEQ ID NO: 43 - DNA, 396 bp, synthetic construct
  source 1..396: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgccaccaacttctccctgctgaagcaggccggcgacgtggaggagaaccccggcccc

SEQ ID NO: 44 - protein, 205 aa, synthetic construct
MILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESS

SEQ ID NO: 45 - DNA, 615 bp, synthetic construct
  source 1..615: mol_type = other DNA
atgatcctgaacgtggagcagagcccccagagcctgcacgtgcaggagggagacagcaccaacttcacctgcagcttccccagcagcaacttctacgccctgcactggtaccggtgggagaccgccaagagccccgaggccctgttcgtgatgaccctgaacggagacgagaagaagaagggacggatcagcgccaccctgaacaccaaggagggatacagctacctgtacatcaagggaagccagcccgaggacagcgccacctacctgtgcgcccggaacaccggaaaccagttctacttcggaaccggaaccagcctgaccgtgatccccaacatccagaaccccgaccccgccgtgtaccagctgcgggacagcaagagcagcgacaagagcgtgtgcctgttcaccgacttcgacagccagaccaacgtgagccagagcaaggacagcgacgcctacatcaccgacaagaccgtgctggacatgcggagcatggacttcaagagcaacagcgccgtggcctggagcaacaagagcgacttcgcctgcgccaacgccttcaacaacagcatcatccccgaggacaccttcttccccagccccgagagcagc

SEQ ID NO: 46 - protein, 622 aa, synthetic construct
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGP

SEQ ID NO: 47 - DNA, 1866 bp, synthetic construct
  source 1..1866: mol_type = other DNA
atggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggccc

SEQ ID NO: 48 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 49 - DNA, 486 bp, synthetic construct
  source 1..486: mol_type = other DNA
atgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 50 - DNA, 3816 bp, synthetic construct
  source 1..3816: mol_type = other DNA
ctcgagggagtgacccagacccccaagttccaggtgctgaagaccggacagagcatgaccctgcagtgcgcccaggacatgaaccacgagtacatgagctggtaccggcaggaccccggaatgggactgcggctgatccactacagcgtgggagccggaatcaccgaccagggagaggtgcccaacggatacaacgtgagccggagcaccaccgaggacttccccctgcggctgctgagcgccgcccccagccagaccagcgtgtacttctgcgccagcagccccgtgaccggaggaatctacggatacaccttcggaagcggaacccggctgaccgtggtggaggacctgaacaaggtgttcccccccgaggtggccgtgttcgagcccagcgaggccgagatcagccacacccagaaggccaccctggtgtgcctggccaccggattcttccccgaccacgtggagctgagctggtgggtgaacggaaaggaggtgcacagcggagtgagcaccgacccccagcccctgaaggagcagcccgccctgaacgacagccggtactgcctgagcagccggctgcgggtgagcgccaccttctggcagaacccccggaaccacttccggtgccaggtgcagttctacggactgagcgagaacgacgagtggacccaggaccgggccaagcccgtgacccagatcgtgagcgccgaggcctggggacgggccgacgccaccaacttcagcctgctgaagcaggccggcgacgtggaggagaaccccggccccatgatcctgaacgtggagcagagcccccagagcctgcacgtgcaggagggagacagcaccaacttcacctgcagcttccccagcagcaacttctacgccctgcactggtaccggtgggagaccgccaagagccccgaggccctgttcgtgatgaccctgaacggagacgagaagaagaagggacggatcagcgccaccctgaacaccaaggagggatacagctacctgtacatcaagggaagccagcccgaggacagcgccacctacctgtgcgcccggaacaccggaaaccagttctacttcggaaccggaaccagcctgaccgtgatccccaacatccagaaccccgaccccgccgtgtaccagctgcgggacagcaagagcagcgacaagagcgtgtgcctgttcaccgacttcgacagccagaccaacgtgagccagagcaaggacagcgacgcctacatcaccgacaagaccgtgctggacatgcggagcatggacttcaagagcaacagcgccgtggcctggagcaacaagagcgacttcgcctgcgccaacgccttcaacaacagcatcatccccgaggacaccttcttccccagccccgagagcagcgagggcagaggcagcctgctgacctgcggcgacgtggaggagaaccccggccccatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccg

SEQ ID NO: 51 - protein, 1272 aa, synthetic construct
LEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSEGRGSLLTCGDVEENPGPMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGP

SEQ ID NO: 52 - protein, 880 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRPGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 53 - protein, 136 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRPGPQCTNYALLKLAGDVESNPGP

SEQ ID NO: 54 - DNA, 399 bp, synthetic construct
  source 1..399: mol_type = other DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 55 - protein, 880 aa, synthetic construct; identical to SEQ ID NO: 52

SEQ ID NO: 56 - DNA, 1794 bp, synthetic construct
  source 1..1794: mol_type = other DNA
atggactcctggaccttctgctgtgtgtccctttgcatcctggtagcaaagcacacagatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagttcggcaaactatggctacaccttcggttcggggaccaggttaaccgttgtagaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggccatggtcaagagaaaggatttcgagggcaggggaagtcttctaacatgcggggacgtggaggaaaatcccgggcccatgctccttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacccagtgatgtaggcatctacttctgtgctggacccatgaaaacctcctacgacaaggtgatatttgggccagggacaagcttatcagtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 57 - protein, 597 aa, synthetic construct
MDSWTFCCVSLCILVAKHTDAGVIQSPRHEVTEMGQEVTLRCKPISGHDYLFWYRQTMMRGLELLIYFNNNVPIDDSGMPEDRFSAKMPNASFSTLKIQPSEPRDSAVYFCASSSANYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFEGRGSLLTCGDVEENPGPMLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGPMKTSYDKVIFGPGTSLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 58 - protein, 971 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 59 - DNA, 2913 bp, synthetic construct
  source 1..2913: mol_type = other DNA
atgctcgagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 60 - protein, 1043 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRICLTSDRLAPAAGLAAPRRQAVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSTTTPAPRPPTPAPTIASQPLSLRPEACRPAAGGAVHTRGLDFACDFWVLVVVGGVLACYSLLVTVAFIIFWV

SEQ ID NO: 61 - protein, 809 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGP

SEQ ID NO: 62 - DNA, 2427 bp, synthetic construct
  source 1..2427: mol_type = other DNA
atgctcgagatgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggccc

SEQ ID NO: 63 - protein, 24 aa, synthetic construct
MRICLTSDRLAPAAGLAAPRRQAV

SEQ ID NO: 64 - DNA, 72 bp, synthetic construct
  source 1..72: mol_type = other DNA
atgcgcatttgcctgaccagcgatcgcctggcgccggcggcgggcctggcggcgccgcgccgccaggcggtg

SEQ ID NO: 65 - protein, 138 aa, synthetic construct
HKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 66 - DNA, 414 bp, synthetic construct
  source 1..414: mol_type = other DNA
cataaatcttcctctcaaggtcaggaccgccatatgattcgaatgcggcagctgattgacatagtcgatcaactgaagaactatgtgaatgatcttgtgcccgagtttttgccagcccctgaagacgtagaaactaattgtgagtggagtgccttttcctgctttcaaaaggcacagctgaaatccgccaacacgggcaataacgaacggataattaacgtatccattaagaagctgaagcggaagccgccctcaaccaatgcgggacggcggcaaaagcatcgcttgacctgtccgtcatgcgacagctacgagaaaaagcccccgaaggagttcttggaacgcttcaagagtctccttcagaaaatgattcaccagcacctgtcctcacggacgcacggaagcgaggacagt

SEQ ID NO: 67 - protein, 45 aa, synthetic construct
TTTPAPRPPTPAPTIASQPLSLRPEACRPAAGGAVHTRGLDFACD

SEQ ID NO: 68 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 69 - protein, 27 aa, synthetic construct
FWVLVVVGGVLACYSLLVTVAFIIFWV

SEQ ID NO: 70 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 71 - protein, 1037 aa, synthetic construct
MLEMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRICLTSDRLAPAAGLAAPRRQAVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSTTTPAPRPPTPAPTIASQPLSLRPEACRPAAGGAVHTRGLDFACDIYIWAPLAGTCGVLLLSLVIT

SEQ ID NO: 72 - protein, 21 aa, synthetic construct
IYIWAPLAGTCGVLLLSLVIT

SEQ ID NO: 73 - DNA, 63 bp, synthetic construct
  source 1..63: mol_type = other DNA
atctacatctgggcgcccttggccgggacttgtggggtccttctcctgtcactggttatcacc

SEQ ID NO: 74 - protein, 1455 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADATNFSLLKQAGDVEENPGPMILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSEGRGSLLTCGDVEENPGPMKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 75 - protein, 264 aa, synthetic construct
MLEGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSPVTGGIYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADATNFSLLKQAGDVEENPGP

SEQ ID NO: 76 - DNA, 735 bp, synthetic construct; identical to SEQ ID NO: 41
  source 1..735: mol_type = other DNA

SEQ ID NO: 77 - protein, 223 aa, synthetic construct
MILNVEQSPQSLHVQEGDSTNFTCSFPSSNFYALHWYRWETAKSPEALFVMTLNGDEKKKGRISATLNTKEGYSYLYIKGSQPEDSATYLCARNTGNQFYFGTGTSLTVIPNIQNPDPAVYQLRDSKSSDKSVCLFTDFDSQTNVSQSKDSDAYITDKTVLDMRSMDFKSNSAVAWSNKSDFACANAFNNSIIPEDTFFPSPESSEGRGSLLTCGDVEENPGP

SEQ ID NO: 78 - DNA, 672 bp, synthetic construct
  source 1..672: mol_type = other DNA
atgatcctgaacgtggagcagagcccccagagcctgcacgtgcaggagggagacagcaccaacttcacctgcagcttccccagcagcaacttctacgccctgcactggtaccggtgggagaccgccaagagccccgaggccctgttcgtgatgaccctgaacggagacgagaagaagaagggacggatcagcgccaccctgaacaccaaggagggatacagctacctgtacatcaagggaagccagcccgaggacagcgccacctacctgtgcgcccggaacaccggaaaccagttctacttcggaaccggaaccagcctgaccgtgatccccaacatccagaaccccgaccccgccgtgtaccagctgcgggacagcaagagcagcgacaagagcgtgtgcctgttcaccgacttcgacagccagaccaacgtgagccagagcaaggacagcgacgcctacatcaccgacaagaccgtgctggacatgcggagcatggacttcaagagcaacagcgccgtggcctggagcaacaagagcgacttcgcctgcgccaacgccttcaacaacagcatcatccccgaggacaccttcttccccagccccgagagcagcgccaccaacttctccctgctgaagcaggccggcgacgtggaggagaaccccggcccc

SEQ ID NO: 79 - protein, 806 aa, synthetic construct
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGP

SEQ ID NO: 80 - DNA, 2418 bp, synthetic construct
  source 1..2418: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggccc

SEQ ID NO: 81 - DNA, 1200 bp, synthetic construct
  source 1..1200: mol_type = other DNA
atgcggtgggccctactggtgcttctagctttcctgtctcctgccagtcaggataaacaacttgatgcagatgtttcccccaagcccactatttttcttccttcgattgctgaaacaaaactccagaaggctggaacatacctttgtcttcttgagaaatttttcccagatattattaagatacattggcaagaaaagaagagcaacacgattctgggatcccaggaggggaacaccatgaagactaacgacacatacatgaaatttagctggttaacggtgccagaagagtcactggacaaagaacacagatgtatcgtcagacatgagaataataaaaacggaattgatcaagaaattatctttcctccaataaagacagatgtcaccacagtggatcccaaatacaattattcaaaggatgcaaatgatgtcatcacaatggatcccaaagacaattggtcaaaagatgcaaatgatacactactgctgcagctcacaaacacctctgcatattacacgtacctcctcctgctcctcaagagtgtggtctattttgccatcatcacctgctgtctgcttagaagaacggctttctgctgcaatggagagaaatcaggaagcggagctactaactttagcctgctgaagcaggctggagatgtggaggagaaccctggacctatgattcttactgtgggctttagctttttgtttttctacaggggcacgctgtgtagtcagcctcataccaaaccatccgtttttgtcatgaaaaatggaacaaatgtcgcttgtctggtgaaggaattctaccccaaggatataagaataaatctcgtgtcatccaagaagataacagagtttgatcctgctattgtcatctctcccagtgggaagtacaatgctgtcaagcttggtaaatatgaagattcaaattcagtgacatgttcagttcaacacgacaataaaactgtgcactccactgactttgaagtgaagacagattctacagatcacgtaaaaccaaaggaaactgaaaacacaaagcaaccttcaaagagctgccataaacccaaagccatagttcataccgagaaggtgaacatgatgtccctcacagtgcttgggctacgaatgctgtttgcaaagactgttgccgtcaattttctcttgactgccaagttatttttcttgtaa

SEQ ID NO: 82 - protein, 399 aa, synthetic construct
MRWALLVLLAFLSPASQDKQLDADVSPKPTIFLPSIAETKLQKAGTYLCLLEKFFPDIIKIHWQEKKSNTILGSQEGNTMKTNDTYMKFSWLTVPEESLDKEHRCIVRHENNKNGIDQEIIFPPIKTDVTTVDPKYNYSKDANDVITMDPKDNWSKDANDTLLLQLTNTSAYYTYLLLLLKSVVYFAIITCCLLRRTAFCCNGEKSGSGATNFSLLKQAGDVEENPGPMILTVGFSFLFFYRGTLCSQPHTKPSVFVMKNGTNVACLVKEFYPKDIRINLVSSKKITEFDPAIVISPSGKYNAVKLGKYEDSNSVTCSVQHDNKTVHSTDFEVKTDSTDHVKPKETENTKQPSKSCHKPKAIVHTEKVNMMSLTVLGLRMLFAKTVAVNFLLTAKLFFL

SEQ ID NO: 83 - protein, 380 aa, synthetic construct
METLLGLLILWLQLQWVSSIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSIGLLCCAALSLLWAGPVNADLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 84 - DNA, 1143 bp, synthetic construct
  source 1..1143: mol_type = other DNA
atggagaccctcttgggcctgcttatcctttggctgcagctgcaatgggtgagcagcatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggaagcggagctactaactttagcctgctgaagcaggctggagatgtggaggagaaccctggacctatgagcatcggcctcctgtgctgtgcagccttgtctctcctgtgggcaggtccagtgaatgctgacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctaa

SEQ ID NO: 85 - DNA, 345 bp, synthetic construct
  source 1..345: mol_type = other DNA
aaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctttatggaggaagctacatacctacatttggaagaggaaccagccttattgttcatccgtat

SEQ ID NO: 86 - DNA, 420 bp, synthetic construct
  source 1..420: mol_type = other DNA
atccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 87 - DNA, 822 bp, synthetic construct
  source 1..822: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctttatggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 88 - protein, 115 aa, synthetic construct
XQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPY

SEQ ID NO: 89 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 90 - protein, 6 aa, synthetic construct
DSAIYN

SEQ ID NO: 91 - protein, 7 aa, synthetic construct
IQSSQRE

SEQ ID NO: 92 - protein, 15 aa, synthetic construct
CAVRPLYGGSYIPTF

SEQ ID NO: 93 - DNA, 336 bp, synthetic construct
  source 1..336: mol_type = other DNA
ggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggag

SEQ ID NO: 94 - DNA, 534 bp, synthetic construct
  source 1..534: mol_type = other DNA
gacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 95 - DNA, 933 bp, synthetic construct
  source 1..933: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggc

SEQ ID NO: 96 - protein, 112 aa, synthetic construct
GVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLE

SEQ ID NO: 97 - protein, 178 aa, synthetic construct
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 98 - protein, 5 aa, synthetic construct
MNHEY

SEQ ID NO: 99 - protein, 6 aa, synthetic construct
SVGAGI

SEQ ID NO: 100 - protein, 14 aa, synthetic construct
CASSYVGNTGELFF

SEQ ID NO: 101 - DNA, 57 bp, synthetic construct
  source 1..57: mol_type = other DNA
atggagaccctcttgggcctgcttatcctttggctgcagctgcaatgggtgagcagc

SEQ ID NO: 102 - DNA, 63 bp, synthetic construct
  source 1..63: mol_type = other DNA
atgagcatcggcctcctgtgctgtgcagccttgtctctcctgtgggcaggtccagtgaatgct

SEQ ID NO: 103 - protein, 19 aa, synthetic construct
METLLGLLILWLQLQWVSS

SEQ ID NO: 104 - protein, 21 aa, synthetic construct
MSIGLLCCAALSLLWAGPVNA

SEQ ID NO: 105 - protein, 9 aa, synthetic construct
SLLMWITQC

SEQ ID NO: 106 - DNA, 624 bp, synthetic construct
  source 1..624: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgctttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 107 - DNA, 675 bp, synthetic construct
  source 1..675: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 108 - DNA, 747 bp, synthetic construct
  source 1..747: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggcccc

SEQ ID NO: 109 - protein, 208 aa, synthetic construct
MKWKALFTAAILQAQLPITEAQSFGLLDPKLCYLLDGILFIYGVILTALFLLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 110 - protein, 225 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 111 - protein, 249 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGP

SEQ ID NO: 112 - protein, 72 aa, synthetic construct
CTTTGCGCACGCCCACGCCGCAGCCCCGCCCAAGAAGATGGCAAAGTCTACATCAACATGCCAGGCAGGGGC

SEQ ID NO: 113 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctca

SEQ ID NO: 114 - DNA, 195 bp, synthetic construct
  source 1..195: mol_type = other DNA
aggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggc

SEQ ID NO: 115 - protein, 24 aa, synthetic construct
LCARPRRSPAQEDGKVYINMPGRG

SEQ ID NO: 116 - protein, 41 aa, synthetic construct
RSKRSRLLHSDYMNMTPRRPGPTRKHYQPYAPPRDFAAYRS

SEQ ID NO: 117 - protein, 65 aa, synthetic construct
RSKRSRLLHSDYMNMTPRRPGPTRKHYQPYAPPRDFAAYRSLCARPRRSPAQEDGKVYINMPGRG

SEQ ID NO: 118 - DNA, 2976 bp, synthetic construct
  source 1..2976: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgctttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 119 - protein, 992 aa, synthetic construct
MKWKALFTAAILQAQLPITEAQSFGLLDPKLCYLLDGILFIYGVILTALFLLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 120 - DNA, 3027 bp, synthetic construct
  source 1..3027: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcaagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 121 - protein, 1009 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 122 - DNA, 3099 bp, synthetic construct
  source 1..3099: mol_type = other DNA
atgaagtggaaggcgcttttcaccgcggccatcctgcaggcacagttgccgattacagaggcacagagctttggcctgctggatcccaaactctgctacctgctggatggaatcctcttcatctatggtgtcattctcactgccttgttcctgaggagtaagaggagcaggctcctgcacagtgactacatgaacatgactccccgccgccccgggcccacccgcaagcattaccagccctatgccccaccacgcgacttcgcagcctatcgctcactttgcgcacgcccacgccgcagccccgcccaagaagatggcaaagtctacatcaacatgccaggcaggggcagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgccagtgcaccaactacgccctgctgaagctggccggcgacgtggagagcaaccccggccccatggaacaggggaagggcctggctgtcctcatcctggctatcattcttcttcaaggtactttggcccagtcaatcaaaggaaaccacttggttaaggtgtatgactatcaagaagatggttcggtacttctgacttgtgatgcagaagccaaaaatatcacatggtttaaagatgggaagatgatcggcttcctaactgaagataaaaaaaaatggaatctgggaagtaatgccaaggaccctcgtgggatgtatcagtgtaaaggatcacagaacaagtcaaaaccactccaagtgtattacagaatgtgtcagaactgcattgaactaaatgcagccaccatatctggctttctctttgctgaaatcgtcagcattttcgtccttgctgttggggtctacttcattgctggacaggatggagttcgccagtcgagagcttcagacaagcagactctgttgcccaatgaccagctctaccagcccctcaaggatcgagaagatgaccagtacagccaccttcaaggaaaccagttgaggaggaatgtgaagcagaccctgaacttcgacctgctgaagctggccggcgacgtggagagcaaccccggccccatggagcacagcaccttcctgagcggcctggtgctggccaccctgctgagccaggtgagccccttcaagatccccatcgaggagctggaggacagagtgttcgtgaactgcaacaccagcatcacctgggtggagggcaccgtgggcaccctgctgagcgacatcaccagactggacctgggcaagagaatcctggaccccagaggcatctacagatgcaacggcaccgacatctacaaggacaaggagagcaccgtgcaggtgcactacagaatgtgccagagctgcgtggagctggaccccgccaccgtggccggcatcatcgtgaccgacgtgatcgccaccctgctgctggccctgggcgtgttctgcttcgccggccacgagaccggcagactgagcggcgccgccgacacccaggccctgctgagaaacgaccaggtgtaccagcccctgagagacagagacgacgcccagtacagccacctgggcggcaactgggccagaaacaaggagggcagaggcagcctgctgacctgcggcgacgtggaggagaaccccggccccatgcagagcggcacccactggagagtgctgggcctgtgcctgctgagcgtgggcgtgtggggccaggacggcaacgaggagatgggcggcatcacccagaccccctacaaggtgagcatcagcggcaccaccgtgatcctgacctgcccccagtaccccggcagcgagatcctgtggcagcacaacgacaagaacatcggcggcgacgaggacgacaagaacatcggcagcgacgaggaccacctgagcctgaaggagttcagcgagctggagcagagcggctactacgtgtgctaccccagaggcagcaagcccgaggacgccaacttctacctgtacctgagagccagagtgtgcgagaactgcatggagatggacgtgatgagcgtggccaccatcgtgatcgtggacatctgcatcaccggcggcctgctgctgctggtgtactactggagcaagaacagaaaggccaaggccaagcccgtgaccagaggcgccggcgccggcggcagacagagaggccagaacaaggagagacccccccccgtgcccaaccccgactacgagcccatcagaaagggccagagagacctgtacagcggcctgaaccagagaagaatcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagc

SEQ ID NO: 123 - protein, 1033 aa, synthetic construct
MKWKALFTAAILQAQLPITEAQSFGLLDPKLCYLLDGILFIYGVILTALFLRSKRSRLLHSDYMNMTPRRPGPTRKHYQPYAPPRDFAAYRSLCARPRRSPAQEDGKVYINMPGRGRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRQCTNYALLKLAGDVESNPGPMEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRNVKQTLNFDLLKLAGDVESNPGPMEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNKEGRGSLLTCGDVEENPGPMQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRIGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 124 - DNA, 3288 bp, synthetic construct
  source 1..3288: mol_type = other DNA
atggagaccctcttgggcctgcttatcctttggctgcagctgcaatgggtgagcagcaaacaggaggtgacacagattcctgcagctctgagtgtcccagaaggagaaaacttggttctcaactgcagtttcactgatagcgctatttacaacctccagtggtttaggcaggaccctgggaaaggtctcacatctctgttgcttattcagtcaagtcagagagagcaaacaagtggaagacttaatgcctcgctggataaatcatcaggacgtagtactttatacattgcagcttctcagcctggtgactcagccacctacctctgtgctgtgaggcccctttatggaggaagctacatacctacatttggaagaggaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagcggaagcggagctactaactttagcctgctgaagcaggctggagatgtggaggagaaccctggacctatgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttacgtcgggaacaccggggagctgttttttggagaaggctctaggctgaccgtactggaggacctgaaaaacgtgttcccacccaaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggcagtggacagtgcaccaactacgccctgctgaagctggccggcgacgtggagagcaaccccggccccatggccttgcccgtcactgcgcttttgctcccgctcgctcttctcctgcatgcagcccgaccatctcaatttagagtttctccactcgacaggacgtggaacctcggcgaaaccgtcgaacttaaatgtcaagtacttctctcaaatccgacttctggttgctcatggctctttcagccgagaggagcagctgccagccccaccttcctgctgtatctctcccagaacaagccgaaggccgccgaagggctcgatactcaacgatttagcgggaagcgactcggggacacgttcgttcttactctcagcgattttagaagagagaacgagggatattatttttgttccgcactctctaacagcatcatgtacttcagtcattttgtaccagtctttctccctgcaaaaccaacgactactccagcaccaagaccgcccactcccgcacctactattgcaagccaacctttgagtctccgaccagaggcatgcagacctgctgctggaggtgcagtacatacgcgagggttggattttgcctgcgatatctatatctgggcccccttggccggcacgtgcggggtgctcctgctgagtctcgtaattactctttattgtaatcatagaaaccgcagaagggtgtgtaagtgtccccggcctgtcgtgaaaagcggggataagcccagtttgtctgctcggtacgtcggaagcggtgagggcaggggaagtcttctaacatgcggggacgtggaggaaaatcccggacccatgaggccacgactttggctgctgctcgctgcacagttgactgtactgcatggcaatagtgtgttgcagcagacacctgcatacatcaaggttcagacaaataagatggttatgctgagttgcgaggcaaaaattagtttgagcaatatgcggatctactggttgcgacagagacaggctcccagtagtgatagtcaccacgaattcctggctctttgggattccgcaaaaggaacgattcatggggaagaagtagagcaggagaagattgcggttttccgcgatgcatctcgctttatccttaatcttacatccgttaagcctgaggacagtgggatctatttttgtatgattgtagggtcccccgaattgacatttgggaagggtacgcagctctccgtagttgactttctgcccacaacggcacaacccactaagaagtccaccctgaagaagcgcgtctgtcgcttgcccagacctgaaacccaaaagggtccactctgttcccctataaccctggggttgttggtggcgggcgtcttggtcctgcttgttagcttgggcgtagccattcatctgtgttgccgaagacgcagagcccgacttagatttatgaagcaattctataagtga

SEQ ID NO: 125 - protein, 1095 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSGATNFSLLKQAGDVEENPGPMSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGSGQCTNYALLKLAGDVESNPGPMALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYVGSGEGRGSLLTCGDVEENPGPMRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 126 - DNA, 633 bp, synthetic construct
  source 1..633: mol_type = other DNA
atgaggccacgactttggctgctgctcgctgcacagttgactgtactgcatggcaatagtgtgttgcagcagacacctgcatacatcaaggttcagacaaataagatggttatgctgagttgcgaggcaaaaattagtttgagcaatatgcggatctactggttgcgacagagacaggctcccagtagtgatagtcaccacgaattcctggctctttgggattccgcaaaaggaacgattcatggggaagaagtagagcaggagaagattgcggttttccgcgatgcatctcgctttatccttaatcttacatccgttaagcctgaggacagtgggatctatttttgtatgattgtagggtcccccgaattgacatttgggaagggtacgcagctctccgtagttgactttctgcccacaacggcacaacccactaagaagtccaccctgaagaagcgcgtctgtcgcttgcccagacctgaaacccaaaagggtccactctgttcccctataaccctggggttgttggtggcgggcgtcttggtcctgcttgttagcttgggcgtagccattcatctgtgttgccgaagacgcagagcccgacttagatttatgaagcaattctataagtga

SEQ ID NO: 127 - DNA, 705 bp, synthetic construct
  source 1..705: mol_type = other DNA
atggccttgcccgtcactgcgcttttgctcccgctcgctcttctcctgcatgcagcccgaccatctcaatttagagtttctccactcgacaggacgtggaacctcggcgaaaccgtcgaacttaaatgtcaagtacttctctcaaatccgacttctggttgctcatggctctttcagccgagaggagcagctgccagccccaccttcctgctgtatctctcccagaacaagccgaaggccgccgaagggctcgatactcaacgatttagcgggaagcgactcggggacacgttcgttcttactctcagcgattttagaagagagaacgagggatattatttttgttccgcactctctaacagcatcatgtacttcagtcattttgtaccagtctttctccctgcaaaaccaacgactactccagcaccaagaccgcccactcccgcacctactattgcaagccaacctttgagtctccgaccagaggcatgcagacctgctgctggaggtgcagtacatacgcgagggttggattttgcctgcgatatctatatctgggcccccttggccggcacgtgcggggtgctcctgctgagtctcgtaattactctttattgtaatcatagaaaccgcagaagggtgtgtaagtgtccccggcctgtcgtgaaaagcggggataagcccagtttgtctgctcggtacgtc

SEQ ID NO: 128 - protein, 210 aa, synthetic construct
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 129 - protein, 235 aa, synthetic construct
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 130 - skipped

SEQ ID NO: 131 - protein, 9 aa, synthetic construct
SLLQHLIGL

SEQ ID NO: 132 - protein, 9 aa, synthetic construct
QLLALLPSL

SEQ ID NO: 133 - DNA, 816 bp, synthetic construct
  source 1..816: mol_type = other DNA
atgcttctggaacacctgctgattatcctgtggatgcaactcacgtgggtctccgggcaacaactgaatcaaagcccccaatccatgtttatacaggagggagaggacgtaagtatgaattgcacatcttcatctatctttaacacctggctgtggtacaaacaagaccccggagaaggtcctgtacttctcatcgcactttacaaagcaggtgagcttaccagtaacgggagactcaccgcacagttcggtattacaagaaaggattcctttctcaacatctccgcttctatcccttcagacgtcggaatttatttttgtgctggtatccctcgagacaattacggtcaaaactttgtatttgggcctgggactcggctgtcagttttgccgtatatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 134 - DNA, 936 bp, synthetic construct
  source 1..936: mol_type = other DNA
atgggcattaggctgctgtgcagagtagcattttgctttctggcagtaggattggtcgatgtaaaggttacacagtcctcacggtacttggtaaagcgcactggtgaaaaggtctttctggaatgtgtacaagatatggatcacgaaaatatgttttggtacaggcaagatcccggccttggacttagactgatatatttctcctacgatgttaaaatgaaggagaagggcgatattccagaaggatattccgtgagccgcgaaaagaaggagcgattcagtttgatactcgaaagtgcctccacaaaccaaacctctatgtacctttgcgcgtcaacgccgtggctggccggtggcaatgaacaattcttcgggccgggtacgcgcctcactgtcctggaggacctcaagaatgtgtttccgcccgaagtcgcggtttttgaaccatcagaagccgagatctctcatacacaaaaggcgacgctcgtatgccttgcgacgggattttatccggaccacgtcgagctttcctggtgggttaatggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 135 - protein, 272 aa, synthetic construct
MLLEHLLIILWMQLTWVSGQQLNQSPQSMFIQEGEDVSMNCTSSSIFNTWLWYKQDPGEGPVLLIALYKAGELTSNGRLTAQFGITRKDSFLNISASIPSDVGIYFCAGIPRDNYGQNFVFGPGTRLSVLPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 136 - protein, 312 aa, synthetic construct
MGIRLLCRVAFCFLAVGLVDVKVTQSSRYLVKRTGEKVFLECVQDMDHENMFWYRQDPGLGLRLIYFSYDVKMKEKGDIPEGYSVSREKKERFSLILESASTNQTSMYLCASTPWLAGGNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 137 - DNA, 822 bp, synthetic construct
  source 1..822: mol_type = other DNA
atgctgctgctgctggtgcccgtgctggaagtgatcttcaccctgggcggcaccagagcccagagcgtgacacagctgggcagccacgtgtccgtgtctgagagggccctggtgctgctgagatgcaactactcttctagcgtgcccccctacctgttttggtacgtgcagtaccccaaccaggggctgcagctgctcctgaagtacaccagcgccgccacactggtgaagggcatcaacggcttcgaggccgagttcaagaagtccgagacaagcttccacctgaccaagcccagcgcccacatgtctgacgccgccgagtacttctgtgccgtgagcggccagaccggcgccaacaacctgttcttcggcaccggcacccggctgacagtgatcccttacatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 138 - DNA, 933 bp, synthetic construct
  source 1..933: mol_type = other DNA
atgggcttccggctgctgtgctgcgtggccttttgtctgctgggagccggacctgtggatagcggcgtgacccagacccccaagcacctgatcaccgccaccggccagagagtgaccctgcgctgcagccctagaagcggcgacctgagcgtgtactggtatcagcagagcctcgaccagggcctgcagttcctgatccagtactacaacggcgaggaacgggccaagggcaacatcctggaacggttcagcgcccagcagttccccgatctgcacagcgagctgaacctgagcagcctggaactgggcgacagcgccctgtacttctgcgccagcgccagatgggatagaggcggcgagcagtacttcggccctggcaccagactgaccgtgaccgaggacctcaagaatgtgtttccgcccgaagtcgcggtttttgaaccatcagaagccgagatctctcatacacaaaaggcgacgctcgtatgccttgcgacgggattttatccggaccacgtcgagctttcctggtgggttaatggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 139 - protein, 274 aa, synthetic construct
MLLLLVPVLEVIFTLGGTRAQSVTQLGSHVSVSERALVLLRCNYSSSVPPYLFWYVQYPNQGLQLLLKYTSAATLVKGINGFEAEFKKSETSFHLTKPSAHMSDAAEYFCAVSGQTGANNLFFGTGTRLTVIPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 140 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASARWDRGGEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 141 - DNA, 819 bp, synthetic construct
  source 1..819: mol_type = other DNA
atgaagagcctgagggtactgctggtgatattgtggcttcagcttagttgggtctggtcacaacaaaaggaagttgagcaaaactcaggaccactgagtgtacccgagggcgctatagcatcactgaactgtacctactcagatcggggaagccaatcctttttctggtacagacagtattccgggaagagtcctgagttgatcatgtttatatactccaatggcgataaggaggatggacgcttcaccgctcagcttaataaagcgtcacagtatgtatccctcctgattcgggactcacaaccatctgactctgcaacatacctttgtgccgtaaaggacaacgccgggaacatgctcacttttggaggaggtacccggcttatggtaaaaccacatatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 142 - protein, 273 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVKDNAGNMLTFGGGTRLMVKPHIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 143 - DNA, 933 bp, synthetic construct
  source 1..933: mol_type = other DNA
atgggattccggcttctttgttgtgtggcattttgtctgttgggtgcgggtccagtcgatagtggtgtaactcagacaccaaaacaccttatcacggcaactgggcaacgagtgacgctccgctgtagcccgaggtccggtgatttgagtgtgtactggtaccagcaatctttggaccagggcttgcagttcctcatacagtattacaatggtgaagaaagagcgaagggtaatatcctggaaagattctccgcacaacagtttcctgatctccacagcgaactgaacctgagttctctcgagctcggggatagtgctttgtacttctgcgcgtcatccgacggtggcggagtctatgaacaatatttcggcccagggactaggcttacggtgacggaggacctcaagaatgtgtttccgcccgaagtcgcggtttttgaaccatcagaagccgagatctctcatacacaaaaggcgacgctcgtatgccttgcgacgggattttatccggaccacgtcgagctttcctggtgggttaatggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 144 - protein, 311 aa, synthetic construct
MGFRLLCCVAFCLLGAGPVDSGVTQTPKHLITATGQRVTLRCSPRSGDLSVYWYQQSLDQGLQFLIQYYNGEERAKGNILERFSAQQFPDLHSELNLSSLELGDSALYFCASSDGGGVYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 145 - DNA, 402 bp, synthetic construct
  source 1..402: mol_type = other DNA
atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgaccttcggcaagggcaccctgctgaccgtgaatcccaat

SEQ ID NO: 146 - DNA, 396 bp, synthetic construct
  source 1..396: mol_type = other DNA
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaac

SEQ ID NO: 147 - DNA, 810 bp, synthetic construct
  source 1..810: mol_type = other DNA
atggagacactgctgaaggtgctgtctggcacactgctgtggcagctgacctgggtccgatctcagcagcctgttcagtctcctcaggccgtgatcctgagagaaggcgaggacgccgtgatcaactgcagcagctctaaggccctgtacagcgtgcactggtacagacagaagcacggcgaggcccctgtgttcctgatgatcctgctgaaaggcggcgagcagaagggccacgagaagatcagcgccagcttcaacgagaagaagcagcagtccagcctgtacctgacagccagccagctgagctacagcggcacctacttttgcggcacagccaatagcggcggcagcaactacaagctgaccttcggcaagggcaccctgctgaccgtgaatcccaatatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 148 - DNA, 804 bp, synthetic construct
  source 1..804: mol_type = other DNA
atgctgctgatcacctccatgctggtgctgtggatgcagctgagccaagtgaacggccagcaagtgatgcagatccctcagtaccagcacgtgcaagaaggcgaggacttcaccacctactgcaacagcagcaccacactgagcaacatccagtggtacaagcagcggcctggcggacaccctgtgtttctgatccagctggtcaagtccggcgaagtgaagaagcagaagcggctgaccttccagttcggcgaggccaagaagaacagcagcctgcacatcaccgccacacagaccaccgatgtgggcacctacttttgtgctggcgccctgcctagagccggcagctatcaactgacattcggcaagggcaccaagctgagcgtgatccccaacatccagaatccggagcccgccgtataccagctgaaggaccctagaagccaggacagcaccctgtgcctgttcaccgacttcgacagccagatcaacgtgcccaagaccatggaaagcggcaccttcatcaccgacaagacagtgctggacatgaaggccatggacagcaagtccaacggcgcaatcgcctggtccaaccagaccagcttcacatgccaggacatcttcaaagagacaaacgccacataccccagcagcgacgtgccctgtgatgccaccctgacagagaagtccttcgagacagacatgaacctgaacttccagaatctgtccgtgatgggcctgagaatcctgctgctgaaggtggccggcttcaatctgctgatgaccctgcggctgtggtccagc

SEQ ID NO: 149 - DNA, 393 bp, synthetic construct
  source 1..393: mol_type = other DNA
atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtg

SEQ ID NO: 150 - DNA, 393 bp, synthetic construct
  source 1..393: mol_type = other DNA
atgctgtgttctctgctggctctgctgctgggcaccttttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctg

SEQ ID NO: 151 - DNA, 912 bp, synthetic construct
  source 1..912: mol_type = other DNA
atgggcaccagactgttcttctacgtggccctgtgtctgctgtggacaggccatgtggatgccggaatcacacagagccccagacacaaagtgaccgagacaggcacccctgtgacactgagatgtcaccagaccgagaaccatcggtacatgtattggtacagacaggaccccggccacggcctgagactgatccactatagctacggcgtgaaggacaccgacaagggcgaagtgtctgacggctacagcgtgtccagaagcaagaccgaggacttcctgctgaccctggaaagcgccacaagcagccagaccagcgtgtacttctgcgccatcagcgactacgagggcaccgaggccttttttggccaaggcacaagactgaccgtggtggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 152 - DNA, 912 bp, synthetic construct
  source 1..912: mol_type = other DNA
atgctgtgttctctgctggctctgctgctgggcaccttttttggcgtcagaagccagaccatccaccagtggcctgctacactggtgcagcctgttggaagccctctgagcctggaatgtaccgtggaaggcaccagcaatcccaacctgtactggtacagacaggccgctggaagaggactgcagctgctgttttacagcgtcggcatcggccagatcagcagcgaggttccacagaatctgagcgccagcagaccccaggacagacagtttatcctgagcagcaagaagctgctgctgagcgacagcggcttctacctgtgtgcttggagcctcggagccggctacaccgacacacagtattttggccctggcaccagactgaccgtgctggaagatctccggaacgtgaccccccctaaagtgaccctgttcgaacccagcaaggccgagatcgccaacaagcagaaagccaccctcgtgtgcctggccagaggcttcttccccgaccatgtggaactgtcttggtgggtcaacggcaaagaggtgcacagcggagtgtccaccgaccctcaggcctacaaagagagcaactacagctactgcctgagcagcagactgcgggtgtccgccaccttctggcacaacccccggaaccacttcagatgccaggtgcagtttcacggcctgagcgaagaggacaagtggcccgaaggctcccccaagcccgtgacccagaatatctctgccgaggcctggggcagagccgactgtggaattaccagcgccagctaccaccagggcgtgctgtctgccaccatcctgtacgagatcctgctgggcaaggccaccctgtacgccgtgctggtgtctggcctggtgctgatggccatggtcaagaagaagaacagc

SEQ ID NO: 153 - protein, 134 aa, synthetic construct
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTANSGGSNYKLTFGKGTLLTVNPN

SEQ ID NO: 154 - protein, 132 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGALPRAGSYQLTFGKGTKLSVIPN

SEQ ID NO: 155 - protein, 136 aa, synthetic construct
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 156 - protein, 140 aa, synthetic construct; identical to SEQ ID NO: 89

SEQ ID NO: 157 - protein, 140 aa, synthetic construct
IQNPDPAVYQLRDSKSSDKSVCLFTDFDSQTNVSQSKDSDVYITDKTVLDMRSMDFKSNSAVAWSNKSDFACANAFNNSIIPEDTFFPSSDVPCDVKLVEKSFETDTNLNFQNLSVIGFRILLLKVAGFNLLMTLRLWSS

SEQ ID NO: 158 - protein, 270 aa, synthetic construct
METLLKVLSGTLLWQLTWVRSQQPVQSPQAVILREGEDAVINCSSSKALYSVHWYRQKHGEAPVFLMILLKGGEQKGHEKISASFNEKKQQSSLYLTASQLSYSGTYFCGTANSGGSNYKLTFGKGTLLTVNPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 159 - protein, 268 aa, synthetic construct
MLLITSMLVLWMQLSQVNGQQVMQIPQYQHVQEGEDFTTYCNSSTTLSNIQWYKQRPGGHPVFLIQLVKSGEVKKQKRLTFQFGEAKKNSSLHITATQTTDVGTYFCAGALPRAGSYQLTFGKGTKLSVIPNIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 160 - protein, 131 aa, synthetic construct
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISDYEGTEAFFGQGTRLTVV

SEQ ID NO: 161 - protein, 131 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSLGAGYTDTQYFGPGTRLTVL

SEQ ID NO: 162 - protein, 173 aa, synthetic construct
EDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 163 - protein, 176 aa, synthetic construct
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 164 - protein, 177 aa, synthetic construct
EDLNKVFPPEVAVFEPSKAEIAHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGITSASYHQGVLSATILYEILLGKATLYAVLVSALVLMAMVKRKDF

SEQ ID NO: 165 - protein, 304 aa, synthetic construct
MGTRLFFYVALCLLWTGHVDAGITQSPRHKVTETGTPVTLRCHQTENHRYMYWYRQDPGHGLRLIHYSYGVKDTDKGEVSDGYSVSRSKTEDFLLTLESATSSQTSVYFCAISDYEGTEAFFGQGTRLTVVEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 166 - protein, 304 aa, synthetic construct
MLCSLLALLLGTFFGVRSQTIHQWPATLVQPVGSPLSLECTVEGTSNPNLYWYRQAAGRGLQLLFYSVGIGQISSEVPQNLSASRPQDRQFILSSKKLLLSDSGFYLCAWSLGAGYTDTQYFGPGTRLTVLEDLRNVTPPKVTLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 167 - protein, 9 aa, synthetic construct
LYVDSLFFL

SEQ ID NO: 168 - protein, 9 aa, synthetic construct
KTWGQYWQV

SEQ ID NO: 169 - DNA, 804 bp, synthetic construct
  source 1..804: mol_type = other DNA
atgaaatccttgagtgtttccctagtggtcctgtggctccagttaaactgggtgaacagccagcagaaggtgcagcagagcccagaatccctcattgtcccagagggagccatgacctctctcaactgcactttcagcgacagtgcttctcagtattttgcatggtacagacagcattctgggaaagcccccaaggcactgatgtccatcttctccaatggtgaaaaagaagaaggcagattcacaattcacctcaataaagccagtctgcatttctcgctacacatcagagactcccagcccagtgactctgctctctacctctgtgcagccaataactatgcccagggattaaccttcggtcttggcaccagagtatctgtgtttccctacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagttga

SEQ ID NO: 170 - DNA, 912 bp, synthetic construct
  source 1..912: mol_type = other DNA
atgggctccagactcttctttgtggttttgattctcctgtgtgcaaaacacatggaggctgcagtcacccaaagtccaagaagcaaggtggcagtaacaggaggaaaggtgacattgagctgtcaccagactaataaccatgactatatgtactggtatcggcaggacacggggcatgggctgaggctgatccattactcatatgtcgctgacagcacggagaaaggagatatccctgatgggtacaaggcctccagaccaagccaagagaatttctctctcattctggagttggcttccctttctcagacagctgtatatttctgtgccagcagccctgggggggggggggaacagtacttcggtcccggcaccaggctcacggttttagaggatctgagaaatgtgactccacccaaggtctccttgtttgagccatcaaaagcagagattgcaaacaaacgaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaattcatga

SEQ ID NO: 171 - protein, 267 aa, synthetic construct
MKSLSVSLVVLWLQLNWVNSQQKVQQSPESLIVPEGAMTSLNCTFSDSASQYFAWYRQHSGKAPKALMSIFSNGEKEEGRFTIHLNKASLHFSLHIRDSQPSDSALYLCAANNYAQGLTFGLGTRVSVFPYIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 172 - protein, 303 aa, synthetic construct
MGSRLFFVVLILLCAKHMEAAVTQSPRSKVAVTGGKVTLSCHQTNNHDYMYWYRQDTGHGLRLIHYSYVADSTEKGDIPDGYKASRPSQENFSLILELASLSQTAVYFCASSPGGGGEQYFGPGTRLTVLEDLRNVTPPKVSLFEPSKAEIANKRKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 173 - protein, 111 aa, synthetic construct
QQKVQQSPESLIVPEGAMTSLNCTFSDSASQYFAWYRQHSGKAPKALMSIFSNGEKEEGRFTIHLNKASLHFSLHIRDSQPSDSALYLCAANNYAQGLTFGLGTRVSVFPY

SEQ ID NO: 174 - protein, 112 aa, synthetic construct
EAAVTQSPRSKVAVTGGKVTLSCHQTNNHDYMYWYRQDTGHGLRLIHYSYVADSTEKGDIPDGYKASRPSQENFSLILELASLSQTAVYFCASSPGGGGEQYFGPGTRLTVL

SEQ ID NO: 175 - protein, 9 aa, synthetic construct
AAGIGILTV

SEQ ID NO: 176 - DNA, 804 bp, synthetic construct
  source 1..804: mol_type = other DNA
atgttgcttgaacatttattaataatcttgtggatgcagctgacatgggtcagtggtcaacagctgaatcagagtcctcaatctatgtttatccaggaaggagaagatgtctccatgaactgcacttcttcaagcatatttaacacctggctatggtacaagcaggaccctggggaaggtcctgtcctcttgatagccttatataaggctggtgaattgacctcaaatggaagactgactgctcagtttggtataaccagaaaggacagcttcctgaatatctcagcatccatacctagtgatgtaggcatctacttctgtgctggtgggaccggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaggtggccgggtttaatctgctcatgacgctgcggctgtggtccagc

SEQ ID NO: 177 - DNA, 921 bp, synthetic construct
  source 1..921: mol_type = other DNA
atgggcacaaggttgttcttctatgtggccctttgtctcctgtggacaggacacatggatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgaggtaggggttgggcagccccagcattttggtgatgggactcgactctccatcctagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcatgtggctttacctcgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 178 - DNA, 822 bp, synthetic construct
  source 1..822: mol_type = other DNA
atgatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacttcggaggaggaaagcttatcttcggacagggaacggagttatctgtgaaacccaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 179 - DNA, 918 bp, synthetic construct
  source 1..918: mol_type = other DNA
atgagaatcaggctcctgtgctgtgtggccttttctctcctgtgggcaggtccagtgattgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagcctaagtttcggcactgaagctttctttggacaaggcaccagactcacagttgtagaggacctgaacaaggtgttcccacccgaggtcgctgtgtttgagccatcagaagcagagatctcccacacccaaaaggccacactggtgtgcctggccacaggcttcttccccgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcatgtggctttacctcgtcctaccagcaaggggtcctgtctgccaccatcctctatgagatcctgctagggaaggccaccctgtatgctgtgctggtcagcgcccttgtgttgatggccatggtcaagagaaaggatttc

SEQ ID NO: 180 - protein, 250 aa, synthetic construct
GQQLNQSPQSMFIQEGEDVSMNCTSSSIFNTWLWYKQDPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 181 - protein, 288 aa, synthetic construct
DAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 182 - protein, 251 aa, synthetic construct
QKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 183 - protein, 287 aa, synthetic construct
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRACGFTSSYQQGVLSATILYEILLGKATLYAVLVSALVLMAMVKRKDF

SEQ ID NO: 184 - protein, 9 aa, synthetic construct
FLWGPRALV

SEQ ID NO: 185 - protein, 9 aa, synthetic construct
KVAELVHFL

SEQ ID NO: 186 - DNA, 369 bp, synthetic construct
  source 1..369: mol_type = other DNA
atgggtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctttcgacacaaatgcttacaaagtcatcttt

SEQ ID NO: 187 - DNA, 363 bp, synthetic construct
  source 1..363: mol_type = other DNA
atgagagttaggctcatctctgctgtggtgctgtgttccctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagttcaacaaacacagaagtcttcttt

SEQ ID NO: 188 - DNA, 813 bp, synthetic construct
  source 1..813: mol_type = other DNA
atgggtcctgtcacctgctcagttcttgtgctcctcctaatgctcaggaggagcaatggcgatggagactccgtgacccagacagaaggcctggtcactctcacagaagggttgcctgtgatgctgaactgcacctatcagactatttactcaaatcctttccttttctggtatgtgcaacatctcaatgaatcccctcggctactcctgaagagcttcacagacaacaagaggaccgagcaccaagggttccacgccactctccataagagcagcagctccttccatctgcagaagtcctcagcgcagctgtcagactctgccctgtactactgtgctttcgacacaaatgcttacaaagtcatctttggaaaagggacacatcttcatgttctccctaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacaccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagttga

SEQ ID NO: 189 - DNA, 912 bp, synthetic construct
  source 1..912: mol_type = other DNA
atgagagttaggctcatctctgctgtggtgctgtgttccctaggaacaggccttgtggacatgaaagtaacccagatgccaagatacctgatcaaaagaatgggagagaatgttttgctggaatgtggacaggacatgagccatgaaacaatgtactggtatcgacaagaccctggtctggggctacagctgatttatatctcatacgatgttgatagtaacagcgaaggagacatccctaaaggatacagggtctcacggaagaagcgggagcatttctccctgattctggattctgctaaaacaaaccagacatctgtgtacttctgtgctagcagttcaacaaacacagaagtcttctttggtaaaggaaccagactcacagttgtagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgcaaccacttccgctgccaagtgcagttccatgggctttcagaggaggacaagtggccagagggctcacccaaacctgtcacacagaacatcagtgcagaggcctggggccgagcagactgtgggattacctcagcatcctatcaacaaggggtcttgtctgccaccatcctctatgagatcctgctagggaaagccaccctgtatgctgtgcttgtcagtacactggtggtgatggctatggtcaaaagaaagaactcgtga

SEQ ID NO: 190 - DNA, 371 bp, synthetic construct
  source 1..371: mol_type = other DNA
atggtcctagtgaccattctgctgctcagcgcgttcttctcactgagaggaaacagtgcccagtccgtggaccagcctgatgctcatgtcacgctctctgaaggagcctccctggagctcagatgcagttattcatacagtgcagcaccttacctcttctggtacgtgcagtatcctggccagagcctccagtttctcctcaaatacatcacaggagacaccgttgttaaaggcaccaagggctttgaggccgagtttaggaagagtaactcctctttcaacctgaagaaatccccagcccattggagcgactcagccaagtacttctgtgcactggagggcccggatacaggaaactacaaatacgtctt

SEQ ID NO: 191 - DNA, 375 bp, synthetic construct
  source 1..375: mol_type = other DNA
atgggcatccagaccctctgttgtgtgatcttttatgttctgatagcaaatcacacagatgctggagttacccagacacccagacatgaggtggcagagaaaggacaaacaataatcctgaagtgtgagccagtttcaggccacaatgaccttttctggtacagacagaccaagatacagggactagagttgctgagctacttccgcagcaagtctcttatggaagatggtggggctttcaaggatcgattcaaagctgagatgctaaattcatccttctccactctgaagattcaacctacagaacccagggactcagctgtgtatctgtgtgccagcagttttgggacagctagtgcagaaacgctgtatttt

SEQ ID NO: 192 - DNA, 816 bp, synthetic construct
  source 1..816: mol_type = other DNA
atggtcctagtgaccattctgctgctcagcgcgttcttctcactgagaggaaacagtgcccagtccgtggaccagcctgatgctcatgtcacgctctctgaaggagcctccctggagctcagatgcagttattcatacagtgcagcaccttacctcttctggtacgtgcagtatcctggccagagcctccagtttctcctcaaatacatcacaggagacaccgttgttaaaggcaccaagggctttgaggccgagtttaggaagagtaactcctctttcaacctgaagaaatccccagcccattggagcgactcagccaagtacttctgtgcactggagggcccggatacaggaaactacaaatacgtctttggagcaggtaccagactgaaggttatagcacacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaacttccaaaacctgtcagttatgggactccgaatcctcctgctgaaagtagccggatttaacctgctcatgacgctgaggctgtggtccagttga

SEQ ID NO: 193 - DNA, 924 bp, synthetic construct
  source 1..924: mol_type = other DNA
atgggcatccagaccctctgttgtgtgatcttttatgttctgatagcaaatcacacagatgctggagttacccagacacccagacatgaggtggcagagaaaggacaaacaataatcctgaagtgtgagccagtttcaggccacaatgaccttttctggtacagacagaccaagatacagggactagagttgctgagctacttccgcagcaagtctcttatggaagatggtggggctttcaaggatcgattcaaagctgagatgctaaattcatccttctccactctgaagattcaacctacagaacccagggactcagctgtgtatctgtgtgccagcagttttgggacagctagtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaggggcttcttcccctgacacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgtcaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctga

SEQ ID NO: 194 - protein, 123 aa, synthetic construct
MGPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCAFDTNAYKVIF

SEQ ID NO: 195 - protein, 120 aa, synthetic construct
MRVRLISAVVLCSLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSSTNTEVF

SEQ ID NO: 196 - protein, 271 aa, synthetic construct
MGPVTCSVLVLLLMLRRSNGDGDSVTQTEGLVTLTEGLPVMLNCTYQTIYSNPFLFWYVQHLNESPRLLLKSFTDNKRTEHQGFHATLHKSSSSFHLQKSSAQLSDSALYYCAFDTNAYKVIFGKGTHLHVLPNIQNPEPAVYQLKDPRSQDSTLCLFTDFDSQINVPKTMESGTFITDKTVLDMKAMDSKSNGAIAWSNQTSFTCQDIFKETNTTYPSSDVPCDATLTEKSFETDMNLNFQNLSVMGLRILLLKVAGFNLLMTLRLWSSL

SEQ ID NO: 197 - protein, 295 aa, synthetic construct
MRVRLISAVVLCSLGTGLVDMKVTQMPRYLIKRMGENVLLECGQDMSHETMYWYRQDPGLGLQLIYISYDVDSNSEGDIPKGYRVSRKKREHFSLILDSAKTNQTSVYFCASSSTNTEVFFGKGTRLTVVEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVM

SEQ ID NO: 198 - protein, 123 aa, synthetic construct
MVLVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGPDTGNYKYV

SEQ ID NO: 199 - protein, 124 aa, synthetic construct
MGIQTLCCVIFYVLIANHTDAGVTQTPRHEVAEKGQTIILKCEPVSGHNDLFWYRQTKIQGLELLSYFRSKSLMEDGGAFKDRFKAEMLNSSFSTLKIQPTEPRDSAVYLCASSFGTASAETLY

SEQ ID NO: 200 - protein, 271 aa, synthetic construct
MVLVTILLLSAFFSLRGNSAQSVDQPDAHVTLSEGASLELRCSYSYSAAPYLFWYVQYPGQSLQFLLKYITGDTVVKGTKGFEAEFRKSNSSFNLKKSPAHWSDSAKYFCALEGPDTGNYKYVFGAGTRLKVIAHIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 201 - protein, 306 aa, synthetic construct
MGIQTLCCVIFYVLIANHTDAGVTQTPRHEVAEKGQTIILKCEPVSGHNDLFWYRQTKIQGLELLSYFRSKSLMEDGGAFKDRFKAEMLNSSFSTLKIQPTEPRDSAVYLCASSFGTASAETLYFGSGTRLTVLEDLRNVTPPKVSLFEPSKAEIANKQKATLVCLARGFFPHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 202 - protein, 10 aa, synthetic construct
GVYDGREHTV

SEQ ID NO: 203 - protein, 9 aa, synthetic construct
FMNKFIYEI

SEQ ID NO: 204 - DNA, 846 bp, synthetic construct
  source 1..846: mol_type = other DNA
atgaagaagcacctgaccacctttctcgtgatcctgtggctgtacttctaccggggcaacggcaagaaccaggtggaacagagcccccagagcctgatcatcctggaaggcaagaactgcaccctgcagtgcaactacaccgtgtcccccttcagcaacctgcggtggtacaagcaggacaccggcagaggccctgtgtccctgaccatcctgaccttcagcgagaacaccaagagcaacggccggtacaccgccaccctggacgccgatacaaagcagagcagcctgcacatcaccgccagccagctgagcgatagcgccagctacatctgcgtggtgtccggcggcacagacagctggggcaagctgcagtttggcgccggaacacaggtggtcgtgacccccgacatccagaaccctgaccctgccgtgtaccagctgcgggacagcaagagcagcgacaagagcgtgtgcctgttcaccgacttcgacagccagaccaacgtgtcccagagcaaggacagcgacgtgtacatcaccgacaagaccgtgctggacatgcggagcatggacttcaagagcaatagcgccgtggcctggtccaacaagagcgacttcgcctgcgccaacgccttcaacaacagcattatccccgaggacacattcttcccaagccccgagagcagctgcgacgtcaagctggtggaaaagagcttcgagacagacaccaacctgaacttccagaacctgagcgtgatcggcttcagaatcctgctgctgaaggtggccggcttcaacctgctgatgaccctgagactgtggtccagcggcagccgggccaagaga

SEQ ID NO: 205 - DNA, 933 bp, synthetic construct
  source 1..933: mol_type = other DNA
atggccagcctgctgttcttctgcggcgccttctacctgctgggcaccggctctatggatgccgacgtgacccagaccccccggaacagaatcaccaagaccggcaagcggatcatgctggaatgctcccagaccaagggccacgaccggatgtactggtacagacaggaccctggcctgggcctgcggctgatctactacagcttcgacgtgaaggacatcaacaagggcgagatcagcgacggctacagcgtgtccagacaggctcaggccaagttcagcctgtccctggaaagcgccatccccaaccagaccgccctgtacttttgtgccacaagcggccagggcgcctacgaggagcagttctttggccctggcacccggctgacagtgctggaagatctgaagaacgtgttccccccagaggtggccgtgttcgagccttctgaggccgaaatcagccacacccagaaagccacactcgtgtgtctggccaccggcttctaccccgaccacgtggaactgtcttggtgggtcaacggcaaagaggtgcacagcggcgtgtccaccgatccccagcctctgaaagaacagcccgccctgaacgacagccggtactgcctgagcagcagactgagagtgtccgccaccttctggcagaaccccagaaaccacttcagatgccaggtgcagttttacggcctgagcgagaacgacgagtggacccaggacagagccaagcccgtgacacagatcgtgtctgccgaagcttgggggcgcgccgattgtggctttaccagcgagagctaccagcagggcgtgctgagcgccaccatcctgtacgagatcctgctgggaaaggccacactgtacgccgtgctggtgtctgccctggtgctgatggccatggtcaagcggaaggacagccggggc

SEQ ID NO: 206 - protein, 282 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPDIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSSGSRAKR

SEQ ID NO: 207 - protein, 136 aa, synthetic construct
MKKHLTTFLVILWLYFYRGNGKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQFGAGTQVVVTPD

SEQ ID NO: 208 - protein, 311 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 209 - protein, 133 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFFGPGTRLTVLE

SEQ ID NO: 210 - protein, 105 aa, synthetic construct
MKNQVEQSPQSLIILEGKNCTLQCNYTVSPFSNLRWYKQDTGRGPVSLTIMTFSENTKSNGRYTATLDADTKQSSLHITASQLSDSASYICVVSGGTDSWGKLQF

SEQ ID NO: 211 - protein, 105 aa, synthetic construct
MKNQVEQSPQSLIILEGKNCTLQCNYTVSPFSNLRWYKQDTGRGPVSLTIVTFSENTKSNGRYTATLDADTKQSSLHITASQLSDSASYICVVSGGTDSWGKLQF

SEQ ID NO: 212 - protein, 105 aa, synthetic construct
MKNQVEQSPQSLIILEGKNCTLQCNYTVSPFSNLRWYKQDTGRGPVSLTILTFSENTKSNGRYTATLDADTKQSSLHITASQLSDSASYICVVSGGTDSWGKLQF

SEQ ID NO: 213 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYNEQFF

SEQ ID NO: 214 - protein, 123 aa, synthetic construct
MASLLFFCGAFYLLGTGSMDADVTQTPRNRITKTGKRIMLECSQTKGHDRMYWYRQDPGLGLRLIYYSFDVKDINKGEISDGYSVSRQAQAKFSLSLESAIPNQTALYFCATSGQGAYEEQFF

SEQ ID NO: 215 - protein, 9 aa, synthetic construct
VLDFAPPGA

SEQ ID NO: 216 - protein, 9 aa, synthetic construct
RMFPNAPYL

SEQ ID NO: 217 - DNA, 396 bp, synthetic construct
  source 1..396: mol_type = other DNA
atggagacactgctgggactactgattctgtggctgcaactgcaatgggtgagcagcaaacaggaggttacccagattcctgctgctctgtctgttcctgaaggcgagaatctggtgctgaactgcagcttcacagatagcgccatctacaacctgcagtggttcagacaggatcctggaaaaggcctgacaagcctgctgctgattcagagctctcagagagagcagacatctggaagactgaatgctagcctggacaagtctagcggcagaagcaccctgtatattgccgcctctcaacctggagattctgccacatacctgtgtgctgtgaaggagacatctggctctagactgacctttggcgagggaacacaactgaccgtgaatcctgac

SEQ ID NO: 218 - DNA, 417 bp, synthetic construct
  source 1..417: mol_type = other DNA
atgaccagagttagcctgttatgggctgtggtggtgagcacatgtctggaatctggaatggcccagacagtgacacagtctcagcctgaaatgtctgtgcaggaagccgaaaccgttacactgagctgcacctacgatacaagcgagaacaactactacctgttctggtacaagcagcccccctctaggcagatgatcctggtgatcagacaggaggcctataaacagcagaatgccacagagaaccggttcagcgtgaacttccagaaagccgccaagagcttcagcctgaagatctctgattctcagctgggcgatacagccatgtacttttgcgccttcatctaccccagctacacaagcggcacatacaagtacatcttcggcaccggcacaagactgaaggttctggccaac

SEQ ID NO: 219 - DNA, 420 bp, synthetic construct
  source 1..420: mol_type = other DNA
atggccatgttactaggagcgagcgtgctgattctgtggttacagcctgattgggtgaactctcagcagaagaacgatgatcagcaggtgaagcagaacagcccctctctgtctgtgcaggaaggcagaatcagcatcctgaattgcgattacaccaacagcatgttcgactacttcctgtggtacaagaagtaccccgccgagggccctacctttctgatcagcatctctagcatcaaggacaagaacgaagatggcagattcaccgtgttcctgaacaagagcgccaagcacctgagcctgcacattgtgccttctcaacctggagattctgccgtgtacttttgtgctgcctctggaacaggcggaagctatatccccacatttggaagaggaacaagcctgatcgtgcacccttac

SEQ ID NO: 220 - DNA, 417 bp, synthetic construct
  source 1..417: mol_type = other DNA
atggccatgttactaggagcgagcgtgctgattctgtggttacagcctgattgggtgaactctcagcagaagaacgatgatcagcaggtgaagcagaacagcccctctctgtctgtgcaggaaggcagaatcagcatcctgaattgcgattacaccaacagcatgttcgactacttcctgtggtacaagaagtaccccgccgagggccctacctttctgatcagcatctctagcatcaaggacaagaacgaagatggcagattcaccgtgttcctgaacaagagcgccaagcacctgagcctgcacattgtgccttctcaacctggagattctgccgtgtacttttgtgctgcctctggcattggcgactacaaactgagctttggagccggcacaacagtgaccgttagagccaat

SEQ ID NO: 221 - DNA, 393 bp, synthetic construct
  source 1..393: mol_type = other DNA
atggtgaagatccggcagttcctcctggctattctgtggctgcaactgtcttgtgtgtctgctgccaagaatgaagtggagcagtctccccagaaccttacagcccaggaaggcgagtttatcaccatcaactgcagctattctgtgggcattagcgccctgcattggctgcagcaacaccctggaggaggaattgtgtctctgtttatgctgtcttctggcaagaagaagcacggccggctgattgccaccatcaacatccaggagaagcactcttctctgcacattacagcctctcatcccagggattctgccgtgtacatctgtgccgtgagaaccagctacgataaggtgattttcggaccaggcacctctctgagcgtgatccccaat

SEQ ID NO: 222 - DNA, 408 bp, synthetic construct
  source 1..408: mol_type = other DNA
atgaagagcctgagagtcctgctggtgattttgtggctgcagctgtcttgggtttggtctcagcagaaagaagtggagcagaatagcggccctctgtctgttcctgaaggcgctattgctagcctgaattgcacatacagcgatagaggatctcagagcttcttctggtaccggcagtacagcggcaagagcccagaactgatcatgttcatctacagcaatggcgacaaggaggatggcaggtttacagcccagctgaacaaggccagccagtatgtttctctgctgatcagagatagccagcctagcgattctgccacctacctgtgtgccgtgaacttacttggagctacaggatactctacactgaccttcggcaaaggcaccatgctgctggtgagccctgat

SEQ ID NO: 223 - DNA, 384 bp, synthetic construct
  source 1..384: mol_type = other DNA
atgtggggcgttttccttctgtatgtgagcatgaagatgggcggcacaacaggccagaacatcgatcagcctaccgagatgacagccacagaaggagctattgttcagatcaactgcacctaccagacaagcggcttcaacggcctgttctggtaccagcagcatgctggagaagctcctacatttctgagctacaatgtgctggatggcctggaggagaaaggcaggtttagcagcttcctgagcaggtctaagggctattcttatctgctgctgaaggagctgcagatgaaggattccgccagctacctgtgtgccgttaggggcatcaatgattacaagctgagctttggagccggaacaacagtgaccgtgagagccaac

SEQ ID NO: 224 - DNA, 393 bp, synthetic construct
  source 1..393: mol_type = other DNA
atggagaagatgctggagtgtgcgttcatcgttctgtggctgcaacttggatggctgtctggagaggatcaggttacacagtctcctgaagccctgagactgcaagaaggagaaagctctagcctgaactgcagctacacagtgtctggactgagaggcctgttctggtacagacaggatcctggaaaaggcccagagttcctgtttaccctgtattctgccggcgaggagaaggagaaagagagactgaaagctaccctgaccaagaaggagagcttcctgcacattaccgcccccaaacctgaggattctgccacatatctgtgtgccgtgattaccggctttcagaagctggtgtttggcacaggcaccagactgctggtttctcccaat

SEQ ID NO: 225 - DNA, 384 bp, synthetic construct
  source 1..384: mol_type = other DNA
atgagactggtggcacgcgtaactgtgtttctgacctttggcaccatcatcgatgccaagacaacccagcctacaagcatggactgtgccgagggaagagctgctaatctgccatgtaatcacagcacaatcagcggcaacgagtacgtgtactggtaccggcagatccactctcaaggacctcagtacatcattcatggcctgaagaacaacgagaccaacgagatggccagcctgatcatcaccgaggacaggaagtcttctaccctgattctgcctcatgctacactgagagataccgccgtgtactactgcattgccggagtgggaagaggccagaatttcgtgtttggacctggaacaagactgagcgttctgccctat

SEQ ID NO: 226 - DNA, 408 bp, synthetic construct
  source 1..408: mol_type = other DNA
atggagaagaaccccttggcagcacctctgcttattctgtggttccacctggattgtgtgagcagcatcctgaatgtggagcagtctcctcagagcctgcatgtgcaagaaggcgatagcaccaatttcacctgcagctttccaagcagcaacttctacgccctgcactggtacagatgggaaaccgccaaatctcctgaagccctgtttgtgatgaccctgaatggcgacgagaagaagaagggcagaattagcgccaccctgaataccaaggagggctacagctacctgtacatcaagggctctcaacctgaggattctgccacctacctttgcgcctttcaccccaatttcggcaacgagaaactgacctttggaaccggaacaaggctgaccatcatccccaac

SEQ ID NO: 227 - DNA, 411 bp, synthetic construct
  source 1..411: mol_type = other DNA
atggagaagatgctggagtgtgcgttcatcgttctgtggctgcaacttggatggctgtctggagaggatcaggttacacagtctcctgaagccctgagactgcaagaaggagaaagctctagcctgaactgcagctacacagtgtctggactgagaggcctgttctggtacagacaggatcctggaaaaggcccagagttcctgtttaccctgtattctgccggcgaggagaaggagaaagagagactgaaagctaccctgaccaagaaggagagcttcctgcacattaccgcccccaaacctgaggattctgccacatatctgtgtgctgttcagcctagaggagatggctctagcaataccggcaagctgatctttggccagggaacaacactgcaggtgaagcctgat

SEQ ID NO: 228 - DNA, 423 bp, synthetic construct
  source 1..423: mol_type = other DNA
atccagaatcccgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 229 - DNA, 440 bp, synthetic construct
  source 1..440: mol_type = other DNA
ctcaataaaagagcccacaacccctcactcggcgcgccaccatgggcacatctcttctctgttgggtggttctgggctttctgggcacagatcatacaggagctggagttagccagtctcctaggtataaggtgaccaagaggggacaggatgtggctctgagatgtgaccctattagcggacatgtgagcctgtactggtacagacaagctctgggacaaggacccgagtttctgacctacttcaactatgaggcccagcaggacaaatctggactgcccaacgacagattcagcgccgaaagaccagaaggctctattagcacactgaccatccagagaacagagcagagggattctgccatgtacagatgcgccagcagcttaacaggctcttacgagcagtactttggacctggcacaagactgacagtgacagag

SEQ ID NO: 230 - DNA, 440 bp, synthetic construct
  source 1..440: mol_type = other DNA
ctcaataaaagagcccacaacccctcactcggcgcgccaccatgctgcttcttctcctccttctcggacctgctggatctggattaggagctgttgtgtctcagcacccttcttgggtgatctgtaaaagcggcacaagcgtgaagatcgagtgcagaagcctggactttcaggccacaaccatgttctggtataggcagttccccaagcagtctctgatgctgatggccacctctaatgagggctctaaggccacatatgaacagggagtggagaaggacaagttcctgatcaaccacgcctctctgaccctgtctaccctgacagttacatctgcccaccctgaggatagcagcttttacatctgtagcgccacacctgaagcctctagcccatatgagcagtactttggccctggcaccagattaacagtgacagag

SEQ ID NO: 231 - DNA, 440 bp, synthetic construct
  source 1..440: mol_type = other DNA
ctcaataaaagagcccacaacccctcactcggcgcgccaccatgggacctggactgcttcattggatggctctgtgtttgctgggaacaggacatggagatgctatggtgatccagaaccccaggtatcaggtgacccagtttggcaaaccagtgacactgagctgttctcagaccctgaaccacaacgtgatgtactggtaccagcagaagtcttctcaggcccctaagctgctgttccactactacgacaaggacttcaacaacgaggccgatacccctgacaatttccagagcaggaggcccaataccagcttctgtttcctggacattagaagccctggactgggagatgctgccatgtacctgtgtgccaccagcaatttacagggaagacaacctcagcactttggcgatggcacaaggctgtctatcctggag

SEQ ID NO: 232 - DNA, 470 bp, synthetic construct
  source 1..470: mol_type = other DNA
ctcaataaaagagcccacaacccctcactcggcgcgccaccatgctgagccctgatctccctgattctgcctggaataccagactgctgtgtcatgtgatgctgtgtctgcttggagccgtttctgtggctgctggcgtgattcaatctcctagacacctgatcaaggagaagagagaaacagccaccctgaagtgctaccccatccccagacacgatacagtgtactggtatcagcaaggacctggacaagatccccagttcctgatcagcttctacgagaagatgcagagcgacaaaggcagcatcccagacagatttagcgcccagcagtttagcgactatcactctgagctgaacatgagcagcctggaactgggcgattctgctctgtacttctgtgcctcttctctgagactgggaagagaaacccagtactttggacccggcacaagactgctggttcttgag

SEQ ID NO: 233 - DNA, 440 bp, synthetic construct
  source 1..440: mol_type = other DNA
ctcaataaaagagcccacaacccctcactcggcgcgccaccatgggcacaagacttctctgctgggtggtgcttggatttctgggcacagatcatacaggagctggagttagccagtctcctaggtacaaagtggccaagagaggacaggatgtggctctgagatgtgaccctattagcggacatgtgagcctgttttggtaccagcaagctctgggacaaggacccgagtttctgacctacttccagaatgaagcccagctggataaatctggactgcctagcgaccggttcttcgccgaaagacctgaaggatctgttagcaccctgaagattcagagaacacagcaggaggactctgccgtgtacctgtgtgcctcttctttaggacaggcctatgagcagtattttggacctggcaccagactgaccgtgacagag

SEQ ID NO: 234 - DNA, 440 bp, synthetic construct
  source 1..440: mol_type = other DNA
ctcaataaaagagcccacaacccctcactcggcgcgccaccatgggcacaagacttctctgctgggtggccttttgtctgctggtggaagagctgattgaagctggagttgtgcagtctcctaggtacaagatcatcgagaagaagcagcccgtggccttctggtgtaatcccatttctggccacaacaccctgtactggtatctgcagaatctgggacagggccctgaactgctgatcagatacgagaacgaagaagccgtggacgattctcaactgcctaaggaccgcttttctgccgagaggctgaaaggagtggattctaccctgaagatccaacctgctgaactgggcgattctgctgtgtacctgtgcgcttctagcctgacaagaggagctgaagccttttttggacagggcacaagactgacagtggtggag

SEQ ID NO: 235 - DNA, 440 bp, synthetic construct
  source 1..440: mol_type = other DNA
ctcaataaaagagcccacaacccctcactcggcgcgccaccatgggacctcagcttcttggatacgttgtgctgtgtctgcttggagctggacctcttgaagctcaggttacccagaaccccagatacctgattaccgtgacaggcaaaaagctgaccgtgacatgtagccagaacatgaaccacgagtacatgagctggtaccggcaggatcctggattaggcctgagacagatctactacagcatgaacgtggaggtgaccgataaaggcgacgtgcctgagggatacaaggtgagcagaaaggagaagaggaatttccccctgatcctggaaagcccaagccccaatcagacaagcctgtacttttgtgccagcagcttttctggcggcacatatgagcagtacttcggccctggcacaagactgacagttacagag

SEQ ID NO: 236 - DNA, 473 bp, synthetic construct
  source 1..473: mol_type = other DNA
ctcaataaaagagcccacaacccctcactcggcgcgccaccatgctgagccctgatctccctgattctgcctggaataccagactgctgtgtcatgtgatgctgtgtctgcttggagccgtttctgtggctgctggcgtgattcaatctcctagacacctgatcaaggagaagagagaaacagccaccctgaagtgctaccccatccccagacacgatacagtgtactggtatcagcaaggacctggacaagatccccagttcctgatcagcttctacgagaagatgcagagcgacaaaggcagcatcccagacagatttagcgcccagcagtttagcgactatcactctgagctgaacatgagcagcctggaactgggcgattctgctctgtacttctgtgccagcagctatagaggaggcagcacatatgagcagtactttggccctggcacaagactgacagtgacagag

SEQ ID NO: 237 - DNA, 446 bp, synthetic construct
  source 1..446: mol_type = other DNA
ctcaataaaagagcccacaacccctcactcggcgcgccaccatgagcaccagactcctttgctggatggctttgtgtctgcttggagctgagctgtctgaagctgaagttgcccagtctcccagatacaagatcaccgagaaatctcaggctgtggccttctggtgtgaccctatttctggacacgccaccctgtactggtataggcaaattctgggacaaggccctgaactgctggtgcaatttcaggatgagagcgtggtggacgattctcaactgcctaaggacaggttttctgccgagcggctgaaaggagttgatagcaccctgaagatccaacctgctgaactgggcgattctgctatgtacctgtgcgcctcttctcagagagatagccctaacgagaagctgttctttggctctggaacccagctgtctgtgctggag

SEQ ID NO: 238 - DNA, 449 bp, synthetic construct
  source 1..449: mol_type = other DNA
ctcaataaaagagcccacaacccctcactcggcgcgccaccatgggctgtagactgttgtgttgtgctgtgctgtgtctgttgggagctgtgcctatggaaacaggcgttacccagacacctagacatctggttatgggcatgaccaacaagaagagcctgaagtgcgagcagcatctgggccataacgccatgtactggtataagcagagcgccaagaaaccactggaactgatgttcgtgtacagcctggaggagagggtggagaataatagcgtgcccagcagatttagccctgagtgcccaaattcttctcacctgttcctgcacctgcacacattacagcccgaggattctgccctgtacctgtgtgcttcttctcaagacccttacaagctgagcggcaataccatctacttcggcgaaggctcttggctgacagtggttgaa

SEQ ID NO: 239 - DNA, 528 bp, synthetic construct
  source 1..528: mol_type = other DNA
gatctgaacaaggtgttccccccagaggtggccgtgttcgagccttctgaggccgagatctcccacacccagaaagccaccctcgtgtgcctggccaccggctttttccccgaccacgtggaactgtcttggtgggtcaacggcaaagaggtgcactccggcgtgtgcaccgatccccagcctctgaaagaacagcccgccctgaacgacagccggtactgcctgagcagcagactgagagtgtccgccaccttctggcagaacccccggaaccacttcagatgccaggtgcagttctacggcctgagcgagaacgacgagtggacccaggacagagccaagcccgtgacacagatcgtgtctgccgaagcctggggcagagccgattgcggctttacctccgtgtcctatcagcagggcgtgctgagcgccacaatcctgtacgagatcctgctgggcaaggccaccctgtacgccgtgctggtgtctgccctggtgctgatggccatggtcaagcggaaggacttc

SEQ ID NO: 240 - DNA, 534 bp, synthetic construct
  source 1..534: mol_type = other DNA
gacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 241 - DNA, 819 bp, synthetic construct
  source 1..819: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgtgaacataggaaaccatgacatgcgctttggagcagggaccagactgacagtaaaaccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 242 - DNA, 819 bp, synthetic construct
  source 1..819: mol_type = other DNA
atggagaaaatgttggagtgtgcattcatagtcttgtggcttcagcttggctggttgagtggagaagaccaggtgacgcagagtcccgaggccctgagactccaggagggagagagtagcagtctcaactgcagttacacagtcagcggtttaagagggctgttctggtataggcaagatcctgggaaaggccctgaattcctcttcaccctgtattcagctggggaagaaaaggagaaagaaaggctaaaagccacattaacaaagaaggaaagctttctgcacatcacagcccctaaacctgaagactcagccacttatctctgtgctgtgcagaccatggacggtaaccagttctattttgggacagggacaagtttgacggtcattccaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 243 - DNA, 825 bp, synthetic construct
  source 1..825: mol_type = other DNA
atggcatgccctggcttcctgtgggcacttgtgatctccacctgtcttgaatttagcatggctcagacagtcactcagtctcaaccagagatgtctgtgcaggaggcagagaccgtgaccctgagctgcacatatgacaccagtgagagtgattattatttattctggtacaagcagcctcccagcaggcagatgattctcgttattcgccaagaagcttataagcaacagaatgcaacagagaatcgtttctctgtgaacttccagaaagcagccaaatccttcagtctcaagatctcagactcacagctgggggatgccgcgatgtatttctgtgcttccagtccaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 244 - DNA, 834 bp, synthetic construct
  source 1..834: mol_type = other DNA
atgacacgagttagcttgctgtgggcagtcgtggtctccacctgtcttgaatccggcatggcccagacagtcactcagtctcaaccagagatgtctgtgcaggaggcagagactgtgaccctgagttgcacatatgacaccagtgagagtaattattatttgttctggtacaaacagcctcccagcaggcagatgattctcgttattcgccaagaagcttataagcaacagaatgcaacggagaatcgtttctctgtgaacttccagaaagcagccaaatccttcagtctcaagatctcagactcacagctgggggacactgcgatgtatttctgtgctttcaacccttgggagaactatggtcagaattttgtctttggtcccggaaccagattgtccgtgctgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 245 - DNA, 819 bp, synthetic construct
  source 1..819: mol_type = other DNA
atgaagagcctgagagtcctgctggtgattttgtggctgcagctgtcttgggtttggtctcagcagaaagaagtggagcagaatagcggccctctgtctgttcctgaaggcgctattgctagcctgaattgcacatacagcgatagaggatctcagagcttcttctggtaccggcagtacagcggcaagagcccagaactgatcatgttcatctacagcaatggcgacaaggaggatggcaggtttacagcccagctgaacaaggccagccagtatgtttctctgctgatcagagatagccagcctagcgattctgccacctacctgtgtgccgtgaacatcggaaatcacgacatgagatttggagccggcacaagactgaccgtgaagcccaatatccagaaccctgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 246 - DNA, 819 bp, synthetic construct
  source 1..819: mol_type = other DNA
atggagaagatgctggagtgtgcgttcatcgttctgtggctgcaacttggatggctgtctggagaggatcaggttacacagtctcctgaagccctgagactgcaagaaggagaaagctctagcctgaactgcagctacacagtgtctggactgagaggcctgttctggtacagacaggatcctggaaaaggcccagagttcctgtttaccctgtattctgccggcgaggagaaggagaaagagagactgaaagctaccctgaccaagaaggagagcttcctgcacattaccgcccccaaacctgaggattctgccacatatctgtgtgctgtgcagaccatggatggcaaccagttctacttcggcacaggcacatctctgaccgttatccccaatatccagaaccctgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 247 - DNA, 825 bp, synthetic construct
  source 1..825: mol_type = other DNA
atggcttgtcctggattcttatgggctctggtgatcagcacctgtctggagttctctatggcccagacagtgacacagtctcagcctgaaatgtctgtgcaggaagccgaaaccgtgacactgtcttgcacctacgatacaagcgagagcgactactacctgttctggtacaagcagcctccctctaggcagatgatcctggtgattagacaggaggcctacaaacagcagaatgccaccgagaaccggtttagcgtgaacttccagaaagccgccaagagcttcagcctgaaaatctctgacagccagctgggagatgctgccatgtacttttgtgccagctctccaggcacctacaagtacatttttggcaccggcaccagactgaaggtgctggccaatatccagaatcccgatcctgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 248 - DNA, 834 bp, synthetic construct
  source 1..834: mol_type = other DNA
atgaccagagttagcctgttatgggctgtggtggtgagcacatgtctggaatctggaatggcccagacagtgacacagtctcagcctgaaatgtctgtgcaggaagccgaaaccgttacactgagctgcacctacgatacaagcgagagcaactactacctgttctggtacaagcagcccccttctaggcagatgatcctggtgatcagacaggaggcctataaacagcagaatgccaccgagaaccggtttagcgtgaacttccagaaagccgccaagagcttcagcctgaaaatctctgacagccagctgggcgatacagccatgtacttttgtgccttcaacccctgggagaactatggccagaatttcgtgttcggccctggcaccagactgtctgttctgccttatatccagaaccccgatcctgctgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcattatccccgaggacacattcttcccaagccccgagagcagctgcgacgtgaagctggtggaaaagagcttcgagacagacaccaacctgaacttccagaacctcagcgtgatcggcttccggatcctgctgctgaaggtggccggcttcaacctgctgatgaccctgcggctgtggtccagctga

SEQ ID NO: 249 - DNA, 939 bp, synthetic construct
  source 1..939: mol_type = other DNA
atgggctgcaggctgctctgctgtgcggttctctgtctcctgggagcagttcccatagacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaagggactagcggggcagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 250 - DNA, 942 bp, synthetic construct
  source 1..942: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactctctttgggaccttcaagagacccagtacttcgggccaggcacgcggctcctggtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 251 - DNA, 945 bp, synthetic construct
  source 1..945: mol_type = other DNA
atgggcaccagcctcctctgctggatggccctgtgtctcctgggggcagatcacgcagatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttttcagacgggggggctacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 252 - DNA, 933 bp, synthetic construct
  source 1..933: mol_type = other DNA
atgctgctgcttctgctgcttctggggccagcaggctccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaccccattctctcacagatacgcagtattttggcccaggcacccggctgacagtgctcgaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtcaagagaaaggattccagaggctag

SEQ ID NO: 253 - DNA, 936 bp, synthetic construct
  source 1..936: mol_type = other DNA
atgggctgtagactgttgtgttgtgctgtgctgtgtctgttgggagctgtgcctatcgatacagaggtgacccagacccctaaacatctggttatgggcatgaccaacaagaagagcctgaagtgcgagcagcacatgggccatagggccatgtattggtataagcagaaggccaagaaacctcctgagctgatgttcgtgtacagctacgagaagctgagcatcaacgagagcgtgcccagcagattttctcctgagtgccctaattctagcctgctgaatctgcacctgcatgctctgcagcctgaggattctgctctgtacctgtgtgcttcttctcagggcacatctggagctgatacacagtacttcggacctggcacaagactgacagtgctggaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 254 - DNA, 939 bp, synthetic construct
  source 1..939: mol_type = other DNA
atgtctatcggtctgctgtgctgtgctgctctttctctgctttgggctggacctgtgaatgctggagttacacaaacccccaagttccaagtgctgaagacaggacagagcatgaccctgcagtgtgctcaggacatgaatcacgagtacatgagctggtacagacaggatcctggaatgggcctgaggctgatccactactctgttggagccggaattacagatcagggagaagtgccaaatggctacaacgtgagcaggagcacaaccgaggacttccccttaagactgttgtctgctgctccatctcagacaagcgtgtacttttgcgccagctcctactctctgtgggatctgcaggaaacccagtactttggaccaggcacaagactgttagtgctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 255 - DNA, 942 bp, synthetic construct
  source 1..942: mol_type = other DNA
atgggcacatctcttctctgctggatggctctttgtctgcttggagccgatcatgccgatacaggagttagccaggatcctagacacaagatcaccaagagaggccagaatgtgaccttccggtgcgatcctatctctgagcacaacaggctgtactggtacagacaaacactgggacaaggacctgagttcctgacctacttccagaacgaagcccagctggagaagtctagacttctgagcgacagatttagcgccgagagacctaaaggcagctttagcaccctggagatccagagaacagaacagggcgattctgccatgtacctgtgtgctagcagcttttctgatggaggcgccaccgatacacagtatttcggacctggcacaagactgacagtgctggaggacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 256 - DNA, 930 bp, synthetic construct
  source 1..930: mol_type = other DNA
atgctgcttcttctcctccttctcggacctgctggatctggattaggagctgttgtgtctcagcacccttcttgggtgatctgtaaaagcggcacaagcgtgaagatcgagtgcagaagcctggactttcaggccacaaccatgttctggtataggcagttccccaagcagtctctgatgctgatggccacctctaatgagggctctaaggccacatatgaacagggagtggagaaggacaagttcctgatcaaccacgcctctctgaccctgtctaccctgacagttacatctgcccaccctgaggatagcagcttttacatctgtagcgccagacctcacagcctgaccgatacacagtactttggccctggcacaagactgacagtgttagaagacctgaagaacgtgttccccccagaggtggccgtgttcgagcctagcgaggccgagatcagccacacccagaaagccaccctcgtgtgcctggccaccggcttttaccccgaccacgtggaactgtcttggtgggtcaacggcaaagaggtgcacagcggcgtctgcaccgacccccagcccctgaaagagcagcccgccctgaacgacagccggtactgtctgagcagcagactgagagtgtccgccaccttctggcagaacccccggaaccacttcagatgccaggtgcagttctacggcctgagcgagaacgacgagtggacccaggaccgggccaagcccgtgacccagatcgtgtctgctgaggcctggggcagagccgattgcggcttcaccagcgagagctaccagcagggcgtgctgagcgccaccatcctgtacgagatcctgctgggcaaggccaccctgtacgccgtgctggtgtccgccctggtgctgatggccatggtcaagcggaaggacagccggggc

SEQ ID NO: 257 - protein, 131 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVKETSGSRLTFGEGTQLTVNP

SEQ ID NO: 258 - protein, 139 aa, synthetic construct
MTRVSLLWAVVVSTCLESGMAQTVTQSQPEMSVQEAETVTLSCTYDTSENNYYLFWYKQPPSRQMILVIRQEAYKQQNATENRFSVNFQKAAKSFSLKISDSQLGDTAMYFCAFIYPSYTSGTYKYIFGTGTRLKVLAN

SEQ ID NO: 259 - protein, 140 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGTGGSYIPTFGRGTSLIVHPY

SEQ ID NO: 260 - protein, 139 aa, synthetic construct
MAMLLGASVLILWLQPDWVNSQQKNDDQQVKQNSPSLSVQEGRISILNCDYTNSMFDYFLWYKKYPAEGPTFLISISSIKDKNEDGRFTVFLNKSAKHLSLHIVPSQPGDSAVYFCAASGIGDYKLSFGAGTTVTVRAN

SEQ ID NO: 261 - protein, 131 aa, synthetic construct
MVKIRQFLLAILWLQLSCVSAAKNEVEQSPQNLTAQEGEFITINCSYSVGISALHWLQQHPGGGIVSLFMLSSGKKKHGRLIATINIQEKHSSLHITASHPRDSAVYICAVRTSYDKVIFGPGTSLSVIPN

SEQ ID NO: 262 - protein, 135 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNLLGATGYSTLTFGKGTMLLVSP

SEQ ID NO: 263 - protein, 128 aa, synthetic construct
MWGVFLLYVSMKMGGTTGQNIDQPTEMTATEGAIVQINCTYQTSGFNGLFWYQQHAGEAPTFLSYNVLDGLEEKGRFSSFLSRSKGYSYLLLKELQMKDSASYLCAVRGINDYKLSFGAGTTVTVRAN

SEQ ID NO: 264 - protein, 131 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVITGFQKLVFGTGTRLLVSPN

SEQ ID NO: 265 - protein, 128 aa, synthetic construct
MRLVARVTVFLTFGTIIDAKTTQPTSMDCAEGRAANLPCNHSTISGNEYVYWYRQIHSQGPQYIIHGLKNNETNEMASLIITEDRKSSTLILPHATLRDTAVYYCIAGVGRGQNFVFGPGTRLSVLPY

SEQ ID NO: 266 - protein, 136 aa, synthetic construct
MEKNPLAAPLLILWFHLDCVSSILNVEQSPQSLHVQEGDSTNFTCSFPSSNFYALHWYRWETAKSPEALFVMTLNGDEKKKGRISATLNTKEGYSYLYIKGSQPEDSATYLCAFHPNFGNEKLTFGTGTRLTIIPN

SEQ ID NO: 267 - protein, 136 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQPRGDGSSNTGKLIFGQGTTLQVKP

SEQ ID NO: 268 - protein, 140 aa, synthetic construct
IQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 269 - protein, 133 aa, synthetic construct
MGTSLLCWVVLGFLGTDHTGAGVSQSPRYKVTKRGQDVALRCDPISGHVSLYWYRQALGQGPEFLTYFNYEAQQDKSGLPNDRFSAERPEGSISTLTIQRTEQRDSAMYRCASSLTGSYEQYFGPGTRLTVTE

SEQ ID NO: 270 - protein, 133 aa, synthetic construct
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSATPEASSPYEQYFGPGTRLTVTE

SEQ ID NO: 271 - protein, 133 aa, synthetic construct
MGPGLLHWMALCLLGTGHGDAMVIQNPRYQVTQFGKPVTLSCSQTLNHNVMYWYQQKSSQAPKLLFHYYDKDFNNEADTPDNFQSRRPNTSFCFLDIRSPGLGDAAMYLCATSNLQGRQPQHFGDGTRLSILE

SEQ ID NO: 272 - protein, 143 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSLRLGRETQYFGPGTRLLVLE

SEQ ID NO: 273 - protein, 133 aa, synthetic construct
MGTRLLCWVVLGFLGTDHTGAGVSQSPRYKVAKRGQDVALRCDPISGHVSLFWYQQALGQGPEFLTYFQNEAQLDKSGLPSDRFFAERPEGSVSTLKIQRTQQEDSAVYLCASSLGQAYEQYFGPGTRLTVTE

SEQ ID NO: 274 - protein, 133 aa, synthetic construct
MGTRLLCWVAFCLLVEELIEAGVVQSPRYKIIEKKQPVAFWCNPISGHNTLYWYLQNLGQGPELLIRYENEEAVDDSQLPKDRFSAERLKGVDSTLKIQPAELGDSAVYLCASSLTRGAEAFFGQGTRLTVVE

SEQ ID NO: 275 - protein, 134 aa, synthetic construct
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSRDREQESPLHFGNGTRLTVTE

SEQ ID NO: 276 - protein, 133 aa, synthetic construct
MGPQLLGYVVLCLLGAGPLEAQVTQNPRYLITVTGKKLTVTCSQNMNHEYMSWYRQDPGLGLRQIYYSMNVEVTDKGDVPEGYKVSRKEKRNFPLILESPSPNQTSLYFCASSFSGGTYEQYFGPGTRLTVTE

SEQ ID NO: 277 - protein, 144 aa, synthetic construct
MLSPDLPDSAWNTRLLCHVMLCLLGAVSVAAGVIQSPRHLIKEKRETATLKCYPIPRHDTVYWYQQGPGQDPQFLISFYEKMQSDKGSIPDRFSAQQFSDYHSELNMSSLELGDSALYFCASSYRGGSTYEQYFGPGTRLTVTE

SEQ ID NO: 278 - protein, 135 aa, synthetic construct
MSTRLLCWMALCLLGAELSEAEVAQSPRYKITEKSQAVAFWCDPISGHATLYWYRQILGQGPELLVQFQDESVVDDSQLPKDRFSAERLKGVDSTLKIQPAELGDSAMYLCASSQRDSPNEKLFFGSGTQLSVLE

SEQ ID NO: 279 - protein, 136 aa, synthetic construct
MGCRLLCCAVLCLLGAVPMETGVTQTPRHLVMGMTNKKSLKCEQHLGHNAMYWYKQSAKKPLELMFVYSLEERVENNSVPSRFSPECPNSSHLFLHLHTLQPEDSALYLCASSQDPYKLSGNTIYFGEGSWLTVVE

SEQ ID NO: 280 - protein, 176 aa, synthetic construct
DLNKVFPPEVAVFEPSEAEISHTQKATLVCLATGFFPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 281 - protein, 178 aa, synthetic construct
DLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 282 - protein, 132 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNIGNHDMRFGAGTRLTVKPN

SEQ ID NO: 283 - protein, 132 aa, synthetic construct
MEKMLECAFIVLWLQLGWLSGEDQVTQSPEALRLQEGESSSLNCSYTVSGLRGLFWYRQDPGKGPEFLFTLYSAGEEKEKERLKATLTKKESFLHITAPKPEDSATYLCAVQTMDGNQFYFGTGTSLTVIPN

SEQ ID NO: 284 - protein, 134 aa, synthetic construct
MACPGFLWALVISTCLEFSMAQTVTQSQPEMSVQEAETVTLSCTYDTSESDYYLFWYKQPPSRQMILVIRQEAYKQQNATENRFSVNFQKAAKSFSLKISDSQLGDAAMYFCASSPGTYKYIFGTGTRLKVLAN

SEQ ID NO: 285 - protein, 137 aa, synthetic construct
MTRVSLLWAVVVSTCLESGMAQTVTQSQPEMSVQEAETVTLSCTYDTSESNYYLFWYKQPPSRQMILVIRQEAYKQQNATENRFSVNFQKAAKSFSLKISDSQLGDTAMYFCAFNPWENYGQNFVFGPGTRLSVLPY

SEQ ID NO: 286 - protein, 140 aa, synthetic construct; identical to SEQ ID NO: 268

SEQ ID NO: 287 - protein, 134 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSQGTSGADTQYFGPGTRLTVLE

SEQ ID NO: 288 - protein, 135 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYSLWDLQETQYFGPGTRLLVLE

SEQ ID NO: 289 - protein, 136 aa, synthetic construct
MGTSLLCWMALCLLGADHADTGVSQDPRHKITKRGQNVTFRCDPISEHNRLYWYRQTLGQGPEFLTYFQNEAQLEKSRLLSDRFSAERPKGSFSTLEIQRTEQGDSAMYLCASSFSDGGATDTQYFGPGTRLTVLE

SEQ ID NO: 290 - protein, 132 aa, synthetic construct
MLLLLLLLGPAGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARPHSLTDTQYFGPGTRLTVLE

SEQ ID NO: 291 - protein, 178 aa, synthetic construct; identical to SEQ ID NO: 281

SEQ ID NO: 292 - protein, 10 aa, Homo sapiens
GADGVGKSAL

SEQ ID NO: 293 - protein, 9 aa, Homo sapiens
GADGVGKSA

SEQ ID NO: 294 - DNA, 390 bp, Homo sapiens
  source 1..390: mol_type = other DNA
atgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgaccccggcactgaagctttctttggacaaggcaccagactcacagttgta

SEQ ID NO: 295 - DNA, 927 bp, Homo sapiens
  source 1..927: mol_type = other DNA
atgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgaccccggcactgaagctttctttggacaaggcaccagactcacagttgtagaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggc

SEQ ID NO: 296 - DNA, 396 bp, Homo sapiens
  source 1..396: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgcggcgatggatagcagctataaattgatcttcgggagtgggaccagactgctggtcaggcct

SEQ ID NO: 297 - DNA, 816 bp, Homo sapiens
  source 1..816: mol_type = other DNA
atgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgcggcgatggatagcagctataaattgatcttcgggagtgggaccagactgctggtcaggcctatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 298 - DNA, 1800 bp, synthetic construct
  source 1..1800: mol_type = other DNA
atgggcaccaggctcttcttctatgtggccctttgtctgctgtgggcaggacacagggatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgaccccggcactgaagctttctttggacaaggcaccagactcacagttgtagaggacctcaagaatgtgtttccgcccgaagtcgcggtttttgaaccatcagaagccgagatctctcatacacaaaaggcgacgctcgtatgcctcgcgacgggattttatccggaccacgtcgagctttcctggtgggttaacggaaaggaggtgcattccggagtttgcacggaccctcagccattgaaggaacagcccgcactgaacgacagtaggtattgcctttcatctcgcctgcgcgtgtctgcgacattctggcaaaacccaagaaatcacttcagatgtcaagttcagttctacggtctcagcgagaatgatgagtggacacaagatagggctaaacccgtgactcaaatagtctctgccgaggcctgggggagggcggattgcggcttcacatcagaatcataccaacaaggagtattgagcgcgacaattctttacgaaattctgcttgggaaagcgactctgtacgcggtgctcgtgtccgctttggttcttatggcaatggttaaacgaaaggatagtaggggcgccaccaatttcagcctgctgaaacaggctggcgacgtggaagagaaccccggacctatgaaatccttgagagttttactagtgatcctgtggcttcagttgagctgggtttggagccaacagaaggaggtggagcagaattctggacccctcagtgttccagagggagccattgcctctctcaactgcacttacagtgaccgaggttcccagtccttcttctggtacagacaatattctgggaaaagccctgagttgataatgttcatatactccaatggtgacaaagaagatggaaggtttacagcacagctcaataaagccagccagtatgtttctctgctcatcagagactcccagcccagtgattcagccacctacctctgtgccgcggcgatggatagcagctataaattgatcttcgggagtgggaccagactgctggtcaggcctatccagaaccccgaccccgccgtgtaccagctgcgggacagcaagagcagcgacaagagcgtgtgcctgttcaccgacttcgacagccagaccaacgtgtcccagagcaaggacagcgacgtgtacatcaccgataagtgcgtgctggacatgcggagcatggacttcaagagcaacagcgccgtggcctggtccaacaagagcgacttcgcctgcgccaacgccttcaacaacagcatcatccccgaggacacattcttcccaagccccgagagcagctgcgacgtgaagctggtggagaagtccttcgagacagacaccaacctgaacttccagaacctgtccgtgatcggcttcagaatcctgctgctgaaagtggccggcttcaacctgctgatgaccctgcggctgtggtccagc

SEQ ID NO: 299 - protein, 130 aa, Homo sapiens
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVV

SEQ ID NO: 300 - protein, 309 aa, Homo sapiens
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 301 - protein, 132 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRP

SEQ ID NO: 302 - protein, 272 aa, Homo sapiens
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 303 - protein, 600 aa, synthetic construct
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRPIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 304 - protein, 129 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSS

SEQ ID NO: 305 - protein, 133 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVV

SEQ ID NO: 306 - protein, 129 aa, Homo sapiens; identical to SEQ ID NO: 304

SEQ ID NO: 307 - protein, 133 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGRASNQPQHFGDGTRLSIL

SEQ ID NO: 308 - protein, 129 aa, Homo sapiens
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDRDQAGTALIFGKGTTLSVSS

SEQ ID NO: 309 - protein, 133 aa, Homo sapiens
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGQSSTYGYTFGSGTRLTVV

SEQ ID NO: 310 - protein, 266 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDMDQAGTALIFGKGTTLSVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 311 - protein, 306 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGEGRVDGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 312 - protein, 266 aa, synthetic construct; identical to SEQ ID NO: 310

SEQ ID NO: 313 - protein, 306 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSLGRASNQPQHFGDGTRLSILEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 314 - protein, 266 aa, synthetic construct
MRQVARVIVFLTLSTLSLAKTTQPISMDSYEGQEVNITCSHNNIATNDYITWYQQFPSQGPRFIIQGYKTKVTNEVASLFIPADRKSSTLSLPRVSLSDTAVYYCLVGDRDQAGTALIFGKGTTLSVSSDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 315 - protein, 306 aa, synthetic construct
MGPGLLCWALLCLLGAGLVDAGVTQSPTHLIKTRGQQVTLRCSPKSGHDTVSWYQQALGQGPQFIFQYYEEEERQRGNFPDRFSGHQFPNYSSELNVNALLLGDSALYLCASSFGQSSTYGYTFGSGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 316 - protein, 269 aa, synthetic construct
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAAAMDSSYKLIFGSGTRLLVRPDIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 317 - protein, 303 aa, synthetic construct
MGTRLFFYVALCLLWAGHRDAGITQSPRYKITETGRQVTLMCHQTWSHSYMFWYRQDLGHGLRLIYYSAAADITDKGEVPDGYVVSRSKTENFPLTLESATRSQTSVYFCASSDPGTEAFFGQGTRLTVVEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 318 - DNA, 1098 bp, Homo sapiens
  source 1..1098: mol_type = other DNA
atgcgggttatggcgccaagaacgttgatcttgctcctgagtggggcgctcgctctgactgagacatgggcatgctcccactccatgagatacttttacaccgccgtgtctcgcccaggacgaggggaacccagatttattgcggttgggtacgtggacgacacacagttcgtacaattcgatagtgacgcagccagtcccagaggtgagcctcgcgcgccgtgggttgaacaggaaggaccagagtattgggatagggagacccaaaaatacaaacgacaggcgcagacagacagggtgtctttgagaaacctcaggggatattacaatcaaagcgaggcaggttcccacactttgcaacggatgtacggctgcgatcttgggcccgatggacggcttctccgcggctacaaccagtttgcctatgatggcaaggactatatcgccctcaatgaagacctccgctcttggacggcggcggacaaagcagcacagatcacccaacgcaaatgggaggctgcccgcgaagctgagcagaggcgagcatatttggagggtacctgcgttgagtggctcaggcggtacctcgagaacggtaaaaaaacgttgcagagggcagagcacccaaaaacccacgttactcatcatccggtgtctgatcacgaagcgacactgcgatgttgggcgctcgggttctaccccgctgagatcaccctcacatggcaacgcgatggagaggatcaaacacaggataccgaactcgtagagacgaggcccgctggtgacgggacattccaaaagtgggccgcggtagttgttcccagcggagaagagcagcgctacacctgccacgttcaacacgaaggactccccgaaccccttactctgagatgggggccttctagccaaccaactatacctattgtcggaatcgtagccgggctggcggtactggcagttctcgctgtgcttggggcggttatggcagtagtcatgtgccgcagaaagtcttcaggtggtaagggtggaagttgctctcaagctgcatcatccaactctgcccaaggcagtgatgagtcactgattgcgtgtaaggcc

SEQ ID NO: 319 - DNA, 357 bp, Homo sapiens
  source 1..357: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 320 - DNA, 1512 bp, synthetic construct
  source 1..1512: mol_type = other DNA
atgcgggttatggcgccaagaacgttgatcttgctcctgagtggggcgctcgctctgactgagacatgggcatgctcccactccatgagatacttttacaccgccgtgtctcgcccaggacgaggggaacccagatttattgcggttgggtacgtggacgacacacagttcgtacaattcgatagtgacgcagccagtcccagaggtgagcctcgcgcgccgtgggttgaacaggaaggaccagagtattgggatagggagacccaaaaatacaaacgacaggcgcagacagacagggtgtctttgagaaacctcaggggatattacaatcaaagcgaggcaggttcccacactttgcaacggatgtacggctgcgatcttgggcccgatggacggcttctccgcggctacaaccagtttgcctatgatggcaaggactatatcgccctcaatgaagacctccgctcttggacggcggcggacaaagcagcacagatcacccaacgcaaatgggaggctgcccgcgaagctgagcagaggcgagcatatttggagggtacctgcgttgagtggctcaggcggtacctcgagaacggtaaaaaaacgttgcagagggcagagcacccaaaaacccacgttactcatcatccggtgtctgatcacgaagcgacactgcgatgttgggcgctcgggttctaccccgctgagatcaccctcacatggcaacgcgatggagaggatcaaacacaggataccgaactcgtagagacgaggcccgctggtgacgggacattccaaaagtgggccgcggtagttgttcccagcggagaagagcagcgctacacctgccacgttcaacacgaaggactccccgaaccccttactctgagatgggggccttctagccaaccaactatacctattgtcggaatcgtagccgggctggcggtactggcagttctcgctgtgcttggggcggttatggcagtagtcatgtgccgcagaaagtcttcaggtggtaagggtggaagttgctctcaagctgcatcatccaactctgcccaaggcagtgatgagtcactgattgcgtgtaaggccgccaccaatttcagcctgctgaaacaggctggcgacgtggaagagaaccccggacctatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatg

SEQ ID NO: 321 - protein, 366 aa, Homo sapiens [sequence omitted]

SEQ ID NO: 322 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 323 - protein, 504 aa, synthetic construct
MRVMAPRTLILLLSGALALTETWACSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADKAAQITQRKWEAAREAEQRRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWGPSSQPTIPIVGIVAGLAVLAVLAVLGAVMAVVMCRRKSSGGKGGSCSQAASSNSAQGSDESLIACKAATNFSLLKQAGDVEENPGPMSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

(19) United States
(12) Patent Application Publication
(10) Pub. No.: US 2026/0021183 A1
(43) Pub. Date: Jan. 22, 2026

(54) CD3-EXPRESSING NATURAL KILLER CELLS WITH ENHANCED FUNCTION FOR ADOPTIVE IMMUNOTHERAPY

(71) Applicant: BOARD OF REGENTS, THE UNIVERSITY OF TEXAS SYSTEM, Austin, TX (US)

(72) Inventors: Katy REZVANI, Houston, TX (US); Bin LIU, Houston, TX (US)

(73) Assignee: BOARD OF REGENTS, THE UNIVERSITY OF TEXAS SYSTEM, Austin, TX (US)

(21) Appl. No.: 18/993,710

(22) PCT Filed: Jul. 21, 2023
(86) PCT No.: PCT/US2023/070687

§ 371 Date: Jan. 13, 2025

Related U.S. Application Data
(60) Provisional application No. 63/481,588, filed on Jan. 25, 2023.

(30) Foreign Application Priority Data
Jul. 22, 2022 (WO) PCT/US2022/074062

(51) Int. Cl. A61K 40/42; A61K 40/15; A61K 40/32; A61P 35/00; C07K 14/725; C07K 16/30; C07K 16/40

(52) U.S. Cl. CPC A61K 40/4269; A61K 40/15; A61K 40/32; A61K 40/4253; A61P 35/00; C07K 14/7051; C07K 16/30; C07K 16/40

(57) ABSTRACT

Embodiments of the disclosure include methods and compositions in which NK cells are modified by the hand of man to express T-cell receptor and CD3 co-receptor on NK cells that do not naturally express them. Such modified NK cells work effectively with monospecific, bispecific or multi-specific antibodies, wherein the bispecific or multi-specific antibodies are tailored to comprise anti-CD3 antibodies that bind the modified NK cells, thereby triggering signaling, activation, and cytotoxicity of target cells to which the antibodies also bind. Thus, the NK cells are specifically configured to be able to work effectively with Bispecific NK cell engagers (BiKEs) as well as Bispecific T cell Engagers (BiTEs).

### CROSS-REFERENCE TO RELATED APPLICATIONS

This application claims priority to International Application Serial No. PCT/US2022/074062, filed Jul. 22, 2022, and U.S. Provisional Patent Application Ser. No. 63/481,588, filed Jan. 25, 2023, each of which are incorporated by reference herein in their entirety.

### SEQUENCE LISTING

The instant application contains a Sequence Listing which has been submitted in ST26 format and is hereby incorporated by reference in its entirety. Said ST26 copy, created on Jul. 21, 2023, is named MDAC_P1347WO_Sequence_Listing.xml and is 422,889 bytes in size.

### I. TECHNICAL FIELD

This disclosure relates at least to the fields of immunology, cell biology, molecular biology, and medicine, including at least cancer medicine.

### II. BACKGROUND

Natural killer (NK) cells have been studied as potential anti-tumor effectors, yet a number of barriers limit their therapeutic exploitation, mainly related to their lack of antigen specificity. One approach to overcome this is to transduce NK cells with a chimeric antigen receptor (CAR) or an engineered T-cell receptor (TCR) to target a desired antigen. In T cells, one can utilize a bispecific or multi-specific antibody, such as a bispecific T cell engager (BiTE) that binds CD3 on the surface of T cells and that also binds an antigen on the surface of cancer cells. CD3 is composed of four distinct chains, and in mammals, the complex contains a CD3γ chain, a CD3δ chain, and two CD3ε chains. These chains associate with the T-cell receptor (TCR) and the ζ-chain (zeta-chain) to generate an activation signal in T lymphocytes. However, NK cells do not naturally express the CD3 receptor complex or TCRs.

The present disclosure satisfies a long-felt need in the art to improve upon immunotherapies including those that utilize NK cells.

### BRIEF SUMMARY

Embodiments of the disclosure include methods and compositions for treatment of an individual with cancer using adoptive cell therapy. In specific embodiments, the individual is provided a therapeutically effective amount of a bipartite therapy that includes both modified NK cells and antibodies that are capable of being able to bind the NK cells to initiate signaling, activation, and killing of target cells. The disclosure concerns NK cells that have been modified to express multiple proteins that are not naturally expressed in NK cells and that work in conjunction together, including heterologous proteins on the surface of the NK cells that are naturally not present in NK cells.

In specific embodiments, NK cells are engineered to express one or more proteins from a CD3 co-receptor complex and optionally a TCR receptor complex, each normally present on the surface of T cells. Such engineering provides greater versatility for the NK cells to be utilized in conjunction with a variety of bispecific or multi-specific antibodies, including those that comprise an anti-CD3 antibody (e.g., an anti-CD3 scFv). In particular embodiments, the modified NK cells are administered to an individual in need thereof in conjunction with one or more monospecific, bispecific or multi-specific antibodies, wherein each bispecific or multi-specific antibodies having one antibody that targets CD3 and one antibody that binds a desired antigen, such as a cancer antigen. As a result, in specific cases the NK cells expressing CD3 are able to bind the anti-CD3 antibody part of the bispecific or multi-specific antibody, and the antibody that binds a cancer antigen binds the cancer antigen on the surface of a cancer cell. Such a coordinated binding between the NK cells and the antibody results in activation of cytotoxicity against the target cancer antigen.

In particular embodiments, the present disclosure concerns modified NK cells that express the full or partial CD3 complex with or without TCRs, and in some cases individual CD3 chain(s) are heterologously linked to an NK-relevant signaling domain, all of which allows the modified NK cells to be utilized with a variety of bispecific antibodies.

Embodiments of the disclosure include compositions comprising NK cells modified to express part or all of a single chain or any combination of CD3δ, CD3ε, CD3γ, or CD3ζ. In some cases, the NK cells are modified to express the T-cell receptor (TCR) αβ chains or the TCR γδ chains. The NK cells may be modified to express part or all of CD3ζ, two of CD3ε, CD3δ, and CD3γ. In some cases, the NK cells are modified to express full length of CD3ζ, CD3ε, CD3δ, and/or CD3γ. In particular cases, any one or more of the CD3ζ, CD3ε, CD3δ, and CD3γ are heterologously linked to one or more intracellular signaling domains. The intracellular signaling domain may be selected from the group consisting of CD16, NKG2D, DAP10, DAP12, 2B4, 4-1BB, CD2, CD28 and a combination thereof. In some embodiments, an intracellular signaling domain is fused to CD3ζ. In some embodiments, an intracellular signaling domain is derived from DAP10. In some embodiments, an intracellular signaling domain is derived from CD28. In some embodiments, an intracellular signaling domain comprises a sequence derived from DAP10 and a sequence derived from CD28. In certain embodiments, an intracellular signaling domain comprises an amino acid sequence at least about 85% identical to SEQ ID NO: 115. In certain embodiments, an intracellular signaling domain comprises an amino acid sequence at least about 85% identical to SEQ ID NO: 116. In certain embodiments, an intracellular signaling domain comprises an amino acid sequence at least about 85% identical to SEQ ID NO: 117. In certain embodiments, an NK cell is modified to express a polynucleotide sequence at least 85% identical to UT-NK15-DAP10 (SEQ ID NO: 118), UT-NK15-28 (SEQ ID NO: 120), or UTNK15-28-DAP10 (SEQ ID NO: 122). In some embodiments, the intracellular signaling domain could also include other costimulatory signals relevant to NK cell function such as but not limited to, 2B4, DNA, 4-1BB, DAP12, NKG2D, etc. In specific embodiments, the composition further comprises one or more monospecific, bispecific or multi-specific antibodies, wherein the bispecific or multi-specific antibody comprises an anti-CD3 antibody. The NK cells may express the antibody and/or are complexed with the antibody. In some embodiments, the antibody is Imgatuzumab, Amivantamab, and/or Cetuximab. In some embodiments, the TCR is directed to a cancer antigen or a viral antigen. In specific embodiments, the NK cells are derived from cord blood (CB), peripheral blood (PB), bone marrow, stem cells, or a mixture thereof. In some embodiments, the TCR is directed to a KRAS antigen. The NK cells may be pre-activated, such as with one or more cytokines, including IL-2, IL-7, IL-12, IL-15, IL-18, IL-21, or a combination thereof, for example. In some embodiments, the NK cells are expanded, such as in the presence of IL-2. In specific embodiments, the NK cells are modified to express one or more heterologous proteins, such as one or more engineered antigen receptors, one or more cytokines, one or more homing receptors, and/or one or more chemokine receptors. In specific cases, the engineered antigen receptor is a chimeric antigen receptor and/or engineered T cell receptor. In some cases, the heterologous protein is a cytokine, such as one selected from the group consisting of IL-15, IL-12, IL-2, IL-18, IL-21, IL-23, GMCSF, or a combination thereof. The cytokine may be membrane-bound, and the membrane-bound cytokine may comprise a transmembrane domain from CD8, CD28, CD27, B7H3, IgG1, IgG4, CD4, DAP10, or DAP12. In specific cases, the NK cell expresses a chimeric antigen receptor and a cytokine. In some cases, the bispecific antibody comprises an antibody that targets a cancer antigen. In some cases, the bispecific antibody comprises Amivantamab. In some cases, the antibody comprises Imgatuzumab and/or Cetuximab. In some cases, the antibody targets EGFR, C-met, and/or TROP-2.

Embodiments of the disclosure include compositions comprising a complex, comprising: (1) NK cells modified to express part or all of the CD3 receptor complex and optionally modified to express the T-cell receptor (TCR) αβ chains or the TCR γδ chains; and (2) a monospecific, bispecific or multi-specific antibody, wherein the bispecific or multi-specific antibody comprises an anti-CD3 antibody that is bound to CD3 on the NK cells. In specific embodiments, the complex is housed in a pharmaceutically acceptable excipient. The complex may be housed in a delivery device.

In particular embodiments, there is a method of treating cancer in an individual, comprising the step of administering to the individual a therapeutically effective amount of any one of the compositions encompassed herein. In some embodiments, the NK cells and the antibody are administered to the individual at the same time. The NK cells and the antibody may or may not be administered in the same formulation. The NK cells and the antibody may be pre-complexed prior to administration to the individual. In specific embodiments, the NK cells and the antibody are administered to the individual at different times. The NK cells and the antibody may be administered by infusion. In specific embodiments, the NK cells are autologous or allogeneic with respect to the individual.

Embodiments of the disclosure include methods of redirecting the specificity of NNK cells against a cancer antigen for treatment of an individual with a monospecific, bispecific or multi-specific, wherein the bispecific or multi-specific antibody comprise an anti-CD3 antibody, comprising the steps of administering to the individual the antibody and NK cells that express part or all of the CD3 receptor complex and that optionally express part or all of TCR αβ chains or the TCR γδ chains. In specific embodiments, the method further comprising the step of modifying NK cells to express part or all of the CD3 receptor complex. In specific embodiments, the method further comprises the step of modifying NK cells to express the TCR αβ chains or the TCR γδ chains. In some cases, the method further comprises the step of modifying the NK cells to express one or more heterologous proteins.

Certain embodiments of the present invention are characterized through the following aspects.

Aspect 1 is a composition, comprising engineered NK cells modified to express one or more transgenic polynucleotides encoding a) a CD3 protein complex comprising part or all of a single chain or any combination of CD3δ, CD3ε, CD3γ, or CD3ζ, b) optionally at least one cytokine, and c) at least one engineered T cell receptor (TCR) comprising, one of more of a TCRα chain, a TCRβ chain, a TCRγ chain, and/or a TCRδ chain, wherein the engineered TCR targets a KRAS antigen.

Aspect 2, is the composition of aspect 1, wherein the NK cells are modified to express the TCR αβ chains or the TCR γδ chains.

Aspect 3, is the composition of aspect 1 or 2, wherein the NK cells are modified to express part or all of CD3ζ, two of CD3ε, CD3δ, and CD3γ.

Aspect 4 is the composition of any one of aspects 1-3, wherein the NK cells are modified to express full length of CD3ζ, CD3ε, CD3δ, and/or CD3γ.

Aspect 5 is the composition of any one of aspects 1-4, wherein any one or more of the CD3ζ, CD3ε, CD3δ, and CD3γ are heterologously linked to one or more intracellular signaling domains.

Aspect 6 is the composition of aspect 5, wherein the intracellular signaling domain is selected from the group consisting of CD16, NKG2D, DAP10, DAP12, 2B4, 4-1BB, CD2, CD28, and a combination thereof.

Aspect 7 is the composition of aspect 5 or 6, wherein the intracellular signaling domain comprises a DAP10 intracellular signaling domain.

Aspect 8 is the composition of aspect 7, wherein the intracellular signaling domain comprises an amino acid sequence at least about 85% identical to SEQ ID NO: 115.

Aspect 9 is the composition of aspect 7, wherein the intracellular signaling domain comprises an amino acid sequence according to SEQ ID NO: 115.

Aspect 10 is the composition of any one of aspects 6-9, wherein the intracellular signaling domain comprises a CD28 intracellular signaling domain.

Aspect 11 is the composition of aspect 10, wherein the intracellular signaling domain comprises an amino acid sequence at least about 85% identical to SEQ ID NO: 116.

Aspect 12 is the composition of aspect 10, wherein the intracellular signaling domain comprises an amino acid sequence according to SEQ ID NO: 116.

Aspect 13 is the composition of any one of aspects 6-13, wherein the intracellular signaling domain comprises a DAP10 and CD28 intracellular signaling domain.

Aspect 14 is the composition of aspect 13, wherein the intracellular signaling domain comprises an amino acid sequence at least about 85% identical to SEQ ID NO: 117.

Aspect 15 is the composition aspect 13, wherein the intracellular signaling domain comprises an amino acid sequence according to SEQ ID NO: 117.

Aspect 16 is the composition of any one of aspects 1-15, wherein the composition further comprises one or more monospecific, bispecific, or multi-specific antibodies, wherein the bispecific or multi-specific antibody comprises an anti-CD3 antibody.

Aspect 17 is the composition of aspect 16, wherein the NK cells express the antibody.

Aspect 18 is the composition of aspect 16 or 17, wherein the NK cells are complexed with the antibody.

Aspect 19 is the composition of any one of aspects 16-18, wherein the antibody is Imgatuzumab, Amivantamab, and/or Cetuximab.

Aspect 20 is the composition of any one of aspects 1-19, wherein the NK cells are derived from cord blood (CB), peripheral blood (PB), bone marrow, stem cells, or a mixture thereof.

Aspect 21 is the composition of any one of aspects 1-20, wherein the NK cells are pre-activated.

Aspect 22 is the composition of aspect 21, wherein the NK cells are pre-activated with one or more cytokines.

Aspect 23 is the composition of aspect 22, wherein the cytokines are IL-2, IL-7, IL-12, IL-15, IL-18, IL-21, or a combination thereof.

Aspect 24 is the composition of any one of aspects 1-23, wherein the NK cells are expanded.

Aspect 25 is the composition of aspect 24, wherein the NK cells are expanded in the presence of IL-2.

Aspect 26 is the composition of any one of aspects 1-25, wherein the NK cells are modified to express one or more additional heterologous protein.

Aspect 27 is the composition of aspect 26, wherein the additional heterologous protein is an engineered antigen receptor, a cytokine, a homing receptor, and/or a chemokine receptor.

Aspect 28 is the composition of any one of aspects aspect 1-27, wherein the TCR comprises a sequence at least 85% identical to SEQ ID NO: 299 and a sequence at least 85% identical to SEQ ID NO: 301.

Aspect 29 is the composition of any one of aspects aspect 1-28, wherein the TCR comprises one or more sequences at least 85% identical to SEQ ID NOs: 299-317.

Aspect 30 is the composition of any one of aspects aspect 1-29, wherein the target KRAS antigen epitope comprises or consists of GADGVGKSA (SEQ ID NO: 293) and/or GADGVGKSAL (SEQ ID NO: 292).

Aspect 31 is the composition of any one of aspects 1-30, wherein the TCR comprises a sequence at least 85% identical to SEQ ID NO: 304 and a sequence at least 85% identical to SEQ ID NO: 305.

Aspect 32 is the composition of any one of aspects 1-31, wherein the TCR comprises a sequence at least 85% identical to SEQ ID NO: 306 and a sequence at least 85% identical to SEQ ID NO: 307.

Aspect 33 is the composition of any one of aspects 1-32, wherein the TCR comprises a sequence at least 85% identical to SEQ ID NO: 308 and a sequence at least 85% identical to SEQ ID NO: 309.

Aspect 34 is the composition of any one of aspects 26-33, wherein the heterologous protein is a cytokine.

Aspect 35 is the composition of aspect 34, wherein the cytokine is selected from the group consisting of IL-15, IL-12, IL-2, IL-18, IL-21, IL-23, IL-7, GMCSF, or a combination thereof.

Aspect 36 is the composition of aspect 34 or 35, wherein the cytokine is membrane-bound.

Aspect 37 is the composition of any one of aspects 34-36, wherein the cytokine is IL-15.

Aspect 38 is the composition of aspect 36 or 37, wherein the membrane-bound cytokine comprises a transmembrane domain from CD8, CD28, CD27, B7H3, IgG1, IgG4, CD4, DAP10, or DAP12.

Aspect 39 is the composition of any one of aspects 27-38, wherein the NK cell expresses a chimeric antigen receptor and a cytokine.

Aspect 40 is the composition of any one of aspects 16-39, wherein the monospecific, bispecific, or multispecific antibody comprises an antibody that targets a cancer antigen.

Aspect 41 is the composition of aspect 40, wherein the cancer antigen is a EGFR, C-met, and/or TROP-2 antigen.

Aspect 42 is a composition comprising a complex, comprising: a) NK cells modified to express part or all of the CD3 receptor complex and modified to express the T-cell receptor (TCR) αβ chains or the TCR γδ chains; and b) a monospecific, bispecific, or multi-specific antibody, wherein the bispecific or multi-specific antibody comprises an anti-CD3 antibody that is bound to CD3 on the NK cells, wherein the TCR targets a KRAS antigen.

Aspect 43 is the composition of aspect 42, wherein the NK cells are modified to express TCR αβ chains that are at least 85% identical to SEQ ID NO: 299 and SEQ ID NO: 301, and the antibody is Imgatuzumab, Amivantamab, and/or Cetuximab.

Aspect 44 is the composition of aspect 42 or 43, wherein the NK cells are modified to express full length CD3ζ, CD3ε, CD3δ, and/or CD3γ.

Aspect 45 is the composition of any one of aspects 42-44, wherein any one or more of CD3ζ, CD3ε, CD3δ, and CD3γ are heterologously linked to one or more intracellular signaling domains.

Aspect 46 is the composition of aspect 45, wherein the intracellular signaling domain is selected from the group consisting of CD16, NKG2D, DAP10, DAP12, 2B4, 4-1BB, CD2, CD28, DNAM, and a combination thereof.

Aspect 47 is the composition of aspect 45 or 46, wherein the intracellular signaling domain comprises a DAP10 intracellular signaling domain.

Aspect 48 is the composition of any one of aspects 45-47, wherein the intracellular signaling domain comprises an amino acid sequence at least about 85% identical to SEQ ID NO: 115.

Aspect 49 is the composition of any one of aspects 45-48, wherein the intracellular signaling domain comprises an amino acid sequence according to SEQ ID NO: 115.

Aspect 50, is the composition of any one of aspects 45-49, wherein the intracellular signaling domain comprises a CD28 intracellular signaling domain.

Aspect 51 is the composition of any one of aspects 45-50, wherein the intracellular signaling domain comprises an amino acid sequence at least about 85% identical to SEQ ID NO: 116.

Aspect 52 is the composition of any one of aspects 45-51, wherein the intracellular signaling domain comprises an amino acid sequence according to SEQ ID NO: 116.

Aspect 53 is the composition of any one of aspects 45-52, wherein the intracellular signaling domain comprises a DAP10 and CD28 intracellular signaling domain.

Aspect 54 is the composition of any one of aspects 45-53, wherein the intracellular signaling domain comprises an amino acid sequence at least about 85% identical to SEQ ID NO: 117.

Aspect 55 is the composition of any one of aspects 45-54, wherein the intracellular signaling domain comprises an amino acid sequence according to SEQ ID NO: 117.

Aspect 56 is the composition of any one of aspects 1-55, wherein the composition is housed in a pharmaceutically acceptable excipient.

Aspect 57 is the composition of any one of aspects 1-56, wherein the composition is housed in a delivery device.

Aspect 58 is a method of treating cancer in an individual, comprising the step of administering to the individual a therapeutically effective amount of any one of the compositions of aspects 1-57.

Aspect 59 is a method of treating cancer in an individual, comprising the step of administering to the individual a therapeutically effective amount of any one of the compositions of aspects 16-57, wherein the NK cells and the antibody are administered to the individual at the same time, and optionally wherein the NK cells and the antibody are administered in the same formulation and/or pre-complexed prior to administration to the individual.

Aspect 60 is the method of aspect 58, wherein the NK cells and the antibody are administered to the individual at different times.

Aspect 61 is the method of any one of aspects 58-60, wherein the NK cells and the antibody are administered by infusion.

Aspect 62 is the method of any one of aspects 58-61, wherein the NK cells are autologous with respect to the individual.

Aspect 63 is the method of any one of aspects 58-61, wherein the NK cells are allogeneic with respect to the individual.

Aspect 64 is a method of redirecting the specificity of NK cells against a cancer antigen for treatment of an individual with a monospecific, bispecific or multi-specific antibody, wherein the bispecific or multi-specific antibody comprises an anti-CD3 antibody, comprising the steps of administering to the individual the antibody and NK cells that express part or all of a CD3 receptor complex and express part or all of TCR αβ chains or the TCR γδ chains, wherein the TCR targets a KRAS antigen.

Aspect 65 is the method of aspect 64, wherein the NK cells are modified to express full length CD3ζ, CD3ε, CD3δ, and/or CD3γ.

Aspect 66 is the method of aspect 64 or 65, wherein any one or more of CD3ζ, CD3ε, CD3δ, and CD3γ are heterologously linked to one or more intracellular signaling domains.

Aspect 67 is the method of any one of aspects 64-66, wherein the intracellular signaling domain is selected from the group consisting of CD16, NKG2D, DAP10, DAP12, 2B4, 4-1BB, CD2, CD28, DNAM, and a combination thereof.

Aspect 68 is the method of aspect 66 or 67, wherein the intracellular signaling domain comprises a DAP10 intracellular signaling domain.

Aspect 69 is the method of any one of aspects 66-68, wherein the intracellular signaling domain comprises an amino acid sequence at least about 85% identical to SEQ ID NO: 115.

Aspect 70 is the method of any one of aspects 66-69, wherein the intracellular signaling domain comprises an amino acid sequence according to SEQ ID NO: 115.

Aspect 71 is the method of any one of aspects 66-70, wherein the intracellular signaling domain comprises a CD28 intracellular signaling domain.

Aspect 72 is the method of any one of aspects 66-71, wherein the intracellular signaling domain comprises an amino acid sequence at least about 85% identical to SEQ ID NO: 116.

Aspect 73 is the method of any one of aspects 66-72, wherein the intracellular signaling domain comprises an amino acid sequence according to SEQ ID NO: 116.

Aspect 74 is the method of any one of aspects 66-73, wherein the intracellular signaling domain comprises a DAP10 and CD28 intracellular signaling domain.

Aspect 75 is the method of any one of aspects 66-74, wherein the intracellular signaling domain comprises an amino acid sequence at least about 85% identical to SEQ ID NO: 117.

Aspect 76 is the method of any one of aspects 66-75, wherein the intracellular signaling domain comprises an amino acid sequence according to SEQ ID NO: 117.

Aspect 77 is the method of any one of aspects 64-76, wherein the TCR chains are TCR αβ chains, and are at least 85% identical to SEQ ID NO: 299 and SEQ ID NO: 301.

Aspect 78 is the method of any one of aspects 64-77, wherein the TCR chains are TCR αβ chains, and are at least 85% identical to one or more of SEQ ID NOs: 299-317.

Aspect 79 is the method of any one of aspects 64-78, wherein the target KRAS antigen epitope comprises or consists of GADGVGKSA (SEQ ID NO: 293) and/or GADGVGKSAL (SEQ ID NO: 292).

Aspect 80 is the method of any one of aspects 64-79, wherein the TCR chains comprise a sequence at least 85% identical to SEQ ID NO: 304 and a sequence at least 85% identical to SEQ ID NO: 305.

Aspect 81 is the method of any one of aspects 64-79, wherein the TCR chains comprise a sequence at least 85% identical to SEQ ID NO: 306 and a sequence at least 85% identical to SEQ ID NO: 307.

Aspect 82 is the method of any one of aspects 64-79, wherein the TCR chains comprise a sequence at least 85% identical to SEQ ID NO: 308 and a sequence at least 85% identical to SEQ ID NO: 309.

Aspect 83 is the method of any one of aspects 64-82, further comprising the step of modifying the NK cells to express one or more additional heterologous proteins

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-1C illustrates various embodiments of NK cells engineered to express CD3, including for use with a variety of heterologous proteins, such as cytokines, bispecific NK cell engagers, and engineered antigen receptors (CAR and/or TCR). FIG. 1B illustrates NK cells accommodated for CD3 and TCR for optimal cancer immunotherapy. FIG. 1C illustrates examples of single chimeric CD3 constructions.

FIGS. 2A-2B illustrates one example of an expression construct for CD3 receptor complex components for transduction or transfection of NK cells. FIG. 2B shows an example of a plasmid map for the representative expression construct.

FIG. 3 provides a table of various TCR/CD3 expression construct designs for NK-TCR engineering.

FIG. 4 shows CD3 expression at day 4 on engineered NK cells after transduction with one example of a CMV-directed TCR complex.

FIG. 5 demonstrates TCR expression at day 4 on engineered NK cells following CMV-directed TCR complex transduction.

FIG. 6 shows TCR/CD3 expression at day 6 on engineered NK cells after transduction of a CMV-directed TCR complex into the cells.

FIG. 7 demonstrates binding at different concentrations of one example of a CD3-CD19 BiTE on NK cells through the CD3/TCR complex on the NK cells.

FIG. 8 shows NK-TCR cytokine production of TNFα and CD107a after stimulation with plate-bound CD3 antibody.

FIG. 9 demonstrates phosphorylation of CD3z in NK TCR/CD3 cells after crosslinking CD3.

FIGS. 10A-10B show that pre-culturing CD3-CD19 BiTEs with TCR/CD3-expressing NK cells increased its killing activity against Raji cells. FIG. 10A represents a 1:1 Effector:Target ratio, and FIG. 10B represents a 1:5 Effector:Target ratio.

FIG. 11 provides a schematic overview of multiple retroviral transductions to generate NK cells expressing CD3, IL-15, and a TCR complex.

FIG. 12 shows expression of NY-ESO TCR on NK cells transduced with uTNK15. WT refers to wild type CD3 molecules with IL-15; A refers to CD3-CD28 with IL-15; B refers to CD3-DAP10 with IL-15; and C refers to CD3-CD28-Dap10 with IL-15.

FIG. 13 shows the number of TCR molecules per cell expressed on NK cells. WT refers to wild type CD3 molecules with IL-15; A refers to CD3-CD28 with IL-15; B refers to CD3-DAP10 with IL-15; and C refers to CD3-CD28-Dap10 with IL-15. Phycoerythrin Fluorescence Quantitation Kit (BD Biosciences) was used to determine the number of molecules of NY-ESO TCR on NK cells.

FIG. 14 shows expression of NY-ESO TCR on T cells.

FIG. 15 shows that NK cells transduced with NY-ESO TCR kill NY-ESO peptide-pulsed target cells in a dose-dependent manner. WT refers to wild type CD3 molecules with IL-15; A refers to CD3-CD28 with IL-15; B refers to CD3-DAP10 with IL-15; and C refers to CD3-CD28-Dap10 with IL-15.

FIG. 16 demonstrates endogenous NY-ESO expression on human tumor cell lines.

FIG. 17 demonstrates that NY-ESO TCR transduced T cells kill NY-ESO expressing tumor targets.

FIG. 18 provides results that NY-ESO TCR transduced NK cells kill NY-ESO expressing tumor targets even at low E:T ratios. WT refers to wild type CD3 molecules with IL-15; A refers to CD3-CD28 with IL-15; B refers to CD3-DAP10 with IL-15; and C refers to CD3-CD28-Dap10 with IL-15.

FIGS. 19A-19B show that NY-ESO transduced NK cells have a similar phenotype (19A) and expression pattern (19B) to NT NK cells. WT refers to wild type CD3 molecules with IL-15; A refers to CD3-CD28 with IL-15; B refers to CD3-DAP10 with IL-15; and C refers to CD3-CD28-Dap10 with IL-15.

FIG. 20 provides a table representing the cellular composition of the expanded uTNK15 product. WT refers to wild type CD3 molecules with IL-15; A refers to CD3-CD28 with IL-15; B refers to CD3-DAP10 with IL-15; and C refers to CD3-CD28-Dap10 with IL-15.

FIGS. 21A-21C shows that NK cells can be successfully transduced with CD3 and TCR constant alpha-beta (TCRCab) (called TCR6 construct) and that the engineered NK cell can bind Blinatumumab (FIG. 21B) and selectively kill CD19+ lymphoma targets (FIG. 21C).

FIGS. 22A-22C shows the in vivo activity of effector cells (e.g., NK cells, or T cells) comprising NY-ESO targeted TCRs. FIG. 22A is a schematic outlining the experimental procedure performed. FIG. 22B displays bioluminescent imaging over time (day 1, day 7, day 14, and day 21) for the mice engrafted with U266B.1 cells transduced with FireFlyluciferase (FFluc) and treated with control, NY-ESO TCR NK cells, or NY-ESO TCR T cells (NK cells comprising WT, #A, or #B UT-NK15-NY ESO TCR constructs respectively; WT refers to wild type CD3 molecules with IL-15; #A refers to CD3-CD28 with IL-15; and #B refers to CD3-DAP10 with IL-15). FIG. 22C is a graphical quantification of the bioluminescence average radiance displayed in FIG. 22B. These results showed that effector cells comprising NY-ESO TCR constructs described herein robustly inhibited tumor growth in vivo.

FIGS. 23A-23B shows the in vitro activity of effector cells (e.g., NK cells or T cells) comprising NY-ESO targeted TCRs and UT-NK15 constructs. FIG. 23A are images of spheroids formed by osteosarcoma tumor cell line Saos-2 stably transduced to express GFP that were used to test the activity of NY-ESO1-specific TCR expressing NK and T cells cytotoxicity. FIG. 23B is a graph showing percentage of cytotoxicity (Y axis) for representative images after 3 days of co-culture. NK cells were co-transduced with NY-ESO-TCR, and the UT-NK15 signaling complex co-expressing different co-stimulatory molecules fused to the CD3ζ signaling chain or the TCR complex without IL-15. T cells were only transduced with NY-ESO TCR. Abbreviation in the graph: 28=CD3ζ fused to a CD28 co-stimulatory domain; 10=CD3ζ fused to a Dap10 co-stimulatory domain; 8=CD8 alpha/beta co-receptor as part of the NY ESO TCR construct; wo IL-15=the construct only contains CD3 zeta, epsilon, gamma and delta TCR complex without co-stimulation or IL-15.

FIGS. 24A-24D shows the in vivo activity of effector cells (e.g., NK cells or T cells) comprising NY-ESO targeted TCRs and UT-NK15 constructs. FIG. 24A depicts a plan for an in vivo study to test the activity of different NY ESO TCR transduced NK and T cells. FIG. 24B depicts BLI imaging results of the test outlined and performed according to FIG. 24A, Mice were injected with U266 tumor cells, and three days later received T cells transduced with NY-ESO-specific TCR, or NK cells co-transduced with NY-ESO TCR and UT-NK15 with CD3ζ fused to CD28 (labelled as NY-ESO NK UT-NK15 CD28 or NY-ESO TCR UTNK-15 CD28 NK cells). The tumor alone group was used as control. FIG. 24C depicts region of interest average radiance intensity for the animals tested according to FIG. 24A and imaged in FIG. 24B. FIG. 24D is a graph depicting the cohort survival curves for the aforementioned animals.

FIG. 25 shows the in vivo activity of effector cells (e.g., NK cells) engineered to express NY ESO TCR and CD3 complex with or without IL-15 transgene comprised in the construct. NSG mice were irradiated (300 cGy) and the next day were injected with 500,000 U266 cells (HLA-A2 positive, NY-ESO-expressing myeloma cell line) via the tail vein. Three days later, mice received 5 million TCR transduced T or NK cells. Mice were monitored for tumor control by BLI imaging. NK cells were transduced with NY-ESO-specific TCR with or without expression of CD8 alpha/beta co-receptors, co-transduced with CD3 complex without IL-15 transgene or with UT-NK15 expressing CD3ζ fused to CD28 (UT-NK15 CD28) or CD3ζ fused to DAP10 (UT-NK15 DAP10) co-stimulatory molecules.

FIGS. 26A-26C shows in vitro expression of Preferentially Expressed Antigen in Melanoma (PRAME) TCRs on effector cells (e.g., NK cells or T cells) and the in vitro activity of said cells. FIG. 26A shows the expression of both UT-NK15 (x-axis, CD3) and PRAME-specific TCRs (y-axis, TCR) in NK cells (TCR clones 46, 54, or DSK3 respectively), or the expression of PRAME-specific TCRs in T cells transduced with the same (TCR clones 46 or 54). FIG. 26B shows the in vitro cytotoxicity of NK cells expressing a PRAME-specific TCR against the U266 myeloma cell line. IncuCyte® live cell imaging was used to measure the cytotoxicity of T cells transduced with PRAME-specific TCR and NK cells transduced with UT-NK15 and PRAME-specific TCR against U266 myeloma cells. GFP-expressing U266 cells were co-cultured with PRAME-specific TCR expressing T cell or NK cells at 1:1 effector:target ratio. A reduction in GFP expression indicated cell death. After 26 hours, a second round of 50,000 tumor cells was added (noted as “rechallenging”) to each well for the tumor rechallenge assay. Open symbols represent T cells, while closed symbols represent NK cells. NT=non-transduced. FIG. 26C shows the in vitro cytotoxicity of NK cells expressing a PRAME-specific TCR against the UA375 melanoma cell line. IncuCyte® live cell imaging was used to measure the cytotoxicity of T cells transduced with PRAME-specific TCR and NK cells transduced with UT-NK15 and PRAME-specific TCR (PRAME-specific TCR clone 46 (TCR-46), PRAME-specific TCR clone 54 (TCR-54), or PRAME-specific TCR clone DSK3 (DSK)) against UA375 melanoma cells. GFP-expressing UA375 cells were co-cultured with PRAME-expressing T cell or NK cells at 1:1 effector:target ratio. A reduction in GFP expression indicated cell death. After 26 hours, a second round of 50,000 tumor cells was added to each well for the tumor rechallenge assay. Open symbols represent T cells, while closed symbols represent NK cells. NT=non-transduced.

FIGS. 27A-27B shows in vitro expression of transgenic Kirsten rat sarcoma virus (KRAS)-specific TCRs on effector cells (e.g., NK cells) and the in vitro activity of said cells. FIG. 27A shows the expression of both UT-NK15 (x-axis, CD3) (e.g., SEQ ID NO: 120) and KRAS-specific TCRs (y-axis, TCRab) (SEQ ID NOs: 300 and 302) in transduced NK cells (3 cord blood donors, CB25, CB26, and CB27 respectively) (bottom) compared to non-transduced (NT) NK cell controls (top). FIG. 27B shows the in vitro cytotoxicity of NK cells expressing a KRAS-specific TCR and UT-NK15 against a BcPC3 cell line (pancreatic cancer cell line BxPC-3 transduced with a construct encoding HLA-Cw08:02 and beta-2-M (SEQ ID NO: 320)) loaded with 1 μg/ml KRAS-G12D-10mer (GADGVGKSAL) peptide (SEQ ID NO: 292; noted as “G12D-10-mer”). Cytotoxicity was measured in real-time using the Xcelligence cell analysis system, the cytotoxicity's of control non-transduced NK cells (cb25-NK-NT; dashed line) or NK cells transduced with KRAS-specific TCR and UT-NK15 (cb25-kras-TCR NK; solid line) against BcPC3 cells with KRAS-G12D-10-mer (HLA-Cw08:02+ BxPC-3+kras-G12D-10-mer) were measured and compared. Cells were co-cultured at a 5:1 effector:target ratio. Assays were performed for NK cells from donors CB25, CB26, and CB27, the graph shows data from the representative KRAS-specific TCR expressing NK cell line from CB25, with triplicate replicates for each treatment. Statistical significance was assessed by two-tailed t-test (****=P<0.0001). The tumor cell index used as a measure of cytotoxicity, and showed the significant superiority of KRAS-specific TCR UT-NK15 expressing NK cells in killing KRAS+HLA-Cw08:02+ BxPC-3 cells relative to non-transduced control NK cells.

### DETAILED DESCRIPTION

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, the term “CD3 receptor complex” or “CD3 co-receptor complex” refers to the protein complex that in nature acts as a T cell co-receptor and is comprised of CD3ζ chain, CD3γ chain, a CD3δ chain, and two CD3ε chains (although in alternatives only one CD3ε chain is used).

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure. In specific embodiments, a vector is engineered through recombinant nucleic acid technologies, and a cell is engineered through transfection or transduction of an engineered vector. Cells may be engineered to express heterologous proteins that are not naturally expressed by the cells, either because the heterologous proteins are recombinant or synthetic or because the cells do not naturally express the proteins.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “subject,” as used herein, generally refers to an individual having a that has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of one or more symptoms of the disease or condition, or the delaying of the progression of the disease or condition. “Treatment” does not necessarily indicate complete eradication or cure of the disease or condition, or associated symptoms thereof. Treating may mean alleviation of at least one symptom of the disease or condition.

As used herein “TCR/CD3 complex” refers to a protein complex naturally found on the surface of T cells and that comprises T-cell receptor (TCR) α and β chains and/or a T-cell receptor γ and δ chains, in addition to CD3ζ, CD3γ, CD3δ, and CD3ε chains.

### I. EMBODIMENTS OF THE DISCLOSURE

Natural killer (NK) cells are an emerging cellular immunotherapy for patients with malignant hematologic disease, as well as solid tumors. The present disclosure specifically relates to NK cells that have been modified to render the NK cells to have enhanced function as an immunotherapy compared to NK cells not so modified. The modifications allow for the NK cells to have greater versatility when used with other therapeutic agents and at least in some embodiments to have T cell-like activity by utilizing the CD3/TCR receptor complex. In specific embodiments, the NK cells are modified to express (i) either a single CD3 chain (CD3zeta, CD3 epsilon, CD3 delta, or CD3 gamma) or part or all of the human CD3 receptor complex (including any combination of CD3 delta, epsilon (one or two copies of epsilon), gamma, and zeta); or (ii) either a single CD3 chain or the human CD3 receptor complex (including any combination of CD3 delta, epsilon (one or two molecules), gamma, and zeta) as a full length protein or as a partial protein heterologously linked to one or more intracellular signaling domains); and (iii) the CD3 complex may or may not include the T-cell receptor (αβ or γδ). The disclosure concerns the use of CD3-expressing NK cells in the diagnosis and treatment of disease, including use of the cells in combination with monospecific, bispecific or multi-specific antibodies in which one epitope of the bispecific and/or multispecific antibody binds CD3 on the CD3-expressing NK cells). The CD3-expressing NK cells can either be pre-complexed ex vivo with the bi/multi-specific antibody to redirect their specificity toward the target antigen and/or combined in vivo. In diagnostic embodiments, labeled NK cells may be loaded with bispecific or multi-specific antibodies of any kind, including that comprise at least an anti-CD3 antibody, and the loaded, labeled NK cells may be monitored for trafficking to the site of the target antigen for which another antibody on the bispecific or multi-specific antibody binds.

In certain embodiments of the disclosure, a TCR recognizes antigens and/or epitopes presented by a multi-histocompatibility complex (MHC). In certain embodiments, an antigen and/or epitope is a peptide, lipid, and/or glycolipid. In certain embodiments, a MHC is a class I MHC. In certain embodiments, a MHC is a class II MHC. In certain embodiments, an MHC is a non-classical MHC.

In certain embodiments, a TCR target antigen can be a primary and/or secondary antigen that provides a transduced effector cell with target antigen specificity. In certain embodiments, a TCR acts primarily as a stabilizer for a CD3 co-receptor complex, while an antibody provides the primary target antigen specificity for a transduced effector cell.

### II. COMPOSITIONS OF THE DISCLOSURE

The disclosure concerns compositions that at least include modified NK cells that express at least parts of the TCR/CD3 complex. In some cases, the compositions also include monospecific, bispecific or multi-specific antibodies, including in the same formulation, although in alternative embodiments the NK cells and antibodies are utilized as physically separate compositions.

### A. NK Cell TCR/CD3 Modifications

In particular embodiments, provided herein are compositions that comprise NK cells that have been modified by the hand of man to express part or all of the TCR receptor complex and part or all of the CD3 co-receptor complex. In specific embodiments, the NK cells are modified to include all components of the CD3 complex, including CD3ζ, CD3ε, CD3γ and CD3δ. Although in particular cases the full lengths of CD3ζ, CD3ε, CD3γ and CD3δ are utilized, including their extracellular domain, transmembrane domain, and intracellular domain, in alternative embodiments only part of one or more of CD3ζ, CD3ε, CD3γ and CD3δ are utilized each of which that may or may not be combined with one or more intracellular signaling domains such as CD16, NKG2D, DAP10, DAP12, CD28, 41BB, 2B4, CD27, OX40, or any combination thereof. The NK cells may also be modified to express the TCR receptor complex, although in alternative embodiments none of the TCR receptor complex components are utilized.

In certain embodiments, an amino acid sequence (e.g., a polypeptide) may comprise an amino acid represented by a single letter “X” or a three letter code “Xaa”. In some embodiments, the amino acid represented by “X” or “Xaa” is any naturally occurring amino acid, such as but not limited to, Arginine (Arg, R), Histidine (His, H), Lysine (Lys, K), Aspartic Acid (Asp, D), Glutamic Acid (Glu, E), Serine (Ser, S), Threonine (Thr, T), Asparagine (Asn, N), Glutamine (Gln, Q), Glycine (Gly, G), Proline (Pro, P), Cysteine (Cys, C), Alanine (Ala, A), Valine (Val, V), Isoleucine (Ile, I), Leucine (Leu, L), Methionine (Met, M), Phenylalanine (Phe, F), Tyrosine (Tyr, Y), or Tryptophan (Trp, W).

In some embodiments, the amino acid represented by “X” or “Xaa” in SEQ ID NO: 25 or SEQ ID NO: 88 is Arginine (Arg, R). In some embodiments, the amino acid represented by “X” or “Xaa” in SEQ ID NO: 25 or SEQ ID NO: 88 is Histidine (His, H). In some embodiments, the amino acid represented by “X” or “Xaa” in SEQ ID NO: 25 or SEQ ID NO: 88 is Lysine (Lys, K). In some embodiments, the amino acid represented by “X” or “Xaa” in SEQ ID NO: 25 or SEQ ID NO: 88 is Aspartic Acid (Asp, D). In some embodiments, the amino acid represented by “X” or “Xaa” in SEQ ID NO: 25 or SEQ ID NO: 88 is Glutamic Acid (Glu, E). In some embodiments, the amino acid represented by “X” or “Xaa” in SEQ ID NO: 25 or SEQ ID NO: 88 is Serine (Ser, S). In some embodiments, the amino acid represented by “X” or “Xaa” in SEQ ID NO: 25 or SEQ ID NO: 88 is Threonine (Thr, T). In some embodiments, the amino acid represented by “X” or “Xaa” in SEQ ID NO: 25 or SEQ ID NO: 88 is Asparagine (Asn, N). In some embodiments, the amino acid represented by “X” or “Xaa” in SEQ ID NO: 25 or SEQ ID NO: 88 is Glutamine (Gln, Q). In some embodiments, the amino acid represented by “X” or “Xaa” in SEQ ID NO: 25 or SEQ ID NO: 88 is Glycine (Gly, G). In some embodiments, the amino acid represented by “X” or “Xaa” in SEQ ID NO: 25 or SEQ ID NO: 88 is Proline (Pro, P). In some embodiments, the amino acid represented by “X” or “Xaa” in SEQ ID NO: 25 or SEQ ID NO: 88 is Cysteine (Cys, C). In some embodiments, the amino acid represented by “X” or “Xaa” in SEQ ID NO: 25 or SEQ ID NO: 88 is Alanine (Ala, A). In some embodiments, the amino acid represented by “X” or “Xaa” in SEQ ID NO: 25 or SEQ ID NO: 88 is Valine (Val, V). In some embodiments, the amino acid represented by “X” or “Xaa” in SEQ ID NO: 25 or SEQ ID NO: 88 is Isoleucine (Ile, I). In some embodiments, the amino acid represented by “X” or “Xaa” in SEQ ID NO: 25 or SEQ ID NO: 88 is Leucine (Leu, L). In some embodiments, the amino acid represented by “X” or “Xaa” in SEQ ID NO: 25 or SEQ ID NO: 88 in SEQ ID NO: 25 or SEQ ID NO: 88 is Methionine (Met, M). In some embodiments, the amino acid represented by “X” or “Xaa” in SEQ ID NO: 25 or SEQ ID NO: 88 is Phenylalanine (Phe, F). In some embodiments, the amino acid represented by “X” or “Xaa” in SEQ ID NO: 25 or SEQ ID NO: 88 is Tyrosine (Tyr, Y). In some embodiments, the amino acid represented by “X” or “Xaa” in SEQ ID NO: 25 or SEQ ID NO: 88 is Tryptophan (Trp, W).

In certain embodiments, particular sequences for any of the CD3 receptor components are utilized, including wildtype or mutants of the components so long as the CD3 receptor having the mutant is able to allow signaling through the CD3 complex leading to activation and killing of targets. In some cases, the following examples of sequences for CD3ε, CD3δ, CD3γ, and CD3ζ and are utilized for modification of the NK cells.

### CD3 Epsilon (UniProtKB—P07766 (CD3E_HUMAN))

### Signal Peptide

(SEQ ID NO: 1)
MQSGTHWRVLGLCLLSVGVW

### Extracellular Domain

sp|P07766|23-126
(SEQ ID NO: 2)
DGNEEMGGITQTPYKVSISGTTVILTCPQYPGSEILWQHNDKNIGGDE
DDKNIGSDEDHLSLKEFSELEQSGYYVCYPRGSKPEDANFYLYLRARV
CENCMEMD

### Transmembrane Domain

sp|P07766|127-152
(SEQ ID NO: 3)
VMSVATIVIVDICITGGLLLLVYYWS

### Intracellular Domain

sp|P07766|153-207
(SEQ ID NO: 4)
KNRKAKAKPVTRGAGAGGRQRGQNKERPPPVPNPDYEPIRKGQRDLYS
GLNQRRI

An Example of a Homo sapiens CD3e Molecule (CD3E), mRNA is at NCBI Reference Sequence: GENBANK® Accession No. NM 000733.4

(SEQ ID NO: 5)
ATGCAGTCGGGCACTCACTGGAGAGTTCTGGGCCTCTGCCTCTTATCAGT
TGGCGTTTGGGGGCAAGATGGTAATGAAGAAATGGGTGGTATTACACAGA
CACCATATAAAGTCTCCATCTCTGGAACCACAGTAATATTGACATGCCCT
CAGTATCCTGGATCTGAAATACTATGGCAACACAATGATAAAAACATAGG
CGGTGATGAGGATGATAAAAACATAGGCAGTGATGAGGATCACCTGTCAC
TGAAGGAATTTTCAGAATTGGAGCAAAGTGGTTATTATGTCTGCTACCCC
AGAGGAAGCAAACCAGAAGATGCGAACTTTTATCTCTACCTGAGGGCAAG
AGTGTGTGAGAACTGCATGGAGATGGATGTGATGTCGGTGGCCACAATTG
TCATAGTGGACATCTGCATCACTGGGGGCTTGCTGCTGCTGGTTTACTAC
TGGAGCAAGAATAGAAAGGCCAAGGCCAAGCCTGTGACACGAGGAGCGGG
TGCTGGCGGCAGGCAAAGGGGACAAAACAAGGAGAGGCCACCACCTGTTC
CCAACCCAGACTATGAGCCCATCCGGAAAGGCCAGCGGGACCTGTATTCT
GGCCTGAATCAGAGACGCATCTGA

Examples of respective nucleic acid and amino acid CD3 epsilon sequences in their entirety are as follows (underlining refers to signal peptide sequence):

(SEQ ID NO: 37)
ATGCAGAGCGGCACCCACTGGAGAGTGCTGGGCCTGTGCCTGCTGAGCGT
GGGCGTGTGGGGCCAGGACGGCAACGAGGAGATGGGCGGCATCACCCAGA
CCCCCTACAAGGTGAGCATCAGCGGCACCACCGTGATCCTGACCTGCCCC
CAGTACCCCGGCAGCGAGATCCTGTGGCAGCACAACGACAAGAACATCGG
CGGCGACGAGGACGACAAGAACATCGGCAGCGACGAGGACCACCTGAGCC
TGAAGGAGTTCAGCGAGCTGGAGCAGAGCGGCTACTACGTGTGCTACCCC
AGAGGCAGCAAGCCCGAGGACGCCAACTTCTACCTGTACCTGAGAGCCAG
AGTGTGCGAGAACTGCATGGAGATGGACGTGATGAGCGTGGCCACCATCG
TGATCGTGGACATCTGCATCACCGGCGGCCTGCTGCTGCTGGTGTACTAC
TGGAGCAAGAACAGAAAGGCCAAGGCCAAGCCCGTGACCAGAGGCGCCGG
CGCCGGCGGCAGACAGAGAGGCCAGAACAAGGAGAGACCCCCCCCCGTGC
CCAACCCCGACTACGAGCCCATCAGAAAGGGCCAGAGAGACCTGTACAGC
GGCCTGAACCAGAGAAGAATC
(SEQ ID NO: 38)
MQSGTHWRVLGLCLLSVGVWGQDGNEEMGGITQTPYKVSISGTTVILTCP
QYPGSEILWQHNDKNIGGDEDDKNIGSDEDHLSLKEFSELEQSGYYVCYP
RGSKPEDANFYLYLRARVCENCMEMDVMSVATIVIVDICITGGLLLLVYY
WSKNRKAKAKPVTRGAGAGGRQRGQNKERPPPVPNPDYEPIRKGQRDLYS
GLNQRRI

### CD3 Delta (UniProtKB—P04234 (CD3D_HUMAN))

### Signal Peptide

(SEO ID NO: 6)
MEHSTFLSGLVLATLLSQVS

### Extracellular Domain

sp|P04234|22-105
(SEQ ID NO: 7)
FKIPIEELEDRVFVNCNTSITWVEGTVGTLLSDITRLDLGKRILDPRG
IYRCNGTDIYKDKESTVQVHYRMCQSCVELDPATVA

### Transmembrane Domain

sp|P04234|106-126
(SEQ ID NO: 8)
GIIVTDVIATLLLALGVFCFA

### Intracellular Domain

sp|P04234|127-171
(SEQ ID NO: 9)
GHETGRLSGAADTQALLRNDQVYQPLRDRDDAQYSHLGGNWARNK

Homo sapiens CD3d Molecule, Delta (CD3-TCR Complex), mRNA (cDNA Clone MGC:88324 IMAGE:30412345), Complete Cds GENBANK®: BC070321.1

(SEQ ID NO: 10)
ATGGAACATAGCACGTTTCTCTCTGGCCTGGTACTGGCTACCCTTCTCTC
GCAAGTGAGCCCCTTCAAGATACCTATAGAGGAACTTGAGGACAGAGTGT
TTGTGAATTGCAATACCAGCATCACATGGGTAGAGGGAACGGTGGGAACA
CTGCTCTCAGACATTACAAGACTGGACCTGGGAAAACGCATCCTGGACCC
ACGAGGAATATATAGGTGTAATGGGACAGATATATACAAGGACAAAGAAT
CTACCGTGCAAGTTCATTATCGAATGTGCCAGAGCTGTGTGGAGCTGGAT
CCAGCCACCGTGGCTGGCATCATTGTCACTGATGTCATTGCCACTCTGCT
CCTTGCTTTGGGAGTCTTCTGCTTTGCTGGACATGAGACTGGAAGGCTGT
CTGGGGCTGCCGACACACAAGCTCTGTTGAGGAATGACCAGGTCTATCAG
CCCCTCCGAGATCGAGATGATGCTCAGTACAGCCACCTTGGAGGAAACTG
GGCTCGGAACAAGTGA

Examples of respective nucleic acid and amino acid CD3 delta sequences in their entirety are as follows (underlining refers to signal peptide sequence):

(SEQ ID NO: 35)
ATGGAGCACAGCACCTTCCTGAGCGGCCTGGTGCTGGCCACCCTGCTGA
GCCAGGTGAGCCCCTTCAAGATCCCCATCGAGGAGCTGGAGGACAGAGT
GTTCGTGAACTGCAACACCAGCATCACCTGGGTGGAGGGCACCGTGGGC
ACCCTGCTGAGCGACATCACCAGACTGGACCTGGGCAAGAGAATCCTGG
ACCCCAGAGGCATCTACAGATGCAACGGCACCGACATCTACAAGGACAA
GGAGAGCACCGTGCAGGTGCACTACAGAATGTGCCAGAGCTGCGTGGAG
CTGGACCCCGCCACCGTGGCCGGCATCATCGTGACCGACGTGATCGCCA
CCCTGCTGCTGGCCCTGGGCGTGTTCTGCTTCGCCGGCCACGAGACCGG
CAGACTGAGCGGCGCCGCCGACACCCAGGCCCTGCTGAGAAACGACCAG
GTGTACCAGCCCCTGAGAGACAGAGACGACGCCCAGTACAGCCACCTGG
GCGGCAACTGGGCCAGAAACAAG
(SEQ ID NO: 36)
MEHSTFLSGLVLATLLSQVSPEKIPIEELEDRVFVNCNTSITWVEGTVG
TLLSDITRLDLGKRILDPRGIYRCNGTDIYKDKESTVQVHYRMCQSCVE
LDPATVAGIIVTDVIATLLLALGVFCFAGHETGRLSGAADTQALLRNDQ
VYQPLRDRDDAQYSHLGGNWARNK

### CD3 Gamma (T-Cell Surface Glycoprotein CD3 Gamma Chain Gene CD3G P09693) Signal Peptide

(SEQ ID NO: 11)
MEQGKGLAVL ILAIILLQGTLA

### Extracellular Domain

sp|P09693|23-116
(SEQ ID NO: 12)
QSIKGNHLVKVYDYQEDGSVLLTCDAEAKNITWFKDGKMIGFLTEDKK
KWNLGSNAKDPRGMYQCKGSQNKSKPLQVYYRMCQNCIELNAATIS

### Transmembrane Domain

sp|P09693|117-137
(SEQ ID NO: 13)
GFLFAEIVSIFVLAVGVYFIA

### Intracellular Domain

sp_P09693|138-182
(SEQ ID NO: 14)
GQDGVRQSRASDKQTLLPNDQLYQPLKDREDDQYSHLQGNQLRRN

Homo sapiens CD3g Molecule (CD3G), mRNA; NM_000073.3:81-629 Homo sapiens CD3g Molecule (CD3G), mRNA

(SEQ ID NO: 15)
ATGGAACAGGGGAAGGGCCTGGCTGTCCTCATCCTGGCTATCATTCTT
CTTCAAGGTACTTTGGCCCAGTCAATCAAAGGAAACCACTTGGTTAAG
GTGTATGACTATCAAGAAGATGGTTCGGTACTTCTGACTTGTGATGCA
GAAGCCAAAAATATCACATGGTTTAAAGATGGGAAGATGATCGGCTTC
CTAACTGAAGATAAAAAAAAATGGAATCTGGGAAGTAATGCCAAGGAC
CCTCGAGGGATGTATCAGTGTAAAGGATCACAGAACAAGTCAAAACCA
CTCCAAGTGTATTACAGAATGTGTCAGAACTGCATTGAACTAAATGCA
GCCACCATATCTGGCTTTCTCTTTGCTGAAATCGTCAGCATTTTCGTC
CTTGCTGTTGGGGTCTACTTCATTGCTGGACAGGATGGAGTTCGCCAG
TCGAGAGCTTCAGACAAGCAGACTCTGTTGCCCAATGACCAGCTCTAC
CAGCCCCTCAAGGATCGAGAAGATGACCAGTACAGCCACCTTCAAGGA
AACCAGTTGAGGAGGAATTGA

Examples of respective nucleic acid and amino acid CD3 gamma sequences in their entirety are as follows (underlining refers to signal peptide sequence):

(SEQ ID NO: 33)
ATGGAACAGGGGAAGGGCCTGGCTGTCCTCATCCTGGCTATCATTCTT
CTTCAAGGTACTTTGGCCCAGTCAATCAAAGGAAACCACTTGGTTAAG
GTGTATGACTATCAAGAAGATGGTTCGGTACTTCTGACTTGTGATGCA
GAAGCCAAAAATATCACATGGTTTAAAGATGGGAAGATGATCGGCTTC
CTAACTGAAGATAAAAAAAAATGGAATCTGGGAAGTAATGCCAAGGAC
CCTCGTGGGATGTATCAGTGTAAAGGATCACAGAACAAGTCAAAACCA
CTCCAAGTGTATTACAGAATGTGTCAGAACTGCATTGAACTAAATGCA
GCCACCATATCTGGCTTTCTCTTTGCTGAAATCGTCAGCATTTTCGTC
CTTGCTGTTGGGGTCTACTTCATTGCTGGACAGGATGGAGTTCGCCAG
TCGAGAGCTTCAGACAAGCAGACTCTGTTGCCCAATGACCAGCTCTAC
CAGCCCCTCAAGGATCGAGAAGATGACCAGTACAGCCACCTTCAAGGA
AACCAGTTGAGGAGGAAT
(SEQ ID NO: 34)
MEQGKGLAVLILAIILLQGTLAQSIKGNHLVKVYDYQEDGSVLLTCDA
EAKNITWFKDGKMIGFLTEDKKKWNLGSNAKDPRGMYQCKGSQNKSKP
LQVYYRMCQNCIELNAATISGFLFAEIVSIFVLAVGVYFIAGQDGVRQ
SRASDKQTLLPNDQLYQPLKDREDDQYSHLQGNQLRRN

### CD3 Zeta

### Signal Peptide

sp|P20963|SP
(SEQ ID NO: 16)
MKWKALFTAAILQAQLPITEA

### Extracellular Domain

sp|P20963|22-30 ECD
(SEQ ID NO: 17)
QSFGLLDPK

### Transmembrane Domain

sp|P20963|31-51 tmd
(SEQ ID NO: 18)
LCYLLDGILFIYGVILTALFL

### Intracellular Domain

sp|P20963|52-164 ICD
(SEQ ID NO: 19)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR

Examples of respective nucleic acid and amino acid CD3 zeta sequences in their entirety are as follows (underlining refers to signal peptide sequence):

(SEQ ID NO: 31)
ATGAAGTGGAAGGCGCTTTTCACCGCGGCCATCCTGCAGGCACAGTTGCC
GATTACAGAGGCACAGAGCTTTGGCCTGCTGGATCCCAAACTCTGCTACC
TGCTGGATGGAATCCTCTTCATCTATGGTGTCATTCTCACTGCCTTGTTC
CTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACG
ATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAG
GACACCTACGACGCCCTTCACATGCAGGCCCTGCCCCCTCGC
(SEQ ID NO: 32)
MKWKALFTAAILQAQLPITEAQSFGLLDPKLCYLLDGILFIYGVILTALF
LRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATK
DTYDALHMQALPPR

Homo sapiens CD247 Molecule (CD247; Also Referred to as CD3 Zeta), Transcript Variant 1, mRNA

### NCBI Reference Sequence: NM_198053.3

NM_198053.3:65-559 Homo sapiens CD247 Molecule (CD247), Transcript Variant 1, mRNA

(SEQ ID NO: 20)
ATGAAGTGGAAGGCGCTTTTCACCGCGGCCATCCTGCAGGCACAGTTGCC
GATTACAGAGGCACAGAGCTTTGGCCTGCTGGATCCCAAACTCTGCTACC
TGCTGGATGGAATCCTCTTCATCTATGGTGTCATTCTCACTGCCTTGTTC
CTGAGAGTGAAGTTCAGCAGGAGCGCAGACGCCCCCGCGTACCAGCAGGG
CCAGAACCAGCTCTATAACGAGCTCAATCTAGGACGAAGAGAGGAGTACG
ATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGA
TAAGATGGCGGAGGCCTACAGTGAGATTGGGATGAAAGGCGAGCGCCGGA
GGGGCAAGGGGCACGATGGCCTTTACCAGGGTCTCAGTACAGCCACCAAG
GACACCTACGACGCCCTTCACATGCAGGCCCTGCCCCCTCGCTAA

In specific embodiments, the NK cells are modified to express one of more of the TCRα chain, the TCRβ chain, the TCRγ chain, and the TCRδ chain, and any combination thereof may be utilized. In a specific case, the NK cells are modified to express the T-cell receptor (TCR) αβ chains or the TCR γδ chains. In certain cases, the NK cells are modified to express part or all of only the constant region of one of more of the TCRα chain, the TCRβ chain, the TCRγ chain, and the TCRδ chain. The NK cells may be modified to express part or all of only the constant region of the T-cell receptor (TCR) αβ chains or the TCR γδ chains. In cases wherein part of the constant region is utilized, the part of the constant region may be at least 50, 75, 100, 125, 150, 175, 200, 225, 250, 275, 300, 325, 350, 375, or 400 amino acids, including contiguous amino acids of any constant region. The part of the constant region may comprise at least 50, 55, 60, 65, 70, 75, 80, 85, 90, 91, 92, 93, 94, 95, 96, 97, 98, or 99% of the amino acids of a constant region, including contiguous amino acids of a constant region.

In specific cases, any sequences encompassed herein are utilized to modify the NK cells, although in other cases sequences that are related to these in identity are utilized. For example, related sequences that are at least 80, 85, 90, 95, 96, 97, 98, 99% identical to any sequence encompassed herein may be utilized in the disclosure.

Particular constructs for the expression of various TCR/CD3 proteins in the NK cells may be utilized, and in a variety of configurations. In specific cases, the NK cells may be transduced or transfected with one or more vectors to express any of the various proteins encompassed herein, including at least any one or more components of the TCR/CD3 complex. In specific cases, the one or more vectors themselves may or may not be multicistronic by being able ultimately to produce more than one separate polypeptide. In cases wherein one or more multicistronic vectors are employed, they may utilize one or more internal ribosome entry sites (IRES) and/or one or more 2A self-cleaving peptide sites. In cases wherein one or more 2A sequences are utilized, the following may be used, where GSG is an optional linker:

T2A
(SEQ ID NO: 21)
(GSG) EGRGSLLTCGDVEENPGP
P2A
(SEQ ID NO: 22)
(GSG) ATNFSLLKQAGDVEENPGP
E2A
(SEQ ID NO: 23)
(GSG) QCTNYALLKLAGDVESNPGP
F2A
(SEQ ID NO: 24)
(GSG) VKQTLNFDLLKLAGDVESNPGP

In situations wherein multiple protein components are expressed from a multicistronic vector, the order in a 5′ to 3′ direction on the polynucleotide vector may be of any order, although in alternative cases they are present on the vector in a particular order. A multicistronic vector may express multiple components of the CD3 receptor complex and no other heterologous protein, or the multicistronic vector may express multiple components of the CD3 receptor complex and one or more other heterologous proteins. A multicistronic vector may express multiple components of the TCR receptor complex and no other heterologous protein, or the multicistronic vector may express multiple components of the TCR receptor complex and one or more other heterologous proteins. A multicistronic vector may or may not express one or more multiple components of the TCR receptor complex and one or more multiple components of the CD3 complex. In a specific embodiment, a multicistronic vector includes one or multiple components of the CD3 receptor complex and one or more heterologous proteins, such as a cytokine and an engineered antigen receptor, such as a CAR.

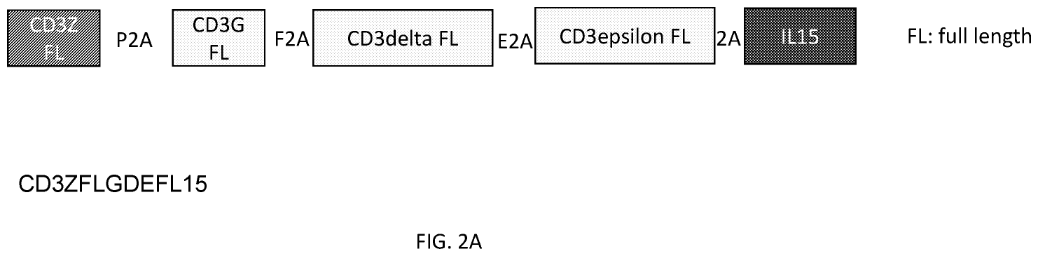

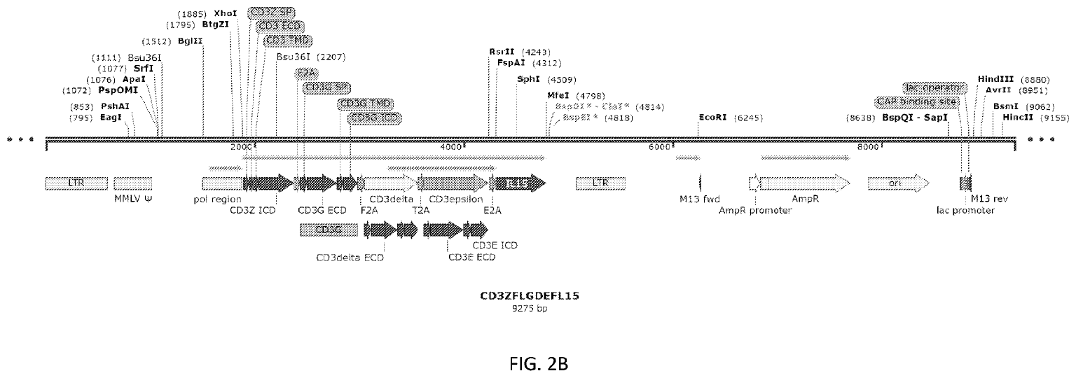

There is an example in FIG. 2A of a multicistronic vector in which full lengths of CD3ε, CD3δ, CD3γ, and CD3ζ are present and separated by the same or different 2A self-cleaving peptide sites. As further noted in the plasmid map of FIG. 2B, a multicistronic vector may include the signal peptide, extracellular domain, transmembrane domain, and intracellular domain of each of CD3ε, CD3δ, CD3γ, and CD3ζ.

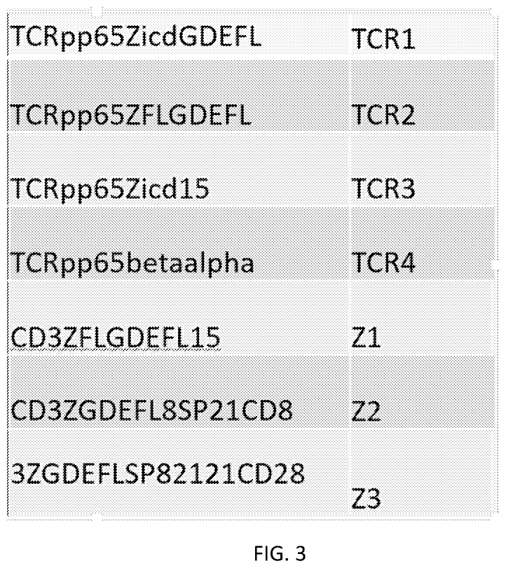

FIG. 3 provides a table showing examples of various TCR expression constructs for engineering of TCR-expressing NK cells. In particular embodiments of the disclosure, CD3 receptor components and TCR receptor components are expressed from different vectors in the NK cells. In any case, the vector(s) may express a TCR directed against a particular antigen, such as a cancer antigen or a viral antigen. The TCR may or may not comprise at least part of CD3ζ, including the intracellular domain of CD3ζ, in addition to the NK cells also expressing CD3ζ as a separate molecule from the TCR and as part of the CD3 receptor complex. Likewise, a CAR may or may not comprise at least part of CD3ζ, including the intracellular domain of CD3ζ, in addition to the NK cells also expressing CD3ζ as a separate molecule from the TCR and as part of the CD3 receptor complex.

In specific embodiments, a TCR of the modified NK cells is utilized not necessarily as a therapeutic aspect for the cells but as a structural support or scaffold to facilitate function or enhanced function of the CD3 receptor complex. That is, the TCR may be any TCR and may not be utilized for its ability to target a particularly desired antigen. In such cases, and as an example, a TCR that targets a viral antigen may be employed for NK cells that will be used for cancers that are not necessarily related to that particular virus. In other cases, the TCR is selected for the ability to target a particular cancer antigen. Examples of antigens to which the TCR may be directed are provided elsewhere herein.

In FIG. 3, the following examples of constructs are noted:

TCR1: refers to TCRpp65 (the TCR against the HLA-A2 restricted CMVpp65) linked to the intracellular CD3zeta domain and full length CD3 gamma, full length CD3 delta, and full length CD3 epsilon, and the construct may also be referred to as TCRpp65ZicdGDEFL that may comprise the following sequence:

(SEQ ID NO: 39)
MLEGVTQTPKFQVLKTGQSMTLQCAQDMNHEYMSWYRQDPGMGLRLIH
YSVGAGITDQGEVPNGYNVSRSTTEDFPLRLLSAAPSQTSVYFCASSP
VTGGIYGYTFGSGTRLTVVEDLNKVFPPEVAVFEPSEAEISHTQKATL
VCLATGFFPDHVELSWWVNGKEVHSGVSTDPQPLKEQPALNDSRYCLS
SRLRVSATFWQNPRNHFRCQVQFYGLSENDEWTQDRAKPVTQIVSAEA
WGRADRVKFSRSADAPAYQQGQNQLYNELNLGRREEYDVLDKRRGRDP
EMGGKPQRRKNPQEGLYNELQKDKMAEAYSEIGMKGERRRGKGHDGLY
QGLSTATKDTYDALHMQALPPRATNFSLLKQAGDVEENPGPMILNVEQ
SPQSLHVQEGDSTNFTCSFPSSNFYALHWYRWETAKSPEALFVMTLNG
DEKKKGRISATLNTKEGYSYLYIKGSQPEDSATYLCARNTGNQFYFGT
GTSLTVIPNIQNPDPAVYQLRDSKSSDKSVCLFTDFDSQTNVSQSKDS
DAYITDKTVLDMRSMDFKSNSAVAWSNKSDFACANAFNNSIIPEDTFF
PSPESSRVKFSRSADAPAYQQGQNQLYNELNLGRREEYDVLDKRRGRD
PEMGGKPQRRKNPQEGLYNELQKDKMAEAYSEIGMKGERRRGKGHDGL
YQGLSTATKDTYDALHMQALPPRQCTNYALLKLAGDVESNPGPMEQGK
GLAVLILAIILLQGTLAQSIKGNHLVKVYDYQEDGSVLLTCDAEAKNI
TWFKDGKMIGFLTEDKKKWNLGSNAKDPRGMYQCKGSQNKSKPLQVYY
RMCQNCIELNAATISGFLFAEIVSIFVLAVGVYFIAGQDGVRQSRASD
KQTLLPNDQLYQPLKDREDDQYSHLQGNQLRRNVKQTLNFDLLKLAGD
VESNPGPMEHSTFLSGLVLATLLSQVSPFKIPIEELEDRVFVNCNTSI
TWVEGTVGTLLSDITRLDLGKRILDPRGIYRCNGTDIYKDKESTVQVH
YRMCQSCVELDPATVAGIIVTDVIATLLLALGVFCFAGHETGRLSGAA
DTQALLRNDQVYQPLRDRDDAQYSHLGGNWARNKEGRGSLLTCGDVEE
NPGPMQSGTHWRVLGLCLLSVGVWGQDGNEEMGGITQTPYKVSISGTT
VILTCPQYPGSEILWQHNDKNIGGDEDDKNIGSDEDHLSLKEFSELEQ
SGYYVCYPRGSKPEDANFYLYLRARVCENCMEMDVMSVATIVIVDICI
TGGLLLLVYYWSKNRKAKAKPVTRGAGAGGRQRGQNKERPPPVPNPDY
EPIRKGQRDLYSGLNQRRIGPQCTNYALLKLAGDVESNPGPMRISKPH
LRSISIQCYLCLLLNSHELTEAGIHVFILGCFSAGLPKTEANWVNVIS
DLKKIEDLIQSMHIDATLYTESDVHPSCKVTAMKCFLLELQVISLESG
DASIHDTVENLIILANNSLSSNGNVTESGCKECEELEEKNIKEFLQSF
VHIVQMFINTS*

In TCRpp65ZicdGDEFL, the corresponding component sequences are as follows, although these particular sequences or others may be utilized in this and/or other constructs:

### TCRb-Extracellular Domain:

(SEQ ID NO: 40)
MLEGVTQTPKFQVLKTGQSMTLQCAQDMNHEYMSWYRQDPGMGLRLIHYS
VGAGITDQGEVPNGYNVSRSTTEDFPLRLLSAAPSQTSVYFCASSPVTGG
IYGYTFGSGTRLTVVEDLNKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRAD
(SEQ ID NO: 41)
ATGCTCGAGGGAGTGACCCAGACCCCCAAGTTCCAGGTGCTGAAGACCGG
ACAGAGCATGACCCTGCAGTGCGCCCAGGACATGAACCACGAGTACATGA
GCTGGTACCGGCAGGACCCCGGAATGGGACTGCGGCTGATCCACTACAGC
GTGGGAGCCGGAATCACCGACCAGGGAGAGGTGCCCAACGGATACAACGT
GAGCCGGAGCACCACCGAGGACTTCCCCCTGCGGCTGCTGAGCGCCGCCC
CCAGCCAGACCAGCGTGTACTTCTGCGCCAGCAGCCCCGTGACCGGAGGA
ATCTACGGATACACCTTCGGAAGCGGAACCCGGCTGACCGTGGTGGAGGA
CCTGAACAAGGTGTTCCCCCCCGAGGTGGCCGTGTTCGAGCCCAGCGAGG
CCGAGATCAGCCACACCCAGAAGGCCACCCTGGTGTGCCTGGCCACCGGA
TTCTTCCCCGACCACGTGGAGCTGAGCTGGTGGGTGAACGGAAAGGAGGT
GCACAGCGGAGTGAGCACCGACCCCCAGCCCCTGAAGGAGCAGCCCGCCC
TGAACGACAGCCGGTACTGCCTGAGCAGCCGGCTGCGGGTGAGCGCCACC
TTCTGGCAGAACCCCCGGAACCACTTCCGGTGCCAGGTGCAGTTCTACGG
ACTGAGCGAGAACGACGAGTGGACCCAGGACCGGGCCAAGCCCGTGACCC
AGATCGTGAGCGCCGAGGCCTGGGGACGGGCCGAC

### CD3 Zeta Intracellular Domain (Z-ICD):

(SEQ ID NO: 42)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRATNFSLLKQAGDVEENPGP
(where the P2A sequence is at the C-terminus)
(SEQ ID NO: 43)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCgccaccaactt
ctccctgctgaagcaggccggcgacgtggaggagaaccccggcccc
(where the lower case sequence is the P2A sequence)

### TCRa-Extracellular Domain:

(SEQ ID NO: 44)
MILNVEQSPQSLHVQEGDSTNFTCSFPSSNFYALHWYRWETAKSPEALFV
MTLNGDEKKKGRISATLNTKEGYSYLYIKGSQPEDSATYLCARNTGNQFY
FGTGTSLTVIPNIQNPDPAVYQLRDSKSSDKSVCLFTDFDSQTNVSQSKD
SDAYITDKTVLDMRSMDFKSNSAVAWSNKSDFACANAFNNSIIPEDTFFP
SPESS
(SEQ ID NO: 45)
ATGATCCTGAACGTGGAGCAGAGCCCCCAGAGCCTGCACGTGCAGGAGGG
AGACAGCACCAACTTCACCTGCAGCTTCCCCAGCAGCAACTTCTACGCCC
TGCACTGGTACCGGTGGGAGACCGCCAAGAGCCCCGAGGCCCTGTTCGTG
ATGACCCTGAACGGAGACGAGAAGAAGAAGGGACGGATCAGCGCCACCCT
GAACACCAAGGAGGGATACAGCTACCTGTACATCAAGGGAAGCCAGCCCG
AGGACAGCGCCACCTACCTGTGCGCCCGGAACACCGGAAACCAGTTCTAC
TTCGGAACCGGAACCAGCCTGACCGTGATCCCCAACATCCAGAACCCCGA
CCCCGCCGTGTACCAGCTGCGGGACAGCAAGAGCAGCGACAAGAGCGTGT
GCCTGTTCACCGACTTCGACAGCCAGACCAACGTGAGCCAGAGCAAGGAC
AGCGACGCCTACATCACCGACAAGACCGTGCTGGACATGCGGAGCATGGA
CTTCAAGAGCAACAGCGCCGTGGCCTGGAGCAACAAGAGCGACTTCGCCT
GCGCCAACGCCTTCAACAACAGCATCATCCCCGAGGACACCTTCTTCCCC
AGCCCCGAGAGCAGC

### CD3 Gamma Delta Epsilon (CD3GDE):

(SEQ ID NO: 46)
MEQGKGLAVLILAIILLQGTLAQSIKGNHLVKVYDYQEDGSVLLTCDAEA
KNITWFKDGKMIGFLTEDKKKWNLGSNAKDPRGMYQCKGSQNKSKPLQVY
YRMCQNCIELNAATISGELFAEIVSIFVLAVGVYFIAGQDGVRQSRASDK
QTLLPNDQLYQPLKDREDDQYSHLQGNQLRRNVKQTLNFDLLKLAGDVES
NPGPMEHSTFLSGLVLATLLSQVSPFKIPIEELEDRVFVNCNTSITWVEG
TVGTLLSDITRLDLGKRILDPRGIYRCNGTDIYKDKESTVQVHYRMCQSC
VELDPATVAGIIVTDVIATLLLALGVFCFAGHETGRLSGAADTQALLRND
QVYQPLRDRDDAQYSHLGGNWARNKEGRGSLLTCGDVEENPGPMQSGTHW
RVLGLCLLSVGVWGQDGNEEMGGITQTPYKVSISGTTVILTCPQYPGSEI
LWQHNDKNIGGDEDDKNIGSDEDHLSLKEFSELEQSGYYVCYPRGSKPED
ANFYLYLRARVCENCMEMDVMSVATIVIVDICITGGLLLLVYYWSKNRKA
KAKPVTRGAGAGGRQRGQNKERPPPVPNPDYEPIRKGQRDLYSGLNQRRI
GPQCTNYALLKLAGDVESNPGP
(where the E2A sequence is at the C-terminus)
(SEQ ID NO: 47)
ATGGAACAGGGGAAGGGCCTGGCTGTCCTCATCCTGGCTATCATTCTTCT
TCAAGGTACTTTGGCCCAGTCAATCAAAGGAAACCACTTGGTTAAGGTGT
ATGACTATCAAGAAGATGGTTCGGTACTTCTGACTTGTGATGCAGAAGCC
AAAAATATCACATGGTTTAAAGATGGGAAGATGATCGGCTTCCTAACTGA
AGATAAAAAAAAATGGAATCTGGGAAGTAATGCCAAGGACCCTCGTGGGA
TGTATCAGTGTAAAGGATCACAGAACAAGTCAAAACCACTCCAAGTGTAT
TACAGAATGTGTCAGAACTGCATTGAACTAAATGCAGCCACCATATCTGG
CTTTCTCTTTGCTGAAATCGTCAGCATTTTCGTCCTTGCTGTTGGGGTCT
ACTTCATTGCTGGACAGGATGGAGTTCGCCAGTCGAGAGCTTCAGACAAG
CAGACTCTGTTGCCCAATGACCAGCTCTACCAGCCCCTCAAGGATCGAGA
AGATGACCAGTACAGCCACCTTCAAGGAAACCAGTTGAGGAGGAATGTGA
AGCAGACCCTGAACTTCGACCTGCTGAAGCTGGCCGGCGACGTGGAGAGC
AACCCCGGCCCCATGGAGCACAGCACCTTCCTGAGCGGCCTGGTGCTGGC
CACCCTGCTGAGCCAGGTGAGCCCCTTCAAGATCCCCATCGAGGAGCTGG
AGGACAGAGTGTTCGTGAACTGCAACACCAGCATCACCTGGGTGGAGGGC
ACCGTGGGCACCCTGCTGAGCGACATCACCAGACTGGACCTGGGCAAGAG
AATCCTGGACCCCAGAGGCATCTACAGATGCAACGGCACCGACATCTACA
AGGACAAGGAGAGCACCGTGCAGGTGCACTACAGAATGTGCCAGAGCTGC
GTGGAGCTGGACCCCGCCACCGTGGCCGGCATCATCGTGACCGACGTGAT
CGCCACCCTGCTGCTGGCCCTGGGCGTGTTCTGCTTCGCCGGCCACGAGA
CCGGCAGACTGAGCGGCGCCGCCGACACCCAGGCCCTGCTGAGAAACGAC
CAGGTGTACCAGCCCCTGAGAGACAGAGACGACGCCCAGTACAGCCACCT
GGGCGGCAACTGGGCCAGAAACAAGGAGGGCAGAGGCAGCCTGCTGACCT
GCGGCGACGTGGAGGAGAACCCCGGCCCCATGCAGAGCGGCACCCACTGG
AGAGTGCTGGGCCTGTGCCTGCTGAGCGTGGGCGTGTGGGGCCAGGACGG
CAACGAGGAGATGGGCGGCATCACCCAGACCCCCTACAAGGTGAGCATCA
GCGGCACCACCGTGATCCTGACCTGCCCCCAGTACCCCGGCAGCGAGATC
CTGTGGCAGCACAACGACAAGAACATCGGCGGCGACGAGGACGACAAGAA
CATCGGCAGCGACGAGGACCACCTGAGCCTGAAGGAGTTCAGCGAGCTGG
AGCAGAGCGGCTACTACGTGTGCTACCCCAGAGGCAGCAAGCCCGAGGAC
GCCAACTTCTACCTGTACCTGAGAGCCAGAGTGTGCGAGAACTGCATGGA
GATGGACGTGATGAGCGTGGCCACCATCGTGATCGTGGACATCTGCATCA
CCGGCGGCCTGCTGCTGCTGGTGTACTACTGGAGCAAGAACAGAAAGGCC
AAGGCCAAGCCCGTGACCAGAGGCGCCGGCGCCGGCGGCAGACAGAGAGG
CCAGAACAAGGAGAGACCCCCCCCCGTGCCCAACCCCGACTACGAGCCCA
TCAGAAAGGGCCAGAGAGACCTGTACAGCGGCCTGAACCAGAGAAGAATC
GGACCGcagtgtactaattatgctctcttgaaattggctggagatgttga
gagcaatcccgggccc
(where the lower case is the E2A sequence)

### IL-15:

(SEQ ID NO: 48)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS*
(SEQ ID NO: 49)
ATGCGCATTAGCAAGCCCCACCTGCGGAGCATCAGCATCCAGTGCTACCT
GTGCCTGCTGCTGAACAGCCACTTCCTGACCGAGGCCGGCATCCACGTGT
TCATCCTGGGCTGCTTCAGCGCCGGACTGCCCAAGACCGAGGCCAACTGG
GTGAACGTGATCAGCGACCTGAAGAAGATCGAGGACCTGATCCAGAGCAT
GCACATCGACGCCACCCTGTACACCGAGAGCGACGTGCACCCCAGCTGCA
AGGTGACCGCCATGAAGTGCTTTCTGCTGGAACTGCAGGTGATCAGCCTG
GAAAGCGGCGACGCCAGCATCCACGACACCGTGGAGAACCTGATCATCCT
GGCCAACAACAGCCTGAGCAGCAACGGCAACGTGACCGAGAGCGGCTGCA
AAGAGTGCGAGGAACTGGAAGAGAAGAACATCAAAGAGTTTCTGCAGAGC
TTCGTGCACATCGTGCAGATGTTCATCAACACCAGC

TCR2: refers to TCRpp65 linked to full length CD3zeta, full length CD3 gamma, full length CD3 delta, and full length CD3 epsilon; it lacks IL-15. Representative sequences are as follows:

(SEQ ID NO: 50)
CTCGAGGGAGTGACCCAGACCCCCAAGTTCCAGGTGCTGAAGACCGGACA
GAGCATGACCCTGCAGTGCGCCCAGGACATGAACCACGAGTACATGAGCT
GGTACCGGCAGGACCCCGGAATGGGACTGCGGCTGATCCACTACAGCGTG
GGAGCCGGAATCACCGACCAGGGAGAGGTGCCCAACGGATACAACGTGAG
CCGGAGCACCACCGAGGACTTCCCCCTGCGGCTGCTGAGCGCCGCCCCCA
GCCAGACCAGCGTGTACTTCTGCGCCAGCAGCCCCGTGACCGGAGGAATC
TACGGATACACCTTCGGAAGCGGAACCCGGCTGACCGTGGTGGAGGACCT
GAACAAGGTGTTCCCCCCCGAGGTGGCCGTGTTCGAGCCCAGCGAGGCCG
AGATCAGCCACACCCAGAAGGCCACCCTGGTGTGCCTGGCCACCGGATTC
TTCCCCGACCACGTGGAGCTGAGCTGGTGGGTGAACGGAAAGGAGGTGCA
CAGCGGAGTGAGCACCGACCCCCAGCCCCTGAAGGAGCAGCCCGCCCTGA
ACGACAGCCGGTACTGCCTGAGCAGCCGGCTGCGGGTGAGCGCCACCTTC
TGGCAGAACCCCCGGAACCACTTCCGGTGCCAGGTGCAGTTCTACGGACT
GAGCGAGAACGACGAGTGGACCCAGGACCGGGCCAAGCCCGTGACCCAGA
TCGTGAGCGCCGAGGCCTGGGGACGGGCCGACGCCACCAACTTCAGCCTG
CTGAAGCAGGCCGGCGACGTGGAGGAGAACCCCGGCCCCATGATCCTGAA
CGTGGAGCAGAGCCCCCAGAGCCTGCACGTGCAGGAGGGAGACAGCACCA
ACTTCACCTGCAGCTTCCCCAGCAGCAACTTCTACGCCCTGCACTGGTAC
CGGTGGGAGACCGCCAAGAGCCCCGAGGCCCTGTTCGTGATGACCCTGAA
CGGAGACGAGAAGAAGAAGGGACGGATCAGCGCCACCCTGAACACCAAGG
AGGGATACAGCTACCTGTACATCAAGGGAAGCCAGCCCGAGGACAGCGCC
ACCTACCTGTGCGCCCGGAACACCGGAAACCAGTTCTACTTCGGAACCGG
AACCAGCCTGACCGTGATCCCCAACATCCAGAACCCCGACCCCGCCGTGT
ACCAGCTGCGGGACAGCAAGAGCAGCGACAAGAGCGTGTGCCTGTTCACC
GACTTCGACAGCCAGACCAACGTGAGCCAGAGCAAGGACAGCGACGCCTA
CATCACCGACAAGACCGTGCTGGACATGCGGAGCATGGACTTCAAGAGCA
ACAGCGCCGTGGCCTGGAGCAACAAGAGCGACTTCGCCTGCGCCAACGCC
TTCAACAACAGCATCATCCCCGAGGACACCTTCTTCCCCAGCCCCGAGAG
CAGCGAGGGCAGAGGCAGCCTGCTGACCTGCGGCGACGTGGAGGAGAACC
CCGGCCCCATGAAGTGGAAGGCGCTTTTCACCGCGGCCATCCTGCAGGCA
CAGTTGCCGATTACAGAGGCACAGAGCTTTGGCCTGCTGGATCCCAAACT
CTGCTACCTGCTGGATGGAATCCTCTTCATCTATGGTGTCATTCTCACTG
CCTTGTTCCTGAGAGTGAAGTTCAGCAGGAGCGCAGACGCCCCCGCGTAC
CAGCAGGGCCAGAACCAGCTCTATAACGAGCTCAATCTAGGACGAAGAGA
GGAGTACGATGTTTTGGACAAGAGACGTGGCCGGGACCCTGAGATGGGGG
GAAAGCCGCAGAGAAGGAAGAACCCTCAGGAAGGCCTGTACAATGAACTG
CAGAAAGATAAGATGGCGGAGGCCTACAGTGAGATTGGGATGAAAGGCGA
GCGCCGGAGGGGCAAGGGGCACGATGGCCTTTACCAGGGTCTCAGTACAG
CCACCAAGGACACCTACGACGCCCTTCACATGCAGGCCCTGCCCCCTCGC
CAGTGCACCAACTACGCCCTGCTGAAGCTGGCCGGCGACGTGGAGAGCAA
CCCCGGCCCCATGGAACAGGGGAAGGGCCTGGCTGTCCTCATCCTGGCTA
TCATTCTTCTTCAAGGTACTTTGGCCCAGTCAATCAAAGGAAACCACTTG
GTTAAGGTGTATGACTATCAAGAAGATGGTTCGGTACTTCTGACTTGTGA
TGCAGAAGCCAAAAATATCACATGGTTTAAAGATGGGAAGATGATCGGCT
TCCTAACTGAAGATAAAAAAAAATGGAATCTGGGAAGTAATGCCAAGGAC
CCTCGTGGGATGTATCAGTGTAAAGGATCACAGAACAAGTCAAAACCACT
CCAAGTGTATTACAGAATGTGTCAGAACTGCATTGAACTAAATGCAGCCA
CCATATCTGGCTTTCTCTTTGCTGAAATCGTCAGCATTTTCGTCCTTGCT
GTTGGGGTCTACTTCATTGCTGGACAGGATGGAGTTCGCCAGTCGAGAGC
TTCAGACAAGCAGACTCTGTTGCCCAATGACCAGCTCTACCAGCCCCTCA
AGGATCGAGAAGATGACCAGTACAGCCACCTTCAAGGAAACCAGTTGAGG
AGGAATGTGAAGCAGACCCTGAACTTCGACCTGCTGAAGCTGGCCGGCGA
CGTGGAGAGCAACCCCGGCCCCATGGAGCACAGCACCTTCCTGAGCGGCC
TGGTGCTGGCCACCCTGCTGAGCCAGGTGAGCCCCTTCAAGATCCCCATC
GAGGAGCTGGAGGACAGAGTGTTCGTGAACTGCAACACCAGCATCACCTG
GGTGGAGGGCACCGTGGGCACCCTGCTGAGCGACATCACCAGACTGGACC
TGGGCAAGAGAATCCTGGACCCCAGAGGCATCTACAGATGCAACGGCACC
GACATCTACAAGGACAAGGAGAGCACCGTGCAGGTGCACTACAGAATGTG
CCAGAGCTGCGTGGAGCTGGACCCCGCCACCGTGGCCGGCATCATCGTGA
CCGACGTGATCGCCACCCTGCTGCTGGCCCTGGGCGTGTTCTGCTTCGCC
GGCCACGAGACCGGCAGACTGAGCGGCGCCGCCGACACCCAGGCCCTGCT
GAGAAACGACCAGGTGTACCAGCCCCTGAGAGACAGAGACGACGCCCAGT
ACAGCCACCTGGGCGGCAACTGGGCCAGAAACAAGGAGGGCAGAGGCAGC
CTGCTGACCTGCGGCGACGTGGAGGAGAACCCCGGCCCCATGCAGAGCGG
CACCCACTGGAGAGTGCTGGGCCTGTGCCTGCTGAGCGTGGGCGTGTGGG
GCCAGGACGGCAACGAGGAGATGGGCGGCATCACCCAGACCCCCTACAAG
GTGAGCATCAGCGGCACCACCGTGATCCTGACCTGCCCCCAGTACCCCGG
CAGCGAGATCCTGTGGCAGCACAACGACAAGAACATCGGCGGCGACGAGG
ACGACAAGAACATCGGCAGCGACGAGGACCACCTGAGCCTGAAGGAGTTC
AGCGAGCTGGAGCAGAGCGGCTACTACGTGTGCTACCCCAGAGGCAGCAA
GCCCGAGGACGCCAACTTCTACCTGTACCTGAGAGCCAGAGTGTGCGAGA
ACTGCATGGAGATGGACGTGATGAGCGTGGCCACCATCGTGATCGTGGAC
ATCTGCATCACCGGCGGCCTGCTGCTGCTGGTGTACTACTGGAGCAAGAA
CAGAAAGGCCAAGGCCAAGCCCGTGACCAGAGGCGCCGGCGCCGGCGGCA
GACAGAGAGGCCAGAACAAGGAGAGACCCCCCCCCGTGCCCAACCCCGAC
TACGAGCCCATCAGAAAGGGCCAGAGAGACCTGTACAGCGGCCTGAACCA
GAGAAGAATCGGACCG
(SEQ ID NO: 51)
LEGVTQTPKFQVLKTGQSMTLQCAQDMNHEYMSWYRQDPGMGLRLIHYSV
GAGITDQGEVPNGYNVSRSTTEDFPLRLLSAAPSQTSVYFCASSPVTGGI
YGYTFGSGTRLTVVEDLNKVFPPEVAVFEPSEAEISHTQKATLVCLATGF
FPDHVELSWWVNGKEVHSGVSTDPQPLKEQPALNDSRYCLSSRLRVSATF
WQNPRNHFRCQVQFYGLSENDEWTQDRAKPVTQIVSAEAWGRADATNFSL
LKQAGDVEENPGPMILNVEQSPQSLHVQEGDSTNFTCSFPSSNFYALHWY
RWETAKSPEALFVMTLNGDEKKKGRISATLNTKEGYSYLYIKGSQPEDSA
TYLCARNTGNQFYFGTGTSLTVIPNIQNPDPAVYQLRDSKSSDKSVCLFT
DFDSQTNVSQSKDSDAYITDKTVLDMRSMDFKSNSAVAWSNKSDFACANA
FNNSIIPEDTFFPSPESSEGRGSLLTCGDVEENPGPMKWKALFTAAILQA
QLPITEAQSFGLLDPKLCYLLDGILFIYGVILTALFLRVKFSRSADAPAY
QQGQNQLYNELNLGRREEYDVLDKRRGRDPEMGGKPQRRKNPQEGLYNEL
QKDKMAEAYSEIGMKGERRRGKGHDGLYQGLSTATKDTYDALHMQALPPR
QCTNYALLKLAGDVESNPGPMEQGKGLAVLILAIILLQGTLAQSIKGNHL
VKVYDYQEDGSVLLTCDAEAKNITWFKDGKMIGFLTEDKKKWNLGSNAKD
PRGMYQCKGSQNKSKPLQVYYRMCQNCIELNAATISGFLFAEIVSIFVLA
VGVYFIAGQDGVRQSRASDKQTLLPNDQLYQPLKDREDDQYSHLQGNQLR
RNVKQTLNFDLLKLAGDVESNPGPMEHSTFLSGLVLATLLSQVSPFKIPI
EELEDRVFVNCNTSITWVEGTVGTLLSDITRLDLGKRILDPRGIYRCNGT
DIYKDKESTVQVHYRMCQSCVELDPATVAGIIVTDVIATLLLALGVFCFA
GHETGRLSGAADTQALLRNDQVYQPLRDRDDAQYSHLGGNWARNKEGRGS
LLTCGDVEENPGPMQSGTHWRVLGLCLLSVGVWGQDGNEEMGGITQTPYK
VSISGTTVILTCPQYPGSEILWQHNDKNIGGDEDDKNIGSDEDHLSLKEF
SELEQSGYYVCYPRGSKPEDANFYLYLRARVCENCMEMDVMSVATIVIVD
ICITGGLLLLVYYWSKNRKAKAKPVTRGAGAGGRQRGQNKERPPPVPNPD
YEPIRKGQRDLYSGLNQRRIGP

TCR3: refers to TCRpp65 linked to the intracellular CD3z domain and IL-15, and it may also be referred to as TCRpp65Zicd15, with a representative sequence as follows:

(SEQ ID NO: 52)
MLEGVTQTPKFQVLKTGQSMTLQCAQDMNHEYMSWYRQDPGMGLRLIHYS
VGAGITDQGEVPNGYNVSRSTTEDFPLRLLSAAPSQTSVYFCASSPVTGG
IYGYTFGSGTRLTVVEDLNKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRADRVKFS
RSADAPAYQQGQNQLYNELNLGRREEYDVLDKRRGRDPEMGGKPQRRKNP
QEGLYNELQKDKMAEAYSEIGMKGERRRGKGHDGLYQGLSTATKDTYDAL
HMQALPPRATNFSLLKQAGDVEENPGPMILNVEQSPQSLHVQEGDSTNFT
CSFPSSNFYALHWYRWETAKSPEALFVMTLNGDEKKKGRISATLNTKEGY
SYLYIKGSQPEDSATYLCARNTGNQFYFGTGTSLTVIPNIQNPDPAVYQL
RDSKSSDKSVCLFTDFDSQTNVSQSKDSDAYITDKTVLDMRSMDFKSNSA
VAWSNKSDFACANAFNNSIIPEDTFFPSPESSRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRPGPQC
TNYALLKLAGDVESNPGPMRISKPHLRSISIQCYLCLLLNSHFLTEAGIH
VFILGCFSAGLPKTEANWVNVISDLKKIEDLIQSMHIDATLYTESDVHPS
CKVTAMKCFLLELQVISLESGDASIHDTVENLIILANNSLSSNGNVTESG
CKECEELEEKNIKEFLQSFVHIVQMFINTS*

In TCRpp65Zicd15, the corresponding component sequences are as follows, although these particular sequences or others may be utilized in this and/or other constructs:

### TCRb-Extracellular Domain:

(SEQ ID NO: 40)
MLEGVTQTPKFQVLKTGQSMTLQCAQDMNHEYMSWYRQDPGMGLRLIHYS
VGAGITDQGEVPNGYNVSRSTTEDFPLRLLSAAPSQTSVYFCASSPVTGG
IYGYTFGSGTRLTVVEDLNKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRAD
(SEQ ID NO: 41)
ATGCTCGAGGGAGTGACCCAGACCCCCAAGTTCCAGGTGCTGAAGACCGG
ACAGAGCATGACCCTGCAGTGCGCCCAGGACATGAACCACGAGTACATGA
GCTGGTACCGGCAGGACCCCGGAATGGGACTGCGGCTGATCCACTACAGC
GTGGGAGCCGGAATCACCGACCAGGGAGAGGTGCCCAACGGATACAACGT
GAGCCGGAGCACCACCGAGGACTTCCCCCTGCGGCTGCTGAGCGCCGCCC
CCAGCCAGACCAGCGTGTACTTCTGCGCCAGCAGCCCCGTGACCGGAGGA
ATCTACGGATACACCTTCGGAAGCGGAACCCGGCTGACCGTGGTGGAGGA
CCTGAACAAGGTGTTCCCCCCCGAGGTGGCCGTGTTCGAGCCCAGCGAGG
CCGAGATCAGCCACACCCAGAAGGCCACCCTGGTGTGCCTGGCCACCGGA
TTCTTCCCCGACCACGTGGAGCTGAGCTGGTGGGTGAACGGAAAGGAGGT
GCACAGCGGAGTGAGCACCGACCCCCAGCCCCTGAAGGAGCAGCCCGCCC
TGAACGACAGCCGGTACTGCCTGAGCAGCCGGCTGCGGGTGAGCGCCACC
TTCTGGCAGAACCCCCGGAACCACTTCCGGTGCCAGGTGCAGTTCTACGG
ACTGAGCGAGAACGACGAGTGGACCCAGGACCGGGCCAAGCCCGTGACCC
AGATCGTGAGCGCCGAGGCCTGGGGACGGGCCGAC

### CD3 Zeta Intracellular Domain (Z-ICD):

(SEQ ID NO: 42)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRATNFSLLKQAGDVEENPGP
(where P2A sequence is at the C-terminus)
(SEQ ID NO: 43)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCgccaccaactt
ctccctgctgaagcaggccggcgacgtggaggagaaccccggcccc
(where the lowercase sequence is P2A sequence)

### TCRa-Extracellular Domain:

(SEQ ID NO: 44)
MILNVEQSPQSLHVQEGDSTNFTCSFPSSNFYALHWYRWETAKSPEALFV
MTLNGDEKKKGRISATLNTKEGYSYLYIKGSQPEDSATYLCARNTGNQFY
FGTGTSLTVIPNIQNPDPAVYQLRDSKSSDKSVCLFTDFDSQTNVSQSKD
SDAYITDKTVLDMRSMDFKSNSAVAWSNKSDFACANAFNNSIIPEDTFFP
SPESS
(SEQ ID NO: 45)
ATGATCCTGAACGTGGAGCAGAGCCCCCAGAGCCTGCACGTGCAGGAGGG
AGACAGCACCAACTTCACCTGCAGCTTCCCCAGCAGCAACTTCTACGCCC
TGCACTGGTACCGGTGGGAGACCGCCAAGAGCCCCGAGGCCCTGTTCGTG
ATGACCCTGAACGGAGACGAGAAGAAGAAGGGACGGATCAGCGCCACCCT
GAACACCAAGGAGGGATACAGCTACCTGTACATCAAGGGAAGCCAGCCCG
AGGACAGCGCCACCTACCTGTGCGCCCGGAACACCGGAAACCAGTTCTAC
TTCGGAACCGGAACCAGCCTGACCGTGATCCCCAACATCCAGAACCCCGA
CCCCGCCGTGTACCAGCTGCGGGACAGCAAGAGCAGCGACAAGAGCGTGT
GCCTGTTCACCGACTTCGACAGCCAGACCAACGTGAGCCAGAGCAAGGAC
AGCGACGCCTACATCACCGACAAGACCGTGCTGGACATGCGGAGCATGGA
CTTCAAGAGCAACAGCGCCGTGGCCTGGAGCAACAAGAGCGACTTCGCCT
GCGCCAACGCCTTCAACAACAGCATCATCCCCGAGGACACCTTCTTCCCC
AGCCCCGAGAGCAGC

### CD3 Zeta Intracellular Domain (Z-ICD)(in Specific Embodiments, Two or More Z-ICD Sequences May be Utilized):

(SEQ ID NO: 53)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRPGPQCTNYALLKLAGDVESNPGP
(SEQ ID NO: 54)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCCAGTGCACCAA
CTACGCCCTGCTGAAGCTGGCCGGCGACGTGGAGAGCAACCCCGGCCCC

### IL-15:

(SEQ ID NO: 48)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS*
(SEQ ID NO: 49)
ATGCGCATTAGCAAGCCCCACCTGCGGAGCATCAGCATCCAGTGCTACCT
GTGCCTGCTGCTGAACAGCCACTTCCTGACCGAGGCCGGCATCCACGTGT
TCATCCTGGGCTGCTTCAGCGCCGGACTGCCCAAGACCGAGGCCAACTGG
GTGAACGTGATCAGCGACCTGAAGAAGATCGAGGACCTGATCCAGAGCAT
GCACATCGACGCCACCCTGTACACCGAGAGCGACGTGCACCCCAGCTGCA
AGGTGACCGCCATGAAGTGCTTTCTGCTGGAACTGCAGGTGATCAGCCTG
GAAAGCGGCGACGCCAGCATCCACGACACCGTGGAGAACCTGATCATCCT
GGCCAACAACAGCCTGAGCAGCAACGGCAACGTGACCGAGAGCGGCTGCA
AAGAGTGCGAGGAACTGGAAGAGAAGAACATCAAAGAGTTTCTGCAGAGC
TTCGTGCACATCGTGCAGATGTTCATCAACACCAGC

TCR4: refers to TCRpp65 that also may be referred to as TCRpp65betaalpha, and a representative sequence is as follows:

(SEQ ID NO: 55)
MLEGVTQTPKFQVLKTGQSMTLQCAQDMNHEYMSWYRQDPGMGLRLIHYS
VGAGITDQGEVPNGYNVSRSTTEDEPLRLLSAAPSQTSVYFCASSPVTGG
IYGYTFGSGTRLTVVEDLNKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRADRVKFS
RSADAPAYQQGQNQLYNELNLGRREEYDVLDKRRGRDPEMGGKPQRRKNP
QEGLYNELQKDKMAEAYSEIGMKGERRRGKGHDGLYQGLSTATKDTYDAL
HMQALPPRATNFSLLKQAGDVEENPGPMILNVEQSPQSLHVQEGDSTNFT
CSFPSSNFYALHWYRWETAKSPEALFVMTLNGDEKKKGRISATLNTKEGY
SYLYIKGSQPEDSATYLCARNTGNQFYFGTGTSLTVIPNIQNPDPAVYQL
RDSKSSDKSVCLFTDFDSQTNVSQSKDSDAYITDKTVLDMRSMDFKSNSA
VAWSNKSDFACANAFNNSIIPEDTFFPSPESSRVKFSRSADAPAYQQGQN
QLYNELNLGRREEYDVLDKRRGRDPEMGGKPQRRKNPQEGLYNELQKDKM
AEAYSEIGMKGERRRGKGHDGLYQGLSTATKDTYDALHMQALPPRPGPQC
TNYALLKLAGDVESNPGPMRISKPHLRSISIQCYLCLLLNSHFLTEAGIH
VFILGCFSAGLPKTEANWVNVISDLKKIEDLIQSMHIDATLYTESDVHPS
CKVTAMKCFLLELQVISLESGDASIHDTVENLIILANNSLSSNGNVTESG
CKECEELEEKNIKEFLQSFVHIVQMFINTS*

For TCRpp65betaalpha, the corresponding component sequences are as follows, although these particular sequences or others may be utilized in this and/or other constructs:

### TCRb-Extracellular Domain:

(SEQ ID NO: 40)
MLEGVTQTPKFQVLKTGQSMTLQCAQDMNHEYMSWYRQDPGMGLRLIHYS
VGAGITDQGEVPNGYNVSRSTTEDFPLRLLSAAPSQTSVYFCASSPVTGG
IYGYTFGSGTRLTVVEDLNKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRAD
(SEQ ID NO: 41)
ATGCTCGAGGGAGTGACCCAGACCCCCAAGTTCCAGGTGCTGAAGACCGG
ACAGAGCATGACCCTGCAGTGCGCCCAGGACATGAACCACGAGTACATGA
GCTGGTACCGGCAGGACCCCGGAATGGGACTGCGGCTGATCCACTACAGC
GTGGGAGCCGGAATCACCGACCAGGGAGAGGTGCCCAACGGATACAACGT
GAGCCGGAGCACCACCGAGGACTTCCCCCTGCGGCTGCTGAGCGCCGCCC
CCAGCCAGACCAGCGTGTACTTCTGCGCCAGCAGCCCCGTGACCGGAGGA
ATCTACGGATACACCTTCGGAAGCGGAACCCGGCTGACCGTGGTGGAGGA
CCTGAACAAGGTGTTCCCCCCCGAGGTGGCCGTGTTCGAGCCCAGCGAGG
CCGAGATCAGCCACACCCAGAAGGCCACCCTGGTGTGCCTGGCCACCGGA
TTCTTCCCCGACCACGTGGAGCTGAGCTGGTGGGTGAACGGAAAGGAGGT
GCACAGCGGAGTGAGCACCGACCCCCAGCCCCTGAAGGAGCAGCCCGCCC
TGAACGACAGCCGGTACTGCCTGAGCAGCCGGCTGCGGGTGAGCGCCACC
TTCTGGCAGAACCCCCGGAACCACTTCCGGTGCCAGGTGCAGTTCTACGG
ACTGAGCGAGAACGACGAGTGGACCCAGGACCGGGCCAAGCCCGTGACCC
AGATCGTGAGCGCCGAGGCCTGGGGACGGGCCGAC

### CD3 Zeta Intracellular Domain (Z-ICD):

(SEQ ID NO: 42)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRATNFSLLKQAGDVEENPGP
(SEQ ID NO: 54)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCCAGTGCACCAA
CTACGCCCTGCTGAAGCTGGCCGGCGACGTGGAGAGCAACCCCGGCCCC

### TCRa-Extracellular Domain:

(SEQ ID NO: 44)
MILNVEQSPQSLHVQEGDSTNFTCSFPSSNFYALHWYRWETAKSPEALFV
MTLNGDEKKKGRISATLNTKEGYSYLYIKGSQPEDSATYLCARNTGNQFY
FGTGTSLTVIPNIQNPDPAVYQLRDSKSSDKSVCLFTDFDSQTNVSQSKD
SDAYITDKTVLDMRSMDFKSNSAVAWSNKSDFACANAFNNSIIPEDTFFP
SPESS
(SEQ ID NO 45)
ATGATCCTGAACGTGGAGCAGAGCCCCCAGAGCCTGCACGTGCAGGAGGG
AGACAGCACCAACTTCACCTGCAGCTTCCCCAGCAGCAACTTCTACGCCC
TGCACTGGTACCGGTGGGAGACCGCCAAGAGCCCCGAGGCCCTGTTCGTG
ATGACCCTGAACGGAGACGAGAAGAAGAAGGGACGGATCAGCGCCACCCT
GAACACCAAGGAGGGATACAGCTACCTGTACATCAAGGGAAGCCAGCCCG
AGGACAGCGCCACCTACCTGTGCGCCCGGAACACCGGAAACCAGTTCTAC
TTCGGAACCGGAACCAGCCTGACCGTGATCCCCAACATCCAGAACCCCGA
CCCCGCCGTGTACCAGCTGCGGGACAGCAAGAGCAGCGACAAGAGCGTGT
GCCTGTTCACCGACTTCGACAGCCAGACCAACGTGAGCCAGAGCAAGGAC
AGCGACGCCTACATCACCGACAAGACCGTGCTGGACATGCGGAGCATGGA
CTTCAAGAGCAACAGCGCCGTGGCCTGGAGCAACAAGAGCGACTTCGCCT
GCGCCAACGCCTTCAACAACAGCATCATCCCCGAGGACACCTTCTTCCCC
AGCCCCGAGAGCAGC

### CD3 Zeta Intracellular Domain (Z-ICD):

(SEQ ID NO: 53)
RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPRPGPQCTNYALLKLAGDVESNPGP
(SEQ ID NO: 54)
AGAGTGAAGTTCAGCAGGAGCGCAGACGCCCCCGCGTACCAGCAGGGCCA
GAACCAGCTCTATAACGAGCTCAATCTAGGACGAAGAGAGGAGTACGATG
TTTTGGACAAGAGACGTGGCCGGGACCCTGAGATGGGGGGAAAGCCGCAG
AGAAGGAAGAACCCTCAGGAAGGCCTGTACAATGAACTGCAGAAAGATAA
GATGGCGGAGGCCTACAGTGAGATTGGGATGAAAGGCGAGCGCCGGAGGG
GCAAGGGGCACGATGGCCTTTACCAGGGTCTCAGTACAGCCACCAAGGAC
ACCTACGACGCCCTTCACATGCAGGCCCTGCCCCCTCGCCAGTGCACCAA
CTACGCCCTGCTGAAGCTGGCCGGCGACGTGGAGAGCAACCCCGGCCCC

### IL-15:

(SEQ ID NO: 48)
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS*
(SEQ ID NO: 49)
ATGCGCATTAGCAAGCCCCACCTGCGGAGCATCAGCATCCAGTGCTACCT
GTGCCTGCTGCTGAACAGCCACTTCCTGACCGAGGCCGGCATCCACGTGT
TCATCCTGGGCTGCTTCAGCGCCGGACTGCCCAAGACCGAGGCCAACTGG
GTGAACGTGATCAGCGACCTGAAGAAGATCGAGGACCTGATCCAGAGCAT
GCACATCGACGCCACCCTGTACACCGAGAGCGACGTGCACCCCAGCTGCA
AGGTGACCGCCATGAAGTGCTTTCTGCTGGAACTGCAGGTGATCAGCCTG
GAAAGCGGCGACGCCAGCATCCACGACACCGTGGAGAACCTGATCATCCT
GGCCAACAACAGCCTGAGCAGCAACGGCAACGTGACCGAGAGCGGCTGCA
AAGAGTGCGAGGAACTGGAAGAGAAGAACATCAAAGAGTTTCTGCAGAGC
TTCGTGCACATCGTGCAGATGTTCATCAACACCAGC

### An Additional Representative Sequence for TCRpp65Betaalpha is as Follows:

(SEQ ID NO: 56)
ATGGACTCCTGGACCTTCTGCTGTGTGTCCCTTTGCATCCTGGTAGCAAAGCACACAGATGC
TGGAGTTATCCAGTCACCCCGGCACGAGGTGACAGAGATGGGACAAGAAGTGACTCTGAGAT
GTAAACCAATTTCAGGACACGACTACCTTTTCTGGTACAGACAGACCATGATGCGGGGACTG
GAGTTGCTCATTTACTTTAACAACAACGTTCCGATAGATGATTCAGGGATGCCCGAGGATCG
ATTCTCAGCTAAGATGCCTAATGCATCATTCTCCACTCTGAAGATCCAGCCCTCAGAACCCA
GGGACTCAGCTGTGTACTTCTGTGCCAGCAGTTCGGCAAACTATGGCTACACCTTCGGTTCG
GGGACCAGGTTAACCGTTGTAGAGGACCTGAACAAGGTGTTCCCACCCGAGGTCGCTGTGTT
TGAGCCATCAGAAGCAGAGATCTCCCACACCCAAAAGGCCACACTGGTGTGCCTGGCCACAG
GCTTCTTCCCTGACCACGTGGAGCTGAGCTGGTGGGTGAATGGGAAGGAGGTGCACAGTGGG
GTCAGCACGGACCCGCAGCCCCTCAAGGAGCAGCCCGCCCTCAATGACTCCAGATACTGCCT
GAGCAGCCGCCTGAGGGTCTCGGCCACCTTCTGGCAGAACCCCCGCAACCACTTCCGCTGTC
AAGTCCAGTTCTACGGGCTCTCGGAGAATGACGAGTGGACCCAGGATAGGGCCAAACCCGTC
ACCCAGATCGTCAGCGCCGAGGCCTGGGGTAGAGCAGACTGTGGCTTTACCTCGGTGTCCTA
CCAGCAAGGGGTCCTGTCTGCCACCATCCTCTATGAGATCCTGCTAGGGAAGGCCACCCTGT
ATGCTGTGCTGGTCAGCGCCCTTGTGTTGATGGCCATGGTCAAGAGAAAGGATTTCGAGGGC
AGGGGAAGTCTTCTAACATGCGGGGACGTGGAGGAAAATCCCGGGCCCATGCTCCTTGAACA
TTTATTAATAATCTTGTGGATGCAGCTGACATGGGTCAGTGGTCAACAGCTGAATCAGAGTC
CTCAATCTATGTTTATCCAGGAAGGAGAAGATGTCTCCATGAACTGCACTTCTTCAAGCATA
TTTAACACCTGGCTATGGTACAAGCAGGACCCTGGGGAAGGTCCTGTCCTCTTGATAGCCTT
ATATAAGGCTGGTGAATTGACCTCAAATGGAAGACTGACTGCTCAGTTTGGTATAACCAGAA
AGGACAGCTTCCTGAATATCTCAGCATCCATACCCAGTGATGTAGGCATCTACTTCTGTGCT
GGACCCATGAAAACCTCCTACGACAAGGTGATATTTGGGCCAGGGACAAGCTTATCAGTCAT
TCCAAATATCCAGAACCCTGACCCTGCCGTGTACCAGCTGAGAGACTCTAAATCCAGTGACA
AGTCTGTCTGCCTATTCACCGATTTTGATTCTCAAACAAATGTGTCACAAAGTAAGGATTCT
GATGTGTATATCACAGACAAAACTGTGCTAGACATGAGGTCTATGGACTTCAAGAGCAACAG
TGCTGTGGCCTGGAGCAACAAATCTGACTTTGCATGTGCAAACGCCTTCAACAACAGCATTA
TTCCAGAAGACACCTTCTTCCCCAGCCCAGAAAGTTCCTGTGATGTCAAGCTGGTCGAGAAA
AGCTTTGAAACAGATACGAACCTAAACTTTCAAAACCTGTCAGTGATTGGGTTCCGAATCCT
CCTCCTGAAAGTGGCCGGGTTTAATCTGCTCATGACGCTGCGGCTGTGGTCCAGCTGA
(SEQ ID NO: 57)
MDSWTFCCVSLCILVAKHTDAGVIQSPRHEVTEMGQEVTLRCKPISGHDYLFWYRQTMMRGL
ELLIYFNNNVPIDDSGMPEDRFSAKMPNASFSTLKIQPSEPRDSAVYFCASSSANYGYTFGS
GTRLTVVEDLNKVFPPEVAVFEPSEAEISHTQKATLVCLATGFFPDHVELSWWVNGKEVHSG
VSTDPQPLKEQPALNDSRYCLSSRLRVSATFWQNPRNHFRCQVQFYGLSENDEWTQDRAKPV
TQIVSAEAWGRADCGFTSVSYQQGVLSATILYEILLGKATLYAVLVSALVLMAMVKRKDFEG
RGSLLTCGDVEENPGPMLLEHLLIILWMQLTWVSGQQLNQSPQSMFIQEGEDVSMNCTSSSI
FNTWLWYKQDPGEGPVLLIALYKAGELTSNGRLTAQFGITRKDSFLNISASIPSDVGIYFCA
GPMKTSYDKVIFGPGTSLSVIPNIQNPDPAVYQLRDSKSSDKSVCLFTDFDSQTNVSQSKDS
DVYITDKTVLDMRSMDFKSNSAVAWSNKSDFACANAFNNSIIPEDTFFPSPESSCDVKLVEK
SFETDTNINFQNLSVIGFRILLLKVAGFNLLMTLRLWSS*

Z1: refers to full length CD3zeta, full length CD3 gamma, full length CD3 delta, and full length CD3 epsilon linked to IL15 (see FIGS. 2A and 2B), and it may also be referred to as CD3ZFLGDEFL15, and representative sequences may be as follows:

(SEQ ID NO: 58)
MLEMKWKALFTAAILQAQLPITEAQSFGLLDPKLCYLLDGILFIYGVILTALFLRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
PMEQGKGLAVLILAIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIELNAATISGFLFAEI
VSIFVLAVGVYFIAGQDGVRQSRASDKQTLLPNDQLYQPLKDREDDQYSHLQGNQLRRNVKQ
TLNFDLLKLAGDVESNPGPMEHSTFLSGLVLATLLSQVSPFKIPIEELEDRVFVNCNTSITW
VEGTVGTLLSDITRLDLGKRILDPRGIYRCNGTDIYKDKESTVQVHYRMCQSCVELDPATVA
GIIVTDVIATLLLALGVFCFAGHETGRLSGAADTQALLRNDQVYQPLRDRDDAQYSHLGGNW
ARNKEGRGSLLTCGDVEENPGPMQSGTHWRVLGLCLLSVGVWGQDGNEEMGGITQTPYKVSI
SGTTVILTCPQYPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSKNRKAKAKPVTR
GAGAGGRQRGQNKERPPPVPNPDYEPIRKGQRDLYSGLNQRRIGPQCTNYALLKLAGDVESN
PGPMRISKPHLRSISIQCYLCLLLNSHFLTEAGIHVFILGCFSAGLPKTEANWVNVISDLKK
IEDLIQSMHIDATLYTESDVHPSCKVTAMKCFLLELQVISLESGDASIHDTVENLIILANNS
LSSNGNVTESGCKECEELEEKNIKEFLQSFVHIVQMFINTS
(SEQ ID NO: 59)
ATGCTCGAGATGAAGTGGAAGGCGCTTTTCACCGCGGCCATCCTGCAGGCACAGTTGCCGAT
TACAGAGGCACAGAGCTTTGGCCTGCTGGATCCCAAACTCTGCTACCTGCTGGATGGAATCC
TCTTCATCTATGGTGTCATTCTCACTGCCTTGTTCCTGAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCCATGGAACAGGGGAAGGGCCTGGCTGTCCTCATCCTGGCTATCATTCTTCTTCAAGGTAC
TTTGGCCCAGTCAATCAAAGGAAACCACTTGGTTAAGGTGTATGACTATCAAGAAGATGGTT
CGGTACTTCTGACTTGTGATGCAGAAGCCAAAAATATCACATGGTTTAAAGATGGGAAGATG
ATCGGCTTCCTAACTGAAGATAAAAAAAAATGGAATCTGGGAAGTAATGCCAAGGACCCTCG
TGGGATGTATCAGTGTAAAGGATCACAGAACAAGTCAAAACCACTCCAAGTGTATTACAGAA
TGTGTCAGAACTGCATTGAACTAAATGCAGCCACCATATCTGGCTTTCTCTTTGCTGAAATC
GTCAGCATTTTCGTCCTTGCTGTTGGGGTCTACTTCATTGCTGGACAGGATGGAGTTCGCCA
GTCGAGAGCTTCAGACAAGCAGACTCTGTTGCCCAATGACCAGCTCTACCAGCCCCTCAAGG
ATCGAGAAGATGACCAGTACAGCCACCTTCAAGGAAACCAGTTGAGGAGGAATGTGAAGCAG
ACCCTGAACTTCGACCTGCTGAAGCTGGCCGGCGACGTGGAGAGCAACCCCGGCCCCATGGA
GCACAGCACCTTCCTGAGCGGCCTGGTGCTGGCCACCCTGCTGAGCCAGGTGAGCCCCTTCA
AGATCCCCATCGAGGAGCTGGAGGACAGAGTGTTCGTGAACTGCAACACCAGCATCACCTGG
GTGGAGGGCACCGTGGGCACCCTGCTGAGCGACATCACCAGACTGGACCTGGGCAAGAGAAT
CCTGGACCCCAGAGGCATCTACAGATGCAACGGCACCGACATCTACAAGGACAAGGAGAGCA
CCGTGCAGGTGCACTACAGAATGTGCCAGAGCTGCGTGGAGCTGGACCCCGCCACCGTGGCC
GGCATCATCGTGACCGACGTGATCGCCACCCTGCTGCTGGCCCTGGGCGTGTTCTGCTTCGC
CGGCCACGAGACCGGCAGACTGAGCGGCGCCGCCGACACCCAGGCCCTGCTGAGAAACGACC
AGGTGTACCAGCCCCTGAGAGACAGAGACGACGCCCAGTACAGCCACCTGGGCGGCAACTGG
GCCAGAAACAAGGAGGGCAGAGGCAGCCTGCTGACCTGCGGCGACGTGGAGGAGAACCCCGG
CCCCATGCAGAGCGGCACCCACTGGAGAGTGCTGGGCCTGTGCCTGCTGAGCGTGGGCGTGT
GGGGCCAGGACGGCAACGAGGAGATGGGCGGCATCACCCAGACCCCCTACAAGGTGAGCATC
AGCGGCACCACCGTGATCCTGACCTGCCCCCAGTACCCCGGCAGCGAGATCCTGTGGCAGCA
CAACGACAAGAACATCGGCGGCGACGAGGACGACAAGAACATCGGCAGCGACGAGGACCACC
TGAGCCTGAAGGAGTTCAGCGAGCTGGAGCAGAGCGGCTACTACGTGTGCTACCCCAGAGGC
AGCAAGCCCGAGGACGCCAACTTCTACCTGTACCTGAGAGCCAGAGTGTGCGAGAACTGCAT
GGAGATGGACGTGATGAGCGTGGCCACCATCGTGATCGTGGACATCTGCATCACCGGCGGCC
TGCTGCTGCTGGTGTACTACTGGAGCAAGAACAGAAAGGCCAAGGCCAAGCCCGTGACCAGA
GGCGCCGGCGCCGGCGGCAGACAGAGAGGCCAGAACAAGGAGAGACCCCCCCCCGTGCCCAA
CCCCGACTACGAGCCCATCAGAAAGGGCCAGAGAGACCTGTACAGCGGCCTGAACCAGAGAA
GAATCGGACCGCAGTGTACTAATTATGCTCTCTTGAAATTGGCTGGAGATGTTGAGAGCAAT
CCCGGGCCCATGCGCATTAGCAAGCCCCACCTGCGGAGCATCAGCATCCAGTGCTACCTGTG
CCTGCTGCTGAACAGCCACTTCCTGACCGAGGCCGGCATCCACGTGTTCATCCTGGGCTGCT
TCAGCGCCGGACTGCCCAAGACCGAGGCCAACTGGGTGAACGTGATCAGCGACCTGAAGAAG
ATCGAGGACCTGATCCAGAGCATGCACATCGACGCCACCCTGTACACCGAGAGCGACGTGCA
CCCCAGCTGCAAGGTGACCGCCATGAAGTGCTTTCTGCTGGAACTGCAGGTGATCAGCCTGG
AAAGCGGCGACGCCAGCATCCACGACACCGTGGAGAACCTGATCATCCTGGCCAACAACAGC
CTGAGCAGCAACGGCAACGTGACCGAGAGCGGCTGCAAAGAGTGCGAGGAACTGGAAGAGAA
GAACATCAAAGAGTTTCTGCAGAGCTTCGTGCACATCGTGCAGATGTTCATCAACACCAGC

Z2: refers to full length CD3zeta, full length CD3 gamma, full length CD3 delta, and full length CD3 epsilon linked to membrane bound IL21 (with CD8 transmembrane domain for the membrane bound IL21), and it may also be referred to as CD3ZGDEFLSP821CD28 and a representative sequence is as follows:

(SEQ ID NO: 60)
MLEMKWKALFTAAILQAQLPITEAQSFGLLDPKLCYLLDGILFIYGVILT
ALFLRVKFSRSADAPAYQQGQNQLYNELNLGRREEYDVLDKRRGRDPEMG
GKPQRRKNPQEGLYNELQKDKMAEAYSEIGMKGERRRGKGHDGLYQGLST
ATKDTYDALHMQALPPRQCTNYALLKLAGDVESNPGPMEQGKGLAVLILA
IILLQGTLAQSIKGNHLVKVYDYQEDGSVLLTCDAEAKNITWFKDGKMIG
FLTEDKKKWNLGSNAKDPRGMYQCKGSQNKSKPLQVYYRMCQNCIELNAA
TISGFLFAEIVSIFVLAVGVYFIAGQDGVRQSRASDKQTLLPNDQLYQPL
KDREDDQYSHLQGNQLRRNVKQTLNFDLLKLAGDVESNPGPMEHSTFLSG
LVLATLLSQVSPFKIPIEELEDRVFVNCNTSITWVEGTVGTLLSDITRLD
LGKRILDPRGIYRCNGTDIYKDKESTVQVHYRMCQSCVELDPATVAGIIV
TDVIATLLLALGVFCFAGHETGRLSGAADTQALLRNDQVYQPLRDRDDAQ
YSHLGGNWARNKEGRGSLLTCGDVEENPGPMQSGTHWRVLGLCLLSVGVW
GQDGNEEMGGITQTPYKVSISGTTVILTCPQYPGSEILWQHNDKNIGGDE
DDKNIGSDEDHLSLKEFSELEQSGYYVCYPRGSKPEDANFYLYLRARVCE
NCMEMDVMSVATIVIVDICITGGLLLLVYYWSKNRKAKAKPVTRGAGAGG
RQRGQNKERPPPVPNPDYEPIRKGQRDLYSGLNQRRIGPQCTNYALLKLA
GDVESNPGPMRICLTSDRLAPAAGLAAPRRQAVHKSSSQGQDRHMIRMRQ
LIDIVDQLKNYVNDLVPEFLPAPEDVETNCEWSAFSCFQKAQLKSANTGN
NERIINVSIKKLKRKPPSTNAGRRQKHRLTCPSCDSYEKKPPKEFLERFK
SLLQKMIHQHLSSRTHGSEDSTTTPAPRPPTPAPTIASQPLSLRPEACRP
AAGGAVHTRGLDFACDFWVLVVVGGVLACYSLLVTVAFIIFWV*

For CD3ZGDEFLSP821CD28, the corresponding component sequences are as follows, although these particular sequences or others may be utilized in this and/or other constructs:

### CD3:

```
  (SEQ ID NO: 61)
MLEMKWKALFTAAILQAQLPITEAQSFGLLDPKLCYLLDGILFIYGVILTALFLRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
PMEQGKGLAVLILAIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIELNAATISGFLFAEI
VSIFVLAVGVYFIAGQDGVRQSRASDKQTLLPNDQLYQPLKDREDDQYSHLQGNQLRRNVKQ
TLNFDLLKLAGDVESNPGPMEHSTFLSGLVLATLLSQVSPEKIPIEELEDRVFVNCNTSITW
VEGTVGTLLSDITRLDLGKRILDPRGIYRCNGTDIYKDKESTVQVHYRMCQSCVELDPATVA
GIIVTDVIATLLLALGVFCFAGHETGRLSGAADTQALLRNDQVYQPLRDRDDAQYSHLGGNW
ARNKEGRGSLLTCGDVEENPGPMQSGTHWRVLGLCLLSVGVWGQDGNEEMGGITQTPYKVSI
SGTTVILTCPQYPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSKNRKAKAKPVTR
GAGAGGRQRGQNKERPPPVPNPDYEPIRKGQRDLYSGLNQRRIGPQCTNYALLKLAGDVESN
PGP
  (SEQ ID NO: 62)
ATGCTCGAGATGAAGTGGAAGGCGCTTTTCACCGCGGCCATCCTGCAGGCACAGTTGCCGAT
TACAGAGGCACAGAGCTTTGGCCTGCTGGATCCCAAACTCTGCTACCTGCTGGATGGAATCC
TCTTCATCTATGGTGTCATTCTCACTGCCTTGTTCCTGAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCCATGGAACAGGGGAAGGGCCTGGCTGTCCTCATCCTGGCTATCATTCTTCTTCAAGGTAC
TTTGGCCCAGTCAATCAAAGGAAACCACTTGGTTAAGGTGTATGACTATCAAGAAGATGGTT
CGGTACTTCTGACTTGTGATGCAGAAGCCAAAAATATCACATGGTTTAAAGATGGGAAGATG
ATCGGCTTCCTAACTGAAGATAAAAAAAAATGGAATCTGGGAAGTAATGCCAAGGACCCTCG
TGGGATGTATCAGTGTAAAGGATCACAGAACAAGTCAAAACCACTCCAAGTGTATTACAGAA
TGTGTCAGAACTGCATTGAACTAAATGCAGCCACCATATCTGGCTTTCTCTTTGCTGAAATC
GTCAGCATTTTCGTCCTTGCTGTTGGGGTCTACTTCATTGCTGGACAGGATGGAGTTCGCCA
GTCGAGAGCTTCAGACAAGCAGACTCTGTTGCCCAATGACCAGCTCTACCAGCCCCTCAAGG
ATCGAGAAGATGACCAGTACAGCCACCTTCAAGGAAACCAGTTGAGGAGGAATGTGAAGCAG
ACCCTGAACTTCGACCTGCTGAAGCTGGCCGGCGACGTGGAGAGCAACCCCGGCCCCATGGA
GCACAGCACCTTCCTGAGCGGCCTGGTGCTGGCCACCCTGCTGAGCCAGGTGAGCCCCTTCA
AGATCCCCATCGAGGAGCTGGAGGACAGAGTGTTCGTGAACTGCAACACCAGCATCACCTGG
GTGGAGGGCACCGTGGGCACCCTGCTGAGCGACATCACCAGACTGGACCTGGGCAAGAGAAT
CCTGGACCCCAGAGGCATCTACAGATGCAACGGCACCGACATCTACAAGGACAAGGAGAGCA
CCGTGCAGGTGCACTACAGAATGTGCCAGAGCTGCGTGGAGCTGGACCCCGCCACCGTGGCC
GGCATCATCGTGACCGACGTGATCGCCACCCTGCTGCTGGCCCTGGGCGTGTTCTGCTTCGC
CGGCCACGAGACCGGCAGACTGAGCGGCGCCGCCGACACCCAGGCCCTGCTGAGAAACGACC
AGGTGTACCAGCCCCTGAGAGACAGAGACGACGCCCAGTACAGCCACCTGGGCGGCAACTGG
GCCAGAAACAAGGAGGGCAGAGGCAGCCTGCTGACCTGCGGCGACGTGGAGGAGAACCCCGG
CCCCATGCAGAGCGGCACCCACTGGAGAGTGCTGGGCCTGTGCCTGCTGAGCGTGGGCGTGT
GGGGCCAGGACGGCAACGAGGAGATGGGCGGCATCACCCAGACCCCCTACAAGGTGAGCATC
AGCGGCACCACCGTGATCCTGACCTGCCCCCAGTACCCCGGCAGCGAGATCCTGTGGCAGCA
CAACGACAAGAACATCGGCGGCGACGAGGACGACAAGAACATCGGCAGCGACGAGGACCACC
TGAGCCTGAAGGAGTTCAGCGAGCTGGAGCAGAGCGGCTACTACGTGTGCTACCCCAGAGGC
AGCAAGCCCGAGGACGCCAACTTCTACCTGTACCTGAGAGCCAGAGTGTGCGAGAACTGCAT
GGAGATGGACGTGATGAGCGTGGCCACCATCGTGATCGTGGACATCTGCATCACCGGCGGCC
TGCTGCTGCTGGTGTACTACTGGAGCAAGAACAGAAAGGCCAAGGCCAAGCCCGTGACCAGA
GGCGCCGGCGCCGGCGGCAGACAGAGAGGCCAGAACAAGGAGAGACCCCCCCCCGTGCCCAA
CCCCGACTACGAGCCCATCAGAAAGGGCCAGAGAGACCTGTACAGCGGCCTGAACCAGAGAA
GAATCGGACCGCAGTGTACTAATTATGCTCTCTTGAAATTGGCTGGAGATGTTGAGAGCAAT
CCCGGGCCC
```

### SP CD8:

(SEQ ID NO: 63)
MRICLTSDRLAPAAGLAAPRRQAV
(SEQ ID NO: 64)
atgcgcatttgcctgaccagcgatcgcctggcgccggcggcgggcctggc
ggcgccgcgccgccaggcggtg

### IL-21:

(SEQ ID NO: 65)
HKSSSQGQDRHMIRMRQLIDIVDQLKNYVNDLVPEFLPAPEDVETNCEWS
AFSCFQKAQLKSANTGNNERIINVSIKKLKRKPPSTNAGRRQKHRLTCPS
CDSYEKKPPKEFLERFKSLLQKMIHQHLSSRTHGSEDS
(SEQ ID NO: 66)
CATAAATCTTCCTCTCAAGGTCAGGACCGCCATATGATTCGAATGCGGCA
GCTGATTGACATAGTCGATCAACTGAAGAACTATGTGAATGATCTTGTGC
CCGAGTTTTTGCCAGCCCCTGAAGACGTAGAAACTAATTGTGAGTGGAGT
GCCTTTTCCTGCTTTCAAAAGGCACAGCTGAAATCCGCCAACACGGGCAA
TAACGAACGGATAATTAACGTATCCATTAAGAAGCTGAAGCGGAAGCCGC
CCTCAACCAATGCGGGACGGCGGCAAAAGCATCGCTTGACCTGTCCGTCA
TGCGACAGCTACGAGAAAAAGCCCCCGAAGGAGTTCTTGGAACGCTTCAA
GAGTCTCCTTCAGAAAATGATTCACCAGCACCTGTCCTCACGGACGCAC
GGAAGCGAGGACAGT

### CD8 Hinge:

(SEQ ID NO: 67)
TTTPAPRPPTPAPTIASQPLSLRPEACRPAAGGAVHTRGLDFACD
(SEQ ID NO: 68)
ACCACGACGCCAGCGCCGCGACCACCAACACCGGCGCCCACCATCGCGTC
GCAGCCCCTGTCCCTGCGCCCAGAGGCGTGCCGGCCAGCGGCGGGGGGCG
CAGTGCACACGAGGGGGCTGGACTTCGCCTGTGAT

### CD28 Transmembrane Domain:

(SEQ ID NO: 69)
FWVLVVVGGVLACYSLLVTVAFIIFWV*
(SEQ ID NO: 70)
TTTTGGGTGCTGGTGGTGGTTGGTGGAGTCCTGGCTTGCTATAGCTTGC
TAGTAACAGTGGCCTTTATTATTTTCTGGGTG

Z3: refers to full length CD3zeta, full length CD3 gamma, full length CD3 delta, and full length CD3 epsilon linked to membrane bound IL21 (with CD28 transmembrane domain for the membrane bound IL21), and it may also be referred to as CD3ZGDEFL8SP21CD8 with a representative sequence as follows:

(SEQ ID NO: 71)
MLEMKWKALFTAAILQAQLPITEAQSFGLLDPKLCYLLDGILFIYGVILTALFLRVKESRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
PMEQGKGLAVLILAIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIELNAATISGELFAEI
VSIFVLAVGVYFIAGQDGVRQSRASDKQTLLPNDQLYQPLKDREDDQYSHLQGNQLRRNVKQ
TLNFDLLKLAGDVESNPGPMEHSTFLSGLVLATLLSQVSPEKIPIEELEDRVFVNCNTSITW
VEGTVGTLLSDITRLDLGKRILDPRGIYRCNGTDIYKDKESTVQVHYRMCQSCVELDPATVA
GIIVTDVIATLLLALGVFCFAGHETGRLSGAADTQALLRNDQVYQPLRDRDDAQYSHLGGNW
ARNKEGRGSLLTCGDVEENPGPMQSGTHWRVLGLCLLSVGVWGQDGNEEMGGITQTPYKVSI
SGTTVILTCPQYPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSKNRKAKAKPVTR
GAGAGGRQRGQNKERPPPVPNPDYEPIRKGQRDLYSGLNQRRIGPQCTNYALLKLAGDVESN
PGPMRICLTSDRLAPAAGLAAPRRQAVHKSSSQGQDRHMIRMRQLIDIVDQLKNYVNDLVPE
FLPAPEDVETNCEWSAFSCFQKAQLKSANTGNNERIINVSIKKLKRKPPSTNAGRRQKHRLT
CPSCDSYEKKPPKEFLERFKSLLQKMIHQHLSSRTHGSEDSTTTPAPRPPTPAPTIASQPLS
LRPEACRPAAGGAVHTRGLDFACDIYIWAPLAGTCGVLLLSLVIT.

For CD3ZGDEFL8SP21CD8, the corresponding component sequences are as follows, although these particular sequences or others may be utilized in this and/or other constructs:

### CD3:

```
  (SEQ ID NO: 61)
MLEMKWKALFTAAILQAQLPITEAQSFGLLDPKLCYLLDGILFIYGVILTALFLRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
PMEQGKGLAVLILAIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIELNAATISGFLFAEI
VSIFVLAVGVYFIAGQDGVRQSRASDKQTLLPNDQLYQPLKDREDDQYSHLQGNQLRRNVKQ
TLNFDLLKLAGDVESNPGPMEHSTFLSGLVLATLLSQVSPFKIPIEELEDRVFVNCNTSITW
VEGTVGTLLSDITRLDLGKRILDPRGIYRCNGTDIYKDKESTVQVHYRMCQSCVELDPATVA
GIIVTDVIATLLLALGVFCFAGHETGRLSGAADTQALLRNDQVYQPLRDRDDAQYSHLGGNW
ARNKEGRGSLLTCGDVEENPGPMQSGTHWRVLGLCLLSVGVWGQDGNEEMGGITQTPYKVSI
SGTTVILTCPQYPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSKNRKAKAKPVTR
GAGAGGRQRGQNKERPPPVPNPDYEPIRKGQRDLYSGLNQRRIGPQCTNYALLKLAGDVESN
PGP
  (SEQ ID NO: 62)
ATGCTCGAGATGAAGTGGAAGGCGCTTTTCACCGCGGCCATCCTGCAGGCACAGTTGCCGAT
TACAGAGGCACAGAGCTTTGGCCTGCTGGATCCCAAACTCTGCTACCTGCTGGATGGAATCC
TCTTCATCTATGGTGTCATTCTCACTGCCTTGTTCCTGAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCCATGGAACAGGGGAAGGGCCTGGCTGTCCTCATCCTGGCTATCATTCTTCTTCAAGGTAC
TTTGGCCCAGTCAATCAAAGGAAACCACTTGGTTAAGGTGTATGACTATCAAGAAGATGGTT
CGGTACTTCTGACTTGTGATGCAGAAGCCAAAAATATCACATGGTTTAAAGATGGGAAGATG
ATCGGCTTCCTAACTGAAGATAAAAAAAAATGGAATCTGGGAAGTAATGCCAAGGACCCTCG
TGGGATGTATCAGTGTAAAGGATCACAGAACAAGTCAAAACCACTCCAAGTGTATTACAGAA
TGTGTCAGAACTGCATTGAACTAAATGCAGCCACCATATCTGGCTTTCTCTTTGCTGAAATC
GTCAGCATTTTCGTCCTTGCTGTTGGGGTCTACTTCATTGCTGGACAGGATGGAGTTCGCCA
GTCGAGAGCTTCAGACAAGCAGACTCTGTTGCCCAATGACCAGCTCTACCAGCCCCTCAAGG
ATCGAGAAGATGACCAGTACAGCCACCTTCAAGGAAACCAGTTGAGGAGGAATGTGAAGCAG
ACCCTGAACTTCGACCTGCTGAAGCTGGCCGGCGACGTGGAGAGCAACCCCGGCCCCATGGA
GCACAGCACCTTCCTGAGCGGCCTGGTGCTGGCCACCCTGCTGAGCCAGGTGAGCCCCTTCA
AGATCCCCATCGAGGAGCTGGAGGACAGAGTGTTCGTGAACTGCAACACCAGCATCACCTGG
GTGGAGGGCACCGTGGGCACCCTGCTGAGCGACATCACCAGACTGGACCTGGGCAAGAGAAT
CCTGGACCCCAGAGGCATCTACAGATGCAACGGCACCGACATCTACAAGGACAAGGAGAGCA
CCGTGCAGGTGCACTACAGAATGTGCCAGAGCTGCGTGGAGCTGGACCCCGCCACCGTGGCC
GGCATCATCGTGACCGACGTGATCGCCACCCTGCTGCTGGCCCTGGGCGTGTTCTGCTTCGC
CGGCCACGAGACCGGCAGACTGAGCGGCGCCGCCGACACCCAGGCCCTGCTGAGAAACGACC
AGGTGTACCAGCCCCTGAGAGACAGAGACGACGCCCAGTACAGCCACCTGGGCGGCAACTGG
GCCAGAAACAAGGAGGGCAGAGGCAGCCTGCTGACCTGCGGCGACGTGGAGGAGAACCCCGG
CCCCATGCAGAGCGGCACCCACTGGAGAGTGCTGGGCCTGTGCCTGCTGAGCGTGGGCGTGT
GGGGCCAGGACGGCAACGAGGAGATGGGCGGCATCACCCAGACCCCCTACAAGGTGAGCATC
AGCGGCACCACCGTGATCCTGACCTGCCCCCAGTACCCCGGCAGCGAGATCCTGTGGCAGCA
CAACGACAAGAACATCGGCGGCGACGAGGACGACAAGAACATCGGCAGCGACGAGGACCACC
TGAGCCTGAAGGAGTTCAGCGAGCTGGAGCAGAGCGGCTACTACGTGTGCTACCCCAGAGGC
AGCAAGCCCGAGGACGCCAACTTCTACCTGTACCTGAGAGCCAGAGTGTGCGAGAACTGCAT
GGAGATGGACGTGATGAGCGTGGCCACCATCGTGATCGTGGACATCTGCATCACCGGCGGCC
TGCTGCTGCTGGTGTACTACTGGAGCAAGAACAGAAAGGCCAAGGCCAAGCCCGTGACCAGA
GGCGCCGGCGCCGGCGGCAGACAGAGAGGCCAGAACAAGGAGAGACCCCCCCCCGTGCCCAA
CCCCGACTACGAGCCCATCAGAAAGGGCCAGAGAGACCTGTACAGCGGCCTGAACCAGAGAA
GAATCGGACCGCAGTGTACTAATTATGCTCTCTTGAAATTGGCTGGAGATGTTGAGAGCAAT
CCCGGGCCC
```

### SP CD8:

(SEQ ID NO: 63)
MRICLTSDRLAPAAGLAAPRRQAV
(SEQ ID NO: 64)
atgcgcatttgcctgaccagcgatcgcctggcgccggcggcgggcctggc
ggcgccgcgccgccaggcggtg

### IL-21:

(SEQ ID NO: 65)
HKSSSQGQDRHMIRMRQLIDIVDQLKNYVNDLVPEFLPAPEDVETNCEWSAFSCFQKAQLKS
ANTGNNERIINVSIKKLKRKPPSTNAGRRQKHRLTCPSCDSYEKKPPKEFLERFKSLLQKMI
HQHLSSRTHGSEDS
(SEQ ID NO: 66)
cataaatcttcctctcaaggtcaggaccgccatatgattcgaatgcggcagctgattgacat
agtcgatcaactgaagaactatgtgaatgatcttgtgcccgagtttttgccagcccctgaag
acgtagaaactaattgtgagtggagtgccttttcctgctttcaaaaggcacagctgaaatcc
gccaacacgggcaataacgaacggataattaacgtatccattaagaagctgaagcggaagcc
gccctcaaccaatgcgggacggggcaaaagcatcgcttgacctgtccgtcatgcgacagct
acgagaaaaagcccccgaaggagttcttggaacgcttcaagagtctccttcagaaaatgatt
caccagcacctgtcctcacggacgcacggaagcgaggacagt

### CD8 Hinge:

(SEQ ID NO: 67)
TTTPAPRPPTPAPTIASQPLSLRPEACRPAAGGAVHTRGLDFACD
(SEQ ID NO: 68)
ACCACGACGCCAGCGCCGCGACCACCAACACCGGCGCCCACCATCGCGT
CGCAGCCCCTGTCCCTGCGCCCAGAGGCGTGCCGGCCAGCGGCGGGGGG
CGCAGTGCACACGAGGGGGCTGGACTTCGCCTGTGAT

### CD8 Transmembrane Domain:

(SEQ ID NO: 72)
IYIWAPLAGTCGVLLLSLVIT*
(SEQ ID NO: 73)
ATCTACATCTGGGCGCCCTTGGCCGGGACTTGTGGGGTCCTTCTC
CTGTCACTGGTTATCACC

In certain embodiments, provided herein are CD3 constructs comprising a fusion with an intracellular co-stimulatory domain derived from CD16, NKG2D, DAP10, DAP12, 2B4, 4-1BB, CD2, CD28, DNAM, or any combination thereof. In certain embodiments, an intracellular co-stimulatory domain is fused to CD3δ, CD3ε, CD3γ, and/or CD3ζ. In certain embodiments, such a CD3 fusion construct comprises a CD3ζ fused to a DAP10 intracellular co-stimulatory domain. In certain embodiments, such a CD3 fusion construct comprises a CD3ζ fused to a CD28 intracellular co-stimulatory domain. In certain embodiments, such a CD3 fusion construct comprises a CD3ζ fused to a DAP10 intracellular co-stimulatory domain and a CD28 intracellular co-stimulatory domain. In certain embodiments, a CD3ζ fused to a DAP10 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 106. In certain embodiments, a CD3ζ fused to a CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 107. In certain embodiments, a CD3ζ fused to a DAP10 intracellular co-stimulatory domain and a CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 108. In certain embodiments, a CD3ζ fused to a DAP10 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 109. In certain embodiments, a CD3ζ fused to a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 110. In certain embodiments, a CD3ζ fused to a DAP10 intracellular co-stimulatory domain and a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 111. In certain embodiments, a CD3ζ fused to an intracellular domain may not comprise a C terminal 2A domain. In certain embodiments, a CD3ζ fused to an intracellular domain may not comprise an N terminal signal peptide domain.

```
  (SEQ ID NO: 106)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGCTTTGCGCACGCCCACGCCGCAGCCCCGCCCAA
GAAGATGGCAAAGTCTACATCAACATGCCAGGCAGGGGCAGAGTGAAGTTCAGCAGGAGCGC
AGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCC
CCTCGCCAGTGCACCAACTACGCCCTGCTGAAGCTGGCCGGCGACGTGGAGAGCAACCCCGG
CCCC
(SEQ ID NO: 107)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCAAGAGTGAAGTTCAGCAGGAGCGCAGACGCCCCCG
CGTACCAGCAGGGCCAGAACCAGCTCTATAACGAGCTCAATCTAGGACGAAGAGAGGAGTAC
GATGTTTTGGACAAGAGACGTGGCCGGGACCCTGAGATGGGGGGAAAGCCGCAGAGAAGGAA
GAACCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTCGCCAGTG
CACCAACTACGCCCTGCTGAAGCTGGCCGGCGACGTGGAGAGCAACCCCGGCCCC
  (SEQ ID NO: 108)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCACTTTGCGCACGCCCACGCCGCAGCCCCGCCCAAG
AAGATGGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCC
  (SEQ ID NO: 109)
MKWKALFTAAILQAQLPITEAQSFGLLDPKLCYLLDGILFIYGVILTALFLLCARPRRSPAQ
EDGKVYINMPGRGRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQALP
PRQCTNYALLKLAGDVESNPGP
  (SEQ ID NO: 110)
MKWKALFTAAILQAQLPITEAQSFGLLDPKLCYLLDGILFIYGVILTALFLRSKRSRLLHSD
YMNMTPRRPGPTRKHYQPYAPPRDFAAYRSRVKESRSADAPAYQQGQNQLYNELNLGRREEY
DVLDKRRGRDPEMGGKPQRRKNPQEGLYNELQKDKMAEAYSEIGMKGERRRGKGHDGLYQGL
STATKDTYDALHMQALPPRQCTNYALLKLAGDVESNPGP
  (SEQ ID NO: 111)
MKWKALFTAAILQAQLPITEAQSFGLLDPKLCYLLDGILFIYGVILTALFLRSKRSRLLHSD
YMNMTPRRPGPTRKHYQPYAPPRDFAAYRSLCARPRRSPAQEDGKVYINMPGRGRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
P
```

In certain embodiments, a DAP10 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 112. In certain embodiments, a CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 113. In certain embodiments, a DAP10 intracellular co-stimulatory domain and CD28 intracellular co-stimulatory domain is represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 114. In certain embodiments, a DAP10 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 115. In certain embodiments, a CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 116. In certain embodiments, a DAP10 intracellular co-stimulatory domain and CD28 intracellular co-stimulatory domain is represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 117.

```
  (SEQ ID NO: 112)
CTTTGCGCACGCCCACGCCGCAGCCCCGCCCAAGAAGATGGCAAAGTCT
ACATCAACATGCCAGGCAGGGGC
  (SEQ ID NO: 113)
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCA
  (SEQ ID NO: 114)
AGGAGTAAGAGGAGCAGGCTCCTGCACAGTGACTACATGAACATGACTC
CCCGCCGCCCCGGGCCCACCCGCAAGCATTACCAGCCCTATGCCCCACC
ACGCGACTTCGCAGCCTATCGCTCACTTTGCGCACGCCCACGCCGCAGC
CCCGCCCAAGAAGATGGCAAAGTCTACATCAACATGCCAGGCAGGGGC
  (SEQ ID NO: 115)
LCARPRRSPAQEDGKVYINMPGRG
  (SEQ ID NO: 116)
RSKRSRLLHSDYMNMTPRRPGPTRKHYQPYAPPRDFAAYRS
(SEQ ID NO: 117)
RSKRSRLLHSDYMNMTPRRPGPTRKHYQPYAPPRDFAAYRSLCARPRRS
PAQEDGKVYINMPGRG
```

UTNK15-DAP10: refers to full length CD3zeta comprising a fusion with an intracellular co-stimulatory domain derived from DAP10, full length CD3 gamma, full length CD3 delta, and full length CD3 epsilon linked to IL15, it may be represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 118. In certain embodiments, a UTNK15-DAP10 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 119.

(SEQ ID NO: 118)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGCTTTGCGCACGCCCACGCCGCAGCCCCGCCCAA
GAAGATGGCAAAGTCTACATCAACATGCCAGGCAGGGGCAGAGTGAAGTTCAGCAGGAGCGC
AGACGCCCCCGCGTACCAGCAGGGCCAGAACCAGCTCTATAACGAGCTCAATCTAGGACGAA
GAGAGGAGTACGATGTTTTGGACAAGAGACGTGGCCGGGACCCTGAGATGGGGGGAAAGCCG
CAGAGAAGGAAGAACCCTCAGGAAGGCCTGTACAATGAACTGCAGAAAGATAAGATGGCGGA
GGCCTACAGTGAGATTGGGATGAAAGGCGAGCGCCGGAGGGGCAAGGGGCACGATGGCCTTT
ACCAGGGTCTCAGTACAGCCACCAAGGACACCTACGACGCCCTTCACATGCAGGCCCTGCCC
CCTCGCCAGTGCACCAACTACGCCCTGCTGAAGCTGGCCGGCGACGTGGAGAGCAACCCCGG
CCCCATGGAACAGGGGAAGGGCCTGGCTGTCCTCATCCTGGCTATCATTCTTCTTCAAGGTA
CTTTGGCCCAGTCAATCAAAGGAAACCACTTGGTTAAGGTGTATGACTATCAAGAAGATGGT
TCGGTACTTCTGACTTGTGATGCAGAAGCCAAAAATATCACATGGTTTAAAGATGGGAAGAT
GATCGGCTTCCTAACTGAAGATAAAAAAAAATGGAATCTGGGAAGTAATGCCAAGGACCCTC
GTGGGATGTATCAGTGTAAAGGATCACAGAACAAGTCAAAACCACTCCAAGTGTATTACAGA
ATGTGTCAGAACTGCATTGAACTAAATGCAGCCACCATATCTGGCTTTCTCTTTGCTGAAAT
CGTCAGCATTTTCGTCCTTGCTGTTGGGGTCTACTTCATTGCTGGACAGGATGGAGTTCGCC
AGTCGAGAGCTTCAGACAAGCAGACTCTGTTGCCCAATGACCAGCTCTACCAGCCCCTCAAG
GATCGAGAAGATGACCAGTACAGCCACCTTCAAGGAAACCAGTTGAGGAGGAATGTGAAGCA
GACCCTGAACTTCGACCTGCTGAAGCTGGCCGGCGACGTGGAGAGCAACCCCGGCCCCATGG
AGCACAGCACCTTCCTGAGCGGCCTGGTGCTGGCCACCCTGCTGAGCCAGGTGAGCCCCTTC
AAGATCCCCATCGAGGAGCTGGAGGACAGAGTGTTCGTGAACTGCAACACCAGCATCACCTG
GGTGGAGGGCACCGTGGGCACCCTGCTGAGCGACATCACCAGACTGGACCTGGGCAAGAGAA
TCCTGGACCCCAGAGGCATCTACAGATGCAACGGCACCGACATCTACAAGGACAAGGAGAGC
ACCGTGCAGGTGCACTACAGAATGTGCCAGAGCTGCGTGGAGCTGGACCCCGCCACCGTGGC
CGGCATCATCGTGACCGACGTGATCGCCACCCTGCTGCTGGCCCTGGGCGTGTTCTGCTTCG
CCGGCCACGAGACCGGCAGACTGAGCGGCGCCGCCGACACCCAGGCCCTGCTGAGAAACGAC
CAGGTGTACCAGCCCCTGAGAGACAGAGACGACGCCCAGTACAGCCACCTGGGCGGCAACTG
GGCCAGAAACAAGGAGGGCAGAGGCAGCCTGCTGACCTGCGGCGACGTGGAGGAGAACCCCG
GCCCCATGCAGAGCGGCACCCACTGGAGAGTGCTGGGCCTGTGCCTGCTGAGCGTGGGCGTG
TGGGGCCAGGACGGCAACGAGGAGATGGGCGGCATCACCCAGACCCCCTACAAGGTGAGCAT
CAGCGGCACCACCGTGATCCTGACCTGCCCCCAGTACCCCGGCAGCGAGATCCTGTGGCAGC
ACAACGACAAGAACATCGGCGGCGACGAGGACGACAAGAACATCGGCAGCGACGAGGACCAC
CTGAGCCTGAAGGAGTTCAGCGAGCTGGAGCAGAGCGGCTACTACGTGTGCTACCCCAGAGG
CAGCAAGCCCGAGGACGCCAACTTCTACCTGTACCTGAGAGCCAGAGTGTGCGAGAACTGCA
TGGAGATGGACGTGATGAGCGTGGCCACCATCGTGATCGTGGACATCTGCATCACCGGCGGC
CTGCTGCTGCTGGTGTACTACTGGAGCAAGAACAGAAAGGCCAAGGCCAAGCCCGTGACCAG
AGGCGCCGGCGCCGGCGGCAGACAGAGAGGCCAGAACAAGGAGAGACCCCCCCCCGTGCCCA
ACCCCGACTACGAGCCCATCAGAAAGGGCCAGAGAGACCTGTACAGCGGCCTGAACCAGAGA
AGAATCGGACCGCAGTGTACTAATTATGCTCTCTTGAAATTGGCTGGAGATGTTGAGAGCAA
TCCCGGGCCCATGCGCATTAGCAAGCCCCACCTGCGGAGCATCAGCATCCAGTGCTACCTGT
GCCTGCTGCTGAACAGCCACTTCCTGACCGAGGCCGGCATCCACGTGTTCATCCTGGGCTGC
TTCAGCGCCGGACTGCCCAAGACCGAGGCCAACTGGGTGAACGTGATCAGCGACCTGAAGAA
GATCGAGGACCTGATCCAGAGCATGCACATCGACGCCACCCTGTACACCGAGAGCGACGTGC
ACCCCAGCTGCAAGGTGACCGCCATGAAGTGCTTTCTGCTGGAACTGCAGGTGATCAGCCTG
GAAAGCGGCGACGCCAGCATCCACGACACCGTGGAGAACCTGATCATCCTGGCCAACAACAG
CCTGAGCAGCAACGGCAACGTGACCGAGAGCGGCTGCAAAGAGTGCGAGGAACTGGAAGAGA
AGAACATCAAAGAGTTTCTGCAGAGCTTCGTGCACATCGTGCAGATGTTCATCAACACCAGC
(SEQ ID NO: 119)
MKWKALFTAAILQAQLPITEAQSFGLLDPKLCYLLDGILFIYGVILTALFLLCARPRRSPAQ
EDGKVYINMPGRGRVKFSRSADAPAYQQGQNQLYNELNLGRREEYDVLDKRRGRDPEMGGKP
QRRKNPQEGLYNELQKDKMAEAYSEIGMKGERRRGKGHDGLYQGLSTATKDTYDALHMQALP
PRQCTNYALLKLAGDVESNPGPMEQGKGLAVLILAIILLQGTLAQSIKGNHLVKVYDYQEDG
SVLLTCDAEAKNITWFKDGKMIGFLTEDKKKWNLGSNAKDPRGMYQCKGSQNKSKPLQVYYR
MCQNCIELNAATISGFLFAEIVSIFVLAVGVYFIAGQDGVRQSRASDKQTLLPNDQLYQPLK
DREDDQYSHLQGNQLRRNVKQTLNFDLLKLAGDVESNPGPMEHSTFLSGLVLATLLSQVSPF
KIPIEELEDRVFVNCNTSITWVEGTVGTLLSDITRLDLGKRILDPRGIYRCNGTDIYKDKES
TVQVHYRMCQSCVELDPATVAGIIVTDVIATLLLALGVFCFAGHETGRLSGAADTQALLRND
QVYQPLRDRDDAQYSHLGGNWARNKEGRGSLLTCGDVEENPGPMQSGTHWRVLGLCLLSVGV
WGQDGNEEMGGITQTPYKVSISGTTVILTCPQYPGSEILWQHNDKNIGGDEDDKNIGSDEDH
LSLKEFSELEQSGYYVCYPRGSKPEDANFYLYLRARVCENCMEMDVMSVATIVIVDICITGG
LLLLVYYWSKNRKAKAKPVTRGAGAGGRQRGQNKERPPPVPNPDYEPIRKGQRDLYSGLNQR
RIGPQCTNYALLKLAGDVESNPGPMRISKPHLRSISIQCYLCLLLNSHELTEAGIHVFILGC
FSAGLPKTEANWVNVISDLKKIEDLIQSMHIDATLYTESDVHPSCKVTAMKCFLLELQVISL
ESGDASIHDTVENLIILANNSLSSNGNVTESGCKECEELEEKNIKEFLQSFVHIVQMFINTS

UTNK15-28: refers to full length CD3zeta comprising a fusion with an intracellular co-stimulatory domain derived from CD28, full length CD3 gamma, full length CD3 delta, and full length CD3 epsilon linked to IL15, it may be represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 120. In certain embodiments, a UTNK15-28 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 121.

```
  (SEQ ID NO: 120)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCAAGAGTGAAGTTCAGCAGGAGCGCAGACGCCCCCG
CGTACCAGCAGGGCCAGAACCAGCTCTATAACGAGCTCAATCTAGGACGAAGAGAGGAGTAC
GATGTTTTGGACAAGAGACGTGGCCGGGACCCTGAGATGGGGGGAAAGCCGCAGAGAAGGAA
GAACCCTCAGGAAGGCCTGTACAATGAACTGCAGAAAGATAAGATGGCGGAGGCCTACAGTG
AGATTGGGATGAAAGGCGAGCGCCGGAGGGGCAAGGGGCACGATGGCCTTTACCAGGGTCTC
AGTACAGCCACCAAGGACACCTACGACGCCCTTCACATGCAGGCCCTGCCCCCTCGCCAGTG
CACCAACTACGCCCTGCTGAAGCTGGCCGGCGACGTGGAGAGCAACCCCGGCCCCATGGAAC
AGGGGAAGGGCCTGGCTGTCCTCATCCTGGCTATCATTCTTCTTCAAGGTACTTTGGCCCAG
TCAATCAAAGGAAACCACTTGGTTAAGGTGTATGACTATCAAGAAGATGGTTCGGTACTTCT
GACTTGTGATGCAGAAGCCAAAAATATCACATGGTTTAAAGATGGGAAGATGATCGGCTTCC
TAACTGAAGATAAAAAAAAATGGAATCTGGGAAGTAATGCCAAGGACCCTCGTGGGATGTAT
CAGTGTAAAGGATCACAGAACAAGTCAAAACCACTCCAAGTGTATTACAGAATGTGTCAGAA
CTGCATTGAACTAAATGCAGCCACCATATCTGGCTTTCTCTTTGCTGAAATCGTCAGCATTT
TCGTCCTTGCTGTTGGGGTCTACTTCATTGCTGGACAGGATGGAGTTCGCCAGTCGAGAGCT
TCAGACAAGCAGACTCTGTTGCCCAATGACCAGCTCTACCAGCCCCTCAAGGATCGAGAAGA
TGACCAGTACAGCCACCTTCAAGGAAACCAGTTGAGGAGGAATGTGAAGCAGACCCTGAACT
TCGACCTGCTGAAGCTGGCCGGCGACGTGGAGAGCAACCCCGGCCCCATGGAGCACAGCACC
TTCCTGAGCGGCCTGGTGCTGGCCACCCTGCTGAGCCAGGTGAGCCCCTTCAAGATCCCCAT
CGAGGAGCTGGAGGACAGAGTGTTCGTGAACTGCAACACCAGCATCACCTGGGTGGAGGGCA
CCGTGGGCACCCTGCTGAGCGACATCACCAGACTGGACCTGGGCAAGAGAATCCTGGACCCC
AGAGGCATCTACAGATGCAACGGCACCGACATCTACAAGGACAAGGAGAGCACCGTGCAGGT
GCACTACAGAATGTGCCAGAGCTGCGTGGAGCTGGACCCCGCCACCGTGGCCGGCATCATCG
TGACCGACGTGATCGCCACCCTGCTGCTGGCCCTGGGCGTGTTCTGCTTCGCCGGCCACGAG
ACCGGCAGACTGAGCGGCGCCGCCGACACCCAGGCCCTGCTGAGAAACGACCAGGTGTACCA
GCCCCTGAGAGACAGAGACGACGCCCAGTACAGCCACCTGGGCGGCAACTGGGCCAGAAACA
AGGAGGGCAGAGGCAGCCTGCTGACCTGCGGCGACGTGGAGGAGAACCCCGGCCCCATGCAG
AGCGGCACCCACTGGAGAGTGCTGGGCCTGTGCCTGCTGAGCGTGGGCGTGTGGGGCCAGGA
CGGCAACGAGGAGATGGGCGGCATCACCCAGACCCCCTACAAGGTGAGCATCAGCGGCACCA
CCGTGATCCTGACCTGCCCCCAGTACCCCGGCAGCGAGATCCTGTGGCAGCACAACGACAAG
AACATCGGCGGCGACGAGGACGACAAGAACATCGGCAGCGACGAGGACCACCTGAGCCTGAA
GGAGTTCAGCGAGCTGGAGCAGAGCGGCTACTACGTGTGCTACCCCAGAGGCAGCAAGCCCG
AGGACGCCAACTTCTACCTGTACCTGAGAGCCAGAGTGTGCGAGAACTGCATGGAGATGGAC
GTGATGAGCGTGGCCACCATCGTGATCGTGGACATCTGCATCACCGGCGGCCTGCTGCTGCT
GGTGTACTACTGGAGCAAGAACAGAAAGGCCAAGGCCAAGCCCGTGACCAGAGGCGCCGGCG
CCGGCGGCAGACAGAGAGGCCAGAACAAGGAGAGACCCCCCCCCGTGCCCAACCCCGACTAC
GAGCCCATCAGAAAGGGCCAGAGAGACCTGTACAGCGGCCTGAACCAGAGAAGAATCGGACC
GCAGTGTACTAATTATGCTCTCTTGAAATTGGCTGGAGATGTTGAGAGCAATCCCGGGCCCA
TGCGCATTAGCAAGCCCCACCTGCGGAGCATCAGCATCCAGTGCTACCTGTGCCTGCTGCTG
AACAGCCACTTCCTGACCGAGGCCGGCATCCACGTGTTCATCCTGGGCTGCTTCAGCGCCGG
ACTGCCCAAGACCGAGGCCAACTGGGTGAACGTGATCAGCGACCTGAAGAAGATCGAGGACC
TGATCCAGAGCATGCACATCGACGCCACCCTGTACACCGAGAGCGACGTGCACCCCAGCTGC
AAGGTGACCGCCATGAAGTGCTTTCTGCTGGAACTGCAGGTGATCAGCCTGGAAAGCGGCGA
CGCCAGCATCCACGACACCGTGGAGAACCTGATCATCCTGGCCAACAACAGCCTGAGCAGCA
ACGGCAACGTGACCGAGAGCGGCTGCAAAGAGTGCGAGGAACTGGAAGAGAAGAACATCAAA
GAGTTTCTGCAGAGCTTCGTGCACATCGTGCAGATGTTCATCAACACCAGC
  (SEQ ID NO: 121)
MKWKALFTAAILQAQLPITEAQQSFGLLDPKLCYLLDGILFIYGVILTALFLRSKRSRLLHSD
YMNMTPRRPGPTRKHYQPYAPPRDFAAYRSRVKFSRSADAPAYQQGQNQLYNELNLGRREEY
DVLDKRRGRDPEMGGKPQRRKNPQEGLYNELQKDKMAEAYSEIGMKGERRRGKGHDGLYQGL
STATKDTYDALHMQALPPRQCTNYALLKLAGDVESNPGPMEQGKGLAVLILAIILLQGTLAQ
SIKGNHLVKVYDYQEDGSVLLTCDAEAKNITWFKDGKMIGFLTEDKKKWNLGSNAKDPRGMY
QCKGSQNKSKPLQVYYRMCQNCIELNAATISGFLFAEIVSIFVLAVGVYFIAGQDGVRQSRA
SDKQTLLPNDQLYQPLKDREDDQYSHLQGNQLRRNVKQTLNFDLLKLAGDVESNPGPMEHST
FLSGLVLATLLSQVSPFKIPIEELEDRVFVNCNTSITWVEGTVGTLLSDITRLDLGKRILDP
RGIYRCNGTDIYKDKESTVQVHYRMCQSCVELDPATVAGIIVTDVIATLLLALGVFCFAGHE
TGRLSGAADTQALLRNDQVYQPLRDRDDAQYSHLGGNWARNKEGRGSLLTCGDVEENPGPMQ
SGTHWRVLGLCLLSVGVWGQDGNEEMGGITQTPYKVSISGTTVILTCPQYPGSEILWQHNDK
NIGGDEDDKNIGSDEDHLSLKEFSELEQSGYYVCYPRGSKPEDANFYLYLRARVCENCMEMD
VMSVATIVIVDICITGGLLLLVYYWSKNRKAKAKPVTRGAGAGGRQRGQNKERPPPVPNPDY
EPIRKGQRDLYSGLNQRRIGPQCTNYALLKLAGDVESNPGPMRISKPHLRSISIQCYLCLLL
NSHFLTEAGIHVFILGCFSAGLPKTEANWVNVISDLKKIEDLIQSMHIDATLYTESDVHPSC
KVTAMKCFLLELQVISLESGDASIHDTVENLIILANNSLSSNGNVTESGCKECEELEEKNIK
EFLQSFVHIVQMFINTS
```

UTNK15-28-DAP10: refers to full length CD3zeta comprising a fusion with an intracellular co-stimulatory domain derived from DAP10 and an intracellular co-stimulatory domain derived from CD28, full length CD3 gamma, full length CD3 delta, and full length CD3 epsilon linked to IL15, it may be represented by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 122. In certain embodiments, a UTNK15-28-DAP10 amino acid sequence may be represented by an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 123.

(SEQ ID NO: 122)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGGAGTAAGAGGAGCAGGCTCCTGCACAGTGAC
TACATGAACATGACTCCCCGCCGCCCCGGGCCCACCCGCAAGCATTACCAGCCCTATGCCCC
ACCACGCGACTTCGCAGCCTATCGCTCACTTTGCGCACGCCCACGCCGCAGCCCCGCCCAAG
AAGATGGCAAAGTCTACATCAACATGCCAGGCAGGGGCAGAGTGAAGTTCAGCAGGAGCGCA
GACGCCCCCGCGTACCAGCAGGGCCAGAACCAGCTCTATAACGAGCTCAATCTAGGACGAAG
AGAGGAGTACGATGTTTTGGACAAGAGACGTGGCCGGGACCCTGAGATGGGGGGAAAGCCGC
AGAGAAGGAAGAACCCTCAGGAAGGCCTGTACAATGAACTGCAGAAAGATAAGATGGCGGAG
GCCTACAGTGAGATTGGGATGAAAGGCGAGCGCCGGAGGGGCAAGGGGCACGATGGCCTTTA
CCAGGGTCTCAGTACAGCCACCAAGGACACCTACGACGCCCTTCACATGCAGGCCCTGCCCC
CTCGCCAGTGCACCAACTACGCCCTGCTGAAGCTGGCCGGCGACGTGGAGAGCAACCCCGGC
CCCATGGAACAGGGGAAGGGCCTGGCTGTCCTCATCCTGGCTATCATTCTTCTTCAAGGTAC
TTTGGCCCAGTCAATCAAAGGAAACCACTTGGTTAAGGTGTATGACTATCAAGAAGATGGTT
CGGTACTTCTGACTTGTGATGCAGAAGCCAAAAATATCACATGGTTTAAAGATGGGAAGATG
ATCGGCTTCCTAACTGAAGATAAAAAAAAATGGAATCTGGGAAGTAATGCCAAGGACCCTCG
TGGGATGTATCAGTGTAAAGGATCACAGAACAAGTCAAAACCACTCCAAGTGTATTACAGAA
TGTGTCAGAACTGCATTGAACTAAATGCAGCCACCATATCTGGCTTTCTCTTTGCTGAAATC
GTCAGCATTTTCGTCCTTGCTGTTGGGGTCTACTTCATTGCTGGACAGGATGGAGTTCGCCA
GTCGAGAGCTTCAGACAAGCAGACTCTGTTGCCCAATGACCAGCTCTACCAGCCCCTCAAGG
ATCGAGAAGATGACCAGTACAGCCACCTTCAAGGAAACCAGTTGAGGAGGAATGTGAAGCAG
ACCCTGAACTTCGACCTGCTGAAGCTGGCCGGCGACGTGGAGAGCAACCCCGGCCCCATGGA
GCACAGCACCTTCCTGAGCGGCCTGGTGCTGGCCACCCTGCTGAGCCAGGTGAGCCCCTTCA
AGATCCCCATCGAGGAGCTGGAGGACAGAGTGTTCGTGAACTGCAACACCAGCATCACCTGG
GTGGAGGGCACCGTGGGCACCCTGCTGAGCGACATCACCAGACTGGACCTGGGCAAGAGAAT
CCTGGACCCCAGAGGCATCTACAGATGCAACGGCACCGACATCTACAAGGACAAGGAGAGCA
CCGTGCAGGTGCACTACAGAATGTGCCAGAGCTGCGTGGAGCTGGACCCCGCCACCGTGGCC
GGCATCATCGTGACCGACGTGATCGCCACCCTGCTGCTGGCCCTGGGCGTGTTCTGCTTCGC
CGGCCACGAGACCGGCAGACTGAGCGGCGCCGCCGACACCCAGGCCCTGCTGAGAAACGACC
AGGTGTACCAGCCCCTGAGAGACAGAGACGACGCCCAGTACAGCCACCTGGGCGGCAACTGG
GCCAGAAACAAGGAGGGCAGAGGCAGCCTGCTGACCTGCGGCGACGTGGAGGAGAACCCCGG
CCCCATGCAGAGCGGCACCCACTGGAGAGTGCTGGGCCTGTGCCTGCTGAGCGTGGGCGTGT
GGGGCCAGGACGGCAACGAGGAGATGGGCGGCATCACCCAGACCCCCTACAAGGTGAGCATC
AGCGGCACCACCGTGATCCTGACCTGCCCCCAGTACCCCGGCAGCGAGATCCTGTGGCAGCA
CAACGACAAGAACATCGGCGGCGACGAGGACGACAAGAACATCGGCAGCGACGAGGACCACC
TGAGCCTGAAGGAGTTCAGCGAGCTGGAGCAGAGCGGCTACTACGTGTGCTACCCCAGAGGC
AGCAAGCCCGAGGACGCCAACTTCTACCTGTACCTGAGAGCCAGAGTGTGCGAGAACTGCAT
GGAGATGGACGTGATGAGCGTGGCCACCATCGTGATCGTGGACATCTGCATCACCGGCGGCC
TGCTGCTGCTGGTGTACTACTGGAGCAAGAACAGAAAGGCCAAGGCCAAGCCCGTGACCAGA
GGCGCCGGCGCCGGCGGCAGACAGAGAGGCCAGAACAAGGAGAGACCCCCCCCCGTGCCCAA
CCCCGACTACGAGCCCATCAGAAAGGGCCAGAGAGACCTGTACAGCGGCCTGAACCAGAGAA
GAATCGGACCGCAGTGTACTAATTATGCTCTCTTGAAATTGGCTGGAGATGTTGAGAGCAAT
CCCGGGCCCATGCGCATTAGCAAGCCCCACCTGCGGAGCATCAGCATCCAGTGCTACCTGTG
CCTGCTGCTGAACAGCCACTTCCTGACCGAGGCCGGCATCCACGTGTTCATCCTGGGCTGCT
TCAGCGCCGGACTGCCCAAGACCGAGGCCAACTGGGTGAACGTGATCAGCGACCTGAAGAAG
ATCGAGGACCTGATCCAGAGCATGCACATCGACGCCACCCTGTACACCGAGAGCGACGTGCA
CCCCAGCTGCAAGGTGACCGCCATGAAGTGCTTTCTGCTGGAACTGCAGGTGATCAGCCTGG
AAAGCGGCGACGCCAGCATCCACGACACCGTGGAGAACCTGATCATCCTGGCCAACAACAGC
CTGAGCAGCAACGGCAACGTGACCGAGAGCGGCTGCAAAGAGTGCGAGGAACTGGAAGAGAA
GAACATCAAAGAGTTTCTGCAGAGCTTCGTGCACATCGTGCAGATGTTCATCAACACCAGC
(SEQ ID NO: 123)
MKWKALFTAAILQAQLPITEAQSFGLLDPKLCYLLDGILFIYGVILTALFLRSKRSRLLHSD
YMNMTPRRPGPTRKHYQPYAPPRDFAAYRSLCARPRRSPAQEDGKVYINMPGRGRVKFSRSA
DAPAYQQGQNQLYNELNLGRREEYDVLDKRRGRDPEMGGKPQRRKNPQEGLYNELQKDKMAE
AYSEIGMKGERRRGKGHDGLYQGLSTATKDTYDALHMQALPPRQCTNYALLKLAGDVESNPG
PMEQGKGLAVLILAIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIELNAATISGELFAEI
VSIFVLAVGVYFIAGQDGVRQSRASDKQTLLPNDQLYQPLKDREDDQYSHLQGNQLRRNVKQ
TLNFDLLKLAGDVESNPGPMEHSTFLSGLVLATLLSQVSPEKIPIEELEDRVFVNCNTSITW
VEGTVGTLLSDITRLDLGKRILDPRGIYRCNGTDIYKDKESTVQVHYRMCQSCVELDPATVA
GIIVTDVIATLLLALGVFCFAGHETGRLSGAADTQALLRNDQVYQPLRDRDDAQYSHLGGNW
ARNKEGRGSLLTCGDVEENPGPMQSGTHWRVLGLCLLSVGVWGQDGNEEMGGITQTPYKVSI
SGTTVILTCPQYPGSEILWQHNDKNIGGDEDDKNIGSDEDHLSLKEFSELEQSGYYVCYPRG
SKPEDANFYLYLRARVCENCMEMDVMSVATIVIVDICITGGLLLLVYYWSKNRKAKAKPVTR
GAGAGGRQRGQNKERPPPVPNPDYEPIRKGQRDLYSGLNQRRIGPQCTNYALLKLAGDVESN
PGPMRISKPHLRSISIQCYLCLLLNSHFLTEAGIHVFILGCFSAGLPKTEANWVNVISDLKK
IEDLIQSMHIDATLYTESDVHPSCKVTAMKCFLLELQVISLESGDASIHDTVENLIILANNS
LSSNGNVTESGCKECEELEEKNIKEFLQSFVHIVQMFINTS

As depicted in FIG. 3 and described above, the term “linked” refers to being present on the same polynucleotide vector and does not necessarily mean that the two polypeptides are expressed as one polypeptide. For example, a cytokine produced from a vector of the disclosure may ultimately be produced as a separate molecule from any one or more TCR/CD3 receptor complex components. Whereas, the term “fused” or “fusion” refers to two polypeptides that comprise a peptide bond conjoining the two molecules, i.e. that the two polypeptides are covalently bound by an amide bond and are not separated by a splitting element, such as a 2A element.

One specific example of a TCR that may be utilized in the cells is NY-ESO TCR, and specific examples of sequences include at least the following:

### TCRα:

(SEQ ID NO: 25)
XQEVTQIPAALSVPEGENLVLNCSFTDSAIYNLQWFRQDPGKGLTSLLL
IQSSQREQTSGRLNASLDKSSGRSTLYIAASQPGDSATYLCAVRPLYGG
SYIPTFGRGTSLIVHPYIQNPDPAVYQLRDSKSSDKSVCLFTDFDSQTN
VSQSKDSDVYITDKTVLDMRSMDFKSNSAVAWSNKSDFACANAFNNSII
PEDTFFPSPESSCDVKLVEKSFETDTNLNFQNLSVIGFRILLLKVAGEN
LLMTLRLWSS

### TCRβ:

(SEQ ID NO: 26)
GVTQTPKFQVLKTGQSMTLQCAQDMNHEYMSWYRQDPGMGLRLIHYSVG
AGITDQGEVPNGYNVSRSTTEDFPLRLLSAAPSQTSVYFCASSYVGNTG
ELFFGEGSRLTVLEDLKNVFPPKVAVFEPSEAEISHTQKATLVCLATGF
YPDHVELSWWVNGKEVHSGVSTDPQPLKEQPALNDSRYCLSSRLRVSAT
FWQNPRNHFRCQVQFYGLSENDEWTQDRAKPVTQIVSAEAWGRADCGFT
SESYQQGVLSATILYEILLGKATLYAVLVSALVLMAMVKRKDSRG

In certain embodiments, a TCR may comprise a TCR alpha chain variable region encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 85.

(SEQ ID NO: 85)
aaacaggaggtgacacagattcctgcagctctgagtgtcccagaaggaga
aaacttggttctcaactgcagtttcactgatagcgctatttacaacctcc
agtggtttaggcaggaccctgggaaaggtctcacatctctgttgcttatt
cagtcaagtcagagagagcaaacaagtggaagacttaatgcctcgctgga
taaatcatcaggacgtagtactttatacattgcagcttctcagcctggtg
actcagccacctacctctgtgctgtgaggcccctttatggaggaagctac
atacctacatttggaagaggaaccagccttattgttcatccgtat

In certain embodiments, a TCR may comprise a TCR alpha chain constant region encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 86.

(SEQ ID NO: 86)
atccagaaccctgaccctgccgtgtaccagctgagagactctaaatccag
tgacaagtctgtctgcctattcaccgattttgattctcaaacaaatgtgt
cacaaagtaaggattctgatgtgtatatcacagacaaaactgtgctagac
atgaggtctatggacttcaagagcaacagtgctgtggcctggagcaacaa
atctgactttgcatgtgcaaacgccttcaacaacagcattattccagaag
acaccttcttccccagcccagaaagttcctgtgatgtcaagctggtcgag
aaaagctttgaaacagatacgaacctaaactttcaaaacctgtcagtgat
tgggttccgaatcctcctcctgaaagtggccgggtttaatctgctcatga
cgctgcggctgtggtccagc

In certain embodiments, a TCR may comprise a TCR alpha chain encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 87.

(SEQ ID NO: 87)
atggagaccctcttgggcctgcttatcctttggctgcagctgcaatgggt
gagcagcaaacaggaggtgacacagattcctgcagctctgagtgtcccag
aaggagaaaacttggttctcaactgcagtttcactgatagcgctatttac
aacctccagtggtttaggcaggaccctgggaaaggtctcacatctctgtt
gcttattcagtcaagtcagagagagcaaacaagtggaagacttaatgcct
cgctggataaatcatcaggacgtagtactttatacattgcagcttctcag
cctggtgactcagccacctacctctgtgctgtgaggcccctttatggagg
aagctacatacctacatttggaagaggaaccagccttattgttcatccgt
atatccagaaccctgaccctgccgtgtaccagctgagagactctaaatcc
agtgacaagtctgtctgcctattcaccgattttgattctcaaacaaatgt
gtcacaaagtaaggattctgatgtgtatatcacagacaaaactgtgctag
acatgaggtctatggacttcaagagcaacagtgctgtggcctggagcaac
aaatctgactttgcatgtgcaaacgccttcaacaacagcattattccaga
agacaccttcttccccagcccagaaagttcctgtgatgtcaagctggtcg
agaaaagctttgaaacagatacgaacctaaactttcaaaacctgtcagtg
attgggttccgaatcctcctcctgaaagtggccgggtttaatctgctcat
gacgctgcggctgtggtccagc

In certain embodiments, a TCR may comprise a TCR alpha chain variable region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 88.

(SEQ ID NO: 88)
XQEVTQIPAALSVPEGENLVLNCSFTDSAIYNLQWFRQDPGKGLTSLLLI
QSSQREQTSGRLNASLDKSSGRSTLYIAASQPGDSATYLCAVRPLYGGSY
IPTFGRGTSLIVHPY

In certain embodiments, a TCR may comprise a TCR alpha chain constant region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 89.

(SEQ ID NO: 89)
IQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVE
KSFETDTNLNFQNLSVIGFRILLLKVAGFNLLMTLRLWSS

In certain embodiments, a TCR may comprise an alpha chain CDR1 amino acid sequence that is at least, or exactly, 80% or 100% identical to SEQ ID NO: 90.
DSAIYN (SEQ ID NO: 90)

In certain embodiments, a TCR may comprise an alpha chain CDR2 amino acid sequence that is at least, or exactly, 80% or 100% identical to SEQ ID NO: 91.
IQSSQRE (SEQ ID NO: 91)

In certain embodiments, a TCR may comprise an alpha chain CDR3 amino acid sequence that is at least, or exactly, 80% or 100% identical to SEQ ID NO: 92.
CAVRPLYGGSYIPTF (SEQ ID NO: 92)

In certain embodiments, a TCR may comprise a TCR beta chain variable encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 93.

(SEQ ID NO: 93)
ggtgtcactcagaccccaaaattccaggtcctgaagacaggacagagcat
gacactgcagtgtgcccaggatatgaaccatgaatacatgtcctggtatc
gacaagacccaggcatggggctgaggctgattcattactcagttggtgct
ggtatcactgaccaaggagaagtccccaatggctacaatgtctccagatc
aaccacagaggatttcccgctcaggctgctgtcggctgctccctcccaga
catctgtgtacttctgtgccagcagttacgtcgggaacaccggggagctg
ttttttggagaaggctctaggctgaccgtactggag

In certain embodiments, a TCR may comprise a TCR beta chain constant region encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 94.

(SEQ ID NO: 94)
Gacctgaaaaacgtgttcccacccaaggtcgctgtgtttgagccatcaga
agcagagatctcccacacccaaaaggccacactggtatgcctggccacag
gcttctaccccgaccacgtggagctgagctggtgggtgaatgggaaggag
gtgcacagtggggtcagcacagacccgcagcccctcaaggagcagcccgc
cctcaatgactccagatactgcctgagcagccgcctgagggtctcggcca
ccttctggcagaacccccgcaaccacttccgctgtcaagtccagttctac
gggctctcggagaatgacgagtggacccaggatagggccaaacccgtcac
ccagatcgtcagcgccgaggcctggggtagagcagactgtggcttcacct
ccgagtcttaccagcaaggggtcctgtctgccaccatcctctatgagatc
ttgctagggaaggccaccttgtatgccgtgctggtcagtgccctcgtgct
gatggccatggtcaagagaaaggattccagaggc

In certain embodiments, a TCR may comprise a TCR beta chain encoded by a nucleotide sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 95.

(SEQ ID NO: 95)
Atgagcatcggcctcctgtgctgtgcagccttgtctctcctgtgggcagg
tccagtgaatgctggtgtcactcagaccccaaaattccaggtcctgaaga
caggacagagcatgacactgcagtgtgcccaggatatgaaccatgaatac
atgtcctggtatcgacaagacccaggcatggggctgaggctgattcatta
ctcagttggtgctggtatcactgaccaaggagaagtccccaatggctaca
atgtctccagatcaaccacagaggatttcccgctcaggctgctgtcggct
gctccctcccagacatctgtgtacttctgtgccagcagttacgtcgggaa
caccggggagctgttttttggagaaggctctaggctgaccgtactggagg
acctgaaaaacgtgttcccacccAaggtcgctgtgtttgagccatcagaa
gcagagatctcccacacccaaaaggccacactggtatgcctggccacagg
cttctaccccgaccacgtggagctgagctggtgggtgaatgggaaggagg
tgcacagtggggtcagcacagacccgcagcccctcaaggagcagcccgcc
ctcaatgactccagatactgcctgagcagccgcctgagggtctcggccac
cttctggcagaacccccgcaaccacttccgctgtcaagtccagttctacg
ggctctcggagaatgacgagtggacccaggatagggccaaacccgtcacc
cagatcgtcagcgccgaggcctggggtagagcagactgtggcttcacctc
cgagtcttaccagcaaggggtcctgtctgccaccatcctctatgagatct
tgctagggaaggccaccttgtatgccgtgctggtcagtgccctcgtgctg
atggccatggtcaagagaaaggattccagaggc

In certain embodiments, a TCR may comprise a TCR beta chain variable region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 96.

(SEQ ID NO: 96)
GVTQTPKFQVLKTGQSMTLQCAQDMNHEYMSWYRQDPGMGLRLIHYSVGA
GITDQGEVPNGYNVSRSTTEDFPLRLLSAAPSQTSVYFCASSYVGNTGEL
FFGEGSRLTVLE

In certain embodiments, a TCR may comprise a TCR beta chain constant region amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 97.

(SEQ ID NO: 97)
DLKNVFPPKVAVFEPSEAEISHTQKATLVCLATGFYPDHVELSWWVNGKE
VHSGVSTDPQPLKEQPALNDSRYCLSSRLRVSATFWQNPRNHFRCQVQFY
GLSENDEWTQDRAKPVTQIVSAEAWGRADCGFTSESYQQGVLSATILYEI
LLGKATLYAVLVSALVLMAMVKRKDSRG

In certain embodiments, a TCR may comprise a beta chain CDR1 amino acid sequence that is at least, or exactly, 80% or 100% identical to SEQ ID NO: 98.

(SEQ ID NO: 98)
MNHEY

In certain embodiments, a TCR may comprise a beta chain CDR2 amino acid sequence that is at least, or exactly, 80% or 100% identical to SEQ ID NO: 99.

(SEQ ID NO: 99)
SVGAGI

In certain embodiments, a TCR may comprise a beta chain CDR3 amino acid sequence that is at least, or exactly, 80% or 100% identical to SEQ ID NO: 100.

(SEQ ID NO: 100)
CASSYVGNTGELFF

In certain embodiments, a TCR (e.g., a TCR alpha, beta, delta, and/or gamma) chain may comprise a signal peptide. In certain embodiments, a signal peptide is encoded by a nucleic acid that is at least, or exactly 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 101 or SEQ ID NO: 102. In certain embodiments, a signal peptide is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100% identical to SEQ ID NO: 103 or SEQ ID NO: 104.

(SEQ ID NO: 101)
atggagaccctcttgggcctgcttatcctttggctgcagctgcaatgggt
gagcagc
(SEQ ID NO: 102)
atgagcatcggcctcctgtgctgtgcagccttgtctctcctgtgggcagg
tccagtgaatgct
(SEQ ID NO: 103)
METLLGLLILWLQLQWVSS
(SEQ ID NO: 104)
MSIGLLCCAALSLLWAGPVNA

In certain embodiments, a TCR recognizes a peptide corresponding to amino acid residues 157-165 of the human cancer testis Ag NY-ESO-1 in the context of the HLA-A*02 class I allele. In certain embodiments, a TCR may target an epitope characterized by the amino acid sequence according to SEQ ID NO: 105.

(SEQ ID NO: 105)
SLIMWITQC

One specific example of a TCR that may be utilized in the cells is TCRpp65alpha, and specific examples of sequences include at least the following (underlining refers to signal peptide sequence):

(SEQ ID NO: 27)
ATGGACTCCTGGACCTTCTGCTGTGTGTCCCTTTGCATCCTGGTAGCAAA
GCACACAGATGCTGGACAACAGCTGAATCAGAGTCCTCAATCTATGTTTA
TCCAGGAAGGAGAAGATGTCTCCATGAACTGCACTTCTTCAAGCATATTT
AACACCTGGCTATGGTACAAGCAGGACCCTGGGGAAGGTCCTGTCCTCTT
GATAGCCTTATATAAGGCTGGTGAATTGACCTCAAATGGAAGACTGACTG
CTCAGTTTGGTATAACCAGAAAGGACAGCTTCCTGAATATCTCAGCATCC
ATACCCAGTGATGTAGGCATCTACTTCTGTGCTGGACCCATGAAAACCTC
CTACGACAAGGTGATATTTGGGCCAGGGACAAGCTTATCAGTCATTCCAA
ATATCCAGAACCCTGACCCTGCCGTGTACCAGCTGAGAGACTCTAAATCC
AGTGACAAGTCTGTCTGCCTATTCACCGATTTTGATTCTCAAACAAATGT
GTCACAAAGTAAGGATTCTGATGTGTATATCACAGACAAAACTGTGCTAG
ACATGAGGTCTATGGACTTCAAGAGCAACAGTGCTGTGGCCTGGAGCAAC
AAATCTGACTTTGCATGTGCAAACGCCTTCAACAACAGCATTATTCCAGA
AGACACCTTCTTCCCCAGCCCAGAAAGTTCCTGTGATGTCAAGCTGGTCG
AGAAAAGCTTTGAAACAGATACGAACCTAAACTTTCAAAACCTGTCAGTG
ATTGGGTTCCGAATCCTCCTCCTGAAAGTGGCCGGGTTTAATCTGCTCAT
GACGCTGCGGCTGTGGTCCAGC
(SEQ ID NO: 28)
MDSWTFCCVSLCILVAKHTDAGQQLNQSPQSMFIQEGEDVSMNCTSSSIF
NTWLWYKQDPGEGPVLLIALYKAGELTSNGRLTAQFGITRKDSFLNISAS
IPSDVGIYFCAGPMKTSYDKVIFGPGTSLSVIPNIQNPDPAVYQLRDSKS
SDKSVCLFTDFDSQTNVSQSKDSDVYITDKTVLDMRSMDFKSNSAVAWSN
KSDFACANAFNNSIIPEDTFFPSPESSCDVKLVEKSFETDINLNFQNLSV
IGFRILLLKVAGFNLLMTLRLWSS

One specific example of a TCR that may be utilized in the cells is TCRpp65beta and specific examples of sequences include at least the following (underlining refers to signal peptide sequence):

(SEQ ID NO: 29)
ATGGACTCCTGGACCTTCTGCTGTGTGTCCCTTTGCATCCTGGTAGCAAA
GCACACAGATGCTGGAGTTATCCAGTCACCCCGGCACGAGGTGACAGAGA
TGGGACAAGAAGTGACTCTGAGATGTAAACCAATTTCAGGACACGACTAC
CTTTTCTGGTACAGACAGACCATGATGCGGGGACTGGAGTTGCTCATTTA
CTTTAACAACAACGTTCCGATAGATGATTCAGGGATGCCCGAGGATCGAT
TCTCAGCTAAGATGCCTAATGCATCATTCTCCACTCTGAAGATCCAGCCC
TCAGAACCCAGGGACTCAGCTGTGTACTTCTGTGCCAGCAGTTCGGCAAA
CTATGGCTACACCTTCGGTTCGGGGACCAGGTTAACCGTTGTAGAGGACC
TGAACAAGGTGTTCCCACCCGAGGTCGCTGTGTTTGAGCCATCAGAAGCA
GAGATCTCCCACACCCAAAAGGCCACACTGGTGTGCCTGGCCACAGGCTT
CTTCCCTGACCACGTGGAGCTGAGCTGGTGGGTGAATGGGAAGGAGGTGC
ACAGTGGGGTCAGCACGGACCCGCAGCCCCTCAAGGAGCAGCCCGCCCTC
AATGACTCCAGATACTGCCTGAGCAGCCGCCTGAGGGTCTCGGCCACCTT
CTGGCAGAACCCCCGCAACCACTTCCGCTGTCAAGTCCAGTTCTACGGGC
TCTCGGAGAATGACGAGTGGACCCAGGATAGGGCCAAACCCGTCACCCAG
ATCGTCAGCGCCGAGGCCTGGGGTAGAGCAGACTGTGGCTTTACCTCGGT
GTCCTACCAGCAAGGGGTCCTGTCTGCCACCATCCTCTATGAGATCCTGC
TAGGGAAGGCCACCCTGTATGCTGTGCTGGTCAGCGCCCTTGTGTTGATG
GCCATGGTCAAGAGAAAGGATTTC
(SEQ ID NO: 30)
MDSWTFCCVSLCILVAKHTDAGVIQSPRHEVTEMGQEVTLRCKPISGHDY
LFWYRQTMMRGLELLIYFNNNVPIDDSGMPEDRFSAKMPNASFSTLKIQP
SEPRDSAVYFCASSSANYGYTFGSGTRLTVVEDLNKVFPPEVAVFEPSEA
EISHTQKATLVCLATGFFPDHVELSWWVNGKEVHSGVSTDPQPLKEQPAL
NDSRYCLSSRLRVSATFWQNPRNHFRCQVQFYGLSENDEWTQDRAKPVTQ
IVSAEAWGRADCGFTSVSYQQGVLSATILYEILLGKATLYAVLVSALVLM
AMVKRKDF

### TCRpp65ZFLGDEFL15

In certain embodiments, one may utilize a construct in which TCRpp65 is linked to full length CD3zeta, full length CD3 gamma, full length CD3 delta, full length CD3 epsilon, and also linked to IL-15 (and may be referred to as TCRpp65ZFLGDEFL15). One representative sequence for such a construct is as follows:

(SEQ ID NO: 74)
MLEGVTQTPKFQVLKTGQSMTLQCAQDMNHEYMSWYRQDPGMGLRLIHYS
VGAGITDQGEVPNGYNVSRSTTEDFPLRLLSAAPSQTSVYFCASSPVTGG
IYGYTFGSGTRLTVVEDLNKVFPPEVAVFEPSEAEISHTQKATLVCLATG
FFPDHVELSWWVNGKEVHSGVSTDPQPLKEQPALNDSRYCLSSRLRVSAT
FWQNPRNHFRCQVQFYGLSENDEWTQDRAKPVTQIVSAEAWGRADATNFS
LLKQAGDVEENPGPMILNVEQSPQSLHVQEGDSTNFTCSFPSSNFYALHW
YRWETAKSPEALFVMTLNGDEKKKGRISATLNTKEGYSYLYIKGSQPEDS
ATYLCARNTGNQFYFGTGTSLTVIPNIQNPDPAVYQLRDSKSSDKSVCLF
TDFDSQTNVSQSKDSDAYITDKTVLDMRSMDFKSNSAVAWSNKSDFACAN
AFNNSIIPEDTFFPSPESSEGRGSLLTCGDVEENPGPMKWKALFTAAILQ
AQLPITEAQSFGLLDPKLCYLLDGILFIYGVILTALFLRVKFSRSADAPA
YQQGQNQLYNELNLGRREEYDVLDKRRGRDPEMGGKPQRRKNPQEGLYNE
LQKDKMAEAYSEIGMKGERRRGKGHDGLYQGLSTATKDTYDALHMQALPP
RQCTNYALLKLAGDVESNPGPMEQGKGLAVLILAIILLQGTLAQSIKGNH
LVKVYDYQEDGSVLLTCDAEAKNITWFKDGKMIGFLTEDKKKWNLGSNAK
DPRGMYQCKGSQNKSKPLQVYYRMCQNCIELNAATISGFLFAEIVSIFVL
AVGVYFIAGQDGVRQSRASDKQTLLPNDQLYQPLKDREDDQYSHLQGNQL
RRNVKQTLNFDLLKLAGDVESNPGPMEHSTFLSGLVLATLLSQVSPFKIP
IEELEDRVFVNCNTSITWVEGTVGTLLSDITRLDLGKRILDPRGIYRCNG
TDIYKDKESTVQVHYRMCQSCVELDPATVAGIIVTDVIATLLLALGVFCF
AGHETGRLSGAADTQALLRNDQVYQPLRDRDDAQYSHLGGNWARNKEGRG
SLLTCGDVEENPGPMQSGTHWRVLGLCLLSVGVWGQDGNEEMGGITQTPY
KVSISGTTVILTCPQYPGSEILWQHNDKNIGGDEDDKNIGSDEDHLSLKE
FSELEQSGYYVCYPRGSKPEDANFYLYLRARVCENCMEMDVMSVATIVIV
DICITGGLLLLVYYWSKNRKAKAKPVTRGAGAGGRQRGQNKERPPPVPNP
DYEPIRKGQRDLYSGLNQRRIGPQCTNYALLKLAGDVESNPGPMRISKPH
LRSISIQCYLCLLLNSHFLTEAGIHVFILGCFSAGLPKTEANWVNVISDL
KKIEDLIQSMHIDATLYTESDVHPSCKVTAMKCFLLELQVISLESGDASI
HDTVENLIILANNSLSSNGNVTESGCKECEELEEKNIKEFLQSFVHIVQM
FINTS*.

In TCRpp65ZFLGDEFL15, the corresponding component sequences are as follows, although these particular sequences or others may be utilized in this and/or other constructs:

### TCRb-Extracellular Domain:

(SEQ ID NO: 75)
MLEGVTQTPKFQVLKTGQSMTLQCAQDMNHEYMSWYRQDPGMGLRLIHY
SVGAGITDQGEVPNGYNVSRSTTEDFPLRLLSAAPSQTSVYFCASSPVT
GGIYGYTFGSGTRLTVVEDLNKVFPPEVAVFEPSEAEISHTQKATLVCL
ATGFFPDHVELSWWVNGKEVHSGVSTDPQPLKEQPALNDSRYCLSSRLR
VSATFWQNPRNHFRCQVQFYGLSENDEWTQDRAKPVTQIVSAEAWGRAD
ATNFSLLKQAGDVEENPGP
(and that includes the P2A sequence at its
C-terminus)
(SEQ ID NO: 76)
ATGCTCGAGGGAGTGACCCAGACCCCCAAGTTCCAGGTGCTGAAGACCG
GACAGAGCATGACCCTGCAGTGCGCCCAGGACATGAACCACGAGTACAT
GAGCTGGTACCGGCAGGACCCCGGAATGGGACTGCGGCTGATCCACTAC
AGCGTGGGAGCCGGAATCACCGACCAGGGAGAGGTGCCCAACGGATACA
ACGTGAGCCGGAGCACCACCGAGGACTTCCCCCTGCGGCTGCTGAGCGC
CGCCCCCAGCCAGACCAGCGTGTACTTCTGCGCCAGCAGCCCCGTGACC
GGAGGAATCTACGGATACACCTTCGGAAGCGGAACCCGGCTGACCGTGG
TGGAGGACCTGAACAAGGTGTTCCCCCCCGAGGTGGCCGTGTTCGAGCC
CAGCGAGGCCGAGATCAGCCACACCCAGAAGGCCACCCTGGTGTGCCTG
GCCACCGGATTCTTCCCCGACCACGTGGAGCTGAGCTGGTGGGTGAACG
GAAAGGAGGTGCACAGCGGAGTGAGCACCGACCCCCAGCCCCTGAAGGA
GCAGCCCGCCCTGAACGACAGCCGGTACTGCCTGAGCAGCCGGCTGCGG
GTGAGCGCCACCTTCTGGCAGAACCCCCGGAACCACTTCCGGTGCCAGG
TGCAGTTCTACGGACTGAGCGAGAACGACGAGTGGACCCAGGACCGGGC
CAAGCCCGTGACCCAGATCGTGAGCGCCGAGGCCTGGGGACGGGCCGAC

### TCRa-Extracellular Domain:

(SEQ ID NO: 77)
MILNVEQSPQSLHVQEGDSTNFTCSFPSSNFYALHWYRWETAKSPEALF
VMTLNGDEKKKGRISATLNTKEGYSYLYIKGSQPEDSATYLCARNTGNQ
FYFGTGTSLTVIPNIQNPDPAVYQLRDSKSSDKSVCLFTDFDSQTNVSQ
SKDSDAYITDKTVLDMRSMDFKSNSAVAWSNKSDFACANAFNNSIIPED
TFFPSPESSEGRGSLLTCGDVEENPGP
(and that includes the T2A sequence at its
C-terminus)
(SEQ ID NO: 78)
ATGATCCTGAACGTGGAGCAGAGCCCCCAGAGCCTGCACGTGCAGGAGG
GAGACAGCACCAACTTCACCTGCAGCTTCCCCAGCAGCAACTTCTACGC
CCTGCACTGGTACCGGTGGGAGACCGCCAAGAGCCCCGAGGCCCTGTTC
GTGATGACCCTGAACGGAGACGAGAAGAAGAAGGGACGGATCAGCGCCA
CCCTGAACACCAAGGAGGGATACAGCTACCTGTACATCAAGGGAAGCCA
GCCCGAGGACAGCGCCACCTACCTGTGCGCCCGGAACACCGGAAACCAG
TTCTACTTCGGAACCGGAACCAGCCTGACCGTGATCCCCAACATCCAGA
ACCCCGACCCCGCCGTGTACCAGCTGCGGGACAGCAAGAGCAGCGACAA
GAGCGTGTGCCTGTTCACCGACTTCGACAGCCAGACCAACGTGAGCCAG
AGCAAGGACAGCGACGCCTACATCACCGACAAGACCGTGCTGGACATGC
GGAGCATGGACTTCAAGAGCAACAGCGCCGTGGCCTGGAGCAACAAGAG
CGACTTCGCCTGCGCCAACGCCTTCAACAACAGCATCATCCCCGAGGAC
ACCTTCTTCCCCAGCCCCGAGAGCAGCGCCACCAACTTCTCCCTGCTGA
AGCAGGCCGGCGACGTGGAGGAGAACCCCGGCCCC

TCR5: referred to TCRCgdZFLGDEFL15, is the constant region of TCR gamma and delta, linked to full length CD3zeta, full length CD3 gamma, full length CD3 delta, and full length CD3 epsilon; and IL-15. Representative sequences are as follows:

### TCR Constant Gamma-Delta (TCRCgd)

(SEQ ID NO: 81)
ATGCGGTGGGCCCTACTGGTGCTTCTAGCTTTCCTGTCTCCTGCCAGTC
AGGATAAACAACTTGATGCAGATGTTTCCCCCAAGCCCACTATTTTTCT
TCCTTCGATTGCTGAAACAAAACTCCAGAAGGCTGGAACATACCTTTGT
CTTCTTGAGAAATTTTTCCCAGATATTATTAAGATACATTGGCAAGAAA
AGAAGAGCAACACGATTCTGGGATCCCAGGAGGGGAACACCATGAAGAC
TAACGACACATACATGAAATTTAGCTGGTTAACGGTGCCAGAAGAGTCA
CTGGACAAAGAACACAGATGTATCGTCAGACATGAGAATAATAAAAACG
GAATTGATCAAGAAATTATCTTTCCTCCAATAAAGACAGATGTCACCAC
AGTGGATCCCAAATACAATTATTCAAAGGATGCAAATGATGTCATCACA
ATGGATCCCAAAGACAATTGGTCAAAAGATGCAAATGATACACTACTGC
TGCAGCTCACAAACACCTCTGCATATTACACGTACCTCCTCCTGCTCCT
CAAGAGTGTGGTCTATTTTGCCATCATCACCTGCTGTCTGCTTAGAAGA
ACGGCTTTCTGCTGCAATGGAGAGAAATCAGGAAGCGGAGCTACTAACT
TTAGCCTGCTGAAGCAGGCTGGAGATGTGGAGGAGAACCCTGGACCTAT
GATTCTTACTGTGGGCTTTAGCTTTTTGTTTTTCTACAGGGGCACGCTG
TGTAGTCAGCCTCATACCAAACCATCCGTTTTTGTCATGAAAAATGGAA
CAAATGTCGCTTGTCTGGTGAAGGAATTCTACCCCAAGGATATAAGAAT
AAATCTCGTGTCATCCAAGAAGATAACAGAGTTTGATCCTGCTATTGTC
ATCTCTCCCAGTGGGAAGTACAATGCTGTCAAGCTTGGTAAATATGAAG
ATTCAAATTCAGTGACATGTTCAGTTCAACACGACAATAAAACTGTGCA
CTCCACTGACTTTGAAGTGAAGACAGATTCTACAGATCACGTAAAACCA
AAGGAAACTGAAAACACAAAGCAACCTTCAAAGAGCTGCCATAAACCCA
AAGCCATAGTTCATACCGAGAAGGTGAACATGATGTCCCTCACAGTGCT
TGGGCTACGAATGCTGTTTGCAAAGACTGTTGCCGTCAATTTTCTCTTG
ACTGCCAAGTTATTTTTCTTGTAA
(SEQ ID NO: 82)
MRWALLVLLAFLSPASQDKQLDADVSPKPTIFLPSIAETKLQKAGTYLC
LLEKFFPDIIKIHWQEKKSNTILGSQEGNTMKINDTYMKFSWLTVPEES
LDKEHRCIVRHENNKNGIDQEIIFPPIKTDVTTVDPKYNYSKDANDVIT
MDPKDNWSKDANDTLLLQLTNTSAYYTYLLLLLKSVVYFAIITCCLLRR
TAFCCNGEKSGSGATNFSLLKQAGDVEENPGPMILTVGFSFLFFYRGTL
CSQPHTKPSVFVMKNGTNVACLVKEFYPKDIRINLVSSKKITEFDPAIV
ISPSGKYNAVKLGKYEDSNSVTCSVQHDNKTVHSTDFEVKTDSTDHVKP
KETENTKQPSKSCHKPKAIVHTEKVNMMSLTVLGLRMLFAKTVAVNFLL
TAKLFFL

CD3:

(SEQ ID NO: 79)
MKWKALFTAAILQAQLPITEAQSFGLLDPKLCYLLDGILFIYGVILTAL
FLRVKFSRSADAPAYQQGQNQLYNELNLGRREEYDVLDKRRGRDPEMGG
KPQRRKNPQEGLYNELQKDKMAEAYSEIGMKGERRRGKGHDGLYQGLST
ATKDTYDALHMQALPPRQCTNYALLKLAGDVESNPGPMEQGKGLAVLIL
AIILLQGTLAQSIKGNHLVKVYDYQEDGSVLLTCDAEAKNITWFKDGKM
IGFLTEDKKKWNLGSNAKDPRGMYQCKGSQNKSKPLQVYYRMCQNCIEL
NAATISGFLFAEIVSIFVLAVGVYFIAGQDGVRQSRASDKQTLLPNDQL
YQPLKDREDDQYSHLQGNQLRRNVKQTLNFDLLKLAGDVESNPGPMEHS
TFLSGLVLATLLSQVSPFKIPIEELEDRVFVNCNTSITWVEGTVGTLLS
DITRLDLGKRILDPRGIYRCNGTDIYKDKESTVQVHYRMCQSCVELDPA
TVAGIIVTDVIATLLLALGVFCFAGHETGRLSGAADTQALLRNDQVYQP
LRDRDDAQYSHLGGNWARNKEGRGSLLTCGDVEENPGPMQSGTHWRVLG
LCLLSVGVWGQDGNEEMGGITQTPYKVSISGTTVILTCPQYPGSEILWQ
HNDKNIGGDEDDKNIGSDEDHLSLKEFSELEQSGYYVCYPRGSKPEDAN
FYLYLRARVCENCMEMDVMSVATIVIVDICITGGLLLLVYYWSKNRKAK
AKPVTRGAGAGGRQRGQNKERPPPVPNPDYEPIRKGQRDLYSGLNQRRI
GPQCTNYALLKLAGDVESNPGP
(SEQ ID NO: 80)
ATGAAGTGGAAGGCGCTTTTCACCGCGGCCATCCTGCAGGCACAGTTGC
CGATTACAGAGGCACAGAGCTTTGGCCTGCTGGATCCCAAACTCTGCTA
CCTGCTGGATGGAATCCTCTTCATCTATGGTGTCATTCTCACTGCCTTG
TTCCTGAGAGTGAAGTTCAGCAGGAGCGCAGACGCCCCCGCGTACCAGC
AGGGCCAGAACCAGCTCTATAACGAGCTCAATCTAGGACGAAGAGAGGA
GTACGATGTTTTGGACAAGAGACGTGGCCGGGACCCTGAGATGGGGGGA
AAGCCGCAGAGAAGGAAGAACCCTCAGGAAGGCCTGTACAATGAACTGC
AGAAAGATAAGATGGCGGAGGCCTACAGTGAGATTGGGATGAAAGGCGA
GCGCCGGAGGGGCAAGGGGCACGATGGCCTTTACCAGGGTCTCAGTACA
GCCACCAAGGACACCTACGACGCCCTTCACATGCAGGCCCTGCCCCCTC
GCCAGTGCACCAACTACGCCCTGCTGAAGCTGGCCGGCGACGTGGAGAG
CAACCCCGGCCCCATGGAACAGGGGAAGGGCCTGGCTGTCCTCATCCTG
GCTATCATTCTTCTTCAAGGTACTTTGGCCCAGTCAATCAAAGGAAACC
ACTTGGTTAAGGTGTATGACTATCAAGAAGATGGTTCGGTACTTCTGAC
TTGTGATGCAGAAGCCAAAAATATCACATGGTTTAAAGATGGGAAGATG
ATCGGCTTCCTAACTGAAGATAAAAAAAAATGGAATCTGGGAAGTAATG
CCAAGGACCCTCGTGGGATGTATCAGTGTAAAGGATCACAGAACAAGTC
AAAACCACTCCAAGTGTATTACAGAATGTGTCAGAACTGCATTGAACTA
AATGCAGCCACCATATCTGGCTTTCTCTTTGCTGAAATCGTCAGCATTT
TCGTCCTTGCTGTTGGGGTCTACTTCATTGCTGGACAGGATGGAGTTCG
CCAGTCGAGAGCTTCAGACAAGCAGACTCTGTTGCCCAATGACCAGCTC
TACCAGCCCCTCAAGGATCGAGAAGATGACCAGTACAGCCACCTTCAAG
GAAACCAGTTGAGGAGGAATGTGAAGCAGACCCTGAACTTCGACCTGCT
GAAGCTGGCCGGCGACGTGGAGAGCAACCCCGGCCCCATGGAGCACAGC
ACCTTCCTGAGCGGCCTGGTGCTGGCCACCCTGCTGAGCCAGGTGAGCC
CCTTCAAGATCCCCATCGAGGAGCTGGAGGACAGAGTGTTCGTGAACTG
CAACACCAGCATCACCTGGGTGGAGGGCACCGTGGGCACCCTGCTGAGC
GACATCACCAGACTGGACCTGGGCAAGAGAATCCTGGACCCCAGAGGCA
TCTACAGATGCAACGGCACCGACATCTACAAGGACAAGGAGAGCACCGT
GCAGGTGCACTACAGAATGTGCCAGAGCTGCGTGGAGCTGGACCCCGCC
ACCGTGGCCGGCATCATCGTGACCGACGTGATCGCCACCCTGCTGCTGG
CCCTGGGCGTGTTCTGCTTCGCCGGCCACGAGACCGGCAGACTGAGCGG
CGCCGCCGACACCCAGGCCCTGCTGAGAAACGACCAGGTGTACCAGCCC
CTGAGAGACAGAGACGACGCCCAGTACAGCCACCTGGGCGGCAACTGGG
CCAGAAACAAGGAGGGCAGAGGCAGCCTGCTGACCTGCGGCGACGTGGA
GGAGAACCCCGGCCCCATGCAGAGCGGCACCCACTGGAGAGTGCTGGGC
CTGTGCCTGCTGAGCGTGGGCGTGTGGGGCCAGGACGGCAACGAGGAGA
TGGGCGGCATCACCCAGACCCCCTACAAGGTGAGCATCAGCGGCACCAC
CGTGATCCTGACCTGCCCCCAGTACCCCGGCAGCGAGATCCTGTGGCAG
CACAACGACAAGAACATCGGCGGCGACGAGGACGACAAGAACATCGGCA
GCGACGAGGACCACCTGAGCCTGAAGGAGTTCAGCGAGCTGGAGCAGAG
CGGCTACTACGTGTGCTACCCCAGAGGCAGCAAGCCCGAGGACGCCAAC
TTCTACCTGTACCTGAGAGCCAGAGTGTGCGAGAACTGCATGGAGATGG
ACGTGATGAGCGTGGCCACCATCGTGATCGTGGACATCTGCATCACCGG
CGGCCTGCTGCTGCTGGTGTACTACTGGAGCAAGAACAGAAAGGCCAAG
GCCAAGCCCGTGACCAGAGGCGCCGGCGCCGGCGGCAGACAGAGAGGCC
AGAACAAGGAGAGACCCCCCCCCGTGCCCAACCCCGACTACGAGCCCAT
CAGAAAGGGCCAGAGAGACCTGTACAGCGGCCTGAACCAGAGAAGAATC
GGACCGCAGTGTACTAATTATGCTCTCTTGAAATTGGCTGGAGATGTTG
AGAGCAATCCCGGGCCC

### IL-15:

(SEQ ID NO: 48)
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS*
(SEQ ID NO: 49)
ATGCGCATTAGCAAGCCCCACCTGCGGAGCATCAGCATCCAGTGCTACC
TGTGCCTGCTGCTGAACAGCCACTTCCTGACCGAGGCCGGCATCCACGT
GTTCATCCTGGGCTGCTTCAGCGCCGGACTGCCCAAGACCGAGGCCAAC
TGGGTGAACGTGATCAGCGACCTGAAGAAGATCGAGGACCTGATCCAGA
GCATGCACATCGACGCCACCCTGTACACCGAGAGCGACGTGCACCCCAG
CTGCAAGGTGACCGCCATGAAGTGCTTTCTGCTGGAACTGCAGGTGATC
AGCCTGGAAAGCGGCGACGCCAGCATCCACGACACCGTGGAGAACCTGA
TCATCCTGGCCAACAACAGCCTGAGCAGCAACGGCAACGTGACCGAGAG
CGGCTGCAAAGAGTGCGAGGAACTGGAAGAGAAGAACATCAAAGAGTTT
CTGCAGAGCTTCGTGCACATCGTGCAGATGTTCATCAACACCAGC

TCR6: also referred to TCRCabZFLGDEFL15, is the constant region of TCR alpha and beta, linked to full length CD3zeta, full length CD3 gamma, full length CD3 delta, and full length CD3 epsilon; and IL-15. Representative sequences are as follows:

### TCR Constant Alpha-Beta (TCRCab)

(SEQ ID NO: 83)
METLLGLLILWLQLQWVSSIQNPDPAVYQLRDSKSSDKSVCLFTDFDSQ
TNVSQSKDSDVYITDKTVLDMRSMDFKSNSAVAWSNKSDFACANAFNNS
IIPEDTFFPSPESSCDVKLVEKSFETDTNLNFQNLSVIGFRILLLKVAG
FNLLMTLRLWSSGSGATNFSLLKQAGDVEENPGPMSIGLLCCAALSLLW
AGPVNADLKNVFPPKVAVFEPSEAEISHTQKATLVCLATGFYPDHVELS
WWVNGKEVHSGVSTDPQPLKEQPALNDSRYCLSSRLRVSATFWQNPRNH
FRCQVQFYGLSENDEWTQDRAKPVTQIVSAEAWGRADCGFTSESYQQGV
LSATILYEILLGKATLYAVLVSALVLMAMVKRKDSRG
(SEQ ID NO: 84)
ATGGAGACCCTCTTGGGCCTGCTTATCCTTTGGCTGCAGCTGCAATGGG
TGAGCAGCATCCAGAACCCTGACCCTGCCGTGTACCAGCTGAGAGACTC
TAAATCCAGTGACAAGTCTGTCTGCCTATTCACCGATTTTGATTCTCAA
ACAAATGTGTCACAAAGTAAGGATTCTGATGTGTATATCACAGACAAAA
CTGTGCTAGACATGAGGTCTATGGACTTCAAGAGCAACAGTGCTGTGGC
CTGGAGCAACAAATCTGACTTTGCATGTGCAAACGCCTTCAACAACAGC
ATTATTCCAGAAGACACCTTCTTCCCCAGCCCAGAAAGTTCCTGTGATG
TCAAGCTGGTCGAGAAAAGCTTTGAAACAGATACGAACCTAAACTTTCA
AAACCTGTCAGTGATTGGGTTCCGAATCCTCCTCCTGAAAGTGGCCGGG
TTTAATCTGCTCATGACGCTGCGGCTGTGGTCCAGCGGAAGCGGAGCTA
CTAACTTTAGCCTGCTGAAGCAGGCTGGAGATGTGGAGGAGAACCCTGG
ACCTATGAGCATCGGCCTCCTGTGCTGTGCAGCCTTGTCTCTCCTGTGG
GCAGGTCCAGTGAATGCTGACCTGAAAAACGTGTTCCCACCCAAGGTCG
CTGTGTTTGAGCCATCAGAAGCAGAGATCTCCCACACCCAAAAGGCCAC
ACTGGTATGCCTGGCCACAGGCTTCTACCCCGACCACGTGGAGCTGAGC
TGGTGGGTGAATGGGAAGGAGGTGCACAGTGGGGTCAGCACAGACCCGC
AGCCCCTCAAGGAGCAGCCCGCCCTCAATGACTCCAGATACTGCCTGAG
CAGCCGCCTGAGGGTCTCGGCCACCTTCTGGCAGAACCCCCGCAACCAC
TTCCGCTGTCAAGTCCAGTTCTACGGGCTCTCGGAGAATGACGAGTGGA
CCCAGGATAGGGCCAAACCCGTCACCCAGATCGTCAGCGCCGAGGCCTG
GGGTAGAGCAGACTGTGGCTTCACCTCCGAGTCTTACCAGCAAGGGGTC
CTGTCTGCCACCATCCTCTATGAGATCTTGCTAGGGAAGGCCACCTTGT
ATGCCGTGCTGGTCAGTGCCCTCGTGCTGATGGCCATGGTCAAGAGAAA
GGATTCCAGAGGCTAA

### CD3:

(SEQ ID NO: 79)
MKWKALFTAAILQAQLPITEAQSFGLLDPKLCYLLDGILFIYGVILTALFLRVKFSRSADAP
AYQQGQNQLYNELNLGRREEYDVLDKRRGRDPEMGGKPQRRKNPQEGLYNELQKDKMAEAYS
EIGMKGERRRGKGHDGLYQGLSTATKDTYDALHMQALPPRQCTNYALLKLAGDVESNPGPME
QGKGLAVLILAIILLQGTLAQSIKGNHLVKVYDYQEDGSVLLTCDAEAKNITWFKDGKMIGF
LTEDKKKWNLGSNAKDPRGMYQCKGSQNKSKPLQVYYRMCQNCIELNAATISGFLFAEIVSI
FVLAVGVYFIAGQDGVRQSRASDKQTLLPNDQLYQPLKDREDDQYSHLQGNQLRRNVKQTLN
FDLLKLAGDVESNPGPMEHSTFLSGLVLATLLSQVSPFKIPIEELEDRVFVNCNTSITWVEG
TVGTLLSDITRLDLGKRILDPRGIYRCNGTDIYKDKESTVQVHYRMCQSCVELDPATVAGII
VTDVIATLLLALGVFCFAGHETGRLSGAADTQALLRNDQVYQPLRDRDDAQYSHLGGNWARN
KEGRGSLLTCGDVEENPGPMQSGTHWRVLGLCLLSVGVWGQDGNEEMGGITQTPYKVSISGT
TVILTCPQYPGSEILWQHNDKNIGGDEDDKNIGSDEDHLSLKEFSELEQSGYYVCYPRGSKP
EDANFYLYLRARVCENCMEMDVMSVATIVIVDICITGGLLLLVYYWSKNRKAKAKPVTRGAG
AGGRQRGQNKERPPPVPNPDYEPIRKGQRDLYSGLNQRRIGPQCTNYALLKLAGDVESNPGP
(SEQ ID NO: 80)
ATGAAGTGGAAGGCGCTTTTCACCGCGGCCATCCTGCAGGCACAGTTGCCGATTACAGAGGC
ACAGAGCTTTGGCCTGCTGGATCCCAAACTCTGCTACCTGCTGGATGGAATCCTCTTCATCT
ATGGTGTCATTCTCACTGCCTTGTTCCTGAGAGTGAAGTTCAGCAGGAGCGCAGACGCCCCC
GCGTACCAGCAGGGCCAGAACCAGCTCTATAACGAGCTCAATCTAGGACGAAGAGAGGAGTA
CGATGTTTTGGACAAGAGACGTGGCCGGGACCCTGAGATGGGGGGAAAGCCGCAGAGAAGGA
AGAACCCTCAGGAAGGCCTGTACAATGAACTGCAGAAAGATAAGATGGCGGAGGCCTACAGT
GAGATTGGGATGAAAGGCGAGCGCCGGAGGGGCAAGGGGCACGATGGCCTTTACCAGGGTCT
CAGTACAGCCACCAAGGACACCTACGACGCCCTTCACATGCAGGCCCTGCCCCCTCGCCAGT
GCACCAACTACGCCCTGCTGAAGCTGGCCGGCGACGTGGAGAGCAACCCCGGCCCCATGGAA
CAGGGGAAGGGCCTGGCTGTCCTCATCCTGGCTATCATTCTTCTTCAAGGTACTTTGGCCCA
GTCAATCAAAGGAAACCACTTGGTTAAGGTGTATGACTATCAAGAAGATGGTTCGGTACTTC
TGACTTGTGATGCAGAAGCCAAAAATATCACATGGTTTAAAGATGGGAAGATGATCGGCTTC
CTAACTGAAGATAAAAAAAAATGGAATCTGGGAAGTAATGCCAAGGACCCTCGTGGGATGTA
TCAGTGTAAAGGATCACAGAACAAGTCAAAACCACTCCAAGTGTATTACAGAATGTGTCAGA
ACTGCATTGAACTAAATGCAGCCACCATATCTGGCTTTCTCTTTGCTGAAATCGTCAGCATT
TTCGTCCTTGCTGTTGGGGTCTACTTCATTGCTGGACAGGATGGAGTTCGCCAGTCGAGAGC
TTCAGACAAGCAGACTCTGTTGCCCAATGACCAGCTCTACCAGCCCCTCAAGGATCGAGAAG
ATGACCAGTACAGCCACCTTCAAGGAAACCAGTTGAGGAGGAATGTGAAGCAGACCCTGAAC
TTCGACCTGCTGAAGCTGGCCGGCGACGTGGAGAGCAACCCCGGCCCCATGGAGCACAGCAC
CTTCCTGAGCGGCCTGGTGCTGGCCACCCTGCTGAGCCAGGTGAGCCCCTTCAAGATCCCCA
TCGAGGAGCTGGAGGACAGAGTGTTCGTGAACTGCAACACCAGCATCACCTGGGTGGAGGGC
ACCGTGGGCACCCTGCTGAGCGACATCACCAGACTGGACCTGGGCAAGAGAATCCTGGACCC
CAGAGGCATCTACAGATGCAACGGCACCGACATCTACAAGGACAAGGAGAGCACCGTGCAGG
TGCACTACAGAATGTGCCAGAGCTGCGTGGAGCTGGACCCCGCCACCGTGGCCGGCATCATC
GTGACCGACGTGATCGCCACCCTGCTGCTGGCCCTGGGCGTGTTCTGCTTCGCCGGCCACGA
GACCGGCAGACTGAGCGGCGCCGCCGACACCCAGGCCCTGCTGAGAAACGACCAGGTGTACC
AGCCCCTGAGAGACAGAGACGACGCCCAGTACAGCCACCTGGGCGGCAACTGGGCCAGAAAC
AAGGAGGGCAGAGGCAGCCTGCTGACCTGCGGCGACGTGGAGGAGAACCCCGGCCCCATGCA
GAGCGGCACCCACTGGAGAGTGCTGGGCCTGTGCCTGCTGAGCGTGGGCGTGTGGGGCCAGG
ACGGCAACGAGGAGATGGGCGGCATCACCCAGACCCCCTACAAGGTGAGCATCAGCGGCACC
ACCGTGATCCTGACCTGCCCCCAGTACCCCGGCAGCGAGATCCTGTGGCAGCACAACGACAA
GAACATCGGCGGCGACGAGGACGACAAGAACATCGGCAGCGACGAGGACCACCTGAGCCTGA
AGGAGTTCAGCGAGCTGGAGCAGAGCGGCTACTACGTGTGCTACCCCAGAGGCAGCAAGCCC
GAGGACGCCAACTTCTACCTGTACCTGAGAGCCAGAGTGTGCGAGAACTGCATGGAGATGGA
CGTGATGAGCGTGGCCACCATCGTGATCGTGGACATCTGCATCACCGGCGGCCTGCTGCTGC
TGGTGTACTACTGGAGCAAGAACAGAAAGGCCAAGGCCAAGCCCGTGACCAGAGGCGCCGGC
GCCGGCGGCAGACAGAGAGGCCAGAACAAGGAGAGACCCCCCCCCGTGCCCAACCCCGACTA
CGAGCCCATCAGAAAGGGCCAGAGAGACCTGTACAGCGGCCTGAACCAGAGAAGAATCGGAC
CGCAGTGTACTAATTATGCTCTCTTGAAATTGGCTGGAGATGTTGAGAGCAATCCCGGGCCC

### IL-15:

(SEQ ID NO: 48)
MRISKPHLRSISIQCYLCLLLNSHFLTEAGIHVFILGCFSAGLPKTEAN
WVNVISDLKKIEDLIQSMHIDATLYTESDVHPSCKVTAMKCFLLELQVI
SLESGDASIHDTVENLIILANNSLSSNGNVTESGCKECEELEEKNIKEF
LQSFVHIVQMFINTS*
(SEQ ID NO: 49)
ATGCGCATTAGCAAGCCCCACCTGCGGAGCATCAGCATCCAGTGCTACC
TGTGCCTGCTGCTGAACAGCCACTTCCTGACCGAGGCCGGCATCCACGT
GTTCATCCTGGGCTGCTTCAGCGCCGGACTGCCCAAGACCGAGGCCAAC
TGGGTGAACGTGATCAGCGACCTGAAGAAGATCGAGGACCTGATCCAGA
GCATGCACATCGACGCCACCCTGTACACCGAGAGCGACGTGCACCCCAG
CTGCAAGGTGACCGCCATGAAGTGCTTTCTGCTGGAACTGCAGGTGATC
AGCCTGGAAAGCGGCGACGCCAGCATCCACGACACCGTGGAGAACCTGA
TCATCCTGGCCAACAACAGCCTGAGCAGCAACGGCAACGTGACCGAGAG
CGGCTGCAAAGAGTGCGAGGAACTGGAAGAGAAGAACATCAAAGAGTTT
CTGCAGAGCTTCGTGCACATCGTGCAGATGTTCATCAACACCAGC

In some embodiments, a TCR construct comprises an NY-ESO-specific TCR and a CD8 alpha/beta co-receptor molecule. In some embodiments, such a construct can comprise a TCR alpha chain variable region signal peptide, a TCR alpha chain variable region, a TCR alpha chain constant region, a 2A element (e.g., P2A element), a TCR beta chain variable region signal peptide, a TCR beta chain variable region, a TCR beta chain constant region, a 2A element (e.g., a E2A element), a CD8-beta polypeptide, a 2A element (e.g., a T2A element), and a CD8-alpha polypeptide. In some embodiments, a TCR construct comprising an NY-ESO-specific TCR and a CD8 alpha/beta co-receptor molecule nucleotide coding sequence is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 124. In some embodiments, a TCR construct comprising an NY-ESO-specific TCR and a CD8 alpha/beta co-receptor molecule amino acid sequence is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 125.

In some embodiments, a CD8 alpha co-receptor molecule is transcriptionally linked to any TCR molecule disclosed herein. In some embodiments, a CD8 alpha co-receptor molecule nucleotide coding sequence is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 126. In some embodiments, a CD8 beta co-receptor molecule nucleotide coding sequence is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 127. In some embodiments, a CD8 alpha co-receptor amino acid sequence is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 128. In some embodiments, a CD8 beta co-receptor amino acid sequence is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 129.

(SEQ ID NO: 124)
ATGGAGACCCTCTTGGGCCTGCTTATCCTTTGGCTGCAGCTGCAATGGGTGAGCAGCAAACA
GGAGGTGACACAGATTCCTGCAGCTCTGAGTGTCCCAGAAGGAGAAAACTTGGTTCTCAACT
GCAGTTTCACTGATAGCGCTATTTACAACCTCCAGTGGTTTAGGCAGGACCCTGGGAAAGGT
CTCACATCTCTGTTGCTTATTCAGTCAAGTCAGAGAGAGCAAACAAGTGGAAGACTTAATGC
CTCGCTGGATAAATCATCAGGACGTAGTACTTTATACATTGCAGCTTCTCAGCCTGGTGACT
CAGCCACCTACCTCTGTGCTGTGAGGCCCCTTTATGGAGGAAGCTACATACCTACATTTGGA
AGAGGAACCAGCCTTATTGTTCATCCGTATATCCAGAACCCTGACCCTGCCGTGTACCAGCT
GAGAGACTCTAAATCCAGTGACAAGTCTGTCTGCCTATTCACCGATTTTGATTCTCAAACAA
ATGTGTCACAAAGTAAGGATTCTGATGTGTATATCACAGACAAAACTGTGCTAGACATGAGG
TCTATGGACTTCAAGAGCAACAGTGCTGTGGCCTGGAGCAACAAATCTGACTTTGCATGTGC
AAACGCCTTCAACAACAGCATTATTCCAGAAGACACCTTCTTCCCCAGCCCAGAAAGTTCCT
GTGATGTCAAGCTGGTCGAGAAAAGCTTTGAAACAGATACGAACCTAAACTTTCAAAACCTG
TCAGTGATTGGGTTCCGAATCCTCCTCCTGAAAGTGGCCGGGTTTAATCTGCTCATGACGCT
GCGGCTGTGGTCCAGCGGAAGCGGAGCTACTAACTTTAGCCTGCTGAAGCAGGCTGGAGATG
TGGAGGAGAACCCTGGACCTATGAGCATCGGCCTCCTGTGCTGTGCAGCCTTGTCTCTCCTG
TGGGCAGGTCCAGTGAATGCTGGTGTCACTCAGACCCCAAAATTCCAGGTCCTGAAGACAGG
ACAGAGCATGACACTGCAGTGTGCCCAGGATATGAACCATGAATACATGTCCTGGTATCGAC
AAGACCCAGGCATGGGGCTGAGGCTGATTCATTACTCAGTTGGTGCTGGTATCACTGACCAA
GGAGAAGTCCCCAATGGCTACAATGTCTCCAGATCAACCACAGAGGATTTCCCGCTCAGGCT
GCTGTCGGCTGCTCCCTCCCAGACATCTGTGTACTTCTGTGCCAGCAGTTACGTCGGGAACA
CCGGGGAGCTGTTTTTTGGAGAAGGCTCTAGGCTGACCGTACTGGAGGACCTGAAAAACGTG
TTCCCACCCAAGGTCGCTGTGTTTGAGCCATCAGAAGCAGAGATCTCCCACACCCAAAAGGC
CACACTGGTATGCCTGGCCACAGGCTTCTACCCCGACCACGTGGAGCTGAGCTGGTGGGTGA
ATGGGAAGGAGGTGCACAGTGGGGTCAGCACAGACCCGCAGCCCCTCAAGGAGCAGCCCGCC
CTCAATGACTCCAGATACTGCCTGAGCAGCCGCCTGAGGGTCTCGGCCACCTTCTGGCAGAA
CCCCCGCAACCACTTCCGCTGTCAAGTCCAGTTCTACGGGCTCTCGGAGAATGACGAGTGGA
CCCAGGATAGGGCCAAACCCGTCACCCAGATCGTCAGCGCCGAGGCCTGGGGTAGAGCAGAC
TGTGGCTTCACCTCCGAGTCTTACCAGCAAGGGGTCCTGTCTGCCACCATCCTCTATGAGAT
CTTGCTAGGGAAGGCCACCTTGTATGCCGTGCTGGTCAGTGCCCTCGTGCTGATGGCCATGG
TCAAGAGAAAGGATTCCAGAGGCAGTGGACAGTGCACCAACTACGCCCTGCTGAAGCTGGCC
GGCGACGTGGAGAGCAACCCCGGCCCCATGGCCTTGCCCGTCACTGCGCTTTTGCTCCCGCT
CGCTCTTCTCCTGCATGCAGCCCGACCATCTCAATTTAGAGTTTCTCCACTCGACAGGACGT
GGAACCTCGGCGAAACCGTCGAACTTAAATGTCAAGTACTTCTCTCAAATCCGACTTCTGGT
TGCTCATGGCTCTTTCAGCCGAGAGGAGCAGCTGCCAGCCCCACCTTCCTGCTGTATCTCTC
CCAGAACAAGCCGAAGGCCGCCGAAGGGCTCGATACTCAACGATTTAGCGGGAAGCGACTCG
GGGACACGTTCGTTCTTACTCTCAGCGATTTTAGAAGAGAGAACGAGGGATATTATTTTTGT
TCCGCACTCTCTAACAGCATCATGTACTTCAGTCATTTTGTACCAGTCTTTCTCCCTGCAAA
ACCAACGACTACTCCAGCACCAAGACCGCCCACTCCCGCACCTACTATTGCAAGCCAACCTT
TGAGTCTCCGACCAGAGGCATGCAGACCTGCTGCTGGAGGTGCAGTACATACGCGAGGGTTG
GATTTTGCCTGCGATATCTATATCTGGGCCCCCTTGGCCGGCACGTGCGGGGTGCTCCTGCT
GAGTCTCGTAATTACTCTTTATTGTAATCATAGAAACCGCAGAAGGGTGTGTAAGTGTCCCC
GGCCTGTCGTGAAAAGCGGGGATAAGCCCAGTTTGTCTGCTCGGTACGTCGGAAGCGGTGAG
GGCAGGGGAAGTCTTCTAACATGCGGGGACGTGGAGGAAAATCCCGGACCCATGAGGCCACG
ACTTTGGCTGCTGCTCGCTGCACAGTTGACTGTACTGCATGGCAATAGTGTGTTGCAGCAGA
CACCTGCATACATCAAGGTTCAGACAAATAAGATGGTTATGCTGAGTTGCGAGGCAAAAATT
AGTTTGAGCAATATGCGGATCTACTGGTTGCGACAGAGACAGGCTCCCAGTAGTGATAGTCA
CCACGAATTCCTGGCTCTTTGGGATTCCGCAAAAGGAACGATTCATGGGGAAGAAGTAGAGC
AGGAGAAGATTGCGGTTTTCCGCGATGCATCTCGCTTTATCCTTAATCTTACATCCGTTAAG
CCTGAGGACAGTGGGATCTATTTTTGTATGATTGTAGGGTCCCCCGAATTGACATTTGGGAA
GGGTACGCAGCTCTCCGTAGTTGACTTTCTGCCCACAACGGCACAACCCACTAAGAAGTCCA
CCCTGAAGAAGCGCGTCTGTCGCTTGCCCAGACCTGAAACCCAAAAGGGTCCACTCTGTTCC
CCTATAACCCTGGGGTTGTTGGTGGCGGGCGTCTTGGTCCTGCTTGTTAGCTTGGGCGTAGC
CATTCATCTGTGTTGCCGAAGACGCAGAGCCCGACTTAGATTTATGAAGCAATTCTATAAGT
GA
(SEQ ID NO: 125)
METLLGLLILWLQLQWVSSKQEVTQIPAALSVPEGFNLVLNCSFTDSAIYNLQWFRQDPGKG
LTSLLLIQSSQREQTSGRLNASLDKSSGRSTLYIAASQPGDSATYLCAVRPLYGGSYIPTFG
RGTSLIVHPYIQNPDPAVYQLRDSKSSDKSVCLFTDFDSQTNVSQSKDSDVYITDKTVLDMR
SMDFKSNSAVAWSNKSDFACANAFNNSIIPEDTFFPSPESSCDVKLVEKSFETDTNLNFQNL
SVIGFRILLLKVAGFNLLMTLRLWSSGSGATNFSLLKQAGDVEENPGPMSIGLLCCAALSLL
WAGPVNAGVTQTPKFQVLKTGQSMTLQCAQDMNHEYMSWYRQDPGMGLRLIHYSVGAGITDQ
GEVPNGYNVSRSTTEDFPLRLLSAAPSQTSVYFCASSYVGNTGELFFGEGSRLTVLEDLKNV
FPPKVAVFEPSEAEISHTQKATLVCLATGFYPDHVELSWWVNGKEVHSGVSTDPQPLKEQPA
LNDSRYCLSSRLRVSATFWQNPRNHFRCQVQFYGLSENDEWTQDRAKPVTQIVSAEAWGRAD
CGFTSESYQQGVLSATILYEILLGKATLYAVLVSALVLMAMVKRKDSRGSGQCTNYALLKLA
GDVESNPGPMALPVTALLLPLALLLHAARPSQFRVSPLDRTWNLGETVELKCQVLLSNPTSG
CSWLFQPRGAAASPTFLLYLSQNKPKAAEGLDTQRFSGKRLGDTFVLTLSDFRRENEGYYFC
SALSNSIMYFSHFVPVFLPAKPTTTPAPRPPTPAPTIASQPLSLRPEACRPAAGGAVHTRGL
DFACDIYIWAPLAGTCGVLLLSLVITLYCNHRNRRRVCKCPRPVVKSGDKPSLSARYVGSGE
GRGSLLTCGDVEENPGPMRPRLWLLLAAQLTVLHGNSVLQQTPAYIKVQTNKMVMLSCEAKI
SLSNMRIYWLRQRQAPSSDSHHEFLALWDSAKGTIHGEEVEQEKIAVFRDASRFILNLTSVK
PEDSGIYFCMIVGSPELTFGKGTQLSVVDFLPTTAQPTKKSTLKKRVCRLPRPETQKGPLCS
PITLGLLVAGVLVLLVSLGVAIHLCCRRRRARLRFMKQFYK*
(SEQ ID NO: 126)
ATGAGGCCACGACTTTGGCTGCTGCTCGCTGCACAGTTGACTGTACTGCATGGCAATAGTGT
GTTGCAGCAGACACCTGCATACATCAAGGTTCAGACAAATAAGATGGTTATGCTGAGTTGCG
AGGCAAAAATTAGTTTGAGCAATATGCGGATCTACTGGTTGCGACAGAGACAGGCTCCCAGT
AGTGATAGTCACCACGAATTCCTGGCTCTTTGGGATTCCGCAAAAGGAACGATTCATGGGGA
AGAAGTAGAGCAGGAGAAGATTGCGGTTTTCCGCGATGCATCTCGCTTTATCCTTAATCTTA
CATCCGTTAAGCCTGAGGACAGTGGGATCTATTTTTGTATGATTGTAGGGTCCCCCGAATTG
ACATTTGGGAAGGGTACGCAGCTCTCCGTAGTTGACTTTCTGCCCACAACGGCACAACCCAC
TAAGAAGTCCACCCTGAAGAAGCGCGTCTGTCGCTTGCCCAGACCTGAAACCCAAAAGGGTC
CACTCTGTTCCCCTATAACCCTGGGGTTGTTGGTGGCGGGCGTCTTGGTCCTGCTTGTTAGC
TTGGGCGTAGCCATTCATCTGTGTTGCCGAAGACGCAGAGCCCGACTTAGATTTATGAAGCA
ATTCTATAAGTGA
(SEQ ID NO: 127)
ATGGCCTTGCCCGTCACTGCGCTTTTGCTCCCGCTCGCTCTTCTCCTGCATGCAGCCCGACC
ATCTCAATTTAGAGTTTCTCCACTCGACAGGACGTGGAACCTCGGCGAAACCGTCGAACTTA
AATGTCAAGTACTTCTCTCAAATCCGACTTCTGGTTGCTCATGGCTCTTTCAGCCGAGAGGA
GCAGCTGCCAGCCCCACCTTCCTGCTGTATCTCTCCCAGAACAAGCCGAAGGCCGCCGAAGG
GCTCGATACTCAACGATTTAGCGGGAAGCGACTCGGGGACACGTTCGTTCTTACTCTCAGCG
ATTTTAGAAGAGAGAACGAGGGATATTATTTTTGTTCCGCACTCTCTAACAGCATCATGTAC
TTCAGTCATTTTGTACCAGTCTTTCTCCCTGCAAAACCAACGACTACTCCAGCACCAAGACC
GCCCACTCCCGCACCTACTATTGCAAGCCAACCTTTGAGTCTCCGACCAGAGGCATGCAGAC
CTGCTGCTGGAGGTGCAGTACATACGCGAGGGTTGGATTTTGCCTGCGATATCTATATCTGG
GCCCCCTTGGCCGGCACGTGCGGGGTGCTCCTGCTGAGTCTCGTAATTACTCTTTATTGTAA
TCATAGAAACCGCAGAAGGGTGTGTAAGTGTCCCCGGCCTGTCGTGAAAAGCGGGGATAAGC
CCAGTTTGTCTGCTCGGTACGTC
(SEQ ID NO: 128)
MRPRLWLLLAAQLTVLHGNSVLQQTPAYIKVQTNKMVMLSCEAKISLSNMRIYWLRQRQAPS
SDSHHEFLALWDSAKGTIHGEEVEQEKIAVFRDASRFILNLTSVKPEDSGIYFCMIVGSPEL
TFGKGTQLSVVDFLPTTAQPTKKSTLKKRVCRLPRPETQKGPLCSPITLGLLVAGVLVLLVS
LGVAIHLCCRRRRARLRFMKQFYK
(SEQ ID NO: 129)
MALPVTALLLPLALLLHAARPSQFRVSPLDRTWNLGETVELKCQVLLSNPTSGCSWLFQPRG
AAASPTFLLYLSQNKPKAAEGLDTQRFSGKRLGDTFVLTLSDERRENEGYYFCSALSNSIMY
FSHFVPVFLPAKPTTTPAPRPPTPAPTIASQPLSLRPEACRPAAGGAVHTRGLDFACDIYIW
APLAGTCGVLLLSLVITLYCNHRNRRRVCKCPRPVVKSGDKPSLSARYV

In some embodiments, a TCR construct comprises PRAME-specific TCR chains. In some embodiments, a TCR construct comprising PRAME-specific TCR chains comprises TCR alpha and TCR beta chains found in PRAME-specific TCR clone 46, clone 54, and/or clone DSK3. In some embodiments, a TCR construct comprising PRAME-specific TCR chains comprises TCR alpha and TCR beta chains that target PRAME epitopes SLLQHLIGL (SEQ ID NO: 131) and/or QLLALLPSL (SEQ ID NO: 132).

In some embodiments, a TCR construct comprising PRAME-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 133 (e.g., TCR clone 46 TCR alpha) and/or 134 (e.g., TCR clone 46 TCR beta). In some embodiments, a TCR construct comprising PRAME-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 135 (e.g., TCR clone 46 TCR alpha) and/or 136 (e.g., TCR clone 46 TCR beta).

(SEQ ID NO: 133)
ATGCTTCTGGAACACCTGCTGATTATCCTGTGGATGCAACTCACGTGGG
TCTCCGGGCAACAACTGAATCAAAGCCCCCAATCCATGTTTATACAGGA
GGGAGAGGACGTAAGTATGAATTGCACATCTTCATCTATCTTTAACACC
TGGCTGTGGTACAAACAAGACCCCGGAGAAGGTCCTGTACTTCTCATCG
CACTTTACAAAGCAGGTGAGCTTACCAGTAACGGGAGACTCACCGCACA
GTTCGGTATTACAAGAAAGGATTCCTTTCTCAACATCTCCGCTTCTATC
CCTTCAGACGTCGGAATTTATTTTTGTGCTGGTATCCCTCGAGACAATT
ACGGTCAAAACTTTGTATTTGGGCCTGGGACTCGGCTGTCAGTTTTGCC
GTATATCCAGAACCCCGACCCCGCCGTGTACCAGCTGCGGGACAGCAAG
AGCAGCGACAAGAGCGTGTGCCTGTTCACCGACTTCGACAGCCAGACCA
ACGTGTCCCAGAGCAAGGACAGCGACGTGTACATCACCGATAAGTGCGT
GCTGGACATGCGGAGCATGGACTTCAAGAGCAACAGCGCCGTGGCCTGG
TCCAACAAGAGCGACTTCGCCTGCGCCAACGCCTTCAACAACAGCATCA
TCCCCGAGGACACATTCTTCCCAAGCCCCGAGAGCAGCTGCGACGTGAA
GCTGGTGGAGAAGTCCTTCGAGACAGACACCAACCTGAACTTCCAGAAC
CTGTCCGTGATCGGCTTCAGAATCCTGCTGCTGAAAGTGGCCGGCTTCA
ACCTGCTGATGACCCTGCGGCTGTGGTCCAGC
(SEQ ID NO: 134)
ATGGGCATTAGGCTGCTGTGCAGAGTAGCATTTTGCTTTCTGGCAGTAG
GATTGGTCGATGTAAAGGTTACACAGTCCTCACGGTACTTGGTAAAGCG
CACTGGTGAAAAGGTCTTTCTGGAATGTGTACAAGATATGGATCACGAA
AATATGTTTTGGTACAGGCAAGATCCCGGCCTTGGACTTAGACTGATAT
ATTTCTCCTACGATGTTAAAATGAAGGAGAAGGGCGATATTCCAGAAGG
ATATTCCGTGAGCCGCGAAAAGAAGGAGCGATTCAGTTTGATACTCGAA
AGTGCCTCCACAAACCAAACCTCTATGTACCTTTGCGCGTCAACGCCGT
GGCTGGCCGGTGGCAATGAACAATTCTTCGGGCCGGGTACGCGCCTCAC
TGTCCTGGAGGACCTCAAGAATGTGTTTCCGCCCGAAGTCGCGGTTTTT
GAACCATCAGAAGCCGAGATCTCTCATACACAAAAGGCGACGCTCGTAT
GCCTTGCGACGGGATTTTATCCGGACCACGTCGAGCTTTCCTGGTGGGT
TAATGGAAAGGAGGTGCATTCCGGAGTTTGCACGGACCCTCAGCCATTG
AAGGAACAGCCCGCACTGAACGACAGTAGGTATTGCCTTTCATCTCGCC
TGCGCGTGTCTGCGACATTCTGGCAAAACCCAAGAAATCACTTCAGATG
TCAAGTTCAGTTCTACGGTCTCAGCGAGAATGATGAGTGGACACAAGAT
AGGGCTAAACCCGTGACTCAAATAGTCTCTGCCGAGGCCTGGGGGAGGG
CGGATTGCGGCTTCACATCAGAATCATACCAACAAGGAGTATTGAGCGC
GACAATTCTTTACGAAATTCTGCTTGGGAAAGCGACTCTGTACGCGGTG
CTCGTGTCCGCTTTGGTTCTTATGGCAATGGTTAAACGAAAGGATAGTA
GGGGC
(SEQ ID NO: 135)
MLLEHLLIILWMQLTWVSGQQLNQSPQSMFIQEGEDVSMNCTSSSIFNT
WLWYKQDPGEGPVLLIALYKAGELTSNGRLTAQFGITRKDSFLNISASI
PSDVGIYFCAGIPRDNYGQNFVFGPGTRLSVLPYIQNPDPAVYQLRDSK
SSDKSVCLFTDFDSQTNVSQSKDSDVYITDKCVLDMRSMDFKSNSAVAW
SNKSDFACANAFNNSIIPEDTFFPSPESSCDVKLVEKSFETDTNLNFQN
LSVIGFRILLLKVAGFNLLMTLRLWSS
(SEQ ID NO: 136)
MGIRLLCRVAFCFLAVGLVDVKVTQSSRYLVKRTGEKVFLECVQDMDHE
NMFWYRQDPGLGLRLIYFSYDVKMKEKGDIPEGYSVSREKKERFSLILE
SASTNQTSMYLCASTPWLAGGNEQFFGPGTRLTVLEDLKNVFPPEVAVF
EPSEAEISHTQKATLVCLATGFYPDHVELSWWVNGKEVHSGVCTDPQPL
KEQPALNDSRYCLSSRLRVSATFWQNPRNHFRCQVQFYGLSENDEWTQD
RAKPVTQIVSAEAWGRADCGFTSESYQQGVLSATILYEILLGKATLYAV
LVSALVLMAMVKRKDSRG

In some embodiments, a TCR construct comprising PRAME-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 137 (e.g., TCR clone 54 TCR alpha) and/or 138 (e.g., TCR clone 54 TCR beta). In some embodiments, a TCR construct comprising PRAME-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 139 (e.g., TCR clone 54 TCR alpha) and/or 140 (e.g., TCR clone 54 TCR beta).

(SEQ ID NO: 137)
ATGCTGCTGCTGCTGGTGCCCGTGCTGGAAGTGATCTTCACCCTGGGCG
GCACCAGAGCCCAGAGCGTGACACAGCTGGGCAGCCACGTGTCCGTGTC
TGAGAGGGCCCTGGTGCTGCTGAGATGCAACTACTCTTCTAGCGTGCCC
CCCTACCTGTTTTGGTACGTGCAGTACCCCAACCAGGGGCTGCAGCTGC
TCCTGAAGTACACCAGCGCCGCCACACTGGTGAAGGGCATCAACGGCTT
CGAGGCCGAGTTCAAGAAGTCCGAGACAAGCTTCCACCTGACCAAGCCC
AGCGCCCACATGTCTGACGCCGCCGAGTACTTCTGTGCCGTGAGCGGCC
AGACCGGCGCCAACAACCTGTTCTTCGGCACCGGCACCCGGCTGACAGT
GATCCCTTACATCCAGAACCCCGACCCCGCCGTGTACCAGCTGCGGGAC
AGCAAGAGCAGCGACAAGAGCGTGTGCCTGTTCACCGACTTCGACAGCC
AGACCAACGTGTCCCAGAGCAAGGACAGCGACGTGTACATCACCGATAA
GTGCGTGCTGGACATGCGGAGCATGGACTTCAAGAGCAACAGCGCCGTG
GCCTGGTCCAACAAGAGCGACTTCGCCTGCGCCAACGCCTTCAACAACA
GCATCATCCCCGAGGACACATTCTTCCCAAGCCCCGAGAGCAGCTGCGA
CGTGAAGCTGGTGGAGAAGTCCTTCGAGACAGACACCAACCTGAACTTC
CAGAACCTGTCCGTGATCGGCTTCAGAATCCTGCTGCTGAAAGTGGCCG
GCTTCAACCTGCTGATGACCCTGCGGCTGTGGTCCAGC
(SEQ ID NO: 138)
ATGGGCTTCCGGCTGCTGTGCTGCGTGGCCTTTTGTCTGCTGGGAGCCG
GACCTGTGGATAGCGGCGTGACCCAGACCCCCAAGCACCTGATCACCGC
CACCGGCCAGAGAGTGACCCTGCGCTGCAGCCCTAGAAGCGGCGACCTG
AGCGTGTACTGGTATCAGCAGAGCCTCGACCAGGGCCTGCAGTTCCTGA
TCCAGTACTACAACGGCGAGGAACGGGCCAAGGGCAACATCCTGGAACG
GTTCAGCGCCCAGCAGTTCCCCGATCTGCACAGCGAGCTGAACCTGAGC
AGCCTGGAACTGGGCGACAGCGCCCTGTACTTCTGCGCCAGCGCCAGAT
GGGATAGAGGCGGCGAGCAGTACTTCGGCCCTGGCACCAGACTGACCGT
GACCGAGGACCTCAAGAATGTGTTTCCGCCCGAAGTCGCGGTTTTTGAA
CCATCAGAAGCCGAGATCTCTCATACACAAAAGGCGACGCTCGTATGCC
TTGCGACGGGATTTTATCCGGACCACGTCGAGCTTTCCTGGTGGGTTAA
TGGAAAGGAGGTGCATTCCGGAGTTTGCACGGACCCTCAGCCATTGAAG
GAACAGCCCGCACTGAACGACAGTAGGTATTGCCTTTCATCTCGCCTGC
GCGTGTCTGCGACATTCTGGCAAAACCCAAGAAATCACTTCAGATGTCA
AGTTCAGTTCTACGGTCTCAGCGAGAATGATGAGTGGACACAAGATAGG
GCTAAACCCGTGACTCAAATAGTCTCTGCCGAGGCCTGGGGGAGGGCGG
ATTGCGGCTTCACATCAGAATCATACCAACAAGGAGTATTGAGCGCGAC
AATTCTTTACGAAATTCTGCTTGGGAAAGCGACTCTGTACGCGGTGCTC
GTGTCCGCTTTGGTTCTTATGGCAATGGTTAAACGAAAGGATAGTAGGG
GC
(SEQ ID NO: 139)
MLLLLVPVLEVIFTLGGTRAQSVTQLGSHVSVSERALVLLRCNYSSSVP
PYLFWYVQYPNQGLQLLLKYTSAATLVKGINGFEAEFKKSETSFHLTKP
SAHMSDAAEYFCAVSGQTGANNLFFGTGTRLTVIPYIQNPDPAVYQLRD
SKSSDKSVCLFTDFDSQTNVSQSKDSDVYITDKCVLDMRSMDFKSNSAV
AWSNKSDFACANAFNNSIIPEDTFFPSPESSCDVKLVEKSFETDINLNF
QNLSVIGFRILLLKVAGFNLLMTLRLWSS
(SEQ ID NO: 140)
MGFRLLCCVAFCLLGAGPVDSGVTQTPKHLITATGQRVTLRCSPRSGDL
SVYWYQQSLDQGLQFLIQYYNGEERAKGNILERFSAQQFPDLHSELNLS
SLELGDSALYFCASARWDRGGEQYFGPGTRLTVTEDLKNVFPPEVAVFE
PSEAEISHTQKATLVCLATGFYPDHVELSWWVNGKEVHSGVCTDPQPLK
EQPALNDSRYCLSSRLRVSATFWQNPRNHFRCQVQFYGLSENDEWTQDR
AKPVTQIVSAEAWGRADCGFTSESYQQGVLSATILYEILLGKATLYAVL
VSALVLMAMVKRKDSRG

In some embodiments, a TCR construct comprising PRAME-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 141 (e.g., TCR clone DSK3 TCR alpha) and/or 142 (e.g., TCR clone DSK3 TCR beta). In some embodiments, a TCR construct comprising PRAME-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to SEQ ID NO: 143 (e.g., TCR clone DSK3 TCR alpha) and/or 144 (e.g., TCR clone DSK3 TCR beta).

(SEQ ID NO: 141)
ATGAAGAGCCTGAGGGTACTGCTGGTGATATTGTGGCTTCAGCTTAGTT
GGGTCTGGTCACAACAAAAGGAAGTTGAGCAAAACTCAGGACCACTGAG
TGTACCCGAGGGCGCTATAGCATCACTGAACTGTACCTACTCAGATCGG
GGAAGCCAATCCTTTTTCTGGTACAGACAGTATTCCGGGAAGAGTCCTG
AGTTGATCATGTTTATATACTCCAATGGCGATAAGGAGGATGGACGCTT
CACCGCTCAGCTTAATAAAGCGTCACAGTATGTATCCCTCCTGATTCGG
GACTCACAACCATCTGACTCTGCAACATACCTTTGTGCCGTAAAGGACA
ACGCCGGGAACATGCTCACTTTTGGAGGAGGTACCCGGCTTATGGTAAA
ACCACATATCCAGAACCCCGACCCCGCCGTGTACCAGCTGCGGGACAGC
AAGAGCAGCGACAAGAGCGTGTGCCTGTTCACCGACTTCGACAGCCAGA
CCAACGTGTCCCAGAGCAAGGACAGCGACGTGTACATCACCGATAAGTG
CGTGCTGGACATGCGGAGCATGGACTTCAAGAGCAACAGCGCCGTGGCC
TGGTCCAACAAGAGCGACTTCGCCTGCGCCAACGCCTTCAACAACAGCA
TCATCCCCGAGGACACATTCTTCCCAAGCCCCGAGAGCAGCTGCGACGT
GAAGCTGGTGGAGAAGTCCTTCGAGACAGACACCAACCTGAACTTCCAG
AACCTGTCCGTGATCGGCTTCAGAATCCTGCTGCTGAAAGTGGCCGGCT
TCAACCTGCTGATGACCCTGCGGCTGTGGTCCAGC
(SEQ ID NO: 142)
MKSLRVLLVILWLQLSWVWSQQKEVEQNSGPLSVPEGAIASLNCTYSDR
GSQSFFWYRQYSGKSPELIMFIYSNGDKEDGRFTAQLNKASQYVSLLIR
DSQPSDSATYLCAVKDNAGNMLTFGGGTRLMVKPHIQNPDPAVYQLRDS
KSSDKSVCLFTDFDSQTNVSQSKDSDVYITDKCVLDMRSMDFKSNSAVA
WSNKSDFACANAFNNSIIPEDTFFPSPESSCDVKLVEKSFETDTNLNFQ
NLSVIGFRILLLKVAGFNLLMTLRLWSS
(SEQ ID NO: 143)
ATGGGATTCCGGCTTCTTTGTTGTGTGGCATTTTGTCTGTTGGGTGCGG
GTCCAGTCGATAGTGGTGTAACTCAGACACCAAAACACCTTATCACGGC
AACTGGGCAACGAGTGACGCTCCGCTGTAGCCCGAGGTCCGGTGATTTG
AGTGTGTACTGGTACCAGCAATCTTTGGACCAGGGCTTGCAGTTCCTCA
TACAGTATTACAATGGTGAAGAAAGAGCGAAGGGTAATATCCTGGAAAG
ATTCTCCGCACAACAGTTTCCTGATCTCCACAGCGAACTGAACCTGAGT
TCTCTCGAGCTCGGGGATAGTGCTTTGTACTTCTGCGCGTCATCCGACG
GTGGCGGAGTCTATGAACAATATTTCGGCCCAGGGACTAGGCTTACGGT
GACGGAGGACCTCAAGAATGTGTTTCCGCCCGAAGTCGCGGTTTTTGAA
CCATCAGAAGCCGAGATCTCTCATACACAAAAGGCGACGCTCGTATGCC
TTGCGACGGGATTTTATCCGGACCACGTCGAGCTTTCCTGGTGGGTTAA
TGGAAAGGAGGTGCATTCCGGAGTTTGCACGGACCCTCAGCCATTGAAG
GAACAGCCCGCACTGAACGACAGTAGGTATTGCCTTTCATCTCGCCTGC
GCGTGTCTGCGACATTCTGGCAAAACCCAAGAAATCACTTCAGATGTCA
AGTTCAGTTCTACGGTCTCAGCGAGAATGATGAGTGGACACAAGATAGG
GCTAAACCCGTGACTCAAATAGTCTCTGCCGAGGCCTGGGGGAGGGCGG
ATTGCGGCTTCACATCAGAATCATACCAACAAGGAGTATTGAGCGCGAC
AATTCTTTACGAAATTCTGCTTGGGAAAGCGACTCTGTACGCGGTGCTC
GTGTCCGCTTTGGTTCTTATGGCAATGGTTAAACGAAAGGATAGTAGGG
GC
(SEQ ID NO: 144)
MGFRLLCCVAFCLLGAGPVDSGVTQTPKHLITATGQRVTLRCSPRSGDL
SVYWYQQSLDQGLQFLIQYYNGEERAKGNILERFSAQQFPDLHSELNLS
SLELGDSALYFCASSDGGGVYEQYFGPGTRLTVTEDLKNVFPPEVAVFE
PSEAEISHTQKATLVCLATGFYPDHVELSWWVNGKEVHSGVCTDPQPLK
EQPALNDSRYCLSSRLRVSATFWQNPRNHFRCQVQFYGLSENDEWTQDR
AKPVTQIVSAEAWGRADCGFTSESYQQGVLSATILYEILLGKATLYAVL
VSALVLMAMVKRKDSRG

In some embodiments, a TCR construct comprising PRAME-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 145-152. In some embodiments, a TCR construct comprising PRAME-specific TCR chains comprises TCR alpha and TCR beta chains found in PRAME-specific TCR clone T116-49 and/or T402-93 and/or modified versions thereof. In some embodiments, a TCR construct comprising PRAME-specific TCR chains comprises TCR alpha and TCR beta chains that target PRAME epitope LYVDSLFFL (SEQ ID NO: 167). In some embodiments, PRAME-specific TCR sequences, TCR variable domain sequences, CDR sequences, and/or TCR constant domain sequences, are described in international patent application publication WO 2022/063966 A1, which is incorporated herein by reference for the purpose described herein. In some embodiments, a TCR construct comprising PRAME-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 153-166.

(SEQ ID NO: 145)
ATGGAGACACTGCTGAAGGTGCTGTCTGGCACACTGCTGTGGCAGCTGACCTGGGTCCGATC
TCAGCAGCCTGTTCAGTCTCCTCAGGCCGTGATCCTGAGAGAAGGCGAGGACGCCGTGATCA
ACTGCAGCAGCTCTAAGGCCCTGTACAGCGTGCACTGGTACAGACAGAAGCACGGCGAGGCC
CCTGTGTTCCTGATGATCCTGCTGAAAGGCGGCGAGCAGAAGGGCCACGAGAAGATCAGCGC
CAGCTTCAACGAGAAGAAGCAGCAGTCCAGCCTGTACCTGACAGCCAGCCAGCTGAGCTACA
GCGGCACCTACTTTTGCGGCACAGCCAATAGCGGCGGCAGCAACTACAAGCTGACCTTCGGC
AAGGGCACCCTGCTGACCGTGAATCCCAAT
(SEQ ID NO: 146)
ATGCTGCTGATCACCTCCATGCTGGTGCTGTGGATGCAGCTGAGCCAAGTGAACGGCCAGCA
AGTGATGCAGATCCCTCAGTACCAGCACGTGCAAGAAGGCGAGGACTTCACCACCTACTGCA
ACAGCAGCACCACACTGAGCAACATCCAGTGGTACAAGCAGCGGCCTGGCGGACACCCTGTG
TTTCTGATCCAGCTGGTCAAGTCCGGCGAAGTGAAGAAGCAGAAGCGGCTGACCTTCCAGTT
CGGCGAGGCCAAGAAGAACAGCAGCCTGCACATCACCGCCACACAGACCACCGATGTGGGCA
CCTACTTTTGTGCTGGCGCCCTGCCTAGAGCCGGCAGCTATCAACTGACATTCGGCAAGGGC
ACCAAGCTGAGCGTGATCCCCAAC
(SEQ ID NO: 147)
ATGGAGACACTGCTGAAGGTGCTGTCTGGCACACTGCTGTGGCAGCTGACCTGGGTCCGATC
TCAGCAGCCTGTTCAGTCTCCTCAGGCCGTGATCCTGAGAGAAGGCGAGGACGCCGTGATCA
ACTGCAGCAGCTCTAAGGCCCTGTACAGCGTGCACTGGTACAGACAGAAGCACGGCGAGGCC
CCTGTGTTCCTGATGATCCTGCTGAAAGGCGGCGAGCAGAAGGGCCACGAGAAGATCAGCGC
CAGCTTCAACGAGAAGAAGCAGCAGTCCAGCCTGTACCTGACAGCCAGCCAGCTGAGCTACA
GCGGCACCTACTTTTGCGGCACAGCCAATAGCGGCGGCAGCAACTACAAGCTGACCTTCGGC
AAGGGCACCCTGCTGACCGTGAATCCCAATATCCAGAATCCGGAGCCCGCCGTATACCAGCT
GAAGGACCCTAGAAGCCAGGACAGCACCCTGTGCCTGTTCACCGACTTCGACAGCCAGATCA
ACGTGCCCAAGACCATGGAAAGCGGCACCTTCATCACCGACAAGACAGTGCTGGACATGAAG
GCCATGGACAGCAAGTCCAACGGCGCAATCGCCTGGTCCAACCAGACCAGCTTCACATGCCA
GGACATCTTCAAAGAGACAAACGCCACATACCCCAGCAGCGACGTGCCCTGTGATGCCACCC
TGACAGAGAAGTCCTTCGAGACAGACATGAACCTGAACTTCCAGAATCTGTCCGTGATGGGC
CTGAGAATCCTGCTGCTGAAGGTGGCCGGCTTCAATCTGCTGATGACCCTGCGGCTGTGGTC
CAGC
(SEQ ID NO: 148)
ATGCTGCTGATCACCTCCATGCTGGTGCTGTGGATGCAGCTGAGCCAAGTGAACGGCCAGCA
AGTGATGCAGATCCCTCAGTACCAGCACGTGCAAGAAGGCGAGGACTTCACCACCTACTGCA
ACAGCAGCACCACACTGAGCAACATCCAGTGGTACAAGCAGCGGCCTGGCGGACACCCTGTG
TTTCTGATCCAGCTGGTCAAGTCCGGCGAAGTGAAGAAGCAGAAGCGGCTGACCTTCCAGTT
CGGCGAGGCCAAGAAGAACAGCAGCCTGCACATCACCGCCACACAGACCACCGATGTGGGCA
CCTACTTTTGTGCTGGCGCCCTGCCTAGAGCCGGCAGCTATCAACTGACATTCGGCAAGGGC
ACCAAGCTGAGCGTGATCCCCAACATCCAGAATCCGGAGCCCGCCGTATACCAGCTGAAGGA
CCCTAGAAGCCAGGACAGCACCCTGTGCCTGTTCACCGACTTCGACAGCCAGATCAACGTGC
CCAAGACCATGGAAAGCGGCACCTTCATCACCGACAAGACAGTGCTGGACATGAAGGCCATG
GACAGCAAGTCCAACGGCGCAATCGCCTGGTCCAACCAGACCAGCTTCACATGCCAGGACAT
CTTCAAAGAGACAAACGCCACATACCCCAGCAGCGACGTGCCCTGTGATGCCACCCTGACAG
AGAAGTCCTTCGAGACAGACATGAACCTGAACTTCCAGAATCTGTCCGTGATGGGCCTGAGA
ATCCTGCTGCTGAAGGTGGCCGGCTTCAATCTGCTGATGACCCTGCGGCTGTGGTCCAGC
(SEQ ID NO: 149)
ATGGGCACCAGACTGTTCTTCTACGTGGCCCTGTGTCTGCTGTGGACAGGCCATGTGGATGC
CGGAATCACACAGAGCCCCAGACACAAAGTGACCGAGACAGGCACCCCTGTGACACTGAGAT
GTCACCAGACCGAGAACCATCGGTACATGTATTGGTACAGACAGGACCCCGGCCACGGCCTG
AGACTGATCCACTATAGCTACGGCGTGAAGGACACCGACAAGGGCGAAGTGTCTGACGGCTA
CAGCGTGTCCAGAAGCAAGACCGAGGACTTCCTGCTGACCCTGGAAAGCGCCACAAGCAGCC
AGACCAGCGTGTACTTCTGCGCCATCAGCGACTACGAGGGCACCGAGGCCTTTTTTGGCCAA
GGCACAAGACTGACCGTGGTG
(SEQ ID NO: 150)
ATGCTGTGTTCTCTGCTGGCTCTGCTGCTGGGCACCTTTTTTGGCGTCAGAAGCCAGACCAT
CCACCAGTGGCCTGCTACACTGGTGCAGCCTGTTGGAAGCCCTCTGAGCCTGGAATGTACCG
TGGAAGGCACCAGCAATCCCAACCTGTACTGGTACAGACAGGCCGCTGGAAGAGGACTGCAG
CTGCTGTTTTACAGCGTCGGCATCGGCCAGATCAGCAGCGAGGTTCCACAGAATCTGAGCGC
CAGCAGACCCCAGGACAGACAGTTTATCCTGAGCAGCAAGAAGCTGCTGCTGAGCGACAGCG
GCTTCTACCTGTGTGCTTGGAGCCTCGGAGCCGGCTACACCGACACACAGTATTTTGGCCCT
GGCACCAGACTGACCGTGCTG
(SEQ ID NO: 151)
ATGGGCACCAGACTGTTCTTCTACGTGGCCCTGTGTCTGCTGTGGACAGGCCATGTGGATGC
CGGAATCACACAGAGCCCCAGACACAAAGTGACCGAGACAGGCACCCCTGTGACACTGAGAT
GTCACCAGACCGAGAACCATCGGTACATGTATTGGTACAGACAGGACCCCGGCCACGGCCTG
AGACTGATCCACTATAGCTACGGCGTGAAGGACACCGACAAGGGCGAAGTGTCTGACGGCTA
CAGCGTGTCCAGAAGCAAGACCGAGGACTTCCTGCTGACCCTGGAAAGCGCCACAAGCAGCC
AGACCAGCGTGTACTTCTGCGCCATCAGCGACTACGAGGGCACCGAGGCCTTTTTTGGCCAA
GGCACAAGACTGACCGTGGTGGAAGATCTCCGGAACGTGACCCCCCCTAAAGTGACCCTGTT
CGAACCCAGCAAGGCCGAGATCGCCAACAAGCAGAAAGCCACCCTCGTGTGCCTGGCCAGAG
GCTTCTTCCCCGACCATGTGGAACTGTCTTGGTGGGTCAACGGCAAAGAGGTGCACAGCGGA
GTGTCCACCGACCCTCAGGCCTACAAAGAGAGCAACTACAGCTACTGCCTGAGCAGCAGACT
GCGGGTGTCCGCCACCTTCTGGCACAACCCCCGGAACCACTTCAGATGCCAGGTGCAGTTTC
ACGGCCTGAGCGAAGAGGACAAGTGGCCCGAAGGCTCCCCCAAGCCCGTGACCCAGAATATC
TCTGCCGAGGCCTGGGGCAGAGCCGACTGTGGAATTACCAGCGCCAGCTACCACCAGGGCGT
GCTGTCTGCCACCATCCTGTACGAGATCCTGCTGGGCAAGGCCACCCTGTACGCCGTGCTGG
TGTCTGGCCTGGTGCTGATGGCCATGGTCAAGAAGAAGAACAGC
(SEQ ID NO: 152)
ATGCTGTGTTCTCTGCTGGCTCTGCTGCTGGGCACCTTTTTTGGCGTCAGAAGCCAGACCAT
CCACCAGTGGCCTGCTACACTGGTGCAGCCTGTTGGAAGCCCTCTGAGCCTGGAATGTACCG
TGGAAGGCACCAGCAATCCCAACCTGTACTGGTACAGACAGGCCGCTGGAAGAGGACTGCAG
CTGCTGTTTTACAGCGTCGGCATCGGCCAGATCAGCAGCGAGGTTCCACAGAATCTGAGCGC
CAGCAGACCCCAGGACAGACAGTTTATCCTGAGCAGCAAGAAGCTGCTGCTGAGCGACAGCG
GCTTCTACCTGTGTGCTTGGAGCCTCGGAGCCGGCTACACCGACACACAGTATTTTGGCCCT
GGCACCAGACTGACCGTGCTGGAAGATCTCCGGAACGTGACCCCCCCTAAAGTGACCCTGTT
CGAACCCAGCAAGGCCGAGATCGCCAACAAGCAGAAAGCCACCCTCGTGTGCCTGGCCAGAG
GCTTCTTCCCCGACCATGTGGAACTGTCTTGGTGGGTCAACGGCAAAGAGGTGCACAGCGGA
GTGTCCACCGACCCTCAGGCCTACAAAGAGAGCAACTACAGCTACTGCCTGAGCAGCAGACT
GCGGGTGTCCGCCACCTTCTGGCACAACCCCCGGAACCACTTCAGATGCCAGGTGCAGTTTC
ACGGCCTGAGCGAAGAGGACAAGTGGCCCGAAGGCTCCCCCAAGCCCGTGACCCAGAATATC
TCTGCCGAGGCCTGGGGCAGAGCCGACTGTGGAATTACCAGCGCCAGCTACCACCAGGGCGT
GCTGTCTGCCACCATCCTGTACGAGATCCTGCTGGGCAAGGCCACCCTGTACGCCGTGCTGG
TGTCTGGCCTGGTGCTGATGGCCATGGTCAAGAAGAAGAACAGC
(SEQ ID NO: 153)
METLLKVLSGTLLWQLTWVRSQQPVQSPQAVILREGEDAVINCSSSKALYSVHWYRQKHGEA
PVFLMILLKGGEQKGHEKISASFNEKKQQSSLYLTASQLSYSGTYFCGTANSGGSNYKLTFG
KGTLLTVNPN
(SEQ ID NO: 154)
MLLITSMLVLWMQLSQVNGQQVMQIPQYQHVQEGEDFTTYCNSSTTLSNIQWYKQRPGGHPV
FLIQLVKSGEVKKQKRLTFQFGEAKKNSSLHITATQTTDVGTYFCAGALPRAGSYQLTFGKG
TKLSVIPN
(SEQ ID NO: 155)
IQNPEPAVYQLKDPRSQDSTLCLFTDFDSQINVPKTMESGTFITDKTVLDMKAMDSKSNGAI
AWSNQTSFTCQDIFKETNATYPSSDVPCDATLTEKSFETDMNLNFQNLSVMGLRILLLKVAG
FNLLMTLRLWSS
(SEQ ID NO: 156)
IQNPDPAVYQLRDSKSSDKSVCLFTDFDSQTNVSQSKDSDVYITDKTVLDMRSMDFKSNSAV
AWSNKSDFACANAFNNSIIPEDTFFPSPESSCDVKLVEKSFETDTNLNFQNLSVIGFRILLL
KVAGFNLLMTLRLWSS
(SEQ ID NO: 157)
IQNPDPAVYQLRDSKSSDKSVCLFTDFDSQTNVSQSKDSDVYITDKTVLDMRSMDFKSNSAV
AWSNKSDFACANAFNNSIIPEDTFFPSSDVPCDVKLVEKSFETDTNLNFQNLSVIGFRILLL
KVAGFNLLMTLRLWSS
(SEQ ID NO: 158)
METLLKVLSGTLLWQLTWVRSQQPVQSPQAVILREGEDAVINCSSSKALYSVHWYRQKHGEA
PVFLMILLKGGEQKGHEKISASFNEKKQQSSLYLTASQLSYSGTYFCGTANSGGSNYKLTFG
KGTLLTVNPNIQNPEPAVYQLKDPRSQDSTLCLFTDFDSQINVPKTMESGTFITDKTVLDMK
AMDSKSNGAIAWSNQTSFTCQDIFKETNATYPSSDVPCDATLTEKSFETDMNLNFQNLSVMG
LRILLLKVAGFNLLMTLRLWSS
(SEQ ID NO: 159)
MLLITSMLVLWMQLSQVNGQQVMQIPQYQHVQEGEDFTTYCNSSTTLSNIQWYKQRPGGHPV
FLIQLVKSGEVKKQKRLTFQFGEAKKNSSLHITATQTTDVGTYFCAGALPRAGSYQLTFGKG
TKLSVIPNIQNPEPAVYQLKDPRSQDSTLCLFTDFDSQINVPKTMESGTFITDKTVLDMKAM
DSKSNGAIAWSNQTSFTCQDIFKETNATYPSSDVPCDATLTEKSFETDMNLNFQNLSVMGLR
ILLLKVAGFNLLMTLRLWSS
(SEQ ID NO: 160)
MGTRLFFYVALCLLWTGHVDAGITQSPRHKVTETGTPVTLRCHQTENHRYMYWYRQDPGHGL
RLIHYSYGVKDTDKGEVSDGYSVSRSKTEDFLLTLESATSSQTSVYFCAISDYEGTEAFFGQ
GTRLTVV
(SEQ ID NO: 161)
MLCSLLALLLGTFFGVRSQTIHQWPATLVQPVGSPLSLECTVEGTSNPNLYWYRQAAGRGLQ
LLFYSVGIGQISSEVPQNLSASRPQDRQFILSSKKLLLSDSGFYLCAWSLGAGYTDTQYFGP
GTRLTVL
(SEQ ID NO: 162)
EDLRNVTPPKVTLFEPSKAEIANKQKATLVCLARGFFPDHVELSWWVNGKEVHSGVSTDPQA
YKESNYSYCLSSRLRVSATFWHNPRNHFRCQVQFHGLSEEDKWPEGSPKPVTQNISAEAWGR
ADCGITSASYHQGVLSATILYEILLGKATLYAVLVSGLVLMAMVKKKNS
(SEQ ID NO: 163)
DLNKVFPPEVAVFEPSEAEISHTQKATLVCLATGFFPDHVELSWWVNGKEVHSGVSTDPQPL
KEQPALNDSRYCLSSRLRVSATFWQNPRNHFRCQVQFYGLSENDEWTQDRAKPVTQIVSAEA
WGRADCGFTSVSYQQGVLSATILYEILLGKATLYAVLVSALVLMAMVKRKDF
(SEQ ID NO: 164)
EDLNKVFPPEVAVFEPSKAEIAHTQKATLVCLATGFFPDHVELSWWVNGKEVHSGVSTDPQP
LKEQPALNDSRYCLSSRLRVSATFWQNPRNHFRCQVQFYGLSENDEWTQDRAKPVTQIVSAE
AWGRADCGITSASYHQGVLSATILYEILLGKATLYAVLVSALVLMAMVKRKDF
(SEQ ID NO: 165)
MGTRLFFYVALCLLWTGHVDAGITQSPRHKVTETGTPVTLRCHQTENHRYMYWYRQDPGHGL
RLIHYSYGVKDTDKGEVSDGYSVSRSKTEDFLLTLESATSSQTSVYFCAISDYEGTEAFFGQ
GTRLTVVEDLRNVTPPKVTLFEPSKAEIANKQKATLVCLARGFFPDHVELSWWVNGKEVHSG
VSTDPQAYKESNYSYCLSSRLRVSATFWHNPRNHFRCQVQFHGLSEEDKWPEGSPKPVTQNI
SAEAWGRADCGITSASYHQGVLSATILYEILLGKATLYAVLVSGLVLMAMVKKKNS
(SEQ ID NO: 166)
MLCSLLALLLGTFFGVRSQTIHQWPATLVQPVGSPLSLECTVEGTSNPNLYWYRQAAGRGLQ
LLFYSVGIGQISSEVPQNLSASRPQDRQFILSSKKLLLSDSGFYLCAWSLGAGYTDTQYFGP
GTRLTVLEDLRNVTPPKVTLFEPSKAETANKQKATLVCLARGFFPDHVELSWWVNGKEVHSG
VSTDPQAYKESNYSYCLSSRLRVSATFWHNPRNHFRCQVQFHGLSEEDKWPEGSPKPVTQNI
SAEAWGRADCGITSASYHQGVLSATILYEILLGKATLYAVLVSGLVLMAMVKKKNS

In some embodiments, a TCR construct comprises gp100-specific TCR chains. In some embodiments, a TCR construct comprising gp100-specific TCR chains comprises TCR alpha and TCR beta chains found in gp100-specific TCR clone Sp(0.01)A and/or modified versions thereof. In some embodiments, a TCR construct comprising gp100-specific TCR chains comprises TCR alpha and TCR beta chains that target gp100 epitope KTWGQYWQV (SEQ ID NO: 168). In some embodiments, gp100-specific TCR sequences, TCR variable domain sequences, CDR sequences, and/or TCR constant domain sequences, are described in patent publication U.S. Pat. No. 8,216,565 B2, which is incorporated herein by reference for the purpose described herein.

In some embodiments, a TCR construct comprising gp100-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 169 and/or 170. In some embodiments, a TCR construct comprising gp100-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 171-174.

(SEQ ID NO: 169)
ATGAAATCCTTGAGTGTTTCCCTAGTGGTCCTGTGGCTCCAGTTAAACTGGGTGAACAGCCA
GCAGAAGGTGCAGCAGAGCCCAGAATCCCTCATTGTCCCAGAGGGAGCCATGACCTCTCTCA
ACTGCACTTTCAGCGACAGTGCTTCTCAGTATTTTGCATGGTACAGACAGCATTCTGGGAAA
GCCCCCAAGGCACTGATGTCCATCTTCTCCAATGGTGAAAAAGAAGAAGGCAGATTCACAAT
TCACCTCAATAAAGCCAGTCTGCATTTCTCGCTACACATCAGAGACTCCCAGCCCAGTGACT
CTGCTCTCTACCTCTGTGCAGCCAATAACTATGCCCAGGGATTAACCTTCGGTCTTGGCACC
AGAGTATCTGTGTTTCCCTACATCCAGAACCCAGAACCTGCTGTGTACCAGTTAAAAGATCC
TCGGTCTCAGGACAGCACCCTCTGCCTGTTCACCGACTTTGACTCCCAAATCAATGTGCCGA
AAACCATGGAATCTGGAACGTTCATCACTGACAAAACTGTGCTGGACATGAAAGCTATGGAT
TCCAAGAGCAATGGGGCCATTGCCTGGAGCAACCAGACAAGCTTCACCTGCCAAGATATCTT
CAAAGAGACCAACGCCACCTACCCCAGTTCAGACGTTCCCTGTGATGCCACGTTGACTGAGA
AAAGCTTTGAAACAGATATGAACCTAAACTTTCAAAACCTGTCAGTTATGGGACTCCGAATC
CTCCTGCTGAAAGTAGCCGGATTTAACCTGCTCATGACGCTGAGGCTGTGGTCCAGTTGA
(SEQ ID NO: 170)
ATGGGCTCCAGACTCTTCTTTGTGGTTTTGATTCTCCTGTGTGCAAAACACATGGAGGCTGC
AGTCACCCAAAGTCCAAGAAGCAAGGTGGCAGTAACAGGAGGAAAGGTGACATTGAGCTGTC
ACCAGACTAATAACCATGACTATATGTACTGGTATCGGCAGGACACGGGGCATGGGCTGAGG
CTGATCCATTACTCATATGTCGCTGACAGCACGGAGAAAGGAGATATCCCTGATGGGTACAA
GGCCTCCAGACCAAGCCAAGAGAATTTCTCTCTCATTCTGGAGTTGGCTTCCCTTTCTCAGA
CAGCTGTATATTTCTGTGCCAGCAGCCCTGGGGGGGGGGGGGAACAGTACTTCGGTCCCGGC
ACCAGGCTCACGGTTTTAGAGGATCTGAGAAATGTGACTCCACCCAAGGTCTCCTTGTTTGA
GCCATCAAAAGCAGAGATTGCAAACAAACGAAAGGCTACCCTCGTGTGCTTGGCCAGGGGCT
TCTTCCCTGACCACGTGGAGCTGAGCTGGTGGGTGAATGGCAAGGAGGTCCACAGTGGGGTC
AGCACGGACCCTCAGGCCTACAAGGAGAGCAATTATAGCTACTGCCTGAGCAGCCGCCTGAG
GGTCTCTGCTACCTTCTGGCACAATCCTCGAAACCACTTCCGCTGCCAAGTGCAGTTCCATG
GGCTTTCAGAGGAGGACAAGTGGCCAGAGGGCTCACCCAAACCTGTCACACAGAACATCAGT
GCAGAGGCCTGGGGCCGAGCAGACTGTGGGATTACCTCAGCATCCTATCAACAAGGGGTCTT
GTCTGCCACCATCCTCTATGAGATCCTGCTAGGGAAAGCCACCCTGTATGCTGTGCTTGTCA
GTACACTGGTGGTGATGGCTATGGTCAAAAGAAAGAATTCATGA
(SEQ ID NO: 171)
MKSLSVSLVVLWLQLNWVNSQQKVQQSPESLIVPEGAMTSLNCTFSDSASQYFAWYRQHSGK
APKALMSIFSNGEKEEGRFTIHLNKASLHFSLHIRDSQPSDSALYLCAANNYAQGLTFGLGT
RVSVFPYIQNPEPAVYQLKDPRSQDSTLCLFTDFDSQINVPKTMESGTFITDKTVLDMKAMD
SKSNGAIAWSNQTSFTCQDIFKETNATYPSSDVPCDATLTEKSFETDMNLNFQNLSVMGLRI
LLLKVAGFNLLMTLRLWSS
(SEQ ID NO: 172)
MGSRLFFVVLILLCAKHMEAAVTQSPRSKVAVTGGKVTLSCHQTNNHDYMYWYRQDTGHGLR
LIHYSYVADSTEKGDIPDGYKASRPSQENFSLILELASLSQTAVYFCASSPGGGGEQYFGPG
TRLTVLEDLRNVTPPKVSLFEPSKAEIANKRKATLVCLARGFFPDHVELSWWVNGKEVHSGV
STDPQAYKESNYSYCLSSRLRVSATFWHNPRNHFRCQVQFHGLSEEDKWPEGSPKPVTQNIS
AEAWGRADCGITSASYQQGVLSATILYEILLGKATLYAVLVSTLVVMAMVKRKNS
(SEQ ID NO: 173)
QQKVQQSPESLIVPEGAMTSLNCTFSDSASQYFAWYRQHSGKAPKALMSIFSNGEKEEGRFT
IHLNKASLHFSLHIRDSQPSDSALYLCAANNYAQGLTFGLGTRVSVFPY
(SEQ ID NO: 174)
EAAVTQSPRSKVAVTGGKVTLSCHQTNNHDYMYWYRQDTGHGLRLIHYSYVADSTEKGDIPD
GYKASRPSQENFSLILELASLSQTAVYFCASSPGGGGEQYFGPGTRLTVL

In some embodiments, a TCR construct comprises MART-1-specific TCR chains. In some embodiments, a TCR construct comprising MART-1-specific TCR chains comprises TCR alpha and TCR beta chains found in MART-1-specific TCR clones F4 and/or F5 and/or modified versions thereof. In some embodiments, a TCR construct comprising MART-1-specific TCR chains comprises TCR alpha and TCR beta chains that target MART-1 epitope AAGIGILTV (SEQ ID NO: 175). In some embodiments, MART-1-specific TCR sequences, TCR variable domain sequences, CDR sequences, and/or TCR constant domain sequences, are described in patent publication U.S. Pat. No. 9,128,080 B2, which is incorporated herein by reference for the purpose described herein.

In some embodiments, a TCR construct comprising MART-1-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 176-179. In some embodiments, a TCR construct comprising MART-1-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 180-183.

(SEQ ID NO: 176)
ATGTTGCTTGAACATTTATTAATAATCTTGTGGATGCAGCTGACATGGGTCAGTGGTCAACA
GCTGAATCAGAGTCCTCAATCTATGTTTATCCAGGAAGGAGAAGATGTCTCCATGAACTGCA
CTTCTTCAAGCATATTTAACACCTGGCTATGGTACAAGCAGGACCCTGGGGAAGGTCCTGTC
CTCTTGATAGCCTTATATAAGGCTGGTGAATTGACCTCAAATGGAAGACTGACTGCTCAGTT
TGGTATAACCAGAAAGGACAGCTTCCTGAATATCTCAGCATCCATACCTAGTGATGTAGGCA
TCTACTTCTGTGCTGGTGGGACCGGTAACCAGTTCTATTTTGGGACAGGGACAAGTTTGACG
GTCATTCCAAATATCCAGAACCCTGACCCTGCCGTGTACCAGCTGAGAGACTCTAAATCCAG
TGACAAGTCTGTCTGCCTATTCACCGATTTTGATTCTCAAACAAATGTGTCACAAAGTAAGG
ATTCTGATGTGTATATCACAGACAAAACTGTGCTAGACATGAGGTCTATGGACTTCAAGAGC
AACAGTGCTGTGGCCTGGAGCAACAAATCTGACTTTGCATGTGCAAACGCCTTCAACAACAG
CATTATTCCAGAAGACACCTTCTTCCCCAGCCCAGAAAGTTCCTGTGATGTCAAGCTGGTCG
AGAAAAGCTTTGAAACAGATACGAACCTAAACTTTCAAAACCTGTCAGTGATTGGGTTCCGA
ATCCTCCTCCTGAAGGTGGCCGGGTTTAATCTGCTCATGACGCTGCGGCTGTGGTCCAGC
(SEQ ID NO: 177)
ATGGGCACAAGGTTGTTCTTCTATGTGGCCCTTTGTCTCCTGTGGACAGGACACATGGATGC
TGGAATCACCCAGAGCCCAAGACACAAGGTCACAGAGACAGGAACACCAGTGACTCTGAGAT
GTCACCAGACTGAGAACCACCGCTATATGTACTGGTATCGACAAGACCCGGGGCATGGGCTG
AGGCTGATCCATTACTCATATGGTGTTAAAGATACTGACAAAGGAGAAGTCTCAGATGGCTA
TAGTGTCTCTAGATCAAAGACAGAGGATTTCCTCCTCACTCTGGAGTCCGCTACCAGCTCCC
AGACATCTGTGTACTTCTGTGCCATCAGTGAGGTAGGGGTTGGGCAGCCCCAGCATTTTGGT
GATGGGACTCGACTCTCCATCCTAGAGGACCTGAACAAGGTGTTCCCACCCGAGGTCGCTGT
GTTTGAGCCATCAGAAGCAGAGATCTCCCACACCCAAAAGGCCACACTGGTGTGCCTGGCCA
CAGGCTTCTTCCCCGACCACGTGGAGCTGAGCTGGTGGGTGAATGGGAAGGAGGTGCACAGT
GGGGTCAGCACGGACCCGCAGCCCCTCAAGGAGCAGCCCGCCCTCAATGACTCCAGATACTG
CCTGAGCAGCCGCCTGAGGGTCTCGGCCACCTTCTGGCAGAACCCCCGCAACCACTTCCGCT
GTCAAGTCCAGTTCTACGGGCTCTCGGAGAATGACGAGTGGACCCAGGATAGGGCCAAACCC
GTCACCCAGATCGTCAGCGCCGAGGCCTGGGGTAGAGCATGTGGCTTTACCTCGTCCTACCA
GCAAGGGGTCCTGTCTGCCACCATCCTCTATGAGATCCTGCTAGGGAAGGCCACCCTGTATG
CTGTGCTGGTCAGCGCCCTTGTGTTGATGGCCATGGTCAAGAGAAAGGATTTC
(SEQ ID NO: 178)
ATGATGAAATCCTTGAGAGTTTTACTAGTGATCCTGTGGCTTCAGTTGAGCTGGGTTTGGAG
CCAACAGAAGGAGGTGGAGCAGAATTCTGGACCCCTCAGTGTTCCAGAGGGAGCCATTGCCT
CTCTCAACTGCACTTACAGTGACCGAGGTTCCCAGTCCTTCTTCTGGTACAGACAATATTCT
GGGAAAAGCCCTGAGTTGATAATGTTCATATACTCCAATGGTGACAAAGAAGATGGAAGGTT
TACAGCACAGCTCAATAAAGCCAGCCAGTATGTTTCTCTGCTCATCAGAGACTCCCAGCCCA
GTGATTCAGCCACCTACCTCTGTGCCGTGAACTTCGGAGGAGGAAAGCTTATCTTCGGACAG
GGAACGGAGTTATCTGTGAAACCCAATATCCAGAACCCTGACCCTGCCGTGTACCAGCTGAG
AGACTCTAAATCCAGTGACAAGTCTGTCTGCCTATTCACCGATTTTGATTCTCAAACAAATG
TGTCACAAAGTAAGGATTCTGATGTGTATATCACAGACAAAACTGTGCTAGACATGAGGTCT
ATGGACTTCAAGAGCAACAGTGCTGTGGCCTGGAGCAACAAATCTGACTTTGCATGTGCAAA
CGCCTTCAACAACAGCATTATTCCAGAAGACACCTTCTTCCCCAGCCCAGAAAGTTCCTGTG
ATGTCAAGCTGGTCGAGAAAAGCTTTGAAACAGATACGAACCTAAACTTTCAAAACCTGTCA
GTGATTGGGTTCCGAATCCTCCTCCTGAAAGTGGCCGGGTTTAATCTGCTCATGACGCTGCG
GCTGTGGTCCAGCTGA
(SEQ ID NO: 179)
ATGAGAATCAGGCTCCTGTGCTGTGTGGCCTTTTCTCTCCTGTGGGCAGGTCCAGTGATTGC
TGGGATCACCCAGGCACCAACATCTCAGATCCTGGCAGCAGGACGGCGCATGACACTGAGAT
GTACCCAGGATATGAGACATAATGCCATGTACTGGTATAGACAAGATCTAGGACTGGGGCTA
AGGCTCATCCATTATTCAAATACTGCAGGTACCACTGGCAAAGGAGAAGTCCCTGATGGTTA
TAGTGTCTCCAGAGCAAACACAGATGATTTCCCCCTCACGTTGGCGTCTGCTGTACCCTCTC
AGACATCTGTGTACTTCTGTGCCAGCAGCCTAAGTTTCGGCACTGAAGCTTTCTTTGGACAA
GGCACCAGACTCACAGTTGTAGAGGACCTGAACAAGGTGTTCCCACCCGAGGTCGCTGTGTT
TGAGCCATCAGAAGCAGAGATCTCCCACACCCAAAAGGCCACACTGGTGTGCCTGGCCACAG
GCTTCTTCCCCGACCACGTGGAGCTGAGCTGGTGGGTGAATGGGAAGGAGGTGCACAGTGGG
GTCAGCACGGACCCGCAGCCCCTCAAGGAGCAGCCCGCCCTCAATGACTCCAGATACTGCCT
GAGCAGCCGCCTGAGGGTCTCGGCCACCTTCTGGCAGAACCCCCGCAACCACTTCCGCTGTC
AAGTCCAGTICTACGGGCTCTCGGAGAATGACGAGTGGACCCAGGATAGGGCCAAACCCGTC
ACCCAGATCGTCAGCGCCGAGGCCTGGGGTAGAGCATGTGGCTTTACCTCGTCCTACCAGCA
AGGGGTCCTGTCTGCCACCATCCTCTATGAGATCCTGCTAGGGAAGGCCACCCTGTATGCTG
TGCTGGTCAGCGCCCTTGTGTTGATGGCCATGGTCAAGAGAAAGGATTTC
(SEQ ID NO: 180)
GQQLNQSPQSMFIQEGEDVSMNCTSSSIFNTWLWYKQDPGEGPVLLIALYKAGELTSNGRLT
AQFGITRKDSFLNISASIPSDVGIYFCAGGTGNQFYFGTGTSLTVIPNIQNPDPAVYQLRDS
KSSDKSVCLFTDFDSQTNVSQSKDSDVYITDKTVLDMRSMDFKSNSAVAWSNKSDFACANAF
NNSIIPEDTFFPSPESSCDVKLVEKSFETDTNLNFQNLSVIGFRILLLKVAGFNLLMTLRLW
SS
(SEQ ID NO: 181)
DAGITQSPRHKVTETGTPVTLRCHQTENHRYMYWYRQDPGHGLRLIHYSYGVKDTDKGEVSD
GYSVSRSKTEDFLLTLESATSSQTSVYFCAISEVGVGQPQHFGDGIRLSILEDLNKVFPPEV
AVFEPSEAFISHTQKATLVCLATGFFPDHVELSWWVNGKEVHSGVSTDPQPLKEQPALNDSR
YCLSSRLRVSATFWQNPRNHFRCQVQFYGLSENDEWTQDRAKPVTQIVSAEAWGRACGFTSS
YQQGVLSATILYEILLGKATLYAVLVSALVLMAMVKRKDF
(SEQ ID NO: 182)
QKEVEQNSGPLSVPEGAIASLNCTYSDRGSQSFFWYRQYSGKSPELIMFIYSNGDKEDGRFT
AQLNKASQYVSLLIRDSQPSDSATYLCAVNFGGGKLIFGQGTELSVKPNIQNPDPAVYQLRD
SKSSDKSVCLFTDFDSQTNVSQSKDSDVYITDKTVLDMRSMDFKSNSAVAWSNKSDFACANA
FNNSIIPEDTFFPSPESSCDVKLVEKSFETDTNLNFQNLSVIGFRILLLKVAGFNLLMTLRL
WSS
(SEQ ID NO: 183)
IAGITQAPTSQILAAGRRMTLRCTQDMRHNAMYWYRQDLGLGLRLIHYSNTAGTTGKGEVPD
GYSVSRANTDDFPLTLASAVPSQTSVYFCASSLSFGTEAFFGQGTRLTVVEDLNKVFPPEVA
VFEPSEAEISHTQKATLVCLATGFFPDHVELSWWVNGKEVHSGVSTDPQPLKEQPALNDSRY
CLSSRLRVSATFWQNPRNHFRCQVQFYGLSENDEWTQDRAKPVTQIVSAEAWGRACGFTSSY
QQGVLSATILYEILLGKATLYAVLVSALVLMAMVKRKDF

In some embodiments, a TCR construct comprises Tyrosinase-specific TCR chains. In some embodiments, a TCR construct comprising Tyrosinase-specific TCR chains comprises TCR alpha and TCR beta chains found in Tyrosinase-specific TCR clone TIL 13831 and/or modified versions thereof. In some embodiments, a TCR construct comprising Tyrosinase-specific TCR chains comprises TCR alpha and TCR beta chains that target Tyrosinase epitope represented by amino acids 368-376 of tyrosinase (reactive against a class I MHC (HLA-A2)-restricted epitope (368-376) of tyrosinase). In some embodiments, Tyrosinase-specific TCR sequences, TCR variable domain sequences, CDR sequences, and/or TCR constant domain sequences, are described in publication Roszkowski et al, Cancer Res. 65(4): 1570-6 (2005), which is incorporated herein by reference for the purpose described herein.

In some embodiments, a TCR construct comprises MAGE-A3-specific TCR chains. In some embodiments, a TCR construct comprising MAGE-A3-specific TCR chains comprises TCR alpha and TCR beta chains that target amino acids 271-279 of MAGE-A3, e.g., the epitope FLWGPRALV (SEQ ID NO: 184). In some embodiments, a TCR construct comprising MAGE-A3-specific TCR chains comprises TCR alpha and TCR beta chains that target amino acids 112-120 of MAGE-A3, e.g., the epitope KVAELVHFL (SEQ ID NO: 185). In some embodiments, MAGE-A3-specific TCR sequences, TCR variable domain sequences, CDR sequences, and/or TCR constant domain sequences, are described in international patent application publication WO 2012/054825 A1, which is incorporated herein by reference for the purpose described herein. In certain embodiments, an anti-MAGE-A3 112-120 TCR comprise an A118T substitution relative to wild type (wherein the 118 position in the alpha chain is threonine). In certain embodiments, an anti-MAGE-A3 112-120 TCR comprises an A118V substitution relative to wild type (wherein the 118 position in the alpha chain is valine).

In some embodiments, a TCR construct comprising MAGE-A3-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 186-193. In some embodiments, a TCR construct comprising MAGE-A3-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 194-201.

(SEQ ID NO: 186)
ATGGGTCCTGTCACCTGCTCAGTTCTTGTGCTCCTCCTAATGCTCAGGAGGAGCAATGGCGA
TGGAGACTCCGTGACCCAGACAGAAGGCCTGGTCACTCTCACAGAAGGGTTGCCTGTGATGC
TGAACTGCACCTATCAGACTATTTACTCAAATCCTTTCCTTTTCTGGTATGTGCAACATCTC
AATGAATCCCCTCGGCTACTCCTGAAGAGCTTCACAGACAACAAGAGGACCGAGCACCAAGG
GTTCCACGCCACTCTCCATAAGAGCAGCAGCTCCTTCCATCTGCAGAAGTCCTCAGCGCAGC
TGTCAGACTCTGCCCTGTACTACTGTGCTTTCGACACAAATGCTTACAAAGTCATCTTT
(SEQ ID NO: 187)
ATGAGAGTTAGGCTCATCTCTGCTGTGGTGCTGTGTTCCCTAGGAACAGGCCTTGTGGACAT
GAAAGTAACCCAGATGCCAAGATACCTGATCAAAAGAATGGGAGAGAATGTTTTGCTGGAAT
GTGGACAGGACATGAGCCATGAAACAATGTACTGGTATCGACAAGACCCTGGTCTGGGGCTA
CAGCTGATTTATATCTCATACGATGTTGATAGTAACAGCGAAGGAGACATCCCTAAAGGATA
CAGGGTCTCACGGAAGAAGCGGGAGCATTTCTCCCTGATTCTGGATTCTGCTAAAACAAACC
AGACATCTGTGTACTTCTGTGCTAGCAGTTCAACAAACACAGAAGTCTTCTTT
(SEQ ID NO: 188)
ATGGGTCCTGTCACCTGCTCAGTTCTTGTGCTCCTCCTAATGCTCAGGAGGAGCAATGGCGA
TGGAGACTCCGTGACCCAGACAGAAGGCCTGGTCACTCTCACAGAAGGGTTGCCTGTGATGC
TGAACTGCACCTATCAGACTATTTACTCAAATCCTTTCCTTTTCTGGTATGTGCAACATCTC
AATGAATCCCCTCGGCTACTCCTGAAGAGCTTCACAGACAACAAGAGGACCGAGCACCAAGG
GTTCCACGCCACTCTCCATAAGAGCAGCAGCTCCTTCCATCTGCAGAAGTCCTCAGCGCAGC
TGTCAGACTCTGCCCTGTACTACTGTGCTTTCGACACAAATGCTTACAAAGTCATCTTTGGA
AAAGGGACACATCTTCATGTTCTCCCTAACATCCAGAACCCAGAACCTGCTGTGTACCAGTT
AAAAGATCCTCGGTCTCAGGACAGCACCCTCTGCCTGTTCACCGACTTTGACTCCCAAATCA
ATGTGCCGAAAACCATGGAATCTGGAACGTTCATCACTGACAAAACTGTGCTGGACATGAAA
GCTATGGATTCCAAGAGCAATGGGGCCATTGCCTGGAGCAACCAGACAAGCTTCACCTGCCA
AGATATCTTCAAAGAGACCAACACCACCTACCCCAGTTCAGACGTTCCCTGTGATGCCACGT
TGACTGAGAAAAGCTTTGAAACAGATATGAACCTAAACTTTCAAAACCTGTCAGTTATGGGA
CTCCGAATCCTCCTGCTGAAAGTAGCCGGATTTAACCTGCTCATGACGCTGAGGCTGTGGTC
CAGTTGA
(SEQ ID NO: 189)
ATGAGAGTTAGGCTCATCTCTGCTGTGGTGCTGTGTTCCCTAGGAACAGGCCTTGTGGACAT
GAAAGTAACCCAGATGCCAAGATACCTGATCAAAAGAATGGGAGAGAATGTTTTGCTGGAAT
GTGGACAGGACATGAGCCATGAAACAATGTACTGGTATCGACAAGACCCTGGTCTGGGGCTA
CAGCTGATTTATATCTCATACGATGTTGATAGTAACAGCGAAGGAGACATCCCTAAAGGATA
CAGGGTCTCACGGAAGAAGCGGGAGCATTTCTCCCTGATTCTGGATTCTGCTAAAACAAACC
AGACATCTGTGTACTTCTGTGCTAGCAGTTCAACAAACACAGAAGTCTTCTTTGGTAAAGGA
ACCAGACTCACAGTTGTAGAGGATCTGAGAAATGTGACTCCACCCAAGGTCTCCTTGTTTGA
GCCATCAAAAGCAGAGATTGCAAACAAACAAAAGGCTACCCTCGTGTGCTTGGCCAGGGGCT
TCTTCCCTGACCACGTGGAGCTGAGCTGGTGGGTGAATGGCAAGGAGGTCCACAGTGGGGTC
AGCACGGACCCTCAGGCCTACAAGGAGAGCAATTATAGCTACTGCCTGAGCAGCCGCCTGAG
GGTCTCTGCTACCTTCTGGCACAATCCTCGCAACCACTTCCGCTGCCAAGTGCAGTTCCATG
GGCTTTCAGAGGAGGACAAGTGGCCAGAGGGCTCACCCAAACCTGTCACACAGAACATCAGT
GCAGAGGCCTGGGGCCGAGCAGACTGTGGGATTACCTCAGCATCCTATCAACAAGGGGTCTT
GTCTGCCACCATCCTCTATGAGATCCTGCTAGGGAAAGCCACCCTGTATGCTGTGCTTGTCA
GTACACTGGTGGTGATGGCTATGGTCAAAAGAAAGAACTCGTGA
(SEQ ID NO: 190)
ATGGTCCTAGTGACCATTCTGCTGCTCAGCGCGTTCTTCTCACTGAGAGGAAACAGTGCCCA
GTCCGTGGACCAGCCTGATGCTCATGTCACGCTCTCTGAAGGAGCCTCCCTGGAGCTCAGAT
GCAGTTATTCATACAGTGCAGCACCTTACCTCTTCTGGTACGTGCAGTATCCTGGCCAGAGC
CTCCAGTTTCTCCTCAAATACATCACAGGAGACACCGTTGTTAAAGGCACCAAGGGCTTTGA
GGCCGAGTTTAGGAAGAGTAACTCCTCTTTCAACCTGAAGAAATCCCCAGCCCATTGGAGCG
ACTCAGCCAAGTACTTCTGTGCACTGGAGGGCCCGGATACAGGAAACTACAAATACGTCTT
(SEQ ID NO: 191)
ATGGGCATCCAGACCCTCTGTTGTGTGATCTTTTATGTTCTGATAGCAAATCACACAGATGC
TGGAGTTACCCAGACACCCAGACATGAGGTGGCAGAGAAAGGACAAACAATAATCCTGAAGT
GTGAGCCAGTTTCAGGCCACAATGACCTTTTCTGGTACAGACAGACCAAGATACAGGGACTA
GAGTTGCTGAGCTACTTCCGCAGCAAGTCTCTTATGGAAGATGGTGGGGCTTTCAAGGATCG
ATTCAAAGCTGAGATGCTAAATTCATCCTTCTCCACTCTGAAGATTCAACCTACAGAACCCA
GGGACTCAGCTGTGTATCTGTGTGCCAGCAGTTTTGGGACAGCTAGTGCAGAAACGCTGTAT
TTT
(SEQ ID NO: 192)
ATGGTCCTAGTGACCATTCTGCTGCTCAGCGCGTTCTTCTCACTGAGAGGAAACAGTGCCCA
GTCCGTGGACCAGCCTGATGCTCATGTCACGCTCTCTGAAGGAGCCTCCCTGGAGCTCAGAT
GCAGTTATTCATACAGTGCAGCACCTTACCTCTTCTGGTACGTGCAGTATCCTGGCCAGAGC
CTCCAGTTTCTCCTCAAATACATCACAGGAGACACCGTTGTTAAAGGCACCAAGGGCTTTGA
GGCCGAGTTTAGGAAGAGTAACTCCTCTTTCAACCTGAAGAAATCCCCAGCCCATTGGAGCG
ACTCAGCCAAGTACTTCTGTGCACTGGAGGGCCCGGATACAGGAAACTACAAATACGTCTTT
GGAGCAGGTACCAGACTGAAGGTTATAGCACACATCCAGAACCCAGAACCTGCTGTGTACCA
GTTAAAAGATCCTCGGTCTCAGGACAGCACCCTCTGCCTGTTCACCGACTTTGACTCCCAAA
TCAATGTGCCGAAAACCATGGAATCTGGAACGTTCATCACTGACAAAACTGTGCTGGACATG
AAAGCTATGGATTCCAAGAGCAATGGGGCCATTGCCTGGAGCAACCAGACAAGCTTCACCTG
CCAAGATATCTTCAAAGAGACCAACGCCACCTACCCCAGTTCAGACGTTCCCTGTGATGCCA
CGTTGACTGAGAAAAGCTTTGAAACAGATATGAACCTAAACTTCCAAAACCTGTCAGTTATG
GGACTCCGAATCCTCCTGCTGAAAGTAGCCGGATTTAACCTGCTCATGACGCTGAGGCTGTG
GTCCAGTTGA
(SEQ ID NO: 193)
ATGGGCATCCAGACCCTCTGTTGTGTGATCTTTTATGTTCTGATAGCAAATCACACAGATGC
TGGAGTTACCCAGACACCCAGACATGAGGTGGCAGAGAAAGGACAAACAATAATCCTGAAGT
GTGAGCCAGTTTCAGGCCACAATGACCTTTTCTGGTACAGACAGACCAAGATACAGGGACTA
GAGTTGCTGAGCTACTTCCGCAGCAAGTCTCTTATGGAAGATGGTGGGGCTTTCAAGGATCG
ATTCAAAGCTGAGATGCTAAATTCATCCTTCTCCACTCTGAAGATTCAACCTACAGAACCCA
GGGACTCAGCTGTGTATCTGTGTGCCAGCAGTTTTGGGACAGCTAGTGCAGAAACGCTGTAT
TTTGGCTCAGGAACCAGACTGACTGTTCTCGAGGATCTGAGAAATGTGACTCCACCCAAGGT
CTCCTTGTTTGAGCCATCAAAAGCAGAGATTGCAAACAAACAAAAGGCTACCCTCGTGTGCT
TGGCCAGGGGCTTCTTCCCCTGACACGTGGAGCTGAGCTGGTGGGTGAATGGCAAGGAGGTC
CACAGTGGGGTCAGCACGGACCCTCAGGCCTACAAGGAGAGCAATTATAGCTACTGCCTGAG
CAGCCGCCTGAGGGTCTCTGCTACCTTCTGGCACAATCCTCGAAACCACTTCCGCTGTCAAG
TGCAGTTCCATGGGCTTTCAGAGGAGGACAAGTGGCCAGAGGGCTCACCCAAACCTGTCACA
CAGAACATCAGTGCAGAGGCCTGGGGCCGAGCAGACTGTGGAATCACTTCAGCATCCTATCA
TCAGGGGGTTCTGTCTGCAACCATCCTCTATGAGATCCTACTGGGGAAGGCCACCCTATATG
CTGTGCTGGTCAGTGGCCTGGTGCTGATGGCCATGGTCAAGAAAAAAAATTCCTGA
(SEQ ID NO: 194)
MGPVTCSVLVLLLMLRRSNGDGDSVTQTEGLVTLTEGLPVMLNCTYQTIYSNPFLFWYVQHL
NESPRLLLKSFTDNKRTEHQGFHATLHKSSSSFHLQKSSAQLSDSALYYCAFDTNAYKVIF
(SEQ ID NO: 195)
MRVRLISAVVLCSLGTGLVDMKVTQMPRYLIKRMGFNVLLECGQDMSHETMYWYRQDPGLGL
QLIYISYDVDSNSEGDIPKGYRVSRKKREHFSLILDSAKTNQTSVYFCASSSTNTEVF
(SEQ ID NO: 196)
MGPVTCSVLVLLLMLRRSNGDGDSVTQTEGLVTLTEGLPVMLNCTYQTIYSNPFLFWYVQHL
NESPRLLLKSFTDNKRTEHQGFHATLHKSSSSFHLQKSSAQLSDSALYYCAFDTNAYKVIFG
KGTHLHVLPNIQNPEPAVYQLKDPRSQDSTLCLFTDFDSQINVPKTMESGTFITDKTVLDMK
AMDSKSNGAIAWSNQTSFTCQDIFKETNTTYPSSDVPCDATLTEKSFETDMNLNFQNLSVMG
LRILLLKVAGFNLLMTLRLWSSL
(SEQ ID NO: 197)
MRVRLISAVVLCSLGTGLVDMKVTQMPRYLIKRMGFNVLLECGQDMSHETMYWYRQDPGLGL
QLIYISYDVDSNSEGDIPKGYRVSRKKREHFSLILDSAKTNQTSVYFCASSSTNTEVFFGKG
TRLTVVEDLRNVTPPKVSLFEPSKAEIANKQKATLVCLARGFFPDHVFLSWWVNGKEVHSGV
STDPQAYKESNYSYCLSSRLRVSATFWHNPRNHFRCQVQFHGLSEEDKWPEGSPKPVTQNIS
AEAWGRADCGITSASYQQGVLSATILYEILLGKATLYAVLVSTLVVM
(SEQ ID NO: 198)
MVLVTILLLSAFFSLRGNSAQSVDQPDAHVTLSEGASLELRCSYSYSAAPYLFWYVQYPGQS
LQFLLKYITGDTVVKGTKGFEAEFRKSNSSFNLKKSPAHWSDSAKYFCALEGPDTGNYKYV
(SEQ ID NO: 199)
MGIQTLCCVIFYVLIANHTDAGVTQTPRHEVAEKGQTIILKCEPVSGHNDLFWYRQTKIQGL
ELLSYFRSKSLMEDGGAFKDRFKAEMLNSSFSTLKIQPTEPRDSAVYLCASSFGTASAETLY
(SEQ ID NO: 200)
MVLVTILLLSAFFSLRGNSAQSVDQPDAHVTLSEGASLELRCSYSYSAAPYLFWYVQYPGQS
LQFLLKYITGDTVVKGTKGFEAEFRKSNSSFNLKKSPAHWSDSAKYFCALEGPDTGNYKYVF
GAGTRLKVIAHIQNPEPAVYQLKDPRSQDSTLCLFTDFDSQINVPKTMESGTFITDKTVLDM
KAMDSKSNGAIAWSNQTSFTCQDIFKETNATYPSSDVPCDATLTEKSFETDMNLNFQNLSVM
GLRILLLKVAGFNLLMTLRLWSS
(SEQ ID NO: 201)
MGIQTLCCVIFYVLIANHTDAGVTQTPRHEVAEKGQTIILKCEPVSGHNDLFWYRQTKIQGL
ELLSYFRSKSLMEDGGAFKDRFKAEMLNSSFSTLKIQPTEPRDSAVYLCASSFGTASAETLY
FGSGTRLTVLEDLRNVTPPKVSLFEPSKAEIANKQKATLVCLARGFFPHVFLSWWVNGKEVH
SGVSTDPQAYKESNYSYCLSSRLRVSATFWHNPRNHFRCQVQFHGLSEEDKWPEGSPKPVTQ
NISAEAWGRADCGITSASYHQGVLSATILYEILLGKATLYAVLVSGLVLMAMVKKKNS

In some embodiments, a TCR construct comprises MAGE-A4-specific TCR chains. In some embodiments, a TCR construct comprising MAGE-A4-specific TCR chains comprises TCR alpha and TCR beta chains that target the epitope GVYDGREHTV (SEQ ID NO: 202). In some embodiments, a TCR construct comprising MAGE-A4-specific TCR chains comprises TCR alpha and TCR beta chains that target the epitope FMNKFIYEI (SEQ ID NO: 203). In some embodiments, MAGE-A4-specific TCR sequences, TCR variable domain sequences, CDR sequences, and/or TCR constant domain sequences, are described in international patent application publications WO 2017/174824 A1 and WO 2021/229212 A1, each of which are incorporated herein by reference for the purpose described herein. In certain embodiments, an anti-MAGE-A4 TCR alpha chain variable domain may have an M4V or an M4L amino acid substitution. In certain embodiments, an anti-MAGE-A4 TCR beta chain variable domain may have a N10E amino acid substitution.

In some embodiments, a TCR construct comprising MAGE-A4-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 204-205. In some embodiments, a TCR construct comprising MAGE-A4-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 206-214.

(SEQ ID NO: 204)
ATGAAGAAGCACCTGACCACCTTTCTCGTGATCCTGTGGCTGTACTTCTACCGGGGCAACGG
CAAGAACCAGGTGGAACAGAGCCCCCAGAGCCTGATCATCCTGGAAGGCAAGAACTGCACCC
TGCAGTGCAACTACACCGTGTCCCCCTTCAGCAACCTGCGGTGGTACAAGCAGGACACCGGC
AGAGGCCCTGTGTCCCTGACCATCCTGACCTTCAGCGAGAACACCAAGAGCAACGGCCGGTA
CACCGCCACCCTGGACGCCGATACAAAGCAGAGCAGCCTGCACATCACCGCCAGCCAGCTGA
GCGATAGCGCCAGCTACATCTGCGTGGTGTCCGGCGGCACAGACAGCTGGGGCAAGCTGCAG
TTTGGCGCCGGAACACAGGTGGTCGTGACCCCCGACATCCAGAACCCTGACCCTGCCGTGTA
CCAGCTGCGGGACAGCAAGAGCAGCGACAAGAGCGTGTGCCTGTTCACCGACTTCGACAGCC
AGACCAACGTGTCCCAGAGCAAGGACAGCGACGTGTACATCACCGACAAGACCGTGCTGGAC
ATGCGGAGCATGGACTTCAAGAGCAATAGCGCCGTGGCCTGGTCCAACAAGAGCGACTTCGC
CTGCGCCAACGCCTTCAACAACAGCATTATCCCCGAGGACACATTCTTCCCAAGCCCCGAGA
GCAGCTGCGACGTCAAGCTGGTGGAAAAGAGCTTCGAGACAGACACCAACCTGAACTTCCAG
AACCTGAGCGTGATCGGCTTCAGAATCCTGCTGCTGAAGGTGGCCGGCTTCAACCTGCTGAT
GACCCTGAGACTGTGGTCCAGCGGCAGCCGGGCCAAGAGA
(SEQ ID NO: 205)
ATGGCCAGCCTGCTGTTCTTCTGCGGCGCCTTCTACCTGCTGGGCACCGGCTCTATGGATGC
CGACGTGACCCAGACCCCCCGGAACAGAATCACCAAGACCGGCAAGCGGATCATGCTGGAAT
GCTCCCAGACCAAGGGCCACGACCGGATGTACTGGTACAGACAGGACCCTGGCCTGGGCCTG
CGGCTGATCTACTACAGCTTCGACGTGAAGGACATCAACAAGGGCGAGATCAGCGACGGCTA
CAGCGTGTCCAGACAGGCTCAGGCCAAGTTCAGCCTGTCCCTGGAAAGCGCCATCCCCAACC
AGACCGCCCTGTACTTTTGTGCCACAAGCGGCCAGGGCGCCTACGAGGAGCAGTTCTTTGGC
CCTGGCACCCGGCTGACAGTGCTGGAAGATCTGAAGAACGTGTTCCCCCCAGAGGTGGCCGT
GTTCGAGCCTTCTGAGGCCGAAATCAGCCACACCCAGAAAGCCACACTCGTGTGTCTGGCCA
CCGGCTTCTACCCCGACCACGTGGAACTGTCTTGGTGGGTCAACGGCAAAGAGGTGCACAGC
GGCGTGTCCACCGATCCCCAGCCTCTGAAAGAACAGCCCGCCCTGAACGACAGCCGGTACTG
CCTGAGCAGCAGACTGAGAGTGTCCGCCACCTTCTGGCAGAACCCCAGAAACCACTTCAGAT
GCCAGGTGCAGTTTTACGGCCTGAGCGAGAACGACGAGTGGACCCAGGACAGAGCCAAGCCC
GTGACACAGATCGTGTCTGCCGAAGCTTGGGGGCGCGCCGATTGTGGCTTTACCAGCGAGAG
CTACCAGCAGGGCGTGCTGAGCGCCACCATCCTGTACGAGATCCTGCTGGGAAAGGCCACAC
TGTACGCCGTGCTGGTGTCTGCCCTGGTGCTGATGGCCATGGTCAAGCGGAAGGACAGCCGG
GGC
(SEQ ID NO: 206)
MKKHLTTFLVILWLYFYRGNGKNQVEQSPQSLIILEGKNCTLQCNYTVSPFSNLRWYKQDTG
RGPVSLTILTFSENTKSNGRYTATLDADTKQSSLHITASQLSDSASYICVVSGGTDSWGKLQ
FGAGTQVVVTPDIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNFQ
NLSVIGFRILLLKVAGFNLLMTLRLWSSGSRAKR
(SEQ ID NO: 207)
MKKHLTTFLVILWLYFYRGNGKNQVEQSPQSLIILEGKNCTLQCNYTVSPFSNLRWYKQDTG
RGPVSLTILTFSENTKSNGRYTATLDADTKQSSLHITASQLSDSASYICVVSGGTDSWGKLQ
FGAGTQVVVTPD
(SEQ ID NO: 208)
MASLLFFCGAFYLLGTGSMDADVTQTPRNRITKTGKRIMLECSQTKGHDRMYWYRQDPGLGL
RLIYYSFDVKDTNKGEISDGYSVSRQAQAKFSLSLESAIPNQTALYFCATSGQGAYEEQFFG
PGTRLTVLEDLKNVFPPEVAVFEPSEAEISHTQKATLVCLATGFYPDHVFLSWWVNGKEVHS
GVSTDPQPLKEQPALNDSRYCLSSRLRVSATFWQNPRNHERCQVQFYGLSENDEWTQDRAKP
VTQIVSAEAWGRADCGFTSESYQQGVLSATILYEILLGKATLYAVLVSALVLMAMVKRKDSR
G
(SEQ ID NO: 209)
MASLLFFCGAFYLLGTGSMDADVTQTPRNRITKTGKRIMLECSQTKGHDRMYWYRQDPGLGL
RLIYYSFDVKDINKGEISDGYSVSRQAQAKFSLSLESAIPNQTALYFCATSGQGAYEEQFFG
PGTRLTVLE
(SEQ ID NO: 210)
MKNQVEQSPQSLIILEGKNCTLQCNYTVSPFSNLRWYKQDTGRGPVSLTIMTFSENTKSNGR
YTATLDADTKQSSLHITASQLSDSASYICVVSGGTDSWGKLQF
(SEQ ID NO: 211)
MKNQVEQSPQSLIILEGKNCTLQCNYTVSPFSNLRWYKQDTGRGPVSLTIVTFSENTKSNGR
YTATLDADTKQSSLHITASQLSDSASYICVVSGGTDSWGKLQF
(SEQ ID NO: 212)
MKNQVEQSPQSLIILEGKNCTLQCNYTVSPFSNLRWYKQDTGRGPVSLTILTFSENTKSNGR
YTATLDADTKQSSLHITASQLSDSASYICVVSGGTDSWGKLQF
(SEQ ID NO: 213)
MASLLFFCGAFYLLGTGSMDADVTQTPRNRITKTGKRIMLECSQTKGHDRMYWYRQDPGLGL
RLIYYSFDVKDINKGEISDGYSVSRQAQAKFSLSLESAIPNQTALYFCATSGQGAYNEQFF
(SEQ ID NO: 214)
MASLLFFCGAFYLLGTGSMDADVTQTPRNRITKTGKRIMLECSQTKGHDRMYWYRQDPGLGL
RLIYYSFDVKDINKGEISDGYSVSRQAQAKFSLSLESAIPNQTALYFCATSGQGAYEEQFF

In some embodiments, a TCR construct comprises Wilms' tumor antigen (WT1) WT1-specific TCR chains. In some embodiments, a TCR construct comprising WT1-specific TCR chains comprises TCR alpha and TCR beta chains that target the epitope VLDFAPPGA (SEQ ID NO: 215). In some embodiments, a TCR construct comprising WT1-specific TCR chains comprises TCR alpha and TCR beta chains that target the epitope RMFPNAPYL (SEQ ID NO: 216). In some embodiments, WT1-specific TCR sequences, TCR variable domain sequences, CDR sequences, and/or TCR constant domain sequences, are described in international patent application publications WO 2020/185796 A1 and WO 2021/034976 A1, each of which are incorporated herein by reference for the purpose described herein. In some embodiments, a leader sequence and/or signal peptide may be removed from a TCR amino acid sequence, and percentage sequence identity may be calculated based on the TCR amino acid sequence without the leader sequence and/or signal peptide.

In some embodiments, a TCR construct comprising WT1-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 217-256. In some embodiments, a TCR construct comprising WT1-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 257-291.

(SEQ ID NO: 217)
ATGGAGACACTGCTGGGACTACTGATTCTGTGGCTGCAACTGCAATGGGTGAGCAGCAAACA
GGAGGTTACCCAGATTCCTGCTGCTCTGTCTGTTCCTGAAGGCGAGAATCTGGTGCTGAACT
GCAGCTTCACAGATAGCGCCATCTACAACCTGCAGTGGTTCAGACAGGATCCTGGAAAAGGC
CTGACAAGCCTGCTGCTGATTCAGAGCTCTCAGAGAGAGCAGACATCTGGAAGACTGAATGC
TAGCCTGGACAAGTCTAGCGGCAGAAGCACCCTGTATATTGCCGCCTCTCAACCTGGAGATT
CTGCCACATACCTGTGTGCTGTGAAGGAGACATCTGGCTCTAGACTGACCTTTGGCGAGGGA
ACACAACTGACCGTGAATCCTGAC
(SEQ ID NO: 218)
ATGACCAGAGTTAGCCTGTTATGGGCTGTGGTGGTGAGCACATGTCTGGAATCTGGAATGGC
CCAGACAGTGACACAGTCTCAGCCTGAAATGTCTGTGCAGGAAGCCGAAACCGTTACACTGA
GCTGCACCTACGATACAAGCGAGAACAACTACTACCTGTTCTGGTACAAGCAGCCCCCCTCT
AGGCAGATGATCCTGGTGATCAGACAGGAGGCCTATAAACAGCAGAATGCCACAGAGAACCG
GTTCAGCGTGAACTTCCAGAAAGCCGCCAAGAGCTTCAGCCTGAAGATCTCTGATTCTCAGC
TGGGCGATACAGCCATGTACTTTTGCGCCTTCATCTACCCCAGCTACACAAGCGGCACATAC
AAGTACATCTTCGGCACCGGCACAAGACTGAAGGTTCTGGCCAAC
(SEQ ID NO: 219)
ATGGCCATGTTACTAGGAGCGAGCGTGCTGATTCTGTGGTTACAGCCTGATTGGGTGAACTC
TCAGCAGAAGAACGATGATCAGCAGGTGAAGCAGAACAGCCCCTCTCTGTCTGTGCAGGAAG
GCAGAATCAGCATCCTGAATTGCGATTACACCAACAGCATGTTCGACTACTTCCTGTGGTAC
AAGAAGTACCCCGCCGAGGGCCCTACCTTTCTGATCAGCATCTCTAGCATCAAGGACAAGAA
CGAAGATGGCAGATTCACCGTGTTCCTGAACAAGAGCGCCAAGCACCTGAGCCTGCACATTG
TGCCTTCTCAACCTGGAGATTCTGCCGTGTACTTTTGTGCTGCCTCTGGAACAGGCGGAAGC
TATATCCCCACATTTGGAAGAGGAACAAGCCTGATCGTGCACCCTTAC
(SEQ ID NO: 220)
ATGGCCATGTTACTAGGAGCGAGCGTGCTGATTCTGTGGTTACAGCCTGATTGGGTGAACTC
TCAGCAGAAGAACGATGATCAGCAGGTGAAGCAGAACAGCCCCTCTCTGTCTGTGCAGGAAG
GCAGAATCAGCATCCTGAATTGCGATTACACCAACAGCATGTTCGACTACTTCCTGTGGTAC
AAGAAGTACCCCGCCGAGGGCCCTACCTTTCTGATCAGCATCTCTAGCATCAAGGACAAGAA
CGAAGATGGCAGATTCACCGTGTTCCTGAACAAGAGCGCCAAGCACCTGAGCCTGCACATTG
TGCCTTCTCAACCTGGAGATTCTGCCGTGTACTTTTGTGCTGCCTCTGGCATTGGCGACTAC
AAACTGAGCTTTGGAGCCGGCACAACAGTGACCGTTAGAGCCAAT
(SEQ ID NO: 221)
ATGGTGAAGATCCGGCAGTTCCTCCTGGCTATTCTGTGGCTGCAACTGTCTTGTGTGTCTGC
TGCCAAGAATGAAGTGGAGCAGTCTCCCCAGAACCTTACAGCCCAGGAAGGCGAGTTTATCA
CCATCAACTGCAGCTATTCTGTGGGCATTAGCGCCCTGCATTGGCTGCAGCAACACCCTGGA
GGAGGAATTGTGTCTCTGTTTATGCTGTCTTCTGGCAAGAAGAAGCACGGCCGGCTGATTGC
CACCATCAACATCCAGGAGAAGCACTCTTCTCTGCACATTACAGCCTCTCATCCCAGGGATT
CTGCCGTGTACATCTGTGCCGTGAGAACCAGCTACGATAAGGTGATTTTCGGACCAGGCACC
TCTCTGAGCGTGATCCCCAAT
(SEQ ID NO: 222)
ATGAAGAGCCTGAGAGTCCTGCTGGTGATTTTGTGGCTGCAGCTGTCTTGGGTTTGGTCTCA
GCAGAAAGAAGTGGAGCAGAATAGCGGCCCTCTGTCTGTTCCTGAAGGCGCTATTGCTAGCC
TGAATTGCACATACAGCGATAGAGGATCTCAGAGCTTCTTCTGGTACCGGCAGTACAGCGGC
AAGAGCCCAGAACTGATCATGTTCATCTACAGCAATGGCGACAAGGAGGATGGCAGGTTTAC
AGCCCAGCTGAACAAGGCCAGCCAGTATGTTTCTCTGCTGATCAGAGATAGCCAGCCTAGCG
ATTCTGCCACCTACCTGTGTGCCGTGAACTTACTTGGAGCTACAGGATACTCTACACTGACC
TTCGGCAAAGGCACCATGCTGCTGGTGAGCCCTGAT
(SEQ ID NO: 223)
ATGTGGGGCGTTTTCCTTCTGTATGTGAGCATGAAGATGGGCGGCACAACAGGCCAGAACAT
CGATCAGCCTACCGAGATGACAGCCACAGAAGGAGCTATTGTTCAGATCAACTGCACCTACC
AGACAAGCGGCTTCAACGGCCTGTTCTGGTACCAGCAGCATGCTGGAGAAGCTCCTACATTT
CTGAGCTACAATGTGCTGGATGGCCTGGAGGAGAAAGGCAGGTTTAGCAGCTTCCTGAGCAG
GTCTAAGGGCTATTCTTATCTGCTGCTGAAGGAGCTGCAGATGAAGGATTCCGCCAGCTACC
TGTGTGCCGTTAGGGGCATCAATGATTACAAGCTGAGCTTTGGAGCCGGAACAACAGTGACC
GTGAGAGCCAAC
(SEQ ID NO: 224)
ATGGAGAAGATGCTGGAGTGTGCGTTCATCGTTCTGTGGCTGCAACTTGGATGGCTGTCTGG
AGAGGATCAGGTTACACAGTCTCCTGAAGCCCTGAGACTGCAAGAAGGAGAAAGCTCTAGCC
TGAACTGCAGCTACACAGTGTCTGGACTGAGAGGCCTGTTCTGGTACAGACAGGATCCTGGA
AAAGGCCCAGAGTTCCTGTTTACCCTGTATTCTGCCGGCGAGGAGAAGGAGAAAGAGAGACT
GAAAGCTACCCTGACCAAGAAGGAGAGCTTCCTGCACATTACCGCCCCCAAACCTGAGGATT
CTGCCACATATCTGTGTGCCGTGATTACCGGCTTTCAGAAGCTGGTGTTTGGCACAGGCACC
AGACTGCTGGTTTCTCCCAAT
(SEQ ID NO: 225)
ATGAGACTGGTGGCACGCGTAACTGTGTTTCTGACCTTTGGCACCATCATCGATGCCAAGAC
AACCCAGCCTACAAGCATGGACTGTGCCGAGGGAAGAGCTGCTAATCTGCCATGTAATCACA
GCACAATCAGCGGCAACGAGTACGTGTACTGGTACCGGCAGATCCACTCTCAAGGACCTCAG
TACATCATTCATGGCCTGAAGAACAACGAGACCAACGAGATGGCCAGCCTGATCATCACCGA
GGACAGGAAGTCTTCTACCCTGATTCTGCCTCATGCTACACTGAGAGATACCGCCGTGTACT
ACTGCATTGCCGGAGTGGGAAGAGGCCAGAATTTCGTGTTTGGACCTGGAACAAGACTGAGC
GTTCTGCCCTAT
(SEQ ID NO: 226)
ATGGAGAAGAACCCCTTGGCAGCACCTCTGCTTATTCTGTGGTTCCACCTGGATTGTGTGAG
CAGCATCCTGAATGTGGAGCAGTCTCCTCAGAGCCTGCATGTGCAAGAAGGCGATAGCACCA
ATTTCACCTGCAGCTTTCCAAGCAGCAACTTCTACGCCCTGCACTGGTACAGATGGGAAACC
GCCAAATCTCCTGAAGCCCTGTTTGTGATGACCCTGAATGGCGACGAGAAGAAGAAGGGCAG
AATTAGCGCCACCCTGAATACCAAGGAGGGCTACAGCTACCTGTACATCAAGGGCTCTCAAC
CTGAGGATTCTGCCACCTACCTTTGCGCCTTTCACCCCAATTTCGGCAACGAGAAACTGACC
TTTGGAACCGGAACAAGGCTGACCATCATCCCCAAC
(SEQ ID NO: 227)
ATGGAGAAGATGCTGGAGTGTGCGTTCATCGTTCTGTGGCTGCAACTTGGATGGCTGTCTGG
AGAGGATCAGGTTACACAGTCTCCTGAAGCCCTGAGACTGCAAGAAGGAGAAAGCTCTAGCC
TGAACTGCAGCTACACAGTGTCTGGACTGAGAGGCCTGTTCTGGTACAGACAGGATCCTGGA
AAAGGCCCAGAGTTCCTGTTTACCCTGTATTCTGCCGGCGAGGAGAAGGAGAAAGAGAGACT
GAAAGCTACCCTGACCAAGAAGGAGAGCTTCCTGCACATTACCGCCCCCAAACCTGAGGATT
CTGCCACATATCTGTGTGCTGTTCAGCCTAGAGGAGATGGCTCTAGCAATACCGGCAAGCTG
ATCTTTGGCCAGGGAACAACACTGCAGGTGAAGCCTGAT
(SEQ ID NO: 228)
ATCCAGAATCCCGATCCTGCTGTGTACCAGCTGCGGGACAGCAAGAGCAGCGACAAGAGCGT
GTGCCTGTTCACCGACTTCGACAGCCAGACCAACGTGTCCCAGAGCAAGGACAGCGACGTGT
ACATCACCGATAAGTGCGTGCTGGACATGCGGAGCATGGACTTCAAGAGCAACAGCGCCGTG
GCCTGGTCCAACAAGAGCGACTTCGCCTGCGCCAACGCCTTCAACAACAGCATTATCCCCGA
GGACACATTCTTCCCAAGCCCCGAGAGCAGCTGCGACGTGAAGCTGGTGGAAAAGAGCTTCG
AGACAGACACCAACCTGAACTTCCAGAACCTCAGCGTGATCGGCTTCCGGATCCTGCTGCTG
AAGGTGGCCGGCTTCAACCTGCTGATGACCCTGCGGCTGTGGTCCAGCTGA
(SEQ ID NO: 229)
CTCAATAAAAGAGCCCACAACCCCTCACTCGGCGCGCCACCATGGGCACATCTCTTCTCTGT
TGGGTGGTTCTGGGCTTTCTGGGCACAGATCATACAGGAGCTGGAGTTAGCCAGTCTCCTAG
GTATAAGGTGACCAAGAGGGGACAGGATGTGGCTCTGAGATGTGACCCTATTAGCGGACATG
TGAGCCTGTACTGGTACAGACAAGCTCTGGGACAAGGACCCGAGTTTCTGACCTACTTCAAC
TATGAGGCCCAGCAGGACAAATCTGGACTGCCCAACGACAGATTCAGCGCCGAAAGACCAGA
AGGCTCTATTAGCACACTGACCATCCAGAGAACAGAGCAGAGGGATTCTGCCATGTACAGAT
GCGCCAGCAGCTTAACAGGCTCTTACGAGCAGTACTTTGGACCTGGCACAAGACTGACAGTG
ACAGAG
(SEQ ID NO: 230)
CTCAATAAAAGAGCCCACAACCCCTCACTCGGCGCGCCACCATGCTGCTTCTTCTCCTCCTT
CTCGGACCTGCTGGATCTGGATTAGGAGCTGTTGTGTCTCAGCACCCTTCTTGGGTGATCTG
TAAAAGCGGCACAAGCGTGAAGATCGAGTGCAGAAGCCTGGACTTTCAGGCCACAACCATGT
TCTGGTATAGGCAGTTCCCCAAGCAGTCTCTGATGCTGATGGCCACCTCTAATGAGGGCTCT
AAGGCCACATATGAACAGGGAGTGGAGAAGGACAAGTTCCTGATCAACCACGCCTCTCTGAC
CCTGTCTACCCTGACAGTTACATCTGCCCACCCTGAGGATAGCAGCTTTTACATCTGTAGCG
CCACACCTGAAGCCTCTAGCCCATATGAGCAGTACTTTGGCCCTGGCACCAGATTAACAGTG
ACAGAG
(SEQ ID NO: 231)
CTCAATAAAAGAGCCCACAACCCCTCACTCGGCGCGCCACCATGGGACCTGGACTGCTTCAT
TGGATGGCTCTGTGTTTGCTGGGAACAGGACATGGAGATGCTATGGTGATCCAGAACCCCAG
GTATCAGGTGACCCAGTTTGGCAAACCAGTGACACTGAGCTGTTCTCAGACCCTGAACCACA
ACGTGATGTACTGGTACCAGCAGAAGTCTTCTCAGGCCCCTAAGCTGCTGTTCCACTACTAC
GACAAGGACTTCAACAACGAGGCCGATACCCCTGACAATTTCCAGAGCAGGAGGCCCAATAC
CAGCTTCTGTTTCCTGGACATTAGAAGCCCTGGACTGGGAGATGCTGCCATGTACCTGTGTG
CCACCAGCAATTTACAGGGAAGACAACCTCAGCACTTTGGCGATGGCACAAGGCTGTCTATC
CTGGAG
(SEQ ID NO: 232)
CTCAATAAAAGAGCCCACAACCCCTCACTCGGCGCGCCACCATGCTGAGCCCTGATCTCCCT
GATTCTGCCTGGAATACCAGACTGCTGTGTCATGTGATGCTGTGTCTGCTTGGAGCCGTTTC
TGTGGCTGCTGGCGTGATTCAATCTCCTAGACACCTGATCAAGGAGAAGAGAGAAACAGCCA
CCCTGAAGTGCTACCCCATCCCCAGACACGATACAGTGTACTGGTATCAGCAAGGACCTGGA
CAAGATCCCCAGTTCCTGATCAGCTTCTACGAGAAGATGCAGAGCGACAAAGGCAGCATCCC
AGACAGATTTAGCGCCCAGCAGTTTAGCGACTATCACTCTGAGCTGAACATGAGCAGCCTGG
AACTGGGCGATTCTGCTCTGTACTTCTGTGCCTCTTCTCTGAGACTGGGAAGAGAAACCCAG
TACTTTGGACCCGGCACAAGACTGCTGGTTCTTGAG
(SEQ ID NO: 233)
CTCAATAAAAGAGCCCACAACCCCTCACTCGGCGCGCCACCATGGGCACAAGACTTCTCTGC
TGGGTGGTGCTTGGATTTCTGGGCACAGATCATACAGGAGCTGGAGTTAGCCAGTCTCCTAG
GTACAAAGTGGCCAAGAGAGGACAGGATGTGGCTCTGAGATGTGACCCTATTAGCGGACATG
TGAGCCTGTTTTGGTACCAGCAAGCTCTGGGACAAGGACCCGAGTTTCTGACCTACTTCCAG
AATGAAGCCCAGCTGGATAAATCTGGACTGCCTAGCGACCGGTTCTTCGCCGAAAGACCTGA
AGGATCTGTTAGCACCCTGAAGATTCAGAGAACACAGCAGGAGGACTCTGCCGTGTACCTGT
GTGCCTCTTCTTTAGGACAGGCCTATGAGCAGTATTTTGGACCTGGCACCAGACTGACCGTG
ACAGAG
(SEQ ID NO: 234)
CTCAATAAAAGAGCCCACAACCCCTCACTCGGCGCGCCACCATGGGCACAAGACTTCTCTGC
TGGGTGGCCTTTTGTCTGCTGGTGGAAGAGCTGATTGAAGCTGGAGTTGTGCAGTCTCCTAG
GTACAAGATCATCGAGAAGAAGCAGCCCGTGGCCTTCTGGTGTAATCCCATTTCTGGCCACA
ACACCCTGTACTGGTATCTGCAGAATCTGGGACAGGGCCCTGAACTGCTGATCAGATACGAG
AACGAAGAAGCCGTGGACGATTCTCAACTGCCTAAGGACCGCTTTTCTGCCGAGAGGCTGAA
AGGAGTGGATTCTACCCTGAAGATCCAACCTGCTGAACTGGGCGATTCTGCTGTGTACCTGT
GCGCTTCTAGCCTGACAAGAGGAGCTGAAGCCTTTTTTGGACAGGGCACAAGACTGACAGTG
GTGGAG
(SEQ ID NO: 235)
CTCAATAAAAGAGCCCACAACCCCTCACTCGGCGCGCCACCATGGGACCTCAGCTTCTTGGA
TACGTTGTGCTGTGTCTGCTTGGAGCTGGACCTCTTGAAGCTCAGGTTACCCAGAACCCCAG
ATACCTGATTACCGTGACAGGCAAAAAGCTGACCGTGACATGTAGCCAGAACATGAACCACG
AGTACATGAGCTGGTACCGGCAGGATCCTGGATTAGGCCTGAGACAGATCTACTACAGCATG
AACGTGGAGGTGACCGATAAAGGCGACGTGCCTGAGGGATACAAGGTGAGCAGAAAGGAGAA
GAGGAATTTCCCCCTGATCCTGGAAAGCCCAAGCCCCAATCAGACAAGCCTGTACTTTTGTG
CCAGCAGCTTTTCTGGCGGCACATATGAGCAGTACTTCGGCCCTGGCACAAGACTGACAGTT
ACAGAG
(SEQ ID NO: 236)
CTCAATAAAAGAGCCCACAACCCCTCACTCGGCGCGCCACCATGCTGAGCCCTGATCTCCCT
GATTCTGCCTGGAATACCAGACTGCTGTGTCATGTGATGCTGTGTCTGCTTGGAGCCGTTTC
TGTGGCTGCTGGCGTGATTCAATCTCCTAGACACCTGATCAAGGAGAAGAGAGAAACAGCCA
CCCTGAAGTGCTACCCCATCCCCAGACACGATACAGTGTACTGGTATCAGCAAGGACCTGGA
CAAGATCCCCAGTTCCTGATCAGCTTCTACGAGAAGATGCAGAGCGACAAAGGCAGCATCCC
AGACAGATTTAGCGCCCAGCAGTTTAGCGACTATCACTCTGAGCTGAACATGAGCAGCCTGG
AACTGGGCGATTCTGCTCTGTACTTCTGTGCCAGCAGCTATAGAGGAGGCAGCACATATGAG
CAGTACTTTGGCCCTGGCACAAGACTGACAGTGACAGAG
(SEQ ID NO: 237)
CTCAATAAAAGAGCCCACAACCCCTCACTCGGCGCGCCACCATGAGCACCAGACTCCTTTGC
TGGATGGCTTTGTGTCTGCTTGGAGCTGAGCTGTCTGAAGCTGAAGTTGCCCAGTCTCCCAG
ATACAAGATCACCGAGAAATCTCAGGCTGTGGCCTTCTGGTGTGACCCTATTTCTGGACACG
CCACCCTGTACTGGTATAGGCAAATTCTGGGACAAGGCCCTGAACTGCTGGTGCAATTTCAG
GATGAGAGCGTGGTGGACGATTCTCAACTGCCTAAGGACAGGTTTTCTGCCGAGCGGCTGAA
AGGAGTTGATAGCACCCTGAAGATCCAACCTGCTGAACTGGGCGATTCTGCTATGTACCTGT
GCGCCTCTTCTCAGAGAGATAGCCCTAACGAGAAGCTGTTCTTTGGCTCTGGAACCCAGCTG
TCTGTGCTGGAG
(SEQ ID NO: 238)
CTCAATAAAAGAGCCCACAACCCCTCACTCGGCGCGCCACCATGGGCTGTAGACTGTTGTGT
TGTGCTGTGCTGTGTCTGTTGGGAGCTGTGCCTATGGAAACAGGCGTTACCCAGACACCTAG
ACATCTGGTTATGGGCATGACCAACAAGAAGAGCCTGAAGTGCGAGCAGCATCTGGGCCATA
ACGCCATGTACTGGTATAAGCAGAGCGCCAAGAAACCACTGGAACTGATGTTCGTGTACAGC
CTGGAGGAGAGGGTGGAGAATAATAGCGTGCCCAGCAGATTTAGCCCTGAGTGCCCAAATTC
TTCTCACCTGTTCCTGCACCTGCACACATTACAGCCCGAGGATTCTGCCCTGTACCTGTGTG
CTTCTTCTCAAGACCCTTACAAGCTGAGCGGCAATACCATCTACTTCGGCGAAGGCTCTTGG
CTGACAGTGGTTGAA
(SEQ ID NO: 239)
GATCTGAACAAGGTGTTCCCCCCAGAGGTGGCCGTGTTCGAGCCTTCTGAGGCCGAGATCTC
CCACACCCAGAAAGCCACCCTCGTGTGCCTGGCCACCGGCTTTTTCCCCGACCACGTGGAAC
TGTCTTGGTGGGTCAACGGCAAAGAGGTGCACTCCGGCGTGTGCACCGATCCCCAGCCTCTG
AAAGAACAGCCCGCCCTGAACGACAGCCGGTACTGCCTGAGCAGCAGACTGAGAGTGTCCGC
CACCTTCTGGCAGAACCCCCGGAACCACTTCAGATGCCAGGTGCAGTTCTACGGCCTGAGCG
AGAACGACGAGTGGACCCAGGACAGAGCCAAGCCCGTGACACAGATCGTGTCTGCCGAAGCC
TGGGGCAGAGCCGATTGCGGCTTTACCTCCGTGTCCTATCAGCAGGGCGTGCTGAGCGCCAC
AATCCTGTACGAGATCCTGCTGGGCAAGGCCACCCTGTACGCCGTGCTGGTGTCTGCCCTGG
TGCTGATGGCCATGGTCAAGCGGAAGGACTTC
(SEQ ID NO: 240)
GACCTGAAGAACGTGTTCCCCCCAGAGGTGGCCGTGTTCGAGCCTAGCGAGGCCGAGATCAG
CCACACCCAGAAAGCCACCCTCGTGTGCCTGGCCACCGGCTTTTACCCCGACCACGTGGAAC
TGTCTTGGTGGGTCAACGGCAAAGAGGTGCACAGCGGCGTCTGCACCGACCCCCAGCCCCTG
AAAGAGCAGCCCGCCCTGAACGACAGCCGGTACTGTCTGAGCAGCAGACTGAGAGTGTCCGC
CACCTTCTGGCAGAACCCCCGGAACCACTTCAGATGCCAGGTGCAGTTCTACGGCCTGAGCG
AGAACGACGAGTGGACCCAGGACCGGGCCAAGCCCGTGACCCAGATCGTGTCTGCTGAGGCC
TGGGGCAGAGCCGATTGCGGCTTCACCAGCGAGAGCTACCAGCAGGGCGTGCTGAGCGCCAC
CATCCTGTACGAGATCCTGCTGGGCAAGGCCACCCTGTACGCCGTGCTGGTGTCCGCCCTGG
TGCTGATGGCCATGGTCAAGCGGAAGGACAGCCGGGGC
(SEQ ID NO: 241)
ATGAAATCCTTGAGAGTTTTACTAGTGATCCTGTGGCTTCAGTTGAGCTGGGTTTGGAGCCA
ACAGAAGGAGGTGGAGCAGAATTCTGGACCCCTCAGTGTTCCAGAGGGAGCCATTGCCTCTC
TCAACTGCACTTACAGTGACCGAGGTTCCCAGTCCTTCTTCTGGTACAGACAATATTCTGGG
AAAAGCCCTGAGTTGATAATGTTCATATACTCCAATGGTGACAAAGAAGATGGAAGGTTTAC
AGCACAGCTCAATAAAGCCAGCCAGTATGTTTCTCTGCTCATCAGAGACTCCCAGCCCAGTG
ATTCAGCCACCTACCTCTGTGCCGTGAACATAGGAAACCATGACATGCGCTTTGGAGCAGGG
ACCAGACTGACAGTAAAACCAAATATCCAGAACCCTGACCCTGCCGTGTACCAGCTGAGAGA
CTCTAAATCCAGTGACAAGTCTGTCTGCCTATTCACCGATTTTGATTCTCAAACAAATGTGT
CACAAAGTAAGGATTCTGATGTGTATATCACAGACAAAACTGTGCTAGACATGAGGTCTATG
GACTTCAAGAGCAACAGTGCTGTGGCCTGGAGCAACAAATCTGACTTTGCATGTGCAAACGC
CTTCAACAACAGCATTATTCCAGAAGACACCTTCTTCCCCAGCCCAGAAAGTTCCTGTGATG
TCAAGCTGGTCGAGAAAAGCTTTGAAACAGATACGAACCTAAACTTTCAAAACCTGTCAGTG
ATTGGGTTCCGAATCCTCCTCCTGAAAGTGGCCGGGTTTAATCTGCTCATGACGCTGCGGCT
GTGGTCCAGCTGA
(SEQ ID NO: 242)
ATGGAGAAAATGTTGGAGTGTGCATTCATAGTCTTGTGGCTTCAGCTTGGCTGGTTGAGTGG
AGAAGACCAGGTGACGCAGAGTCCCGAGGCCCTGAGACTCCAGGAGGGAGAGAGTAGCAGTC
TCAACTGCAGTTACACAGTCAGCGGTTTAAGAGGGCTGTTCTGGTATAGGCAAGATCCTGGG
AAAGGCCCTGAATTCCTCTTCACCCTGTATTCAGCTGGGGAAGAAAAGGAGAAAGAAAGGCT
AAAAGCCACATTAACAAAGAAGGAAAGCTTTCTGCACATCACAGCCCCTAAACCTGAAGACT
CAGCCACTTATCTCTGTGCTGTGCAGACCATGGACGGTAACCAGTTCTATTTTGGGACAGGG
ACAAGTTTGACGGTCATTCCAAATATCCAGAACCCTGACCCTGCCGTGTACCAGCTGAGAGA
CTCTAAATCCAGTGACAAGTCTGTCTGCCTATTCACCGATTTTGATTCTCAAACAAATGTGT
CACAAAGTAAGGATTCTGATGTGTATATCACAGACAAAACTGTGCTAGACATGAGGTCTATG
GACTTCAAGAGCAACAGTGCTGTGGCCTGGAGCAACAAATCTGACTTTGCATGTGCAAACGC
CTTCAACAACAGCATTATTCCAGAAGACACCTTCTTCCCCAGCCCAGAAAGTTCCTGTGATG
TCAAGCTGGTCGAGAAAAGCTTTGAAACAGATACGAACCTAAACTTTCAAAACCTGTCAGTG
ATTGGGTTCCGAATCCTCCTCCTGAAAGTGGCCGGGTTTAATCTGCTCATGACGCTGCGGCT
GTGGTCCAGCTGA
(SEQ ID NO: 243)
ATGGCATGCCCTGGCTTCCTGTGGGCACTTGTGATCTCCACCTGTCTTGAATTTAGCATGGC
TCAGACAGTCACTCAGTCTCAACCAGAGATGTCTGTGCAGGAGGCAGAGACCGTGACCCTGA
GCTGCACATATGACACCAGTGAGAGTGATTATTATTTATTCTGGTACAAGCAGCCTCCCAGC
AGGCAGATGATTCTCGTTATTCGCCAAGAAGCTTATAAGCAACAGAATGCAACAGAGAATCG
TTTCTCTGTGAACTTCCAGAAAGCAGCCAAATCCTTCAGTCTCAAGATCTCAGACTCACAGC
TGGGGGATGCCGCGATGTATTTCTGTGCTTCCAGTCCAGGAACCTACAAATACATCTTTGGA
ACAGGCACCAGGCTGAAGGTTTTAGCAAATATCCAGAACCCTGACCCTGCCGTGTACCAGCT
GAGAGACTCTAAATCCAGTGACAAGTCTGTCTGCCTATTCACCGATTTTGATTCTCAAACAA
ATGTGTCACAAAGTAAGGATTCTGATGTGTATATCACAGACAAAACTGTGCTAGACATGAGG
TCTATGGACTTCAAGAGCAACAGTGCTGTGGCCTGGAGCAACAAATCTGACTTTGCATGTGC
AAACGCCTTCAACAACAGCATTATTCCAGAAGACACCTTCTTCCCCAGCCCAGAAAGTTCCT
GTGATGTCAAGCTGGTCGAGAAAAGCTTTGAAACAGATACGAACCTAAACTTTCAAAACCTG
TCAGTGATTGGGTTCCGAATCCTCCTCCTGAAAGTGGCCGGGTTTAATCTGCTCATGACGCT
GCGGCTGTGGTCCAGCTGA
(SEQ ID NO: 244)
ATGACACGAGTTAGCTTGCTGTGGGCAGTCGTGGTCTCCACCTGTCTTGAATCCGGCATGGC
CCAGACAGTCACTCAGTCTCAACCAGAGATGTCTGTGCAGGAGGCAGAGACTGTGACCCTGA
GTTGCACATATGACACCAGTGAGAGTAATTATTATTTGTTCTGGTACAAACAGCCTCCCAGC
AGGCAGATGATTCTCGTTATTCGCCAAGAAGCTTATAAGCAACAGAATGCAACGGAGAATCG
TTTCTCTGTGAACTTCCAGAAAGCAGCCAAATCCTTCAGTCTCAAGATCTCAGACTCACAGC
TGGGGGACACTGCGATGTATTTCTGTGCTTTCAACCCTTGGGAGAACTATGGTCAGAATTTT
GTCTTTGGTCCCGGAACCAGATTGTCCGTGCTGCCCTATATCCAGAACCCTGACCCTGCCGT
GTACCAGCTGAGAGACTCTAAATCCAGTGACAAGTCTGTCTGCCTATTCACCGATTTTGATT
CTCAAACAAATGTGTCACAAAGTAAGGATTCTGATGTGTATATCACAGACAAAACTGTGCTA
GACATGAGGTCTATGGACTTCAAGAGCAACAGTGCTGTGGCCTGGAGCAACAAATCTGACTT
TGCATGTGCAAACGCCTTCAACAACAGCATTATTCCAGAAGACACCTTCTTCCCCAGCCCAG
AAAGTTCCTGTGATGTCAAGCTGGTCGAGAAAAGCTTTGAAACAGATACGAACCTAAACTTT
CAAAACCTGTCAGTGATTGGGTTCCGAATCCTCCTCCTGAAAGTGGCCGGGTTTAATCTGCT
CATGACGCTGCGGCTGTGGTCCAGCTGA
(SEQ ID NO: 245)
ATGAAGAGCCTGAGAGTCCTGCTGGTGATTTTGTGGCTGCAGCTGTCTTGGGTTTGGTCTCA
GCAGAAAGAAGTGGAGCAGAATAGCGGCCCTCTGTCTGTTCCTGAAGGCGCTATTGCTAGCC
TGAATTGCACATACAGCGATAGAGGATCTCAGAGCTTCTTCTGGTACCGGCAGTACAGCGGC
AAGAGCCCAGAACTGATCATGTTCATCTACAGCAATGGCGACAAGGAGGATGGCAGGTTTAC
AGCCCAGCTGAACAAGGCCAGCCAGTATGTTTCTCTGCTGATCAGAGATAGCCAGCCTAGCG
ATTCTGCCACCTACCTGTGTGCCGTGAACATCGGAAATCACGACATGAGATTTGGAGCCGGC
ACAAGACTGACCGTGAAGCCCAATATCCAGAACCCTGATCCTGCTGTGTACCAGCTGCGGGA
CAGCAAGAGCAGCGACAAGAGCGTGTGCCTGTTCACCGACTTCGACAGCCAGACCAACGTGT
CCCAGAGCAAGGACAGCGACGTGTACATCACCGATAAGTGCGTGCTGGACATGCGGAGCATG
GACTTCAAGAGCAACAGCGCCGTGGCCTGGTCCAACAAGAGCGACTTCGCCTGCGCCAACGC
CTTCAACAACAGCATTATCCCCGAGGACACATTCTTCCCAAGCCCCGAGAGCAGCTGCGACG
TGAAGCTGGTGGAAAAGAGCTTCGAGACAGACACCAACCTGAACTTCCAGAACCTCAGCGTG
ATCGGCTTCCGGATCCTGCTGCTGAAGGTGGCCGGCTTCAACCTGCTGATGACCCTGCGGCT
GTGGTCCAGCTGA
(SEQ ID NO: 246)
ATGGAGAAGATGCTGGAGTGTGCGTTCATCGTTCTGTGGCTGCAACTTGGATGGCTGTCTGG
AGAGGATCAGGTTACACAGTCTCCTGAAGCCCTGAGACTGCAAGAAGGAGAAAGCTCTAGCC
TGAACTGCAGCTACACAGTGTCTGGACTGAGAGGCCTGTTCTGGTACAGACAGGATCCTGGA
AAAGGCCCAGAGTTCCTGTTTACCCTGTATTCTGCCGGCGAGGAGAAGGAGAAAGAGAGACT
GAAAGCTACCCTGACCAAGAAGGAGAGCTTCCTGCACATTACCGCCCCCAAACCTGAGGATT
CTGCCACATATCTGTGTGCTGTGCAGACCATGGATGGCAACCAGTTCTACTTCGGCACAGGC
ACATCTCTGACCGTTATCCCCAATATCCAGAACCCTGATCCTGCCGTGTACCAGCTGCGGGA
CAGCAAGAGCAGCGACAAGAGCGTGTGCCTGTTCACCGACTTCGACAGCCAGACCAACGTGT
CCCAGAGCAAGGACAGCGACGTGTACATCACCGATAAGTGCGTGCTGGACATGCGGAGCATG
GACTTCAAGAGCAACAGCGCCGTGGCCTGGTCCAACAAGAGCGACTTCGCCTGCGCCAACGC
CTTCAACAACAGCATTATCCCCGAGGACACATTCTTCCCAAGCCCCGAGAGCAGCTGCGACG
TGAAGCTGGTGGAAAAGAGCTTCGAGACAGACACCAACCTGAACTTCCAGAACCTCAGCGTG
ATCGGCTTCCGGATCCTGCTGCTGAAGGTGGCCGGCTTCAACCTGCTGATGACCCTGCGGCT
GTGGTCCAGCTGA
(SEQ ID NO: 247)
ATGGCTTGTCCTGGATTCTTATGGGCTCTGGTGATCAGCACCTGTCTGGAGTTCTCTATGGC
CCAGACAGTGACACAGTCTCAGCCTGAAATGTCTGTGCAGGAAGCCGAAACCGTGACACTGT
CTTGCACCTACGATACAAGCGAGAGCGACTACTACCTGTTCTGGTACAAGCAGCCTCCCTCT
AGGCAGATGATCCTGGTGATTAGACAGGAGGCCTACAAACAGCAGAATGCCACCGAGAACCG
GTTTAGCGTGAACTTCCAGAAAGCCGCCAAGAGCTTCAGCCTGAAAATCTCTGACAGCCAGC
TGGGAGATGCTGCCATGTACTTTTGTGCCAGCTCTCCAGGCACCTACAAGTACATTTTTGGC
ACCGGCACCAGACTGAAGGTGCTGGCCAATATCCAGAATCCCGATCCTGCCGTGTACCAGCT
GCGGGACAGCAAGAGCAGCGACAAGAGCGTGTGCCTGTTCACCGACTTCGACAGCCAGACCA
ACGTGTCCCAGAGCAAGGACAGCGACGTGTACATCACCGATAAGTGCGTGCTGGACATGCGG
AGCATGGACTTCAAGAGCAACAGCGCCGTGGCCTGGTCCAACAAGAGCGACTTCGCCTGCGC
CAACGCCTTCAACAACAGCATTATCCCCGAGGACACATTCTTCCCAAGCCCCGAGAGCAGCT
GCGACGTGAAGCTGGTGGAAAAGAGCTTCGAGACAGACACCAACCTGAACTTCCAGAACCTC
AGCGTGATCGGCTTCCGGATCCTGCTGCTGAAGGTGGCCGGCTTCAACCTGCTGATGACCCT
GCGGCTGTGGTCCAGCTGA
(SEQ ID NO: 248)
ATGACCAGAGTTAGCCTGTTATGGGCTGTGGTGGTGAGCACATGTCTGGAATCTGGAATGGC
CCAGACAGTGACACAGTCTCAGCCTGAAATGTCTGTGCAGGAAGCCGAAACCGTTACACTGA
GCTGCACCTACGATACAAGCGAGAGCAACTACTACCTGTTCTGGTACAAGCAGCCCCCTTCT
AGGCAGATGATCCTGGTGATCAGACAGGAGGCCTATAAACAGCAGAATGCCACCGAGAACCG
GTTTAGCGTGAACTTCCAGAAAGCCGCCAAGAGCTTCAGCCTGAAAATCTCTGACAGCCAGC
TGGGCGATACAGCCATGTACTTTTGTGCCTTCAACCCCTGGGAGAACTATGGCCAGAATTTC
GTGTTCGGCCCTGGCACCAGACTGTCTGTTCTGCCTTATATCCAGAACCCCGATCCTGCTGT
GTACCAGCTGCGGGACAGCAAGAGCAGCGACAAGAGCGTGTGCCTGTTCACCGACTTCGACA
GCCAGACCAACGTGTCCCAGAGCAAGGACAGCGACGTGTACATCACCGATAAGTGCGTGCTG
GACATGCGGAGCATGGACTTCAAGAGCAACAGCGCCGTGGCCTGGTCCAACAAGAGCGACTT
CGCCTGCGCCAACGCCTTCAACAACAGCATTATCCCCGAGGACACATTCTTCCCAAGCCCCG
AGAGCAGCTGCGACGTGAAGCTGGTGGAAAAGAGCTTCGAGACAGACACCAACCTGAACTTC
CAGAACCTCAGCGTGATCGGCTTCCGGATCCTGCTGCTGAAGGTGGCCGGCTTCAACCTGCT
GATGACCCTGCGGCTGTGGTCCAGCTGA
(SEQ ID NO: 249)
ATGGGCTGCAGGCTGCTCTGCTGTGCGGTTCTCTGTCTCCTGGGAGCAGTTCCCATAGACAC
TGAAGTTACCCAGACACCAAAACACCTGGTCATGGGAATGACAAATAAGAAGTCTTTGAAAT
GTGAACAACATATGGGGCACAGGGCTATGTATTGGTACAAGCAGAAAGCTAAGAAGCCACCG
GAGCTCATGTTTGTCTACAGCTATGAGAAACTCTCTATAAATGAAAGTGTGCCAAGTCGCTT
CTCACCTGAATGCCCCAACAGCTCTCTCTTAAACCTTCACCTACACGCCCTGCAGCCAGAAG
ACTCAGCCCTGTATCTCTGCGCCAGCAGCCAAGGGACTAGCGGGGCAGATACGCAGTATTTT
GGCCCAGGCACCCGGCTGACAGTGCTCGAGGACCTGAAAAACGTGTTCCCACCCGAGGTCGC
TGTGTTTGAGCCATCAGAAGCAGAGATCTCCCACACCCAAAAGGCCACACTGGTGTGCCTGG
CCACAGGCTTCTACCCCGACCACGTGGAGCTGAGCTGGTGGGTGAATGGGAAGGAGGTGCAC
AGTGGGGTCAGCACAGACCCGCAGCCCCTCAAGGAGCAGCCCGCCCTCAATGACTCCAGATA
CTGCCTGAGCAGCCGCCTGAGGGTCTCGGCCACCTTCTGGCAGAACCCCCGCAACCACTTCC
GCTGTCAAGTCCAGTTCTACGGGCTCTCGGAGAATGACGAGTGGACCCAGGATAGGGCCAAA
CCTGTCACCCAGATCGTCAGCGCCGAGGCCTGGGGTAGAGCAGACTGTGGCTTCACCTCCGA
GTCTTACCAGCAAGGGGTCCTGTCTGCCACCATCCTCTATGAGATCTTGCTAGGGAAGGCCA
CCTTGTATGCCGTGCTGGTCAGTGCCCTCGTGCTGATGGCCATGGTCAAGAGAAAGGATTCC
AGAGGCTAG
(SEQ ID NO: 250)
ATGAGCATCGGCCTCCTGTGCTGTGCAGCCTTGTCTCTCCTGTGGGCAGGTCCAGTGAATGC
TGGTGTCACTCAGACCCCAAAATTCCAGGTCCTGAAGACAGGACAGAGCATGACACTGCAGT
GTGCCCAGGATATGAACCATGAATACATGTCCTGGTATCGACAAGACCCAGGCATGGGGCTG
AGGCTGATTCATTACTCAGTTGGTGCTGGTATCACTGACCAAGGAGAAGTCCCCAATGGCTA
CAATGTCTCCAGATCAACCACAGAGGATTTCCCGCTCAGGCTGCTGTCGGCTGCTCCCTCCC
AGACATCTGTGTACTTCTGTGCCAGCAGTTACTCTCTTTGGGACCTTCAAGAGACCCAGTAC
TTCGGGCCAGGCACGCGGCTCCTGGTGCTCGAGGACCTGAAAAACGTGTTCCCACCCGAGGT
CGCTGTGTTTGAGCCATCAGAAGCAGAGATCTCCCACACCCAAAAGGCCACACTGGTGTGCC
TGGCCACAGGCTTCTACCCCGACCACGTGGAGCTGAGCTGGTGGGTGAATGGGAAGGAGGTG
CACAGTGGGGTCAGCACAGACCCGCAGCCCCTCAAGGAGCAGCCCGCCCTCAATGACTCCAG
ATACTGCCTGAGCAGCCGCCTGAGGGTCTCGGCCACCTTCTGGCAGAACCCCCGCAACCACT
TCCGCTGTCAAGTCCAGTTCTACGGGCTCTCGGAGAATGACGAGTGGACCCAGGATAGGGCC
AAACCTGTCACCCAGATCGTCAGCGCCGAGGCCTGGGGTAGAGCAGACTGTGGCTTCACCTC
CGAGTCTTACCAGCAAGGGGTCCTGTCTGCCACCATCCTCTATGAGATCTTGCTAGGGAAGG
CCACCTTGTATGCCGTGCTGGTCAGTGCCCTCGTGCTGATGGCCATGGTCAAGAGAAAGGAT
TCCAGAGGCTAG
(SEQ ID NO: 251)
ATGGGCACCAGCCTCCTCTGCTGGATGGCCCTGTGTCTCCTGGGGGCAGATCACGCAGATAC
TGGAGTCTCCCAGGACCCCAGACACAAGATCACAAAGAGGGGACAGAATGTAACTTTCAGGT
GTGATCCAATTTCTGAACACAACCGCCTTTATTGGTACCGACAGACCCTGGGGCAGGGCCCA
GAGTTTCTGACTTACTTCCAGAATGAAGCTCAACTAGAAAAATCAAGGCTGCTCAGTGATCG
GTTCTCTGCAGAGAGGCCTAAGGGATCTTTCTCCACCTTGGAGATCCAGCGCACAGAGCAGG
GGGACTCGGCCATGTATCTCTGTGCCAGCAGCTTTTCAGACGGGGGGGCTACAGATACGCAG
TATTTTGGCCCAGGCACCCGGCTGACAGTGCTCGAGGACCTGAAAAACGTGTTCCCACCCGA
GGTCGCTGTGTTTGAGCCATCAGAAGCAGAGATCTCCCACACCCAAAAGGCCACACTGGTGT
GCCTGGCCACAGGCTTCTACCCCGACCACGTGGAGCTGAGCTGGTGGGTGAATGGGAAGGAG
GTGCACAGTGGGGTCAGCACAGACCCGCAGCCCCTCAAGGAGCAGCCCGCCCTCAATGACTC
CAGATACTGCCTGAGCAGCCGCCTGAGGGTCTCGGCCACCTTCTGGCAGAACCCCCGCAACC
ACTTCCGCTGTCAAGTCCAGTTCTACGGGCTCTCGGAGAATGACGAGTGGACCCAGGATAGG
GCCAAACCTGTCACCCAGATCGTCAGCGCCGAGGCCTGGGGTAGAGCAGACTGTGGCTTCAC
CTCCGAGTCTTACCAGCAAGGGGTCCTGTCTGCCACCATCCTCTATGAGATCTTGCTAGGGA
AGGCCACCTTGTATGCCGTGCTGGTCAGTGCCCTCGTGCTGATGGCCATGGTCAAGAGAAAG
GATTCCAGAGGCTAG
(SEQ ID NO: 252)
ATGCTGCTGCTTCTGCTGCTTCTGGGGCCAGCAGGCTCCGGGCTTGGTGCTGTCGTCTCTCA
ACATCCGAGCTGGGTTATCTGTAAGAGTGGAACCTCTGTGAAGATCGAGTGCCGTTCCCTGG
ACTTTCAGGCCACAACTATGTTTTGGTATCGTCAGTTCCCGAAACAGAGTCTCATGCTGATG
GCAACTTCCAATGAGGGCTCCAAGGCCACATACGAGCAAGGCGTCGAGAAGGACAAGTTTCT
CATCAACCATGCAAGCCTGACCTTGTCCACTCTGACAGTGACCAGTGCCCATCCTGAAGACA
GCAGCTTCTACATCTGCAGTGCTAGACCCCATTCTCTCACAGATACGCAGTATTTTGGCCCA
GGCACCCGGCTGACAGTGCTCGAGGACCTGAAAAACGTGTTCCCACCCGAGGTCGCTGTGTT
TGAGCCATCAGAAGCAGAGATCTCCCACACCCAAAAGGCCACACTGGTGTGCCTGGCCACAG
GCTTCTACCCCGACCACGTGGAGCTGAGCTGGTGGGTGAATGGGAAGGAGGTGCACAGTGGG
GTCAGCACAGACCCGCAGCCCCTCAAGGAGCAGCCCGCCCTCAATGACTCCAGATACTGCCT
GAGCAGCCGCCTGAGGGTCTCGGCCACCTTCTGGCAGAACCCCCGCAACCACTTCCGCTGTC
AAGTCCAGTTCTACGGGCTCTCGGAGAATGACGAGTGGACCCAGGATAGGGCCAAACCTGTC
ACCCAGATCGTCAGCGCCGAGGCCTGGGGTAGAGCAGACTGTGGCTTCACCTCCGAGTCTTA
CCAGCAAGGGGTCCTGTCTGCCACCATCCTCTATGAGATCTTGCTAGGGAAGGCCACCTTGT
ATGCCGTGCTGGTCAGTGCCCTCGTGCTGATGGCCATGGTCAAGAGAAAGGATTCCAGAGGC
TAG
(SEQ ID NO: 253)
ATGGGCTGTAGACTGTTGTGTTGTGCTGTGCTGTGTCTGTTGGGAGCTGTGCCTATCGATAC
AGAGGTGACCCAGACCCCTAAACATCTGGTTATGGGCATGACCAACAAGAAGAGCCTGAAGT
GCGAGCAGCACATGGGCCATAGGGCCATGTATTGGTATAAGCAGAAGGCCAAGAAACCTCCT
GAGCTGATGTTCGTGTACAGCTACGAGAAGCTGAGCATCAACGAGAGCGTGCCCAGCAGATT
TTCTCCTGAGTGCCCTAATTCTAGCCTGCTGAATCTGCACCTGCATGCTCTGCAGCCTGAGG
ATTCTGCTCTGTACCTGTGTGCTTCTTCTCAGGGCACATCTGGAGCTGATACACAGTACTTC
GGACCTGGCACAAGACTGACAGTGCTGGAAGACCTGAAGAACGTGTTCCCCCCAGAGGTGGC
CGTGTTCGAGCCTAGCGAGGCCGAGATCAGCCACACCCAGAAAGCCACCCTCGTGTGCCTGG
CCACCGGCTTTTACCCCGACCACGTGGAACTGTCTTGGTGGGTCAACGGCAAAGAGGTGCAC
AGCGGCGTCTGCACCGACCCCCAGCCCCTGAAAGAGCAGCCCGCCCTGAACGACAGCCGGTA
CTGTCTGAGCAGCAGACTGAGAGTGTCCGCCACCTTCTGGCAGAACCCCCGGAACCACTTCA
GATGCCAGGTGCAGTTCTACGGCCTGAGCGAGAACGACGAGTGGACCCAGGACCGGGCCAAG
CCCGTGACCCAGATCGTGTCTGCTGAGGCCTGGGGCAGAGCCGATTGCGGCTTCACCAGCGA
GAGCTACCAGCAGGGCGTGCTGAGCGCCACCATCCTGTACGAGATCCTGCTGGGCAAGGCCA
CCCTGTACGCCGTGCTGGTGTCCGCCCTGGTGCTGATGGCCATGGTCAAGCGGAAGGACAGC
CGGGGC
(SEQ ID NO: 254)
ATGTCTATCGGTCTGCTGTGCTGTGCTGCTCTTTCTCTGCTTTGGGCTGGACCTGTGAATGC
TGGAGTTACACAAACCCCCAAGTTCCAAGTGCTGAAGACAGGACAGAGCATGACCCTGCAGT
GTGCTCAGGACATGAATCACGAGTACATGAGCTGGTACAGACAGGATCCTGGAATGGGCCTG
AGGCTGATCCACTACTCTGTTGGAGCCGGAATTACAGATCAGGGAGAAGTGCCAAATGGCTA
CAACGTGAGCAGGAGCACAACCGAGGACTTCCCCTTAAGACTGTTGTCTGCTGCTCCATCTC
AGACAAGCGTGTACTTTTGCGCCAGCTCCTACTCTCTGTGGGATCTGCAGGAAACCCAGTAC
TTTGGACCAGGCACAAGACTGTTAGTGCTGGAGGACCTGAAGAACGTGTTCCCCCCAGAGGT
GGCCGTGTTCGAGCCTAGCGAGGCCGAGATCAGCCACACCCAGAAAGCCACCCTCGTGTGCC
TGGCCACCGGCTTTTACCCCGACCACGTGGAACTGTCTTGGTGGGTCAACGGCAAAGAGGTG
CACAGCGGCGTCTGCACCGACCCCCAGCCCCTGAAAGAGCAGCCCGCCCTGAACGACAGCCG
GTACTGTCTGAGCAGCAGACTGAGAGTGTCCGCCACCTTCTGGCAGAACCCCCGGAACCACT
TCAGATGCCAGGTGCAGTTCTACGGCCTGAGCGAGAACGACGAGTGGACCCAGGACCGGGCC
AAGCCCGTGACCCAGATCGTGTCTGCTGAGGCCTGGGGCAGAGCCGATTGCGGCTTCACCAG
CGAGAGCTACCAGCAGGGCGTGCTGAGCGCCACCATCCTGTACGAGATCCTGCTGGGCAAGG
CCACCCTGTACGCCGTGCTGGTGTCCGCCCTGGTGCTGATGGCCATGGTCAAGCGGAAGGAC
AGCCGGGGC
(SEQ ID NO: 255)
ATGGGCACATCTCTTCTCTGCTGGATGGCTCTTTGTCTGCTTGGAGCCGATCATGCCGATAC
AGGAGTTAGCCAGGATCCTAGACACAAGATCACCAAGAGAGGCCAGAATGTGACCTTCCGGT
GCGATCCTATCTCTGAGCACAACAGGCTGTACTGGTACAGACAAACACTGGGACAAGGACCT
GAGTTCCTGACCTACTTCCAGAACGAAGCCCAGCTGGAGAAGTCTAGACTTCTGAGCGACAG
ATTTAGCGCCGAGAGACCTAAAGGCAGCTTTAGCACCCTGGAGATCCAGAGAACAGAACAGG
GCGATTCTGCCATGTACCTGTGTGCTAGCAGCTTTTCTGATGGAGGCGCCACCGATACACAG
TATTTCGGACCTGGCACAAGACTGACAGTGCTGGAGGACCTGAAGAACGTGTTCCCCCCAGA
GGTGGCCGTGTTCGAGCCTAGCGAGGCCGAGATCAGCCACACCCAGAAAGCCACCCTCGTGT
GCCTGGCCACCGGCTTTTACCCCGACCACGTGGAACTGTCTTGGTGGGTCAACGGCAAAGAG
GTGCACAGCGGCGTCTGCACCGACCCCCAGCCCCTGAAAGAGCAGCCCGCCCTGAACGACAG
CCGGTACTGTCTGAGCAGCAGACTGAGAGTGTCCGCCACCTTCTGGCAGAACCCCCGGAACC
ACTTCAGATGCCAGGTGCAGTTCTACGGCCTGAGCGAGAACGACGAGTGGACCCAGGACCGG
GCCAAGCCCGTGACCCAGATCGTGTCTGCTGAGGCCTGGGGCAGAGCCGATTGCGGCTTCAC
CAGCGAGAGCTACCAGCAGGGCGTGCTGAGCGCCACCATCCTGTACGAGATCCTGCTGGGCA
AGGCCACCCTGTACGCCGTGCTGGTGTCCGCCCTGGTGCTGATGGCCATGGTCAAGCGGAAG
GACAGCCGGGGC
(SEQ ID NO: 256)
ATGCTGCTTCTTCTCCTCCTTCTCGGACCTGCTGGATCTGGATTAGGAGCTGTTGTGTCTCA
GCACCCTTCTTGGGTGATCTGTAAAAGCGGCACAAGCGTGAAGATCGAGTGCAGAAGCCTGG
ACTTTCAGGCCACAACCATGTTCTGGTATAGGCAGTTCCCCAAGCAGTCTCTGATGCTGATG
GCCACCTCTAATGAGGGCTCTAAGGCCACATATGAACAGGGAGTGGAGAAGGACAAGTTCCT
GATCAACCACGCCTCTCTGACCCTGTCTACCCTGACAGTTACATCTGCCCACCCTGAGGATA
GCAGCTTTTACATCTGTAGCGCCAGACCTCACAGCCTGACCGATACACAGTACTTTGGCCCT
GGCACAAGACTGACAGTGTTAGAAGACCTGAAGAACGTGTTCCCCCCAGAGGTGGCCGTGTT
CGAGCCTAGCGAGGCCGAGATCAGCCACACCCAGAAAGCCACCCTCGTGTGCCTGGCCACCG
GCTTTTACCCCGACCACGTGGAACTGTCTTGGTGGGTCAACGGCAAAGAGGTGCACAGCGGC
GTCTGCACCGACCCCCAGCCCCTGAAAGAGCAGCCCGCCCTGAACGACAGCCGGTACTGTCT
GAGCAGCAGACTGAGAGTGTCCGCCACCTTCTGGCAGAACCCCCGGAACCACTTCAGATGCC
AGGTGCAGTTCTACGGCCTGAGCGAGAACGACGAGTGGACCCAGGACCGGGCCAAGCCCGTG
ACCCAGATCGTGTCTGCTGAGGCCTGGGGCAGAGCCGATTGCGGCTTCACCAGCGAGAGCTA
CCAGCAGGGCGTGCTGAGCGCCACCATCCTGTACGAGATCCTGCTGGGCAAGGCCACCCTGT
ACGCCGTGCTGGTGTCCGCCCTGGTGCTGATGGCCATGGTCAAGCGGAAGGACAGCCGGGGC
(SEQ ID NO: 257)
METLLGLLILWLQLQWVSSKQEVTQIPAALSVPEGFNLVLNCSFTDSAIYNLQWFRQDPGKG
LTSLLLIQSSQREQTSGRLNASLDKSSGRSTLYIAASQPGDSATYLCAVKETSGSRLTFGEG
TQLTVNP
(SEQ ID NO: 258)
MTRVSLLWAVVVSTCLESGMAQTVTQSQPEMSVQEAETVTLSCTYDTSENNYYLFWYKQPPS
RQMILVIRQEAYKQQNATENRFSVNFQKAAKSFSLKISDSQLGDTAMYFCAFIYPSYTSGTY
KYIFGTGTRLKVLAN
(SEQ ID NO: 259)
MAMLLGASVLILWLQPDWVNSQQKNDDQQVKQNSPSLSVQEGRISILNCDYTNSMFDYFLWY
KKYPAEGPTFLISISSIKDKNEDGRFTVFLNKSAKHLSLHIVPSQPGDSAVYFCAASGTGGS
YIPTFGRGTSLIVHPY
(SEQ ID NO: 260)
MAMLLGASVLILWLQPDWVNSQQKNDDQQVKQNSPSLSVQEGRISILNCDYTNSMFDYFLWY
KKYPAEGPTFLISISSIKDKNEDGRFTVFLNKSAKHLSLHIVPSQPGDSAVYFCAASGIGDY
KLSFGAGTTVTVRAN
(SEQ ID NO: 261)
MVKIRQFLLAILWLQLSCVSAAKNEVEQSPQNLTAQEGEFITINCSYSVGISALHWLQQHPG
GGIVSLFMLSSGKKKHGRLIATINIQEKHSSLHITASHPRDSAVYICAVRTSYDKVIFGPGT
SLSVIPN
(SEQ ID NO: 262)
MKSLRVLLVILWLQLSWVWSQQKEVEQNSGPLSVPEGAIASLNCTYSDRGSQSFFWYRQYSG
KSPELIMFIYSNGDKEDGRFTAQLNKASQYVSLLIRDSQPSDSATYLCAVNLLGATGYSTLT
FGKGTMLLVSP
(SEQ ID NO: 263)
MWGVFLLYVSMKMGGTTGQNIDQPTEMTATEGAIVQINCTYQTSGFNGLFWYQQHAGEAPTF
LSYNVLDGLEEKGRFSSFLSRSKGYSYLLLKELQMKDSASYLCAVRGINDYKLSFGAGTTVT
VRAN
(SEQ ID NO: 264)
MEKMLECAFIVLWLQLGWLSGEDQVTQSPEALRLQEGESSSLNCSYTVSGLRGLFWYRQDPG
KGPEFLFTLYSAGEEKEKERLKATLTKKESFLHITAPKPEDSATYLCAVITGFQKLVFGTGT
RLLVSPN
(SEQ ID NO: 265)
MRLVARVTVFLTFGTIIDAKTTQPTSMDCAEGRAANLPCNHSTISGNEYVYWYRQIHSQGPQ
YIIHGLKNNETNEMASLIITEDRKSSTLILPHATLRDTAVYYCIAGVGRGQNFVFGPGTRLS
VLPY
(SEQ ID NO: 266)
MEKNPLAAPLLILWFHLDCVSSILNVEQSPQSLHVQEGDSTNFTCSFPSSNFYALHWYRWET
AKSPEALFVMTLNGDEKKKGRISATLNTKEGYSYLYIKGSQPEDSATYLCAFHPNFGNEKLT
FGTGTRLTIIPN
(SEQ ID NO: 267)
MEKMLECAFIVLWLQLGWLSGEDQVTQSPEALRLQEGESSSLNCSYTVSGLRGLFWYRQDPG
KGPEFLFTLYSAGEEKEKERLKATLTKKESFLHITAPKPEDSATYLCAVQPRGDGSSNTGKL
IFGQGTTLQVKP
(SEQ ID NO: 268)
IQNPDPAVYQLRDSKSSDKSVCLFTDFDSQTNVSQSKDSDVYITDKCVLDMRSMDFKSNSAV
AWSNKSDFACANAFNNSIIPEDTFFPSPESSCDVKLVEKSFETDTNLNFQNLSVIGFRILLL
KVAGFNLLMTLRLWSS
(SEQ ID NO: 269)
MGTSLLCWVVLGFLGTDHTGAGVSQSPRYKVTKRGQDVALRCDPISGHVSLYWYRQALGQGP
EFLTYFNYEAQQDKSGLPNDRFSAERPEGSISTLTIQRTEQRDSAMYRCASSLTGSYEQYFG
PGTRLTVTE
(SEQ ID NO: 270)
MLLLLLLLGPAGSGLGAVVSQHPSWVICKSGTSVKIECRSLDFQATTMFWYRQFPKQSLMLM
ATSNEGSKATYEQGVEKDKFLINHASLTLSTLTVTSAHPEDSSFYICSATPEASSPYEQYFG
PGTRLTVTE
(SEQ ID NO: 271)
MGPGLLHWMALCLLGTGHGDAMVIQNPRYQVTQFGKPVTLSCSQTLNHNVMYWYQQKSSQAP
KLLFHYYDKDENNEADTPDNFQSRRPNTSFCFLDIRSPGLGDAAMYLCATSNLQGRQPQHFG
DGTRLSILE
(SEQ ID NO: 272)
MLSPDLPDSAWNTRLLCHVMLCLLGAVSVAAGVIQSPRHLIKEKRETATLKCYPIPRHDTVY
WYQQGPGQDPQFLISFYEKMQSDKGSIPDRFSAQQFSDYHSELNMSSLELGDSALYFCASSL
RLGRETQYFGPGTRLLVLE
(SEQ ID NO: 273)
MGTRLLCWVVLGFLGTDHTGAGVSQSPRYKVAKRGQDVALRCDPISGHVSLFWYQQALGQGP
EFLTYFQNEAQLDKSGLPSDRFFAERPEGSVSTLKIQRTQQEDSAVYLCASSLGQAYEQYFG
PGTRLTVTE
(SEQ ID NO: 274)
MGTRLLCWVAFCLLVEELIEAGVVQSPRYKIIEKKQPVAFWCNPISGHNTLYWYLQNLGQGP
ELLIRYENEEAVDDSQLPKDRFSAERLKGVDSTLKIQPAELGDSAVYLCASSLTRGAEAFFG
QGTRLTVVE
(SEQ ID NO: 275)
MSNQVLCCVVLCFLGANTVDGGITQSPKYLFRKEGQNVTLSCEQNLNHDAMYWYRQDPGQGL
RLIYYSQIVNDFQKGDIAEGYSVSREKKESFPLTVTSAQKNPTAFYLCASSRDRFQESPLHF
GNGTRLTVTE
(SEQ ID NO: 276)
MGPQLLGYVVLCLLGAGPLEAQVTQNPRYLITVTGKKLTVTCSQNMNHEYMSWYRQDPGLGL
RQIYYSMNVEVTDKGDVPEGYKVSRKEKRNFPLILESPSPNQTSLYFCASSFSGGTYEQYFG
PGTRLTVTE
(SEQ ID NO: 277)
MLSPDLPDSAWNTRLLCHVMLCLLGAVSVAAGVIQSPRHLIKEKRETATLKCYPIPRHDTVY
WYQQGPGQDPQFLISFYEKMQSDKGSIPDRFSAQQFSDYHSELNMSSLELGDSALYFCASSY
RGGSTYEQYFGPGTRLTVTE
(SEQ ID NO: 278)
MSTRLLCWMALCLLGAELSEAEVAQSPRYKITEKSQAVAFWCDPISGHATLYWYRQILGQGP
ELLVQFQDESVVDDSQLPKDRFSAERLKGVDSTLKIQPAELGDSAMYLCASSQRDSPNEKLF
FGSGTQLSVLE
(SEQ ID NO: 279)
MGCRLLCCAVLCLLGAVPMETGVTQTPRHLVMGMTNKKSLKCEQHLGHNAMYWYKQSAKKPL
ELMFVYSLEERVENNSVPSRFSPECPNSSHLFLHLHTLQPEDSALYLCASSQDPYKLSGNTI
YFGEGSWLTVVE
(SEQ ID NO: 280)
DLNKVFPPEVAVFEPSEAEISHTQKATLVCLATGFFPDHVFLSWWVNGKEVHSGVCTDPQPL
KEQPALNDSRYCLSSRLRVSATFWQNPRNHFRCQVQFYGLSENDEWTQDRAKPVTQIVSAEA
WGRADCGFTSVSYQQGVLSATILYEILLGKATLYAVLVSALVLMAMVKRKDF
(SEQ ID NO: 281)
DLKNVEPPEVAVFEPSEAEISHTQKATLVCLATGFYPDHVFLSWWVNGKEVHSGVCTDPQPL
KEQPALNDSRYCLSSRLRVSATFWQNPRNHFRCQVQFYGLSENDEWTQDRAKPVTQIVSAEA
WGRADCGFTSESYQQGVLSATILYEILLGKATLYAVLVSALVLMAMVKRKDSRG
(SEQ ID NO: 282)
MKSLRVLLVILWLQLSWVWSQQKEVEQNSGPLSVPEGAIASLNCTYSDRGSQSFFWYRQYSG
KSPELIMFIYSNGDKEDGRFTAQLNKASQYVSLLIRDSQPSDSATYLCAVNIGNHDMRFGAG
TRLTVKPN
(SEQ ID NO: 283)
MEKMLECAFIVLWLQLGWLSGEDQVTQSPEALRLQEGESSSLNCSYTVSGLRGLFWYRQDPG
KGPEFLFTLYSAGEEKEKERLKATLTKKESFLHITAPKPEDSATYLCAVQTMDGNQFYFGTG
TSLTVIPN
(SEQ ID NO: 284)
MACPGFLWALVISTCLEFSMAQTVTQSQPEMSVQEAETVTLSCTYDTSESDYYLFWYKQPPS
RQMILVIRQEAYKQQNATENRFSVNFQKAAKSFSLKISDSQLGDAAMYFCASSPGTYKYIFG
TGTRLKVLAN
(SEQ ID NO: 285)
MTRVSLLWAVVVSTCLESGMAQTVTQSQPEMSVQEAETVTLSCTYDTSESNYYLFWYKQPPS
RQMILVIRQEAYKQQNATENRFSVNFQKAAKSFSLKISDSQLGDTAMYFCAFNPWENYGQNF
VFGPGTRLSVLPY
(SEQ ID NO: 286)
IQNPDPAVYQLRDSKSSDKSVCLFTDFDSQTNVSQSKDSDVYITDKCVLDMRSMDFKSNSAV
AWSNKSDFACANAFNNSIIPEDTFFPSPESSCDVKLVEKSFETDINLNFQNLSVIGFRILLL
KVAGFNLLMTLRLWSS
(SEQ ID NO: 287)
MGCRLLCCAVLCLLGAVPIDTEVTQTPKHLVMGMTNKKSLKCEQHMGHRAMYWYKQKAKKPP
ELMFVYSYEKLSINESVPSRFSPECPNSSLLNLHLHALQPEDSALYLCASSQGTSGADTQYF
GPGTRLTVLE
(SEQ ID NO: 288)
MSIGLLCCAALSLLWAGPVNAGVTQTPKFQVLKTGQSMTLQCAQDMNHEYMSWYRQDPGMGL
RLIHYSVGAGITDQGEVPNGYNVSRSTTEDFPLRLLSAAPSQTSVYFCASSYSLWDLQETQY
FGPGTRLLVLE
(SEQ ID NO: 289)
MGTSLLCWMALCLLGADHADTGVSQDPRHKITKRGQNVTFRCDPISEHNRLYWYRQTLGQGP
EFLTYFQNEAQLEKSRLLSDRFSAERPKGSFSTLEIQRTEQGDSAMYLCASSFSDGGATDTQ
YFGPGTRLTVLE
(SEQ ID NO: 290)
MLLLLLLLGPAGSGLGAVVSQHPSWVICKSGTSVKIECRSLDFQATTMFWYRQFPKQSLMLM
ATSNEGSKATYEQGVEKDKFLINHASLTLSTLTVTSAHPEDSSFYICSARPHSLTDTQYFGP
GTRLTVLE
(SEQ ID NO: 291)
DLKNVFPPEVAVFEPSEAEISHTQKATLVCLATGFYPDHVFLSWWVNGKEVHSGVCTDPQPL
KEQPALNDSRYCLSSRLRVSATFWQNPRNHFRCQVQFYGLSENDEWTQDRAKPVTQIVSAEA
WGRADCGFTSESYQQGVLSATILYEILLGKATLYAVLVSALVLMAMVKRKDSRG

In some embodiments, a TCR construct comprises Human papilloma virus (HPV)-specific TCR chains. In some embodiments, a TCR construct comprising an HPV-specific TCR chains comprises TCR alpha and TCR beta chains that target the HPV 18 E6 protein, and/or HPV 18 E7 protein. In some embodiments, an HPV 18 E6 epitope is amino acids 121-135 and/or amino acids 77-91 of the HPV 18 E6 protein. In some embodiments, a TCR construct comprising an HPV-specific TCR chains comprises TCR alpha and TCR beta chains that target the HPV 18 E7 protein. In some embodiments, an HPV 18 E7 epitope is amino acids 11-19. In some embodiments, HPV-specific TCR sequences, TCR variable domain sequences, CDR sequences, and/or TCR constant domain sequences, are described in international patent application publications WO 2015/009604 A1, which is incorporated herein by reference for the purpose described herein.

In some embodiments, a TCR construct comprises Kirsten rat sarcoma virus (KRAS)-specific (KRAS-specific) TCR chains. In some embodiments, a TCR construct comprising KRAS-specific TCR chains comprises TCR alpha and TCR beta chains that target the epitope GADGVGKSA (SEQ ID NO: 293). In some embodiments, a TCR construct comprising KRAS-specific TCR chains comprises TCR alpha and TCR beta chains that target the epitope GADGVGKSAL (SEQ ID NO: 292). In some embodiments, KRAS-specific TCR sequences, TCR variable domain sequences, CDR sequences, and/or TCR constant domain sequences, are described in U.S. Pat. No. 10,611,816 B2, which is incorporated herein by reference for the purpose described herein. In some embodiments, a leader sequence and/or signal peptide may be removed from a TCR amino acid sequence, and percentage sequence identity may be calculated based on the TCR amino acid sequence without the leader sequence and/or signal peptide. In some embodiments, a KRAS-specific TCR targets an KRAS antigen associated with a cancerous state, such as but not limited to G12C, G12D, and/or G12R.

In some embodiments, a TCR construct comprising KRAS-specific TCR chains comprises a nucleotide coding sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 294-298. In some embodiments, a TCR construct comprising KRAS-specific TCR chains comprises an amino acid sequence that is at least, or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 299-317). In some embodiments, a TCR construct comprising KRAS-specific TCR chains comprises an amino acid sequence that is at least or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 299-303).

(SEQ ID NO: 2947)
atgggcaccaggctcttcttctatgtggccctttgtctgctgtgggcaggacacagggatgc
tggaatcacccagagcccaagatacaagatcacagagacaggaaggcaggtgaccttgatgt
gtcaccagacttggagccacagctatatgttctggtatcgacaagacctgggacatgggctg
aggctgatctattactcagcagctgctgatattacagataaaggagaagtccccgatggcta
tgttgtctccagatccaagacagagaatttccccctcactctggagtcagctacccgctccc
agacatctgtgtatttctgcgccagcagtgaccccggcactgaagctttctttggacaaggc
accagactcacagttgta
(SEQ ID NO: 295)
atgggcaccaggctcttcttctatgtggccctttgtctgctgtgggcaggacacagggatgc
tggaatcacccagagcccaagatacaagatcacagagacaggaaggcaggtgaccttgatgt
gtcaccagacttggagccacagctatatgttctggtatcgacaagacctgggacatgggctg
aggctgatctattactcagcagctgctgatattacagataaaggagaagtccccgatggcta
tgttgtctccagatccaagacagagaatttccccctcactctggagtcagctacccgctccc
agacatctgtgtatttctgcgccagcagtgaccccggcactgaagctttctttggacaaggc
accagactcacagttgtaGAGGACCTCAAGAATGTGTTTCCGCCCGAAGTCGCGGTTTTTGA
ACCATCAGAAGCCGAGATCTCTCATACACAAAAGGCGACGCTCGTATGCCTCGCGACGGGAT
TTTATCCGGACCACGTCGAGCTTTCCTGGTGGGTTAACGGAAAGGAGGTGCATTCCGGAGTT
TGCACGGACCCTCAGCCATTGAAGGAACAGCCCGCACTGAACGACAGTAGGTATTGCCTTTC
ATCTCGCCTGCGCGTGTCTGCGACATTCTGGCAAAACCCAAGAAATCACTTCAGATGTCAAG
TTCAGTTCTACGGTCTCAGCGAGAATGATGAGTGGACACAAGATAGGGCTAAACCCGTGACT
CAAATAGTCTCTGCCGAGGCCTGGGGGAGGGCGGATTGCGGCTTCACATCAGAATCATACCA
ACAAGGAGTATTGAGCGCGACAATTCTTTACGAAATTCTGCTTGGGAAAGCGACTCTGTACG
CGGTGCTCGTGTCCGCTTTGGTTCTTATGGCAATGGTTAAACGAAAGGATAGTAGGGGC
(SEQ ID NO: 296)
atgaaatccttgagagttttactagtgatcctgtggcttcagttgagctgggtttggagcca
acagaaggaggtggagcagaattctggacccctcagtgttccagagggagccattgcctctc
tcaactgcacttacagtgaccgaggttcccagtccttcttctggtacagacaatattctggg
aaaagccctgagttgataatgttcatatactccaatggtgacaaagaagatggaaggtttac
agcacagctcaataaagccagccagtatgtttctctgctcatcagagactcccagcccagtg
attcagccacctacctctgtgccgcggcgatggatagcagctataaattgatcttcgggagt
gggaccagactgctggtcaggcct
(SEQ ID NO: 297)
atgaaatccttgagagttttactagtgatcctgtggcttcagttgagctgggtttggagcca
acagaaggaggtggagcagaattctggacccctcagtgttccagagggagccattgcctctc
tcaactgcacttacagtgaccgaggttcccagtccttcttctggtacagacaatattctggg
aaaagccctgagttgataatgttcatatactccaatggtgacaaagaagatggaaggtttac
agcacagctcaataaagccagccagtatgtttctctgctcatcagagactcccagcccagtg
attcagccacctacctctgtgccgcggcgatggatagcagctataaattgatcttcgggagt
gggaccagactgctggtcaggcctATCCAGAACCCCGACCCCGCCGTGTACCAGCTGCGGGA
CAGCAAGAGCAGCGACAAGAGCGTGTGCCTGTTCACCGACTTCGACAGCCAGACCAACGTGT
CCCAGAGCAAGGACAGCGACGTGTACATCACCGATAAGTGCGTGCTGGACATGCGGAGCATG
GACTTCAAGAGCAACAGCGCCGTGGCCTGGTCCAACAAGAGCGACTTCGCCTGCGCCAACGC
CTTCAACAACAGCATCATCCCCGAGGACACATTCTTCCCAAGCCCCGAGAGCAGCTGCGACG
TGAAGCTGGTGGAGAAGTCCTTCGAGACAGACACCAACCTGAACTTCCAGAACCTGTCCGTG
ATCGGCTTCAGAATCCTGCTGCTGAAAGTGGCCGGCTTCAACCTGCTGATGACCCTGCGGCT
GTGGTCCAGC
(SEQ ID NO: 298)
atgggcaccaggctcttcttctatgtggccctttgtctgctgtgggcaggacacagggatgc
tggaatcacccagagcccaagatacaagatcacagagacaggaaggcaggtgaccttgatgt
gtcaccagacttggagccacagctatatgttctggtatcgacaagacctgggacatgggctg
aggctgatctattactcagcagctgctgatattacagataaaggagaagtccccgatggcta
tgttgtctccagatccaagacagagaatttccccctcactctggagtcagctacccgctccc
agacatctgtgtatttctgcgccagcagtgaccccggcactgaagctttctttggacaaggc
accagactcacagttgtaGAGGACCTCAAGAATGTGTTTCCGCCCGAAGTCGCGGTTTTTGA
ACCATCAGAAGCCGAGATCTCTCATACACAAAAGGCGACGCTCGTATGCCTcGCGACGGGAT
TTTATCCGGACCACGTCGAGCTTTCCTGGTGGGTTAACGGAAAGGAGGTGCATTCCGGAGTT
TGCACGGACCCTCAGCCATTGAAGGAACAGCCCGCACTGAACGACAGTAGGTATTGCCTTTC
ATCTCGCCTGCGCGTGTCTGCGACATTCTGGCAAAACCCAAGAAATCACTTCAGATGTCAAG
TTCAGTTCTACGGTCTCAGCGAGAATGATGAGTGGACACAAGATAGGGCTAAACCCGTGACT
CAAATAGTCTCTGCCGAGGCCTGGGGGAGGGCGGATTGCGGCTTCACATCAGAATCATACCA
ACAAGGAGTATTGAGCGCGACAATTCTTTACGAAATTCTGCTTGGGAAAGCGACTCTGTACG
CGGTGCTCGTGTCCGCTTTGGTTCTTATGGCAATGGTTAAACGAAAGGATAGTAGGGGCGCC
ACCAATTTCAGCCTGCTGAAACAGGCTGGCGACGTGGAAGAGAACCCCGGACCTatgaaatc
cttgagagttttactagtgatcctgtggcttcagttgagctgggtttggagccaacagaagg
aggtggagcagaattctggacccctcagtgttccagagggagccattgcctctctcaactgc
acttacagtgaccgaggttcccagtccttcttctggtacagacaatattctgggaaaagccc
tgagttgataatgttcatatactccaatggtgacaaagaagatggaaggtttacagcacagc
tcaataaagccagccagtatgtttctctgctcatcagagactcccagcccagtgattcagcc
acctacctctgtgccgcggcgatggatagcagctataaattgatcttcgggagtgggaccag
actgctggtcaggcctATCCAGAACCCCGACCCCGCCGTGTACCAGCTGCGGGACAGCAAGA
GCAGCGACAAGAGCGTGTGCCTGTTCACCGACTTCGACAGCCAGACCAACGTGTCCCAGAGC
AAGGACAGCGACGTGTACATCACCGATAAGTGCGTGCTGGACATGCGGAGCATGGACTTCAA
GAGCAACAGCGCCGTGGCCTGGTCCAACAAGAGCGACTTCGCCTGCGCCAACGCCTTCAACA
ACAGCATCATCCCCGAGGACACATTCTTCCCAAGCCCCGAGAGCAGCTGCGACGTGAAGCTG
GTGGAGAAGTCCTTCGAGACAGACACCAACCTGAACTTCCAGAACCTGTCCGTGATCGGCTT
CAGAATCCTGCTGCTGAAAGTGGCCGGCTTCAACCTGCTGATGACCCTGCGGCTGTGGTCCA
GC
(SEQ ID NO: 299)
MGTRLFFYVALCLLWAGHRDAGITQSPRYKITETGRQVTLMCHQTWSHSYMFWYRQDLGHGL
RLIYYSAAADITDKGEVPDGYVVSRSKTENFPLTLESATRSQTSVYFCASSDPGTEAFFGQG
TRLTVV
(SEQ ID NO: 300)
MGTRLFFYVALCLLWAGHRDAGITQSPRYKITETGRQVTLMCHQTWSHSYMFWYRQDLGHGL
RLIYYSAAADITDKGEVPDGYVVSRSKTENFPLTLESATRSQTSVYFCASSDPGTEAFFGQG
TRLTVVEDLKNVFPPEVAVFEPSEAEISHTQKATLVCLATGFYPDHVFLSWWVNGKEVHSGV
CTDPQPLKEQPALNDSRYCLSSRLRVSATFWQNPRNHFRCQVQFYGLSENDEWTQDRAKPVT
QIVSAEAWGRADCGFTSESYQQGVLSATILYEILLGKATLYAVLVSALVLMAMVKRKDSRG
(SEQ ID NO: 301)
MKSLRVLLVILWLQLSWVWSQQKEVEQNSGPLSVPEGAIASLNCTYSDRGSQSFFWYRQYSG
KSPELIMFIYSNGDKEDGRFTAQLNKASQYVSLLIRDSQPSDSATYLCAAAMDSSYKLIFGS
GTRLLVRP
(SEQ ID NO: 302)
MKSLRVLLVILWLQLSWVWSQQKEVEQNSGPLSVPEGAIASLNCTYSDRGSQSFFWYRQYSG
KSPELIMFIYSNGDKEDGRFTAQLNKASQYVSLLIRDSQPSDSATYLCAAAMDSSYKLIFGS
GTRLLVRPIQNPDPAVYQLRDSKSSDKSVCLFTDFDSQTNVSQSKDSDVYITDKCVLDMRSM
DFKSNSAVAWSNKSDFACANAFNNSIIPEDTFFPSPESSCDVKLVEKSFETDINLNFQNLSV
IGFRILLLKVAGFNLLMTLRLWSS
(SEQ ID NO: 303)
MGTRLFFYVALCLLWAGHRDAGITQSPRYKITETGRQVTLMCHQTWSHSYMFWYRQDLGHGL
RLIYYSAAADITDKGEVPDGYVVSRSKTENFPLTLESATRSQTSVYFCASSDPGTEAFFGQG
TRLTVVEDLKNVFPPEVAVFEPSEAEISHTQKATLVCLATGFYPDHVFLSWWVNGKEVHSGV
CTDPQPLKEQPALNDSRYCLSSRLRVSATFWQNPRNHFRCQVQFYGLSENDEWTQDRAKPVT
QIVSAEAWGRADCGFTSESYQQGVLSATILYEILLGKATLYAVLVSALVLMAMVKRKDSRGA
TNFSLLKQAGDVEENPGPMKSLRVLLVILWLQLSWVWSQQKEVEQNSGPLSVPEGAIASLNC
TYSDRGSQSFFWYRQYSGKSPELIMFIYSNGDKEDGRFTAQLNKASQYVSLLIRDSQPSDSA
TYLCAAAMDSSYKLIFGSGTRLLVRPIQNPDPAVYQLRDSKSSDKSVCLFTDFDSQTNVSQS
KDSDVYITDKCVLDMRSMDFKSNSAVAWSNKSDFACANAFNNSIIPEDTFFPSPESSCDVKL
VEKSFETDTNLNFQNLSVIGFRILLLKVAGFNLLMTLRLWSS
(SEQ ID NO: 304)
MRQVARVIVFLTLSTLSLAKTTQPISMDSYEGQEVNITCSHNNIATNDYITWYQQFPSQGPR
FIIQGYKTKVTNEVASLFIPADRKSSTLSLPRVSLSDTAVYYCLVGDMDQAGTALIFGKGTT
LSVSS
(SEQ ID NO: 305)
MGPGLLCWALLCLLGAGLVDAGVTQSPTHLIKTRGQQVTLRCSPKSGHDTVSWYQQALGQGP
QFIFQYYEEEERQRGNFPDRFSGHQFPNYSSELNVNALLLGDSALYLCASSLGEGRVDGYTF
GSGTRLTVV
(SEQ ID NO: 306)
MRQVARVIVFLTLSTLSLAKTTQPISMDSYEGQEVNITCSHNNIATNDYITWYQQFPSQGPR
FIIQGYKTKVTNEVASLFIPADRKSSTLSLPRVSLSDTAVYYCLVGDMDQAGTALIFGKGTT
LSVSS
(SEQ ID NO: 307)
MGPGLLCWALLCLLGAGLVDAGVTQSPTHLIKTRGQQVTLRCSPKSGHDTVSWYQQALGQGP
QFIFQYYEEEERQRGNFPDRFSGHQFPNYSSELNVNALLLGDSALYLCASSLGRASNQPQHE
GDGTRLSIL
(SEQ ID NO: 308)
MRQVARVIVFLTLSTLSLAKTTQPISMDSYEGQEVNITCSHNNIATNDYITWYQQFPSQGPR
FIIQGYKTKVTNEVASLFIPADRKSSTLSLPRVSLSDTAVYYCLVGDRDQAGTALIFGKGTT
LSVSS
(SEQ ID NO: 309)
MGPGLLCWALLCLLGAGLVDAGVTQSPTHLIKTRGQQVTLRCSPKSGHDTVSWYQQALGQGP
QFIFQYYEEEERQRGNFPDRFSGHQFPNYSSELNVNALLLGDSALYLCASSFGQSSTYGYTF
GSGTRLTVV
(SEQ ID NO: 310)
MRQVARVIVFLTLSTLSLAKTTQPISMDSYEGQEVNITCSHNNIAINDYITWYQQFPSQGPR
FIIQGYKTKVTNEVASLFIPADRKSSTLSLPRVSLSDTAVYYCLVGDMDQAGTALIFGKGTT
LSVSSDIQNPEPAVYQLKDPRSQDSTLCLFTDFDSQINVPKTMESGTFITDKCVLDMKAMDS
KSNGAIAWSNQTSFTCQDIFKETNATYPSSDVPCDATLTEKSFETDMNLNFQNLLVIVLRIL
LLKVAGFNLLMTLRLWSS
(SEQ ID NO: 311)
MGPGLLCWALLCLLGAGLVDAGVTQSPTHLIKTRGQQVTLRCSPKSGHDTVSWYQQALGQGP
QFIFQYYEEEERQRGNFPDRFSGHQFPNYSSELNVNALLLGDSALYLCASSLGEGRVDGYTF
GSGTRLTVVEDLRNVTPPKVSLFEPSKAEIANKQKATLVCLARGFFPDHVFLSWWVNGKEVH
SGVCTDPQAYKESNYSYCLSSRLRVSATFWHNPRNHFRCQVQFHGLSEEDKWPEGSPKPVTQ
NISAEAWGRADCGITSASYQQGVLSATILYEILLGKATLYAVLVSTLVVMAMVKRKNS
(SEQ ID NO: 312)
MRQVARVIVFLTLSTLSLAKTTQPISMDSYEGQEVNITCSHNNIATNDYITWYQQFPSQGPR
FIIQGYKTKVTNEVASLFIPADRKSSTLSLPRVSLSDTAVYYCLVGDMDQAGTALIFGKGTT
LSVSSDIQNPEPAVYQLKDPRSQDSTLCLFTDFDSQINVPKTMESGTFITDKCVLDMKAMDS
KSNGAIAWSNQTSFTCQDIFKETNATYPSSDVPCDATLTEKSFETDMNLNFQNLLVIVLRIL
LLKVAGFNLLMTLRLWSS
(SEQ ID NO: 313)
MGPGLLCWALLCLLGAGLVDAGVTQSPTHLIKTRGQQVTLRCSPKSGHDTVSWYQQALGQGP
QFIFQYYEEEERQRGNFPDRFSGHQFPNYSSELNVNALLLGDSALYLCASSLGRASNQPQHF
GDGTRLSILEDLRNVTPPKVSLFEPSKAEIANKQKATLVCLARGFFPDHVFLSWWVNGKEVH
SGVCTDPQAYKESNYSYCLSSRLRVSATFWHNPRNHFRCQVQFHGLSEEDKWPEGSPKPVTQ
NISAEAWGRADCGITSASYQQGVLSATILYEILLGKATLYAVLVSTLVVMAMVKRKNS
(SEQ ID NO: 314)
MRQVARVIVFLTLSTLSLAKTTQPISMDSYEGQEVNITCSHNNIATNDYITWYQQFPSQGPR
FIIQGYKTKVTNEVASLFIPADRKSSTLSLPRVSLSDTAVYYCLVGDRDQAGTALIFGKGTT
LSVSSDIQNPEPAVYQLKDPRSQDSTLCLFTDFDSQINVPKTMESGTFITDKCVLDMKAMDS
KSNGAIAWSNQTSFTCQDIFKETNATYPSSDVPCDATLTEKSFETDMNLNFQNLLVIVLRIL
LLKVAGFNLLMTLRLWSS
(SEQ ID NO: 315)
MGPGLLCWALLCLLGAGLVDAGVTQSPTHLIKTRGQQVTLRCSPKSGHDTVSWYQQALGQGP
QFIFQYYEEEERQRGNFPDRFSGHQFPNYSSELNVNALLLGDSALYLCASSFGQSSTYGYTF
GSGTRLTVVEDLRNVTPPKVSLFEPSKAEIANKQKATLVCLARGFFPDHVFLSWWVNGKEVH
SGVCTDPQAYKESNYSYCLSSRLRVSATFWHNPRNHFRCQVQFHGLSEEDKWPEGSPKPVTQ
NISAEAWGRADCGITSASYQQGVLSATILYEILLGKATLYAVLVSTLVVMAMVKRKNS
(SEQ ID NO: 316)
MKSLRVLLVILWLQLSWVWSQQKEVEQNSGPLSVPEGAIASLNCTYSDRGSQSFFWYRQYSG
KSPELIMFIYSNGDKEDGRFTAQLNKASQYVSLLIRDSQPSDSATYLCAAAMDSSYKLIFGS
GTRLLVRPDIQNPEPAVYQLKDPRSQDSTLCLFTDFDSQINVPKTMESGTFITDKCVLDMKA
MDSKSNGAIAWSNQTSFTCQDIFKETNATYPSSDVPCDATLTEKSFETDMNLNFQNLLVIVL
RILLLKVAGFNLLMTLRLWSS
(SEQ ID NO: 317)
MGTRLFFYVALCLLWAGHRDAGITQSPRYKITETGRQVTLMCHQTWSHSYMFWYRQDLGHGL
RLIYYSAAADITDKGEVPDGYVVSRSKTENFPLTLESATRSQTSVYFCASSDPGTEAFFGQG
TRLTVVEDLRNVTPPKVSLFEPSKAEIANKQKATLVCLARGFFPDHVFLSWWVNGKEVHSGV
CTDPQAYKESNYSYCLSSRLRVSATFWHNPRNHFRCQVQFHGLSEEDKWPEGSPKPVTQNIS
AEAWGRADCGITSASYQQGVLSATILYEILLGKATLYAVLVSTLVVMAMVKRKNS

### B. NK Cells

The NK cells that are modified to express the TCR/CD3 receptor complex may be obtained from any suitable source, including fresh or frozen. In certain embodiments, NK cells are not NK cells obtained from iPSC differentiation. In certain embodiments, NK cells are derived from human peripheral blood mononuclear cells (PBMC), unstimulated leukapheresis products (PBSC), NK cell lines (e.g., NK-92), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, stem cells, NK cell lines, or a mixture thereof. In particular embodiments, the NK cells are isolated from pooled CB. The CB may be pooled from 2, 3, 4, 5, 6, 7, 8, 9, 10, or more units. The NK cells may be autologous or allogeneic with respect to a recipient individual. The isolated NK cells may or may not be haplotype matched for the subject to be administered the cell therapy. NK cells can be detected by specific surface markers, such as CD16 and CD56 in humans, for example. In some cases, the source of the NK cells is cord blood and the NK cells may be in the cord blood in a heterogeneous mixture of cells and may be depleted of certain cells expressing CD3. In other methods, umbilical CB is used to derive NK cells by the isolation of CD34+ cells.

The NK cells may be pre-activated with one or more inflammatory cytokines, and they may be expanded or non-expanded. In some cases, the NK cells are pre-activated either prior to modification to express CD3±TCR or following modification to express CD3±TCR complex. In specific embodiments, pre-activation of the NK cells may comprise culturing the isolated NK cells in the presence of one or more cytokines. The NK cells may be stimulated with IL-2, or other cytokines that bind the common gamma-chain (e.g., IL-7, IL-12, IL-15, IL-18, IL-21, and others). In particular embodiments, the pre-activation cytokines may be selected from the group consisting of IL-12, IL-15, IL-18, and a combination thereof. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, specifically about 16 hours. The pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/mL, specifically about 10 ng/mL. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/mL, specifically about 50 ng/mL.

In some cases, the NK cells are expanded either prior to modification to express CD3±TCR complex or following modification to express CD3±TCR complex. Pre-activated NK cells may be expanded in the presence of artificial antigen presenting cells (aAPCs) and/or feeders/fragments or NK activating beads. The pre-activated NK cells may be washed prior to expansion, such as 2, 3, 4, or 5 times, specifically 3 times. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58). In particular, the aAPCs may be K562 cells, such as K562 cells engineered to express CD137 ligand and mIL-21. The aAPCs may be irradiated. In some embodiments, fragments of APC can be used to expand the NK cells. The engineering may be by any method known in the art, such as retroviral transduction. Retroviral transduction may be at least, at most, or about 1, 2, 3, 4, 5, 6, 7, 8, 9, 10, 11, 12, 13, 14, 15, 16, 17, 18, 19, or 20 days following NK co-culturing with an antigen presenting cell. In some embodiments, retroviral transduction comprises co-transduction of more than one construct. In some embodiments, retroviral transduction occurs after or at about 5 days of co-culturing with an antigen presenting cell. In some embodiments, co-culturing with an antigen presenting cell continues following transduction of an NK cell. The expansion may be for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise cytokines to promote expansion, such as IL-2. The IL-2 may be present at a concentration of about 10-500 U/mL, such as 100-300 U/mL, particularly about 200 U/mL. The IL-2 may be replenished in the expansion culture, such as every 2-3 days. The aAPCs may be added to the culture at least a second time, such as at about 7 days of expansion.

In particular embodiments, the NK cells are transfected or transduced with one or more membrane bound cytokines, including IL-21, IL-12, IL-18, IL-23, IL-7, or IL-15, either secreted by NK cells or tethered to the NK cell membrane. In such cases, the membrane bound cytokine may be tethered to the NK cell membrane with a particular transmembrane domain, such as the transmembrane domain of CD8, CD28, CD27, B7H3, IgG1, IgG4, CD4, DAP10, DAP12, for example.

Following preparation, the modified NK cells may be immediately infused (including with an effective amount of one or more monospecific, bispecific or multi-specific antibodies, or the NK cells may be stored, such as by cryopreservation. In some cases, when the NK cells are source from cryopreservation, the NK cells were deactivated pre-cryopreservation using a deactivating agent (e.g., a kinase inhibitor, e.g., Dasatinib, nilotinib, rapamycin, etc.). In certain aspects, the cells may be propagated for days, weeks, or months ex vivo as a bulk population within about 1, 2, 3, 4, or 5 days.

### C. Loading of NK Cells

In particular embodiments, the NK cells are loaded with antibodies prior to use. The NK cells may be loaded in any specific manner, including in culture or immediately before infusion, for example, to produce a complex of NK cells with the antibodies. The conditions are suitable enough to allow for an effective amount of antibody to bind to the surface of the NK cells. In the case of use of monospecific antibodies, the Fc region of the monospecific antibody binds the NK cell while the antigen binding domain of the monospecific antibody is free to bind its target antigen. In certain embodiments of cases of use of multispecific antibodies, one or more antigen binding domains of the antibody can bind the surface of the NK cells, such as through an antigen on the surface of the NK cells, (for example but not limited to, CD3, NKp30, NKp44, NKp46, CD16, CD32, CD64, KIRs, and the like), and the other antigen binding domain is free to bind its target antigen. In certain embodiments of cases of use of multispecific antibodies, one or more antigen binding domains of the antibody can bind one or more target antigens.

The culture conditions by which the NK cells become loaded may or may not be of a particular type having one or more specific parameters. In particular embodiments, the loading of the NK cells occurs in culture at a specific temperature, such as 37° C., although in alternative embodiments the temperature is 36° C. or 38° C., or lower or higher. The duration of the loading step may be for any suitable amount of time, such as in a range of one minute to 24 hours or longer. For example, the range may be in the range of 1 min to 24 hrs, 1 min to 18 hrs, 1 min to 12 hours, 1 min to 6 hrs, 1 min to 1 hr, 30 min to 24 hrs, 30 min to 18 hrs, 30 min to 12 hrs, 30 min to 6 hrs, 30 min to 1 hr, 1-24 hrs, 1-18 hrs, 1-12 hrs, 1-6 hrs, 6-24 hrs, 6-18 hrs, 6-12 hrs, 12-24 hrs, 12-18 hrs, or 18-24 hrs. In some embodiments, the duration of the loading step may be greater than or equal to approximately 6, 8, 10, 12, 14, 16, 18, 20, 22, 24, 26, 28, 30, 32, 34, 36, 38, 40, 42, 44, 46, or 48 hours, or any range derivable therein. In specific embodiments, the cell culture media is basal media or complex media. In some cases, the culture comprises one or more reagents that were utilized during pre-activation and/or expansion steps, while in other cases the culture does not. In specific embodiments, the culture comprises one or more cytokines, including one or more of IL-12, IL-15, IL-2, and IL-18, for example. In some embodiments, the culture comprises APCs of any kind.

In certain embodiments, antibodies of compositions described herein are subjected in an effective amount to an effective amount of NK cells of the disclosure, thereby producing a complex that is “chimeric antigen receptor-like.” In particular, an antigen binding domain of the antibody binds to the NK cells, such as through the antigen that is a cell surface protein. A plurality of antibodies may be subjected to a plurality of NK cells such that there are multiple complexes of cell/antibody. The antibodies may be of any type, including monospecific, bispecific, or multispecific, and in specific cases the antibody engages both the NK cell and a target antigen through an antigen binding domain of the antibody (such as with engagers in the art that are fusion proteins consisting of two single-chain variable fragments (scFvs) of different antibodies). In cases wherein the antibody is monospecific, an antigen binding domain of the antibody binds a target antigen, such as a cancer antigen, and another part of the antibody binds the NK cells, such as an Fc region of the antibody. In cases wherein the antibody is multispecific, one or more antigen binding domains of the antibody binds the NK cell (such as through an NK cell surface antigen, either naturally occurring, or transgenic, e.g., CD3) and one or more antigen binding domains of the antibody binds one or more target antigens. The multispecific antibody may be bispecific, trispecific, or tetraspecific, for example. In cases wherein the antibody is trispecific or tetraspecific, the additional antigen binding domains may bind other cells, such as stem cells.

In particular embodiments, the antibodies may bind any NK cell surface antigen (that may or may not be receptors) on NK cells, such as CD16 (including CD16a or CD16b), CD32, CD56, CD64, a c-type lectin such as NKG2D, NKG2C, a costimulatory molecule such as CS1, DNAM, 2B4, CD2, an NCR, NKp30, NKp44, NKp46, or KIR, and redirect the NK cells to a target, thus increasing the response and specificity against different tumors. In certain embodiments, the antibodies may bind to a transgenic NK cell surface antigen, such as CD3.

In some embodiments, the antibodies may bind any suitable antigen (e.g., antigens described herein, such as those that are described as targets of CARs and/or TCRs). In particular embodiments, an antibody targets CD19. In particular embodiments, an antibody targets CD20. In particular embodiments, an antibody targets CD123. In particular embodiments, an antibody targets EGFR. In particular embodiments, an antibody targets EGFR2. In particular embodiments, an antibody targets C-met (tyrosine-protein kinase MET, aka mesenchymal epithelial transition factor). In particular embodiments, an antibody targets EGFR and C-met. In particular embodiments, an antibody targets TROP-2 (tumor-associated calcium signal transducer 2).

In certain embodiments, generation of loaded NK cells may be by any suitable means, such that the conditions are sufficient for the appropriate region of the antibody to bind the appropriate surface region of the NK cell. Any particular medium may be utilized, in certain instances. In specific cases, Plasma-Lyte A and/or human serum albumin are utilized, wherein in other cases they are not. Once the complexes are formed in culture, they may or may not be washed prior to administration to the subject, such as through infusion. In alternative embodiments, the NK cells and the antibodies are administered separately, and the complexes form in vivo.

### D. Pre-Activation

In some embodiments, the NK cells are pre-activated prior to administration to a recipient individual. The pre-activation step may or may not occur before any expansion step. In specific embodiments, the NK cells are pre-activated with one or more cytokines, and in specific embodiments, the NK cells are pre-activated with one or more of IL-12, IL-15, IL-2, and IL-18 and including two, three, or more. In cases wherein less than all three of IL-12, IL-15, IL-2, and IL-18 are utilized, it may be that IL-12 and IL-15 but not IL-18; or IL-12 and IL-18 but not IL-15; or IL-15 and IL-18 but not IL-12. IL-2 may or may not be substituted for IL-15.

In particular embodiments, the pre-activation cytokines may be IL-12, IL-15, and IL-18. One or more additional cytokines may be used for the pre-activation step. The pre-activation may be for a short period of time such as 5-72 hours, such as 10-50 hours, particularly 10-20 hours, such as 12, 13, 14, 15, 16, 17, 18, 19, or 20 hours, and specifically about 16 hours in some cases. The pre-activation culture may comprise IL-18 and/or IL-15 at a concentration of 10-100 ng/mL, such as 40-60 ng/mL, particular 45, 46, 47, 48, 49, 50, 51, 52, 53, 54, or 55 ng/mL, specifically about 50 ng/mL. In some cases, the pre-activation culture comprises IL-12 at a concentration of 0.1-150 ng/mL, including at a concentration of 1-20 ng/mL, such as a concentration of 10 ng/mL. In alternative embodiments the NK cells may be stimulated with IL-2, or other cytokines that bind the common gamma-chain (e.g., IL-7, IL-21, and others), and this may be in addition to IL-12, IL-15, and IL-18 or as an alternative to one or more of them. In such cases, the pre-activation culture may comprise IL-12 at a concentration of 0.1-150 ng/mL, such as 0.5-50 ng/mL, particularly 1-20 ng/mL, such as 2, 3, 4, 5, 6, 7, 8, 9, 10, 11, 12, 13, 14, or 15 ng/mL, specifically about 10 ng/mL.

### E. Expansion

In particular embodiments, NK cells are expanded to increase their quantity prior to administration to an individual in need thereof. The expanded cells may or may not be derived from pre-activated NK cells such that a pre-activation step may occur before an expansion step. The NK cell expansion step may be of any suitable such that the NK cell population is expanded, but in specific cases the expansion step utilizes particular one or more reagents, such as in culture, to enhance their expansion. In certain cases the NK cells may not be expanded. IL-2 or IL-15 or IL-18 or any combination of the cytokines may be added to the expansion culture before or during expansion. The NK cells can be expanded ex vivo in flasks or in one of several different bioreactor configurations with continuous perfusion of media/additives, in specific embodiments.

In specific cases, the NK cells (whether pre-activated or not) may be washed (e.g., with PBS or Plasma Lyte or human serum albumin or culture media or combinations thereof) prior to and/or after expansion, such as 1, 2, 3, 4, or 5 times. In some embodiments, cells are washed specifically 3 times. In particular embodiments, the NK cells are expanded in the presence of artificial antigen presenting cells (aAPCs). In particular embodiments, the NK cells are expanded in the presence of fragments of aAPCs. The aAPCs may be engineered to express CD137 ligand and/or a membrane-bound cytokine. The membrane-bound cytokine may be membrane-bound IL-21 (mIL-21) or membrane-bound IL-15 (mIL-15). In particular embodiments, the aAPCs are engineered to express CD137 ligand and mIL-21. The aAPCs may be derived from cancer cells, such as leukemia cells. The aAPCs may not express endogenous HLA class I, II, or CD1d molecules. They may express ICAM-1 (CD54) and LFA-3 (CD58) or CD48. In particular, the aAPCs may be K562 cells, such as K562 cells engineered to express CD137 ligand and mIL-21. The engineering may be by any method known in the art, such as retroviral transduction, although any viral or non-viral vector may be utilized. The aAPCs may or may not be irradiated. The expansion may be for a particular duration in time, such as for about 2-30 days, such as 3-20 days, particularly 12-16 days, such as 12, 13, 14, 15, 16, 17, 18, or 19 days, specifically about 14 days. The pre-activated NK cells and aAPCs may be present at a ratio of about 3:1-1:3, such as 2:1, 1:1, 1:2, specifically about 1:2. The expansion culture may further comprise one or more cytokines to promote expansion, such as IL-2. The IL-2 may be present at a concentration of about 10-500 U/mL, such as 100-300 U/mL, particularly about 200 U/mL. The IL-2 may be replenished in the expansion culture, including at a certain frequency, such as every 2-3 days. The aAPCs may be added to the culture at least a second time, such as at about 7 days of expansion. Any cytokine(s) used in the pre-activation and/or expansion steps may be recombinant human cytokines.

In some embodiments, following expansion, the NK cells may be immediately utilized in any manner, such as complexed with one or more antibodies, or they may be stored, such as by cryopreservation. In certain aspects, the cells may be propagated for days, weeks, or months ex vivo as a bulk population within about 1, 2, 3, 4, or 5 days.

Activated and/or expanded NK cells can secrete type I cytokines, such as interferon-7, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

Thus, with respect to particular pre-activation and expansion aspects of the disclosure, in specific embodiments the NK cells pre-activated with any combination of IL-12, IL15, and/or IL-18 followed by expansion with aAPCs, such as K562 cells expressing mIL-21 and CD137 ligand, provide a highly potent cellular product. Thus, methods are provided using the present NK cells for the treatment of various diseases, such as immunotherapy of patients with cancer. In an exemplary method, the isolated NK cells may be subjected to a brief period, such as about 16 hours, of pre-activation with a combination of cytokines, such as interleukin-12 (IL-12), IL-15, and/or IL-18, followed by expansion using artificial antigen presenting cells (aAPCs), such as K562 feeder cells expressing membrane-bound IL-21 and CD137 ligand, and/or exogenous IL-2. IL-2 or IL-15 or IL-18 or any combination of the cytokines may be added to the expansion culture at least a second time.

### F. Cryopreservation

In particular cases, NK cells and/or antibodies of the disclosure are preserved in a cryopreservation medium composition comprising at least one cryoprotectant, a serum (human or animal serum) or a non-serum alternative to serum (not human serum or animal serum), and at least one cytokine and/or at least one growth factor. In some cases, the cryoprotectant is dimethyl sulfoxide (DMSO), glycerin, glycerol, hydroxyethyl starch, or a combination thereof. The non-serum alternative may be of any kind, including at least platelet lysate and/or a blood product lysate (for example, human serum albumin). In embodiments of the composition wherein one or more (including two or more) cytokines are utilized, the cytokine may be a natural or a recombinant or a synthetic protein. At least one of the cytokines may be an Food and Drug Administration (FDA)-approved cytokine. Examples of cytokines and growth factors include at least IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof. For serum embodiments, the serum may be an animal-derived serum, such as human serum (including human AB serum) or bovine serum. DMSO and other cryoprotectants, when utilized may comprise 4-10%, 4-6%, 4-8%, 5-10%, 5-8%, 6-10%, 6-8%, 8-10%, and so forth, of the composition. For embodiments wherein serum is employed, the serum may comprise 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of serum. In specific embodiments, the composition comprises platelet lysate that may be at any concentration in the composition, but in certain embodiments the platelet lysate comprises 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%, 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of platelet lysate.

The composition may have certain concentrations of components, including cytokines and/or growth factors. In specific cases, any cytokine, including IL-2, IL-21, and/or IL-15, for example, are present in the composition in a particular concentration. The IL-2 may be present at a concentration of 1-5000, 1-1000, 1-500, 1-100, 100-5000, 100-500, 500-5000, 500-1000, or 1000-5000 U/mL, for example. In a specific case, the IL-2 is present at a concentration in the composition of at least or no more than 100, 200, 300, 400, 500, 600, 700, 800, 900, or 1000 U/mL. In specific embodiments, IL-21 is present in the composition at a concentration of 10-3000, 10-2000, 10-1000, 10-500, 10-100, 100-3000, 100-2000, 100-1000, 500-3000, 500-2000, 500-1000, 1000-3000, 1000-2000, or 2000-3000 ng/mL. The IL-21 may be in a concentration in the composition of at least or nor more than 1, 5, 10, 20, 30, 40, 50, 60, 70, 80, 90, 100, 150, 200, 250, 300, 350, 400, 450, 500, 750, 1000, 1250, 1500, 1750, 2000, 2250, 2500, 2750, or 3000 ng/mL. IL-15 may be present in the composition at a concentration of 1-2000, 1-1000, 1-500, 1-100, 100-2000, 100-1000, 100-500, 500-2000, 500-1000, or 1000-2000 ng/mL. IL-15 may be present in the composition at a concentration of at least or no more than 10, 50, 100, 500, 1000, 1500, or 2000 ng/mL.

Compositions as encompassed herein that comprise at least one cryoprotectant, a serum or a non-serum alternative to serum, and at least one cytokine and/or at least one growth factor may further comprise a plurality of immune cells and/or stem cells, each of any kind. In specific embodiments, the cells are NK cells, T cells, B cells, NKT cells derived from mature bone marrow or peripheral blood cells, cell lines such as tumor cell lines (e.g., NK92 or other NK lines), hematopoietic stem cells, induced pluripotent stem cells, MSCs (a population of cells alternatively called “mesenchymal stem cells” and “mesenchymal stromal cells” in the literature), or a mixture thereof, which can be derived from bone marrow, peripheral blood, skin, adipose tissue, or a combination thereof. In embodiments wherein NK cells are utilized, the NK cells may or may not be expanded NK cells. Embodiments of the disclosure also encompass pharmaceutical compositions that comprise any composition of the disclosure and a suitable pharmaceutically acceptable carrier.

In certain embodiments, cells and/or antibodies are treated with one or more deactivating agents (e.g., a kinase inhibitor, e.g., Dasatinib, Nilotinib, Rapamycin, etc.) pre-cryopreservation.

In some embodiments, technologies described herein comprise deactivating a NK cell, comprising treating an NK cell with an effective amount of one or more deactivating agents under conditions to produce a deactivated NK cell. In some embodiments, a deactivating agent is a kinase inhibitor. In some embodiments, a deactivating agent is a mechanistic target of rapamycin (mTOR) inhibitor. In some embodiments, the mTOR inhibitor is rapamycin, everolimus, and/or temsirolimus. In some embodiments, the mTOR inhibitor is rapamycin. In some embodiments, the deactivating agent is a tyrosine kinase (TK) inhibitor. In some embodiments, the TK inhibitor is Lorlatinib, Brigatinib, Ceritinib, Alectinib, Crizotinib, Bosutinib, Ponatinib, Nilotinib, Dasatinib, Imatinib, Zanubrutinib, Acalabrutinib, Ibrutinib, Capmatinib, Pexidartinib, Dacomitinib, Osimertinib, Erlotinib, Gefitinib, Lapatinib, Afatinib, Pemigatinib, Erdafitinib, Nintedanib, Gilteritinib, Midostaurin, Tucatinib, Neratinib, Baricitinib, Ruxolitinib, Fedratinib, Tofacitinib, Ripretinib, Selumetinib, Binimetinib, Cobimetinib, Trametinib, Upadacitinib, Avapritinib, Selpercatinib, Cabozantinib, Fostamatinib, Larotrectinib, Entrectinib, Axitinib, Regorafenib, Pazopanib, Sorafenib, Lenvatinib, Vandetanib, and/or Sunitinib. In some embodiments, the TK inhibitor is a BCR-Abl inhibitor. In some embodiments, the TK inhibitor is Bosutinib, Ponatinib, Nilotinib, Dasatinib, and/or Imatinib. In some embodiments, the TK inhibitor is Dasatinib and/or Nilotinib. In some embodiments, the TK inhibitor is Dasatinib.

In some embodiments, treatment with a deactivating agent is at any point during culturing of the NK cell. In some embodiments, the treatment is for about 24 to about 96 hours, about 36 to about 84 hours, or about 48 to about 72 hours. In some embodiments, the treatment is for about 24 hours, about 48 hours, or about 72 hours. In some embodiments, the NK cell is treated with the deactivating agent at a concentration of about 1 to about 1000 nM. In some embodiments, the NK cell is treated with the deactivating agent at a concentration of about 5 to about 500 nM. In some embodiments, the NK cell is treated with the deactivating agent at a concentration of about 20 to about 200 nM. In some embodiments, the NK cell is treated with the deactivating agent at a concentration of about 30 to about 100 nM. In some embodiments, the deactivated NK cell has an increased expression of one or more of C-kit, CCR-5, CD62L and/or CXCR4, and/or decreased expression of one or more of NKG2D, DNAM, OX-40, TRAIL, HLA-DR, CD2, CD25, ICOS, and/or CD95 relative to an activated NK cell.

In some embodiments, technologies described herein comprise methods of maintaining the viability of a population of cells over at least 50% percent following cryopreservation of the population, comprising the step of subjecting the population to an effective amount of one or more deactivating agents (e.g., a tyrosine kinase inhibitor) to deactivate the cells prior to cryopreservation, cryopreserving the cells, and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population.

### III. HETEROLOGOUS PROTEINS AND MUTATIONS

In specific embodiments, the NK cells are modified not only to express one or more components of the TCR/CD3 complex, but they are also modified to express one or more other heterologous proteins. The heterologous proteins may facilitate activity of the NK cells in any manner, including at least their activation, persistence, expansion, homing, and/or cytotoxicity.

### A. Monospecific, Bispecific or Multi-Specific Antibodies

In some embodiments, the NK cells are modified to express one or more monospecific, bispecific or multi-specific antibodies, although in other cases the NK cells do not express the antibodies but the antibodies are utilized in conjunction with the NK cells.

In cases wherein the NK cells are modified to express the antibodies, the antibodies may be engagers that bridge a particular immune effector cell with a particular target cell for destruction of the target cell. The present disclosure allows the modified NK cells to be used with standard T-cell engagers (BiTEs) because they have been modified to express CD3 that in many cases is the T cell antigen to which the BiTE engager binds. In such cases, the BiTE used in the invention may also target a cancer or viral antigen that may be tailored to the medical condition of an intended recipient individual. For example, the BiTE may be tailored to bind a cancer antigen that is characteristic of the cancer cells of a cancer of the individual. The anti-CD3 antibody of the BiTE may target the CD3γ chain, CD3δ chain, CD3ε chain, or CD3ζ chain.

In some cases, in addition to expressing the CD3 complex (with or without TCR) that allows the NK cells to be utilized as a therapy with BiTEs, the NK cells may be modified to express (or not to express but instead used in conjunction with) one or more bispecific NK engagers (BiKEs). The BiKE comprises an antibody that binds a surface protein on the NK cell, including a naturally expressed surface protein on NK cells (for example but not limited to, NKp30, NKp44, NKp46, CD16, CD32, CD64, KIRs, and the like), and also comprises an antibody that binds a desired target antigen. The BiKE may target the NK cells through an antibody an NK surface protein such as CD16, CS1, CD32, CD64, CD56, NKG2D, NKG2C, DNAM, 2B4, CD2, an NCR, NKp30, NKp44, NKp46, or KIR, for example. In such cases, the BiKE used in the invention may also target a cancer or viral antigen that may be tailored to the medical condition of an intended recipient individual. For example, the BiKE may be tailored to bind a cancer antigen that is characteristic of the cancer cells of a cancer of the individual.

In certain embodiments, an antibody is Blinatumomab. In certain embodiments, an antibody is Tebentafusp. In certain embodiments, an antibody is Mosunetuzumab. In certain embodiments, an antibody is Teclistamab. In certain embodiments, an antibody is Glofitamab. In certain embodiments, an antibody is Epcoritamab. In some embodiments, an antibody is Flotetuzumab. In some embodiments, an antibody is APV0436. In some embodiments, an antibody is TNB383B. In certain embodiments of cases of use of multispecific antibodies, one or more antigen binding domains of the antibody can bind one or more target antigens. In certain embodiments, an antibody is Amivantamab. In certain embodiments, an antibody is Cetuximab. In certain embodiments, an antibody is Imgatuzumab.

In embodiments wherein an NK cell expresses the CD3 complex (with or without TCR) and one or more BiKEs, one or more vectors may be utilized to transfect or transduce the cells with the CD3 complex components (with or without TCR) and one or more BiKEs. In some cases, one or more of the CD3 complex components (with or without TCR) and the BiKE may or may not be on the same multicistronic vector.

### B. Engineered Receptors

In specific embodiments, the NK cells are engineered to express one or more engineered receptors. In some cases, the engineered receptors are engineered antigen receptors that target a cancer or viral antigen of any kind. The receptor may be tailored to target a desired antigen based on a medical condition of an intended recipient individual.

In some embodiments, the engineered antigen receptor is a chimeric antigen receptor (CAR). The NK cells may be modified to encode at least one CAR, and the CAR may be first generation, second generation, or third or a subsequent generation, for example. The CAR may or may not be bispecific for two or more different antigens. The CAR may comprise one or more costimulatory domains. Each costimulatory domain may comprise the costimulatory domain of any one or more of, for example, members of the TNFR superfamily, CD28, CD137 (4-1BB), CD134 (OX40), DAP10, DAP12, CD27, CD2, CD5, ICAM-1, LFA-1 (CD11a/CD18), Lck, TNFR-I, TNFR-II, Fas, CD30, CD27, NKG2D, 2B4M, CD40 or combinations thereof, for example. In specific embodiments, the CAR comprises CD3zeta. In certain embodiments, the CAR lacks one or more specific costimulatory domains; for example, the CAR may lack 4-1BB and/or lack CD28.

In particular embodiments, the CAR polypeptide in the cells comprises an extracellular spacer domain that links the antigen binding domain and the transmembrane domain, and this may be referred to as a hinge. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, CD28, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha or CD4, (v) a hinge, CH2 and CH3 regions of IgG1, (vi) a hinge region of IgG1 or (vii) a hinge and CH2 of IgG1, (viii) a hinge region of CD28, or a combination thereof. In specific embodiments, the hinge is from IgG1 and in certain aspects the CAR polypeptide comprises a particular IgG1 hinge amino acid sequence or is encoded by a particular IgG1 hinge nucleic acid sequence.

The transmembrane domain in the CAR may be derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules, such as DAP10 or DAP12. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine may be found at each end of a synthetic transmembrane domain.

In some embodiments, the engineered receptors utilize one or more homing receptors (that can home to a target not necessarily because of a signal release, such as in the event that they utilize adhesion molecules) and/or one or more chemokine receptors. Examples of chemokine receptors include CXC chemokine receptors, CC chemokine receptors, CX3C chemokine receptors and XC chemokine receptors. In specific cases, the chemokine receptor is a receptor for CCR2, CCR3, CCR5, CCR8, CCR7, CXCR3, L-selectin (CD62L) CXCR1, CXCR2, or CX3CR1.

### C. Cytokines

In some embodiments, the cells expressing the NK cells are engineered to express one or more heterologous cytokines and/or are engineered to upregulate normal expression of one or more heterologous cytokines. The cells may or may not be transduced or transfected for one or more cytokines on the same vector as other genes. In certain embodiments, NK cells may be modified to express one or more cytokines, cytokine receptors, chemokines, chemokine receptors, and/or suicide genes.

One or more cytokines may be co-expressed from a vector, including as a separate polypeptide from any component of the TCR/CD3 complex. Interleukin-15 (IL-15), for example, is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death (AICD). In addition to IL-15, other cytokines are envisioned. These include, but are not limited to, cytokines, chemokines, and other molecules that contribute to the activation and proliferation of cells used for human application. NK cells expressing IL-15 are capable of continued supportive cytokine signaling, which is useful for their survival post-infusion.

In some embodiments, the cells express one or more exogenously provided engineered receptors, wherein the engineered receptor comprises a chemokine receptor and/or a cytokine receptor. In some embodiments, a cytokine receptor is an IL-15 receptor. In some embodiments, a cytokine receptor is a non-naturally occurring variant of a cytokine receptor. In some embodiments, a cytokine receptor is an IL-15, IL-12, IL-2, IL-18, IL-21, IL-23, or GMCSF receptor, or a combination thereof.

In specific embodiments, the cells express one or more exogenously provided cytokines. As one example, the cytokine is IL-15, IL-12, IL-2, IL-18, IL-21, IL-23, GMCSF, or a combination thereof. The cytokine may be exogenously provided to the NK cells because it is expressed from an expression vector within the cell. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as one or more components of the CD3 complex with or without the TCR complex.

In some embodiments, a specific sequence of IL-15 is utilized, such as those that follow (underlining refers to signal peptide sequence):

(SEQ ID NO: 49)
ATGCGCATTAGCAAGCCCCACCTGCGGAGCATCAGCATCCAGTGCTAC
CTGTGCCTGCTGCTGAACAGCCACTTCCTGACCGAGGCCGGCATCCAC
GTGTTCATCCTGGGCTGCTTCAGCGCCGGACTGCCCAAGACCGAGGCC
AACTGGGTGAACGTGATCAGCGACCTGAAGAAGATCGAGGACCTGATC
CAGAGCATGCACATCGACGCCACCCTGTACACCGAGAGCGACGTGCAC
CCCAGCTGCAAGGTGACCGCCATGAAGTGCTTTCTGCTGGAACTGCAG
GTGATCAGCCTGGAAAGCGGCGACGCCAGCATCCACGACACCGTGGAG
AACCTGATCATCCTGGCCAACAACAGCCTGAGCAGCAACGGCAACGTG
ACCGAGAGCGGCTGCAAAGAGTGCGAGGAACTGGAAGAGAAGAACATC
AAAGAGTTTCTGCAGAGCTTCGTGCACATCGTGCAGATGTTCATCAAC
ACCAGC
(SEQ ID NO: 48)
MRISKPHLRSISIQCYLCLLLNSHFLTEAGIHVFILGCFSAGLPKTEA
NWVNVISDLKKIEDLIQSMHIDATLYTESDVHPSCKVTAMKCFLLELQ
VISLESGDASIHDTVENLIILANNSLSSNGNVTESGCKECEELEEKNI
KEFLQSFVHIVQMFINTS

### D. Antigens

The modified NK cells of the disclosure are utilized with monospecific, bispecific or multi-specific antibodies that target one or more particular antigens. In addition, the NK cells may be modified with engineered antigen receptors that target one or more particular antigens. In cases wherein the NK cells are modified with one or more engineered antigen receptors, the antigen targeted by the monospecific, bispecific or multi-specific antibody, and the antigen targeted by the one or more engineered antigen receptors may or may not be the same antigen. In some cases, the antigen targeted by the monospecific, bispecific or multi-specific antibody, and the antigen targeted by the one or more engineered antigen receptors are different antigens but are associated with the same type of cancer. In some embodiments, the antigen targeted by the monospecific, bispecific or multispecific antibody, and the antigen targeted by the one or more engineered antigen receptors are different antigens, but are each associated with solid tumors.

Among the antigens targeted by the antibodies and/or engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

In some embodiments, an antigen may be presented in the context of an HLA protein. In some embodiments, an antigen may be presented in the context of a HLA-Cw0802 protein. In some embodiments, a target cell expressing an antigen of interest may transgenically express an antigen, transgenically express proteins necessary for antigen presentation, and/or be loaded with antigen. In some embodiments, a target cell is transduced with an HLA-Cw0802 and/or b2m construct comprising a polynucleotide sequence or encoding an amino acid sequence at least or exactly, 80%, 81%, 82%, 83%, 84%, 85%, 86%, 87%, 88%, 89%, 90%, 91%, 92%, 93%, 94%, 95%, 96%, 97%, 98%, 99%, or 100%, identical to one or more of SEQ ID NOs: 318-323. In some embodiments, a target cell is loaded with a peptide comprising epitope GADGVGKSA (SEQ ID NO: 293) and/or GADGVGKSAL (SEQ ID NO: 294).

(SEQ ID NO: 318)
ATGCGGGTTATGGCGCCAAGAACGTTGATCTTGCTCCTGAGTGGGGCGCTCGCTCTGACTGAGACATG
GGCATGCTCCCACTCCATGAGATACTTTTACACCGCCGTGTCTCGCCCAGGACGAGGGGAACCCAGAT
TTATTGCGGTTGGGTACGTGGACGACACACAGTTCGTACAATTCGATAGTGACGCAGCCAGTCCCAGA
GGTGAGCCTCGCGCGCCGTGGGTTGAACAGGAAGGACCAGAGTATTGGGATAGGGAGACCCAAAAATA
CAAACGACAGGCGCAGACAGACAGGGTGTCTTTGAGAAACCTCAGGGGATATTACAATCAAAGCGAGG
CAGGTTCCCACACTTTGCAACGGATGTACGGCTGCGATCTTGGGCCCGATGGACGGCTTCTCCGCGGC
TACAACCAGTTTGCCTATGATGGCAAGGACTATATCGCCCTCAATGAAGACCTCCGCTCTTGGACGGC
GGCGGACAAAGCAGCACAGATCACCCAACGCAAATGGGAGGCTGCCCGCGAAGCTGAGCAGAGGCGAG
CATATTTGGAGGGTACCTGCGTTGAGTGGCTCAGGCGGTACCTCGAGAACGGTAAAAAAACGTTGCAG
AGGGCAGAGCACCCAAAAACCCACGTTACTCATCATCCGGTGTCTGATCACGAAGCGACACTGCGATG
TTGGGCGCTCGGGTTCTACCCCGCTGAGATCACCCTCACATGGCAACGCGATGGAGAGGATCAAACAC
AGGATACCGAACTCGTAGAGACGAGGCCCGCTGGTGACGGGACATTCCAAAAGTGGGCCGCGGTAGTT
GTTCCCAGCGGAGAAGAGCAGCGCTACACCTGCCACGTTCAACACGAAGGACTCCCCGAACCCCTTAC
TCTGAGATGGGGGCCTTCTAGCCAACCAACTATACCTATTGTCGGAATCGTAGCCGGGCTGGCGGTAC
TGGCAGTTCTCGCTGTGCTTGGGGCGGTTATGGCAGTAGTCATGTGCCGCAGAAAGTCTTCAGGTGGT
AAGGGTGGAAGTTGCTCTCAAGCTGCATCATCCAACTCTGCCCAAGGCAGTGATGAGTCACTGATTGC
GTGTAAGGCC
(SEQ ID NO: 319)
ATGTCTCGCTCCGTGGCCTTAGCTGTGCTCGCGCTACTCTCTCTTTCTGGCCTGGAGGCTATCCAGCG
TACTCCAAAGATTCAGGTTTACTCACGTCATCCAGCAGAGAATGGAAAGTCAAATTTCCTGAATTGCT
ATGTGTCTGGGTTTCATCCATCCGACATTGAAGTTGACTTACTGAAGAATGGAGAGAGAATTGAAAAA
GTGGAGCATTCAGACTTGTCTTTCAGCAAGGACTGGTCTTTCTATCTCTTGTACTACACTGAATTCAC
CCCCACTGAAAAAGATGAGTATGCCTGCCGTGTGAACCATGTGACTTTGTCACAGCCCAAGATAGTTA
AGTGGGATCGAGACATG
(SEQ ID NO: 320)
ATGCGGGTTATGGCGCCAAGAACGTTGATCTTGCTCCTGAGTGGGGCGCTCGCTCTGACTGAGACATG
GGCATGCTCCCACTCCATGAGATACTTTTACACCGCCGTGTCTCGCCCAGGACGAGGGGAACCCAGAT
TTATTGCGGTTGGGTACGTGGACGACACACAGTTCGTACAATTCGATAGTGACGCAGCCAGTCCCAGA
GGTGAGCCTCGCGCGCCGTGGGTTGAACAGGAAGGACCAGAGTATTGGGATAGGGAGACCCAAAAATA
CAAACGACAGGCGCAGACAGACAGGGTGTCTTTGAGAAACCTCAGGGGATATTACAATCAAAGCGAGG
CAGGTTCCCACACTTTGCAACGGATGTACGGCTGCGATCTTGGGCCCGATGGACGGCTTCTCCGCGGC
TACAACCAGTTTGCCTATGATGGCAAGGACTATATCGCCCTCAATGAAGACCTCCGCTCTTGGACGGC
GGCGGACAAAGCAGCACAGATCACCCAACGCAAATGGGAGGCTGCCCGCGAAGCTGAGCAGAGGCGAG
CATATTTGGAGGGTACCTGCGTTGAGTGGCTCAGGCGGTACCTCGAGAACGGTAAAAAAACGTTGCAG
AGGGCAGAGCACCCAAAAACCCACGTTACTCATCATCCGGTGTCTGATCACGAAGCGACACTGCGATG
TTGGGCGCTCGGGTTCTACCCCGCTGAGATCACCCTCACATGGCAACGCGATGGAGAGGATCAAACAC
AGGATACCGAACTCGTAGAGACGAGGCCCGCTGGTGACGGGACATTCCAAAAGTGGGCCGCGGTAGTT
GTTCCCAGCGGAGAAGAGCAGCGCTACACCTGCCACGTTCAACACGAAGGACTCCCCGAACCCCTTAC
TCTGAGATGGGGGCCTTCTAGCCAACCAACTATACCTATTGTCGGAATCGTAGCCGGGCTGGCGGTAC
TGGCAGTTCTCGCTGTGCTTGGGGCGGTTATGGCAGTAGTCATGTGCCGCAGAAAGTCTTCAGGTGGT
AAGGGTGGAAGTTGCTCTCAAGCTGCATCATCCAACTCTGCCCAAGGCAGTGATGAGTCACTGATTGC
GTGTAAGGCCGCCACCAATTTCAGCCTGCTGAAACAGGCTGGCGACGTGGAAGAGAACCCCGGACCTA
TGTCTCGCTCCGTGGCCTTAGCTGTGCTCGCGCTACTCTCTCTTTCTGGCCTGGAGGCTATCCAGCGT
ACTCCAAAGATTCAGGTTTACTCACGTCATCCAGCAGAGAATGGAAAGTCAAATTTCCTGAATTGCTA
TGTGTCTGGGTTTCATCCATCCGACATTGAAGTTGACTTACTGAAGAATGGAGAGAGAATTGAAAAAG
TGGAGCATTCAGACTTGTCTTTCAGCAAGGACTGGTCTTTCTATCTCTTGTACTACACTGAATTCACC
CCCACTGAAAAAGATGAGTATGCCTGCCGTGTGAACCATGTGACTTTGTCACAGCCCAAGATAGTTAA
GTGGGATCGAGACATG
(SEQ ID NO: 321)
MRVMAPRTLILLLSGALALTETWACSHSMRYFYTAVSRPGRGEPRFIAVGYVDDTQFVQFDSDAASPR
GEPRAPWVEQEGPEYWDRFTQKYKRQAQTDRVSLRNLRGYYNQSEAGSHTLQRMYGCDLGPDGRLLRG
YNQFAYDGKDYIALNEDLRSWTAADKAAQITQRKWEAAREAEQRRAYLEGTCVEWLRRYLENGKKTLQ
RAEHPKTHVTHHPVSDHEATLRCWALGFYPAEITLTWQRDGEDQTQDTELVETRPAGDGTFQKWAAVV
VPSGEEQRYTCHVQHEGLPEPLTLRWGPSSQPTIPIVGIVAGLAVLAVLAVLGAVMAVVMCRRKSSGG
KGGSCSQAASSNSAQGSDESLIACKA
(SEQ ID NO: 322)
MSRSVALAVLALLSLSGLEAIQRTPKIQVYSRHPAENGKSNFLNCYVSGFHPSDIEVDLLKNGERIEK
VEHSDLSFSKDWSFYLLYYTEFTPTEKDEYACRVNHVTLSQPKIVKWDRDM
(SEQ ID NO: 323)
MRVMAPRTLILLLSGALALTETWACSHSMRYFYTAVSRPGRGEPRFIAVGYVDDTQFVQFDSDAASPR
GEPRAPWVEQEGPEYWDRFTQKYKRQAQTDRVSLRNLRGYYNQSEAGSHTLQRMYGCDLGPDGRLLRG
YNQFAYDGKDYIALNEDLRSWTAADKAAQITQRKWEAAREAEQRRAYLEGTCVEWLRRYLENGKKTLQ
RAEHPKTHVTHHPVSDHEATLRCWALGFYPAEITLTWQRDGEDQTQDTELVETRPAGDGTFQKWAAVV
VPSGEEQRYTCHVQHEGLPEPLTLRWGPSSQPTIPIVGIVAGLAVLAVLAVLGAVMAVVMCRRKSSGG
KGGSCSQAASSNSAQGSDESLIACKAATNESLLKQAGDVEENPGPMSRSVALAVLALLSLSGLEAIQR
TPKIQVYSRHPAENGKSNFLNCYVSGFHPSDIEVDLLKNGERIEKVEHSDLSFSKDWSFYLLYYTEFT
PTEKDEYACRVNHVTLSQPKIVKWDRDM

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include KRAS, TROP-2, NY-ESO, CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular embodiments, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notch1-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAG1B, SUNC1, and LRRN1. Examples of sequences for antigens are known in the art, for example, in the GENBANK® database: KRAS (Accession No. NC_000012.12), CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); KRAS (such as those disclosed in U.S. Pat. No. 10,611,816); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, mutated KRAS, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### E. Suicide Gene

In particular embodiments, a suicide gene is utilized in conjunction with the NK cell therapy to control its use and allow for termination of the cell therapy at a desired event and/or time. The suicide gene is employed in transduced cells for the purpose of eliciting death for the transduced cells when needed. The cells of the present disclosure that have been modified to harbor one or more vectors encompassed by the disclosure that may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

In some cases, the cell therapy may be subject to utilization of one or more suicide genes of any kind when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the suicide gene may be part of a planned protocol for a therapy or may be used only upon a recognized need for its use. In some cases the cell therapy is terminated by use of agent(s) that targets the suicide gene or a gene product therefrom because the therapy is no longer required.

Utilization of the suicide gene may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin.

Examples of suicide genes include engineered nonsecretable (including membrane bound) tumor necrosis factor (TNF)-alpha mutant polypeptides (see PCT/US19/62009, which is incorporated by reference herein in its entirety), and they may be affected by delivery of an antibody that binds the TNF-alpha mutant. Examples of suicide gene/prodrug combinations that may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidylate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be utilized. Other suicide genes include CD20, CD52, inducible caspase 9, purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), EGFRv3, and Thymidine phosphorylase (TP), as examples.

### F. Knockout or Knockdown of Endogenous Genes

In certain embodiments, NK cells of the disclosure may include gene editing of the NK cells to remove 1, 2, 3, 4, 5, 6, 7, 8, 9, 10, or more endogenous genes in the NK cells. In some cases the gene editing occurs in NK cells expressing one or more heterologous transgenes (e.g., CD3, TCR, etc.), whereas in other cases the gene editing occurs in NK cells that do not express a heterologous transgene but that ultimately will express one or more heterologous transgenes, in at least some cases. In particular embodiments, the NK cells that are gene edited are expanded NK cells.

In particular cases, one or more endogenous genes of the NK cells are modified, such as disrupted in expression where the expression is reduced in part or in full. In specific cases, one or more genes are knocked down or knocked out using processes of the disclosure. In specific cases, multiple genes are knocked down or knocked out in the same step as processes of the disclosure. The genes that are edited in the NK cells may be of any kind, but in specific embodiments the genes are genes whose gene products inhibit activity and/or proliferation of NK cells. In specific cases the genes that are edited in the NK cells allow the NK cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, TDAG8, CD5, CD7, SLAMF7, CD38, LAG3, TCR, beta2-microglobulin, HLA, CD73, CREB, CREM, ICER, and CD39. In specific embodiments, the TGFBR2 gene is knocked out or knocked down in the NK cells. In specific embodiments, the CISH gene is knocked out or knocked down in the NK cells. In specific embodiments, the CD38 gene is knocked out or knocked down in the NK cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus. Methods of utilizing a CRISPR system are well known in the art

### IV. ADMINISTRATION OF THERAPEUTIC COMPOSITIONS

The CD3-expressing NK cells and the monospecific, bispecific or multi-specific antibodies are administered to an individual in need thereof, including in such a way as to be in proximity for the anti-CD3 antibody of the bispecific or multi-specific antibody to be able to bind CD3 on the CD3-expressing NK cells. In some cases, the two components are administered separately to an individual, whereas in other cases the two components are complexed together prior to administration, such as in an ex vivo manner. In another embodiment, the NK cells express the antibodies. In some cases, the two components are not pre-complexed prior to administration, but are co-administered by any suitable route of administration, such as by co-infusion to the patient.

Embodiments of the present disclosure concern methods for the use of the compositions comprising NK cells and antibodies provided herein for treating or preventing a medical disease or disorder. The method includes administering to the subject a therapeutically effective amount of the CD3 (±TCR)-modified NK cells with the antibodies, thereby treating or preventing the disease in the subject, including reducing the risk of, reducing the severity of, and/or delaying the onset of the disease. In certain embodiments of the present disclosure, cancer or infection is treated by transfer of a composition comprising the NK cell population and corresponding antibodies. In at least some cases, because of their release of pro-inflammatory cytokines, NK cells may reverse the anti-inflammatory tumor microenvironment and increase adaptive immune responses by promoting differentiation, activation, and/or recruitment of accessory immune cell to sites of malignancy. In certain embodiments, a providing step may comprise culturing the NK cells with antibody molecules for a specific duration of time (e.g., about 5 minutes to about 24 hours or more) and storing the NK cells and the antibody molecules for a period of time (e.g., about 1, 2, 3, 4, 5 days, or greater than 5 days) prior to infusion/administration.

Cancers for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma. In certain embodiments, the cancer is a solid tumor cancer. In certain embodiments, the cancer is pancreatic cancer. In certain embodiments, the cancer is ovarian cancer. In certain embodiments, the cancer is lung cancer. In certain embodiments, the cancer is colorectal cancer. In certain embodiments, the cancer is a cancer associated with KRAS positive tumors.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AMIL); and chronic myeloblastic leukemia.

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition. Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed. Examples of therapies other than those of the present disclosure include surgery, chemotherapy, drug therapy, radiation, hormone therapy, immunotherapy (other than that of the present disclosure), or a combination thereof.

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### V. KITS

Certain aspects of the present disclosure also concern kits comprising compositions of the invention or compositions to implement methods of the invention. In particular embodiments, the kit comprises NK cells, fresh or frozen, and that may or may not have been pre-activated or expanded. The NK cells may or may not already express one or more components of the TCR/CD3 complex. In cases wherein the NK cells do not already express one or more components of the TCR/CD3 complex, the kit may comprise reagents for corresponding transfection or transduction of the NK cells, including reagents such as vectors that express the component(s), primers for amplification of the component(s), and so forth. In some cases, the NK cells may or may not also express one or more heterologous proteins as defined herein, and when they do not, the kit may comprise vectors that express the heterologous protein(s), primers for amplification of the heterologous protein(s), and so forth.

Kits may comprise components which may be individually packaged or placed in a container, such as a tube, bottle, vial, syringe, or other suitable container means. Individual components may also be provided in a kit in concentrated amounts; in some embodiments, a component is provided individually in the same concentration as it would be in a solution with other components. Concentrations of components may be provided as 1×, 2×, 5×, 10×, or 20× or more.

### VI. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Preparation and Effective Use of CD3-Expressing NK Cells

The present example concerns cancer immunotherapeutics as a strategy to redirect the specificity of NK cells against one or more target antigens by ‘arming’ or pre-complexing them with bispecific or multi-specific antibodies, such as either prior to infusion or by co-infusing the two products separately. The NK cells are transduced with one or multiple CD3 chains, including CD3ζ, CD3γ, CD3δ and CD3ε chains and can be from any source. The cells can be expanded or non-expanded, they can be pre-activated with one or more inflammatory cytokines, such as IL12/15/18, and/or they can be genetically modified to express one or more heterologous proteins, including, for example, engineered antigen receptors, such as chimeric antigen receptor or a TCR, and/or a cytokine gene and/or a homing/chemokine receptor.

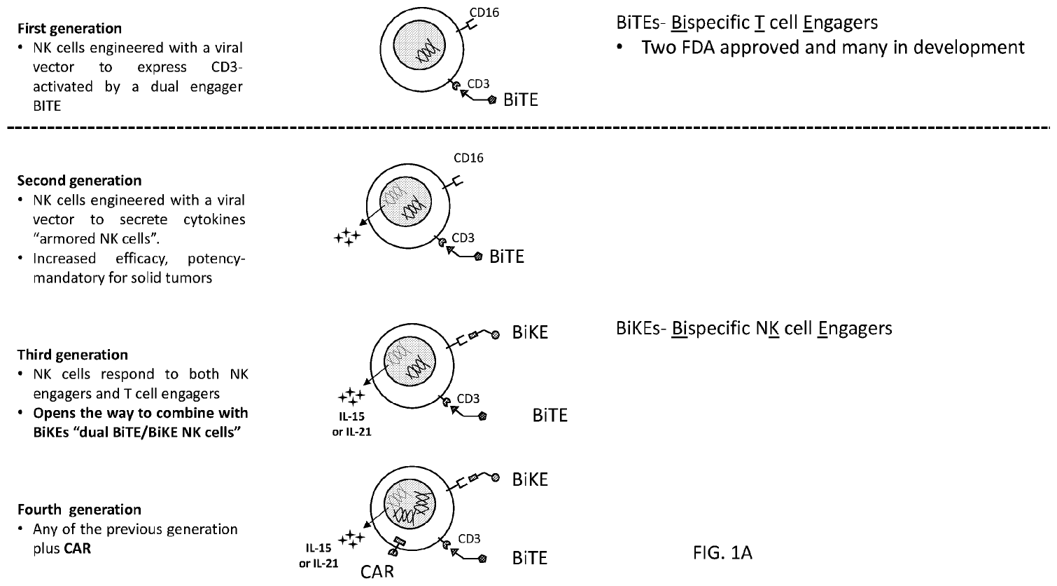

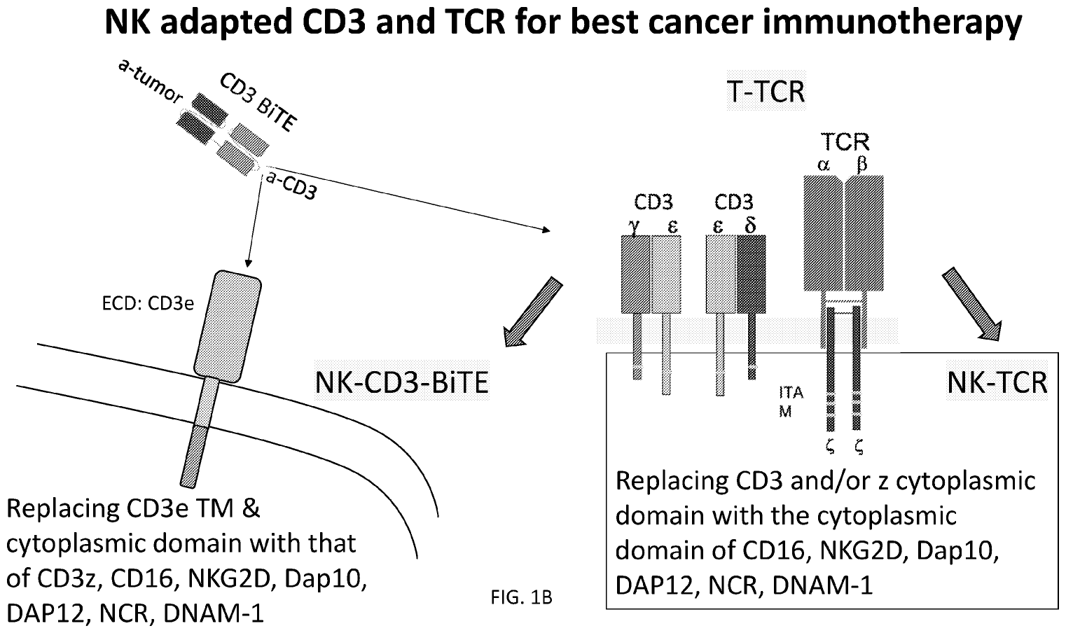

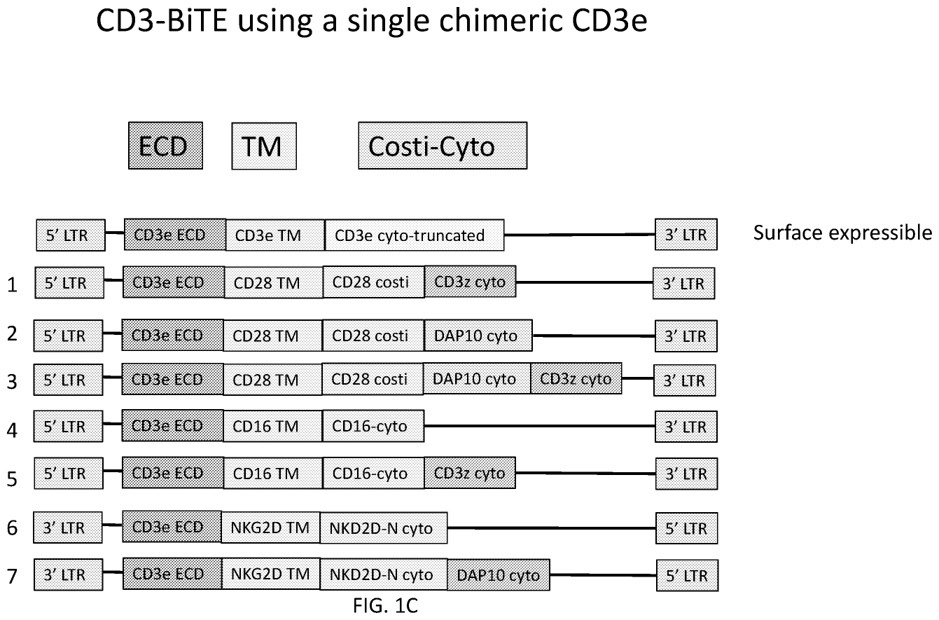

FIGS. 1A and 1B illustrate different embodiments of NK cells engineered to be utilized with bispecific or multi-specific antibodies. As shown in FIG. 1A, in a first generation of NK cells, the cells are engineered to express CD3 that may be activated with a bispecific or multi-specific antibody, including a bispecific T cell engager (BiTE) that comprises an anti-CD3 antibody that binds heterologous CD3 expressed on the surface of the NK cells. In another embodiment, CD3-expressing NK cells are able to be bound by a BiTE that comprises an anti-CD3 antibody, and the NK cells are also expressing one or more particular cytokines (e.g., IL-15 and/or IL-21), resulting in increased efficacy and potency that are particularly useful for treating solid tumors. In another embodiment, the NK cells are engineered to express not only CD3 to be able to be activated by a BiTE that comprises an anti-CD3 antibody but also are utilized with a bispecific or multi-specific antibody (e.g., bispecific NK cell engager, or BiKE) that comprises an antibody that binds a surface antigen naturally present on NK cells, such as CD16, CS1, CD56, NKG2D, NKG2C, DNAM, 2B4, CD2, an NCR, or KIR, for example. In this manner, the NK cells respond to both NK engagers and T cell engagers. In another embodiment, the NK cells in addition to expressing CD3 to engage with T cell engagers also express an engineered antigen receptor, such as a CAR or engineered TCR.

FIG. 1B illustrates different embodiments wherein the NK cells are modified to express both CD3 and a TCR. On the right, T cell TCR is illustrated having α and β chains with an antigen binding site wherein the TCR is complexed with CD3ζ to effect signal transduction. The T cell TCR is co-complexed with two CD3ε chains, a CD3δ chain, and a CD3γ chain. In some embodiments, the NK cells express a TCR in which one or more of the cytoplasmic domains of any of the CD3 molecules is a heterologous intracellular domain, such as one from CD16, NKG2D, DAP10, DAP12, NCR, and DNAM-1. As shown on the left of FIG. 1B, the NK cells are configured to express a CD3 co-receptor component, and in one example the CD3 component is CD3ε. In such a case, a standard BiTE (top left that comprises an antibody against a tumor antigen and an antibody against CD3) normally utilized with T cells that naturally express CD3 may be utilized in conjunction with the CD3-expressing NK cells. In this particular example, the NK cells express a polypeptide that comprises the extracellular domain of CD3ε (although the extracellular domains of other CD3 components may be utilized) and the extracellular domain of CD3ε is linked to a transmembrane domain and/or cytoplasmic domain of another molecule, such as the transmembrane domain and/or cytoplasmic domain of CD3ζ, CD16, NKG2D, DAP10, DAP12, NCR, or DNAM-1, for example.

As described above, FIG. 1C. schematically depicts the generation of surface-expressible single chimeric CD3 constructs that can be used in conjunction with anti-CD3 BiTEs. As one example, the CD3 epsilon extracellular domain (ECD) is fused with CD28, CD16, or NKG2D transmembrane (TM), and CD28, CD16, or NKG2D intracellular domain (ICD), with or without CD3 zeta and/or DAP10 intracellular domains. In one example, the constructs are encompassed within the Moloney murine virus-derived SFG retroviral vector backbone, which may be used with packing plasmids for viral production. In instances wherein the CD3-BiTE is used with such constructs in FIG. 1C, the antibody will bind the extracellular domain of CD3ε accordingly.

Embodiments of the disclosure utilize part or all of the CD3 receptor complex. As illustrated in FIGS. 2A and 2B, the NK cells may be transfected or transduced with full length CD3zeta, CD3 gamma, CD3 delta, and CD3 epsilon. In such cases, the full length of each of CD3zeta, CD3 gamma, CD3 delta, and CD3 epsilon include the extracellular domain, the transmembrane domain, and the intracellular domain. When the different components of the receptor are expressed from the same vector, they may be configured to be produced as separate polypeptides, such as utilizing IRES or 2A elements. In any case, any expression construct may be configured to express one or more cytokines, including at least IL-15.

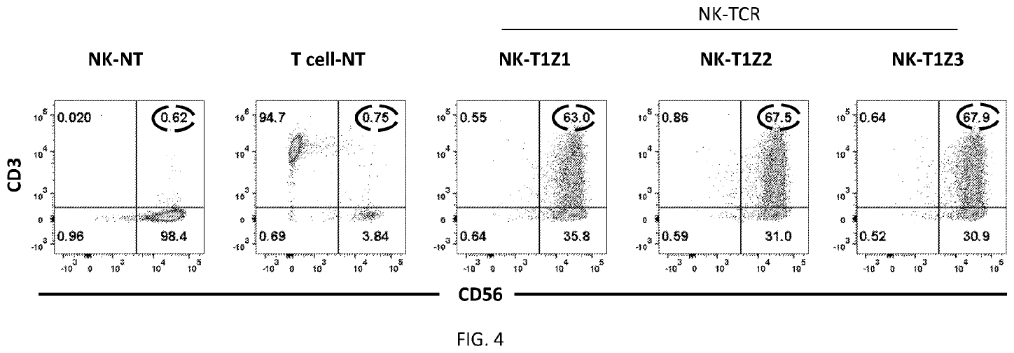

FIG. 4 demonstrates CD3 expression on NK cells after CMV TCR complex transduction, at day 4. The figure provides FACS plots showing CD3 expression on NK cells 4 days after CMV TCR complex transduction. Non transduced (NT) NK cells (CD56+ CD3−) serve as a negative control and T cells (CD3+ CD56−) serve as a positive control.

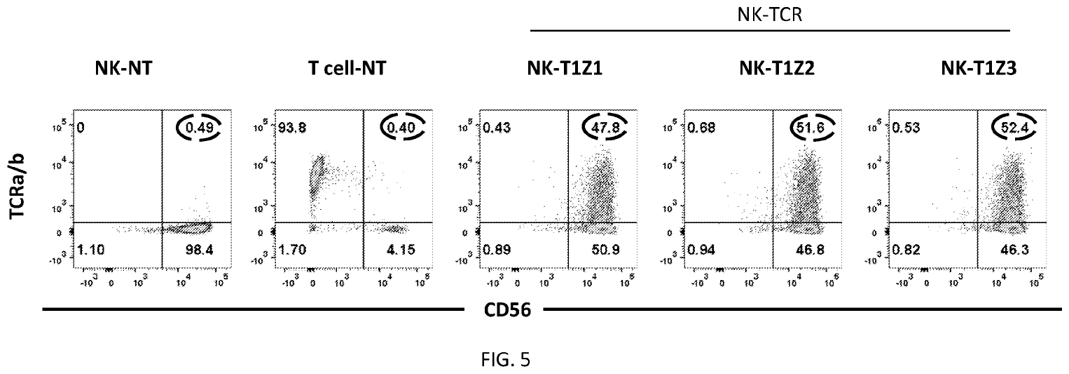

FIG. 5 demonstrates TCR expression on NK cells after CMV TCR complex transduction of NK, day 4. In particular, provided are FACS plots showing TCRa/b expression on NK cells 4 days after CMV TCR complex transduction. Non transduced (NT) NK cells (CD56+ CD3−TCRa/b−) serve as a negative control and T cells (CD3+TCRa/b+CD56−) serve as a positive control.

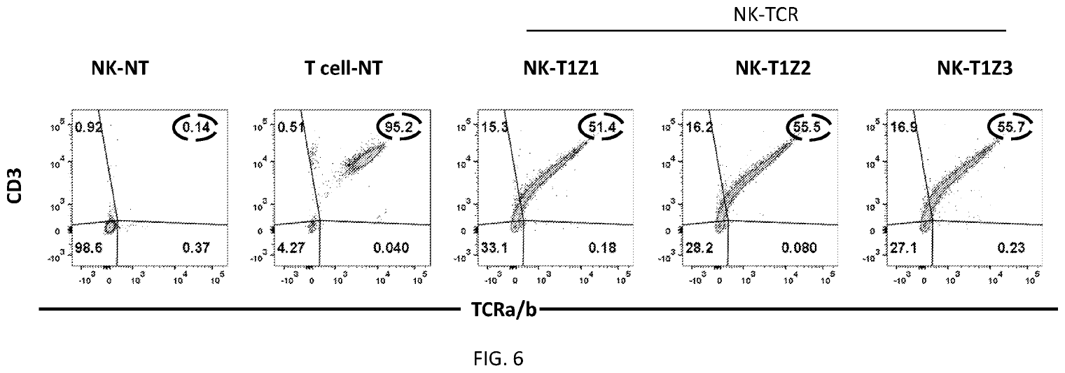

FIG. 6 shows TCR/CD3 expression on NK after CMV TCR complex transduction, day 6. Specifically, FACS plots show dual CD3 and TCRa/b expression on NK cells 6 days after CMV TCR complex transduction. Non transduced (NT) NK cells (CD56+ CD3−TCRa/b−) serve as a negative control and T cells (CD3+TCRa/b+ CD56−) serve as a positive control.

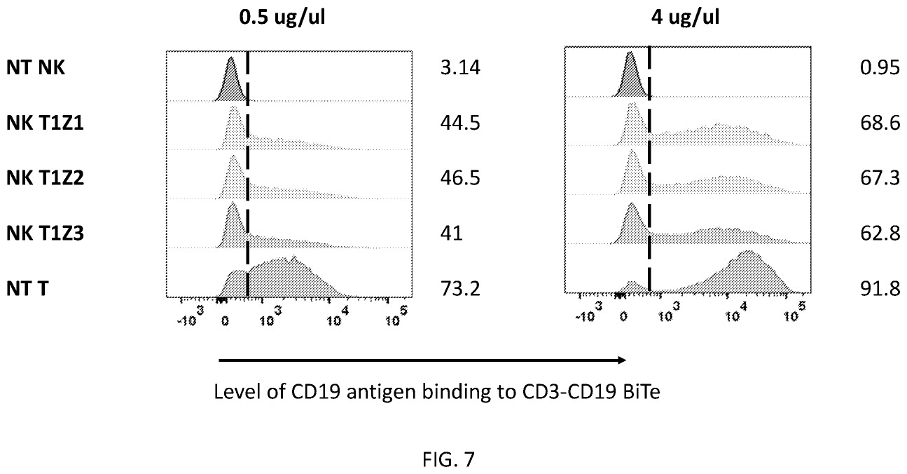

In FIG. 7, shown are the binding of CD3-CD19 BiTE on NK cells through the CD3/TCR at different concentrations. Specifically, the various cells (non-transduced (NT) NK cells, T cells, or the three different NK-TCR cells) were incubated with blinatumomab a CD3-CD19 bispecific engager (BiTe) for one hour at 37° C. using two different concentrations (0.5 μg/μl or 4 μg/μl). Then, a biotin-labeled CD19 antigen (CD19 CAR Detection Reagent from Miltenyi Biotech™) was added for 20 min followed by an anti-biotin antibody for 15 min at room temperature. This strategy was used to detect any BiTe engaged with a CD3+ cell. The Histograms in FIG. 7 show the level of CD19 binding to CD3-CD19 bispecific engager (BiTe) that correlates with CD3 expression on NK-TCR and T cells.

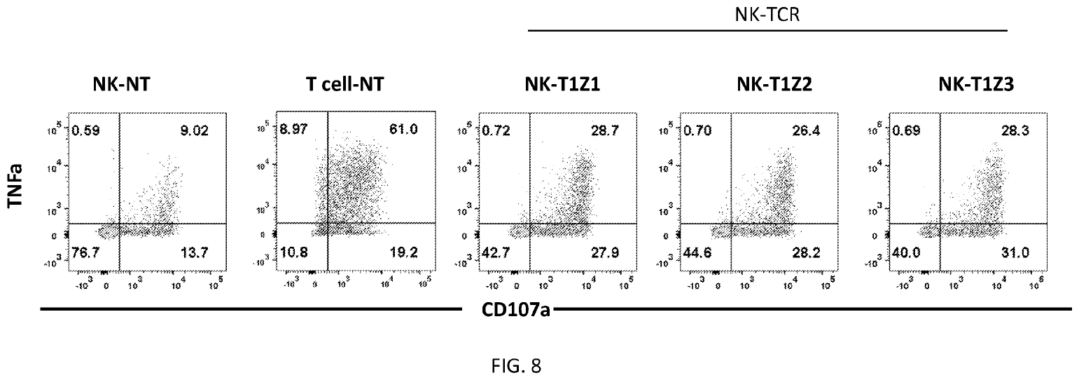

FIG. 8 shows NK-TCR cytokine production after stimulation with a plate-bound CD3 antibody. In particular, CD3-OKT3 clone 20 μg/ml was incubated overnight in flat bottom 96-well plates at 4° C. to form a plate-bound antigen. The next day, T cells or NK cells (NT or TCR-transduced) were added to the wells for 4 hrs and with Brefeldin A (that prevents the cytokine from being released, trapping it in the cytoplasm such that it can be detected by intracellular cytokine staining). They were then harvested for surface and intracellular staining to assess cytokine production and degranulation (TNFα and CD107a). FACS plots in FIG. 8 show TNFα and CD107a double-positive populations in NK cells transduced with TCR. Non-transduced (NT) NK cells (CD56+ CD3−) serve as a negative control and T cells (CD3+ CD56-) serve as a positive control.

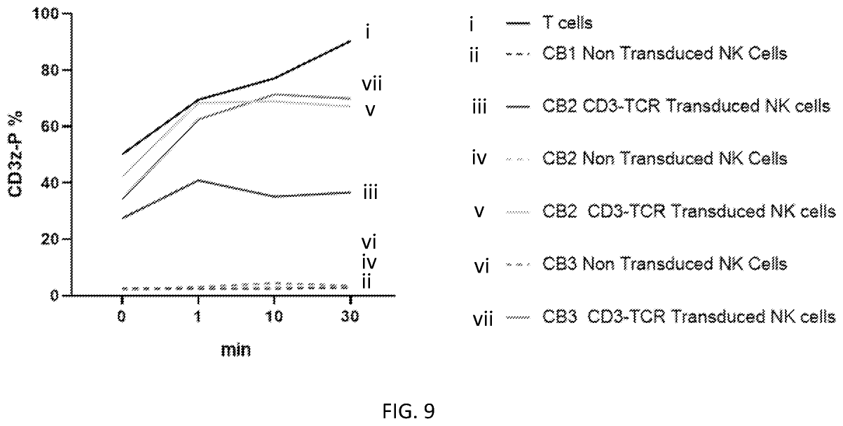

FIG. 9 demonstrates phosphorylation of CD3ζ in NK TCR/CD3 cells after crosslinking CD3. The various cells tested included non-transduced (NT) NK cells; non-transduced (NT) T cells, or three different CD3-TCR transduced NK cells (where CD1, CD2, or CD3 represent different donors). Each of the NK cell groups were transduced with CD3ZFLGDEFL15 (see FIGS. 2A and 2B). The NK cells were incubated with CD3 OKT3 clone (Miltenyi, 130-093-387) at 20 μg/ml concentration for 20 min on ice. Cells were then cross-linked with Fab2 IgG1 antibody for various time points and stained to check for CD3z phosphorylation. This analysis of CD3ζ is useful because, as an internalization signal from the surface, it would only be able to be crosslinked with a CD3 monoclonal antibody if the NK cells expressed it. NK cells that are not transduced with CD3 will not show any phosphorylation or activation after the stimulation.

NK cells transduced with CD3-TCR also show basal level of tonic signaling, which increases upon stimulation with CD3 OKT3 and is similar to T cells, while non-transduced NK cells did not show any CD3ζ phosphorylation neither at basal nor upon CD3 OKT3 stimulation.

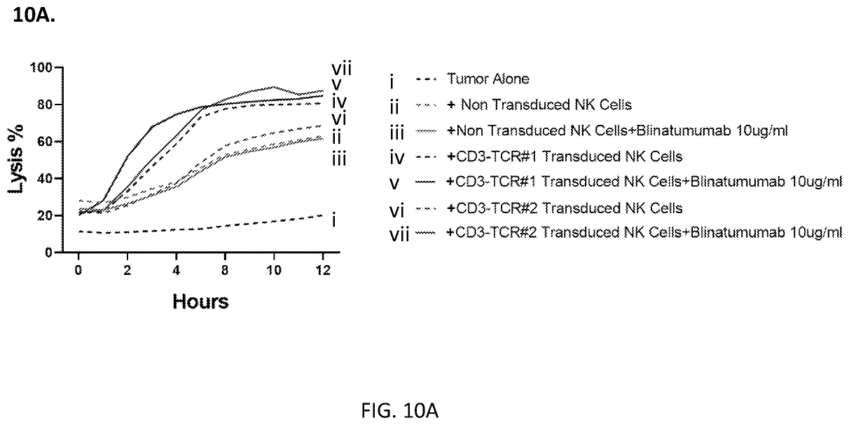

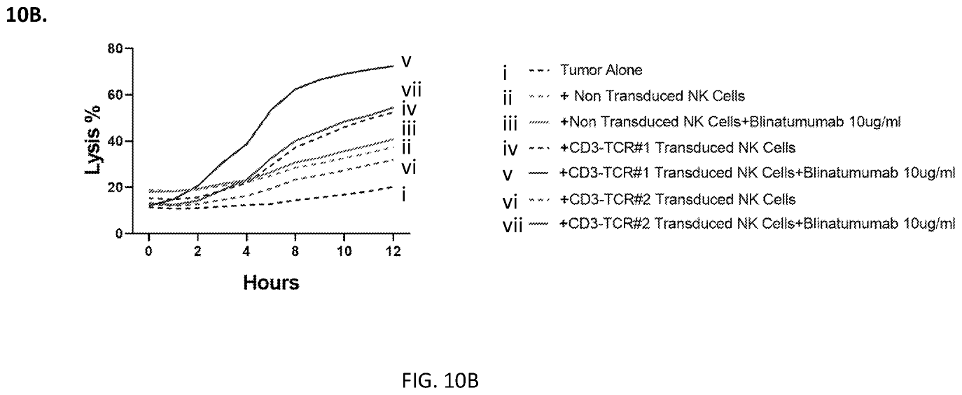

FIG. 10 shows that pre-culturing CD3-CD19 BiTEs with TCR/CD3-expressing NK cells increased its killing activity against Raji cells. NK cells were either transduced with CD3-TCR #1 (CD3ZFLGDEFL15 (see FIGS. 2A and 2B)) or CD3-TCR #2 (Z2, also called CD3ZGDEFL8SP21CD8, that includes full length CD3ζ, full length CD3γ, full length CD3δ, and full length CD3 ε linked to membrane bound IL21 (with CD8 transmembrane domain for the membrane bound IL21). NK cells transduced with the CD3/TCR constructs or non-transduced NK cells were loaded with Blinatumumab and incubated for 1 hour and washed with PBS. They were then co-cultured with CD19+ B cell lymphoma cells at different Effector cell:Target cell ratios (FIG. 10A is a 1:1 ratio, and FIG. 10B is a 1:5 ratio) for various time points. As utilized herein, Effector cells are the CD-3-TCR NK Cells, and Target cells are the Raji cells. Blinatumumab-loaded CD3-TCR transduced NK cells showed enhanced anti-tumor activity compared to Blinatumumab-loaded non-transduced NK cells or CD3/TCR transduced NK cells, but not loaded with Blinatumomab at both E:T ratios.

### Example 2

### NY-ESO TCRs in NK Cells

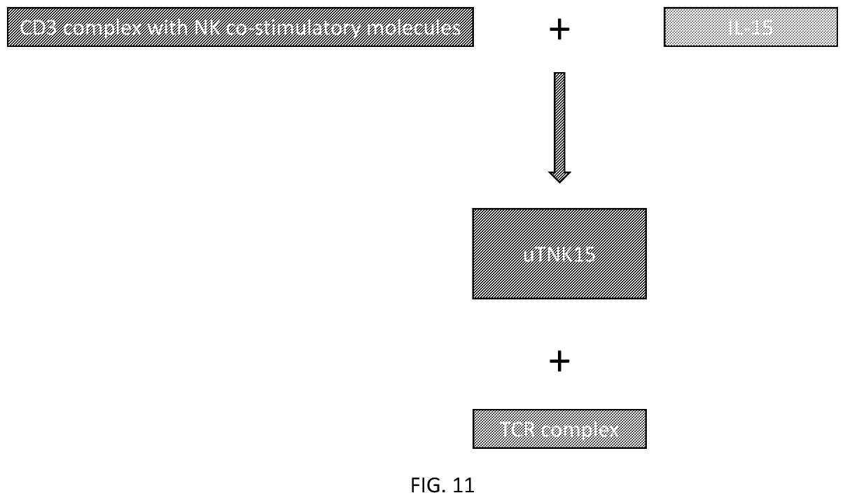

The present examples concern generation and use of NY-ESO TCRs in NK cells. In FIG. 11, there is one example for production of the cells. The schematic overview shows one case wherein the NK cells are first transduced with the uTNK15 construct that incorporates signaling domains from the CD3 complex, NK costimulatory molecules and IL-15, followed by a second transduction step that introduces the TCR molecule, thus generating NK cells that co-express CD3 and NK signaling molecules, IL-15, and a TCR complex. In one embodiment, NK cells were derived from cord blood and were expanded with irradiated (100 Gy) universal antigen presenting cells (uAPC) feeder cells (2:1 feeder cell:NK ratio) and recombinant human IL-2 (200 U/ml) in complete media. To generate a universal T cell-like NK cell (uTNK15 cells) that can secrete IL-15, NK cells were purified and transduced with a retroviral construct containing a CD3 complex with NK co-stimulatory molecules and an IL-15 gene 4 days after isolation. Forty-eight hours after the initial transduction, NK cells expressing uTNK15 were then transduced with a TCR targeting an antigen of choice.

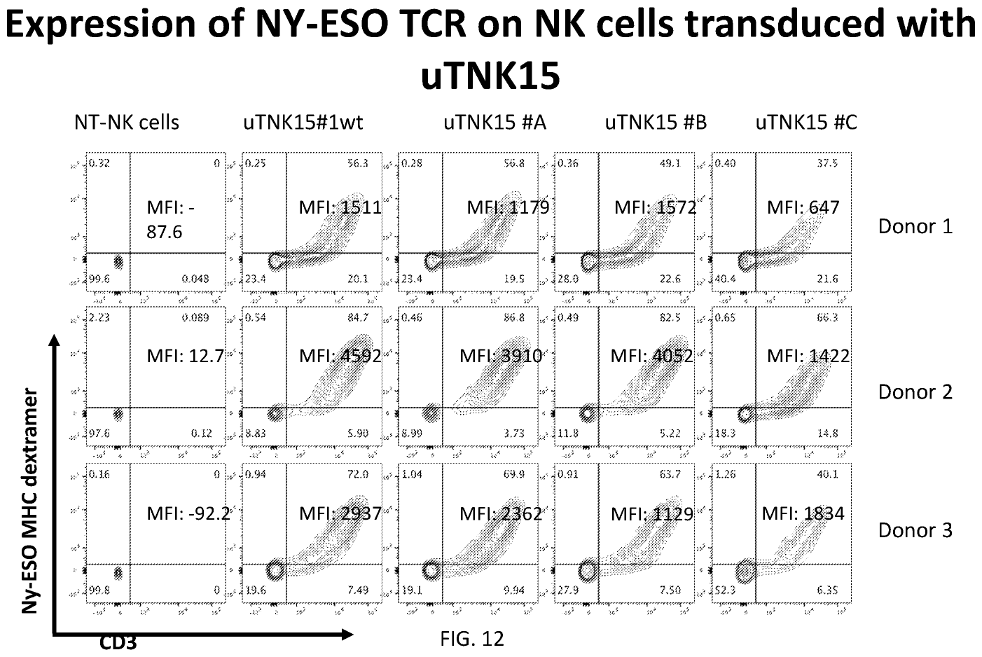

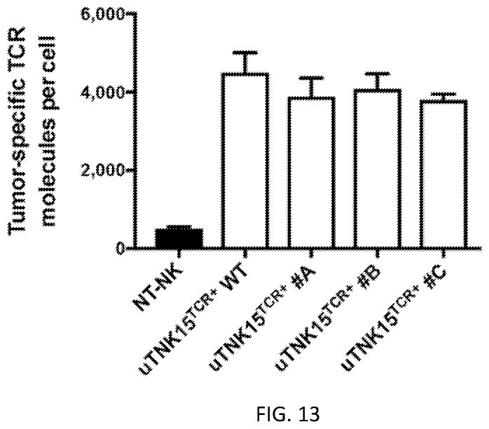

Expression of NY-ESO TCR on NK cells transduced with uTNK15 is shown in FIG. 12. NK cells were derived from cord blood and were expanded with irradiated (100 Gy) universal antigen presenting cells (uAPC) feeder cells (2:1 feeder cell:NK ratio) and recombinant human IL-2 (200 U/ml) in complete media. To generate a universal T cell-like NK cell that can secrete IL-15, NK cells were purified and transduced with a retroviral construct containing a CD3 complex with NK co-stimulatory molecules and an IL-15 gene 4 days after isolation. Forty-eight hours after the initial transduction, uTNK15 cells were then transduced with a TCR complex targeting an antigen of choice. Forty-eight hours later, flow cytometry was used to assess the expression of CD3 and NY-ESO TCR on the various uTNK15 constructs. Non transduced (NT) NK cells served as negative control. CD3 and NY-ESO TCR were highly expressed on all uTNK15 cells compared to NT NK cells. The number of tumor specific TCR molecules expressed on TCR engineered NK cells using the various TCR constructs are provided in FIG. 13, and NT NK cells were used as negative control.

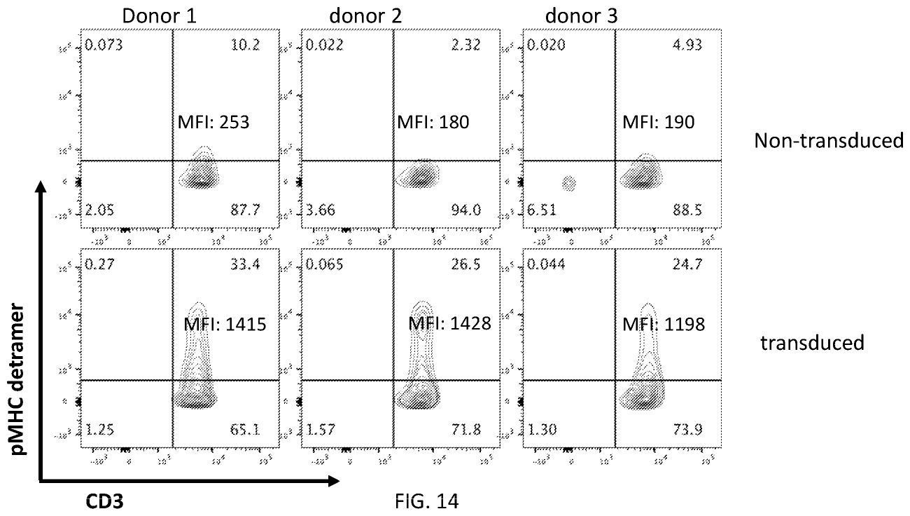

FIG. 14 demonstrates NY-ESO TCR expression on non-transduced and transduced T cells. T cells were isolated from cord blood (the same donor as NK cells to serve as a paired positive control) and were activated with CD3/CD28 microbeads at a concentration of 25 μl/1 million for 48 hours in RPMI complete media. T cells were then transduced with a retroviral construct containing NY-ESO TCR. Forty-eight hours after transduction, flow cytometry revealed that NY-ESO TCR was highly expressed on transduced T cells compared to non-transduced T cells.

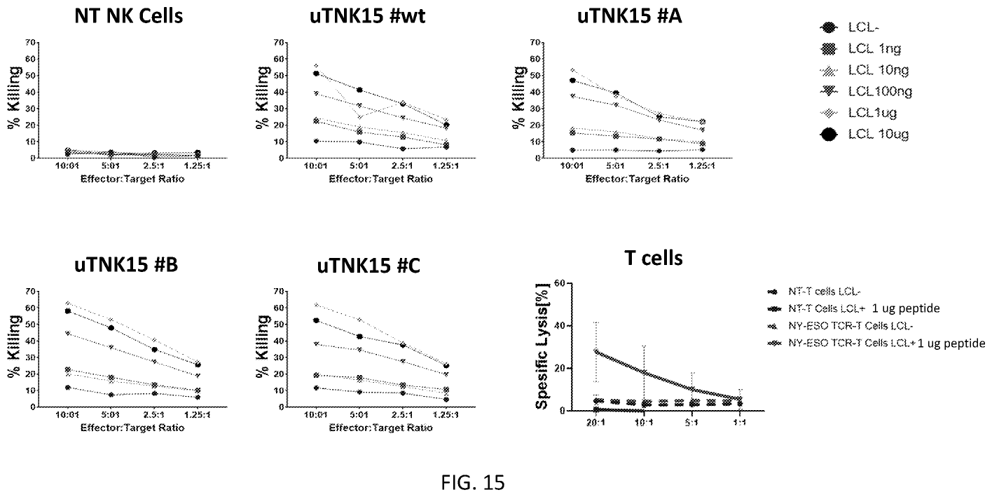

NK cells transduced with NY-ESO TCR kill NY-ESO peptide-pulsed target cells in a dose-dependent manner (FIG. 15). Chromium ⁵¹CR killing assay was performed 7 days following TCR transduction to determine the killing capacity of TCR-engineered NK and T cells against LCL cells loaded with different concentrations of NY-ESO peptide for 2 hours. NY-ESO TCR transduced uTNK15 cells show enhanced killing of peptide-pulsed LCL cells compared to non-transduced NK cells. NY-ESO TCR transduced T cells show enhanced killing of peptide-pulsed LCL cells compared to non-transduced T cells.

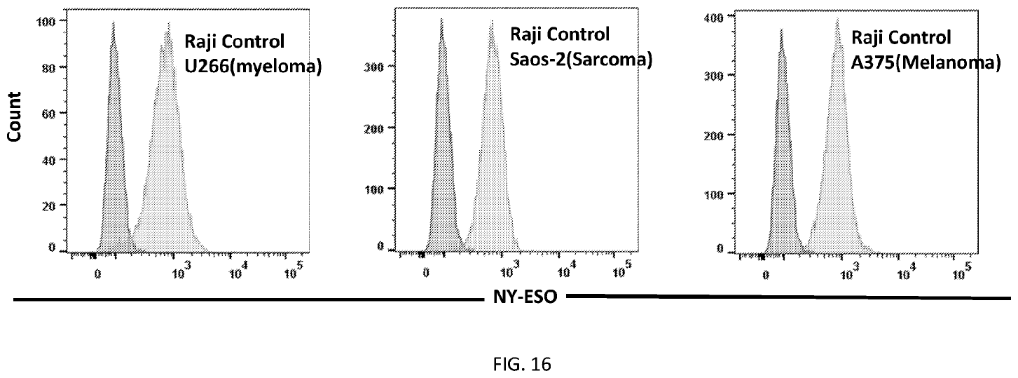

FIG. 16 shows that NY-ESO is expressed endogenously on myeloma, sarcoma, and melanoma cell lines. Flow cytometry was used to determine the expression of NY-ESO on U266 (myeloma), Saos-2 (Sarcoma), and A375 (melanoma) cell lines. U266, Saos-2, and A375 cell lines showed higher levels of NY-ESO expression compared to the Raji cell line which served as negative control.

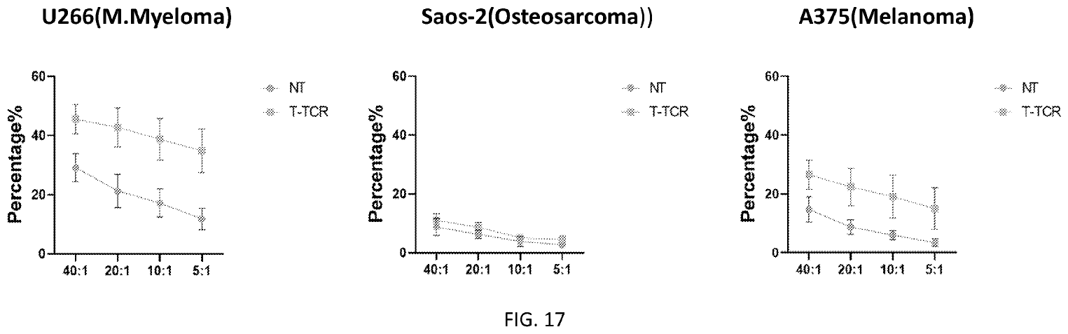

NY-ESO TCR-transduced T cells kill NY-ESO expressing tumor targets at higher E:T ratios (FIG. 17). Chromium ⁵¹CR killing assay was performed 7 days following TCR transduction to determine the killing capacity of NY-ESO TCR-engineered T cells against NY-ESO expressing myeloma, osteosarcoma and melanoma cell lines. NY-ESO TCR transduced T cells show enhanced killing of NY-ESO positive cell lines compared to non-transduced T cells.

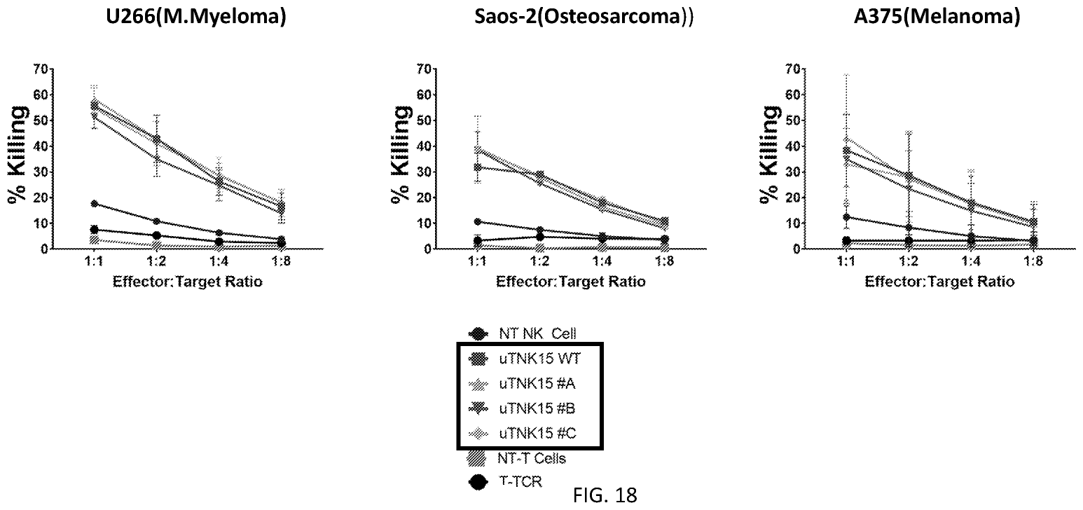

FIG. 18 demonstrates that NY-ESO TCR transduced NK cells kill NY-ESO expressing tumor targets even at low E:T ratios. Chromium ⁵¹CR killing assay was performed 7 days following TCR transduction to determine the killing capacity of NY-ESO TCR-engineered NK cells against NY-ESO-expressing myeloma, osteosarcoma and melanoma cell lines. NY-ESO TCR-transduced NK cells show enhanced killing of NY-ESO positive cell lines compared to non-transduced NK cells even at very low effector:target ratios.

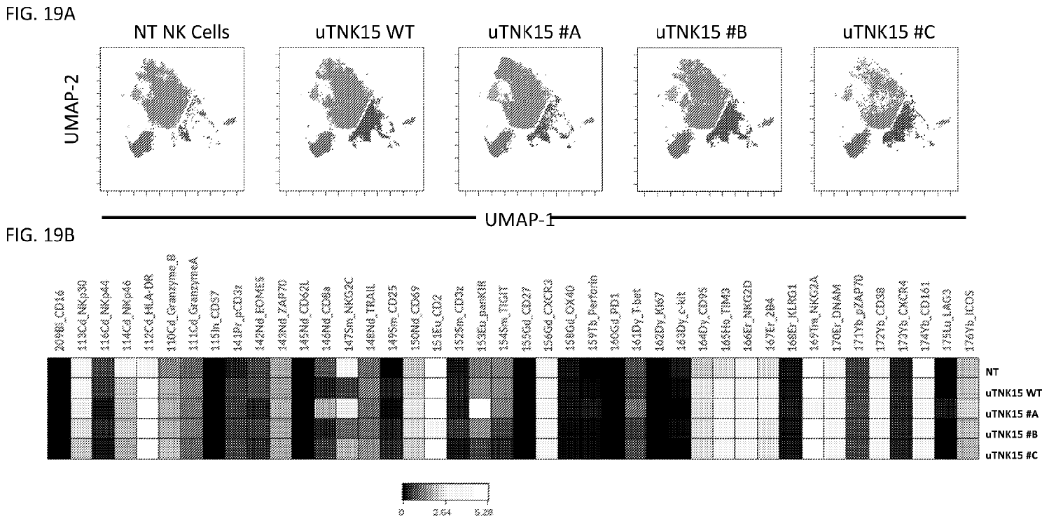

FIG. 19 shows that NY-ESO transduced NK cells have a similar phenotype to NT NK cells. CytoF imaging revealed that non-transduced NK cells and NY-ESO TCR transduced uTNK15 cells share a similar phenotype. FIG. 19A shows a u-map plot with similar clusters, and FIG. 19B shows a heat map with similar expression of various markers on NT and NY-ESO TCR transduced uTNK15 cells.

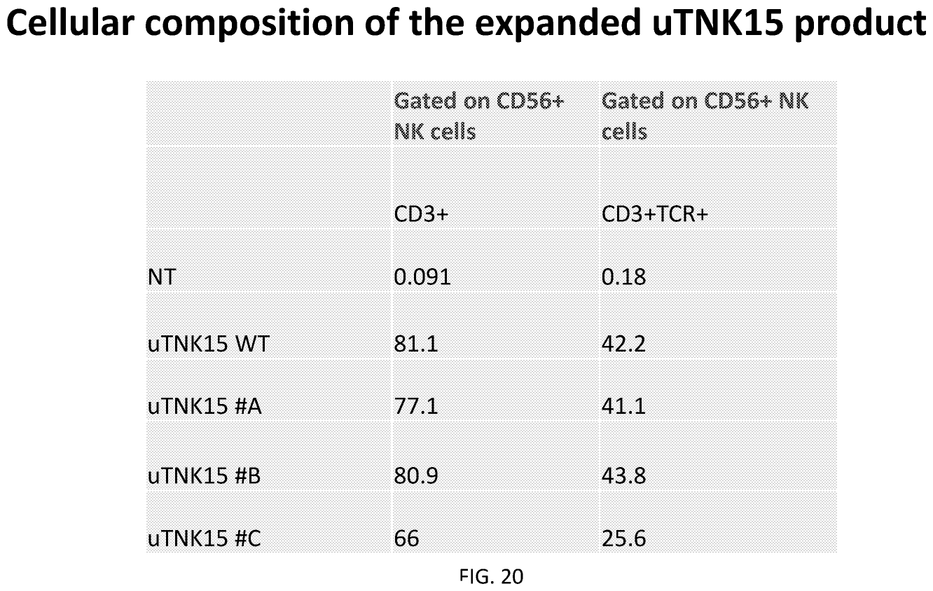

FIG. 20 provides a table representing the percentage of CD3+ and CD3+TCR+NK cells in each uTNK15 product. Flow cytometry was used to assess the composition of single positive CD3 NK cells (CD3+) and double positive CD3/TCR NK cells (CD3+TCR+). Non transduced NK cells are comprised of less than 1% CD3+ and CD3+TCR+ NK cells, while the TCR transduced uTNK15 cell products are comprised of over 60% CD3+ and over 25% CD3+TCR+ NK cells.

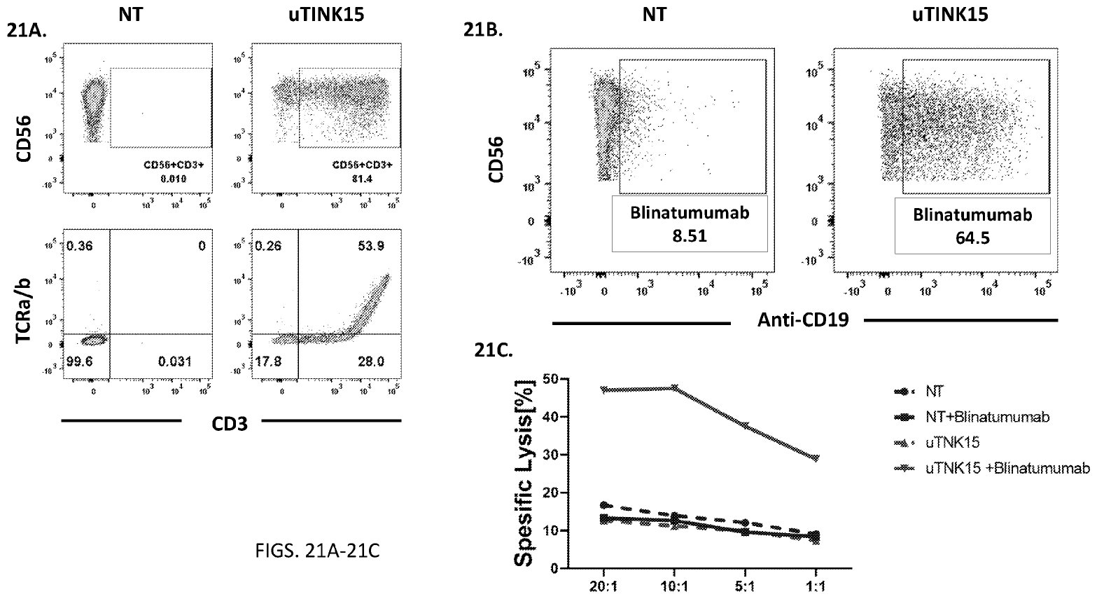

FIG. 21A provides FACS plots that show successful CD3 expression on NK cells 4 days after transduction with TCR constant alpha-beta (TCRCab; TCR6 construct). Non transduced (NT) NK cells (CD56+ CD3−) serve as negative control. In FIG. 21B, NT NK and uTNK15 NK cells were incubated with Blinatumumab, a CD3-CD19 bispecific engager (BiTe), for one hour at 37° C. using 10 μg/μl. Then, a biotin-labeled CD19 antigen (CD19 CAR Detection Reagent from Miltenyi) was added for 20 min, followed by an anti-biotin antibody for 15 min at room temperature. This strategy was used to detect any BiTe engaged with a CD3+ cell. The histograms in this figure are showing the level of CD19 binding to CD3-CD19 bispecific engager (BiTe) that correlates with CD3 expression on uTINK15 NK cells. In FIG. 21C, CD3/TCR transduced or non-transduced NK cells were loaded with Blinatumumab and incubated for 1 hour and washed with PBS. They were then co-cultured with LCL cells at different E:T ratios (A.1:1,B.1:5) for various time points. Blinatumumab-loaded CD3-TCR transduced NK cells showed enhanced anti-tumor activity compared to Blinatumumab-loaded non-transduced NK cells or CD3/TCR transduced NK cells but not loaded with Blinatumumab at both E:T ratios.

### Example 3

### NY-ESO TCRs in CD3 Expressing NK Cells In Vivo

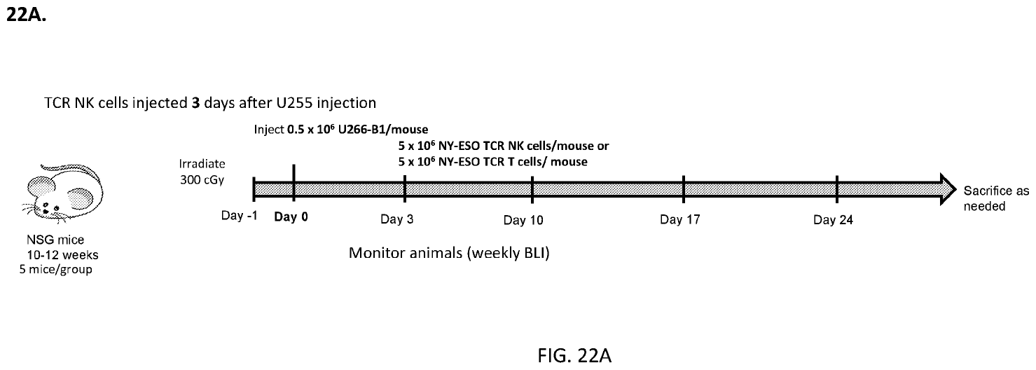

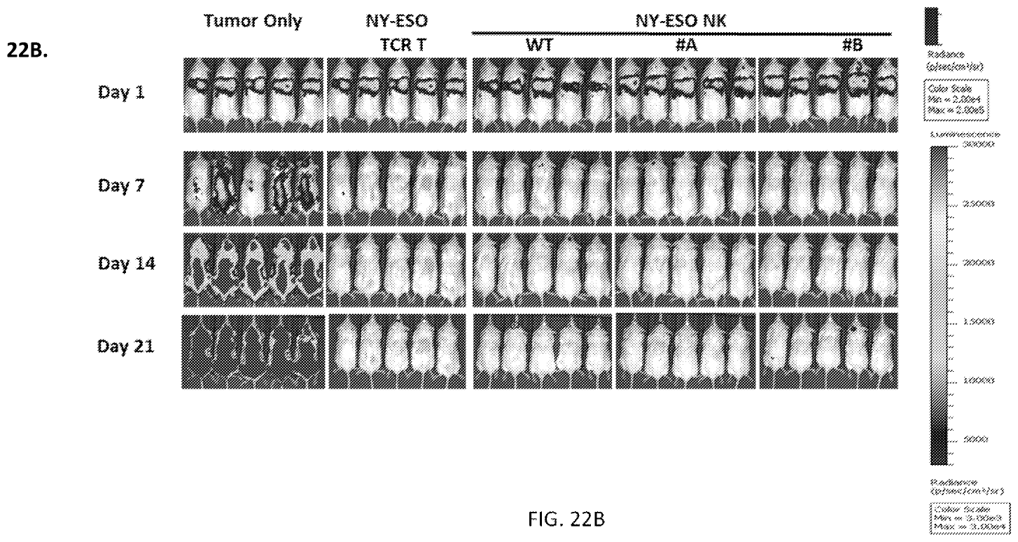

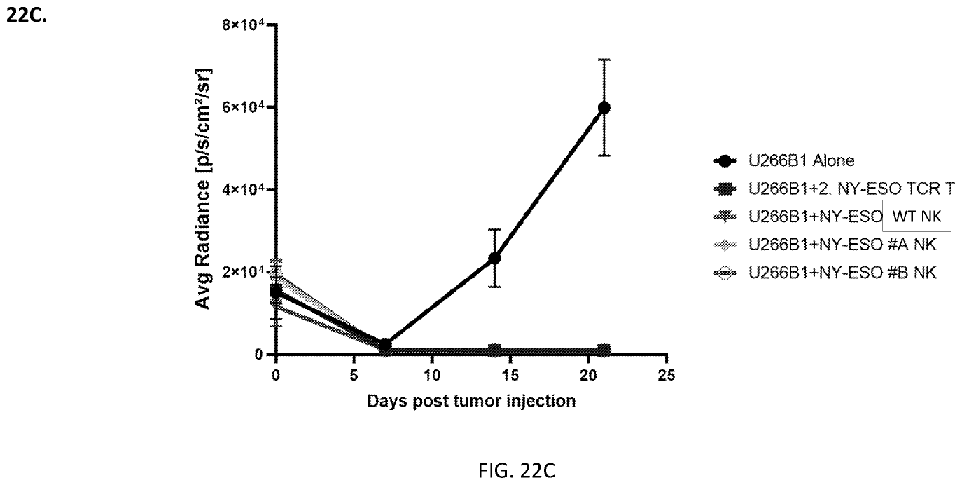

As shown in FIGS. 22A-22C, NK cells comprising constructs described herein were tested in-vivo and found to robustly inhibit tumor growth. Shown in FIG. 22A is a schematic outlining the experimental procedure performed. In brief, NSG mice were irradiated with 300 cGy on day −1, then on day 0 individual mice received tail vein injections of 0.5×10⁶ U266-B1 cells (a myeloma cell line that expresses both HLA-A2 and NY-ESO antigens) that were transduced with FireFlyluciferase (FFluc), on day 3 mice were infused with 5×10⁶ effector cells (NY-ESO TCR NK cells with WT, #A, or #B UT-NK15-NY ESO TCR constructs respectively; WT refers to wild type CD3 molecules with IL-15; #A refers to CD3-CD28 with IL-15 (e.g., UT-NK15-28); and #B refers to CD3-DAP10 with IL-15 (e.g., UT-NK15-DAP10); or NY-ESO TCR T cells), animals were then monitored over time and sacrificed as appropriate (N=5 mice per group). FIG. 22B displays the results of the monitoring of the experiment described in FIG. 22A as a function of bioluminescent imaging over time (displayed are representative images from day 1, day 7, day 14, and day 21 respectively). FIG. 22C is a graphical quantification of the bioluminescence average radiance displayed in FIG. 22B, the Y axis denotes average radiance in p/s/cm²/sr, while the X axis denotes time.

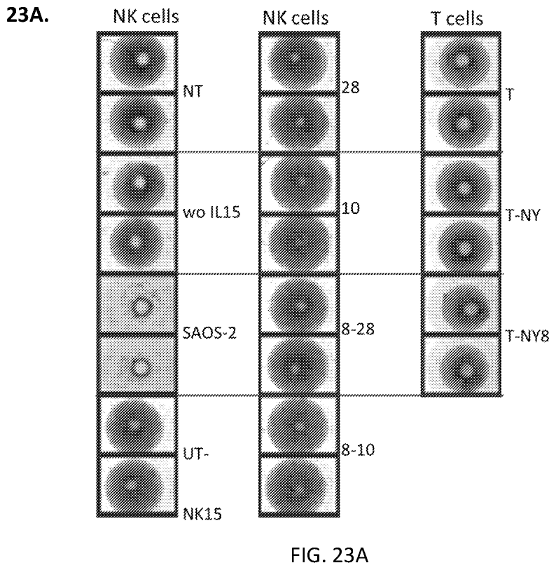

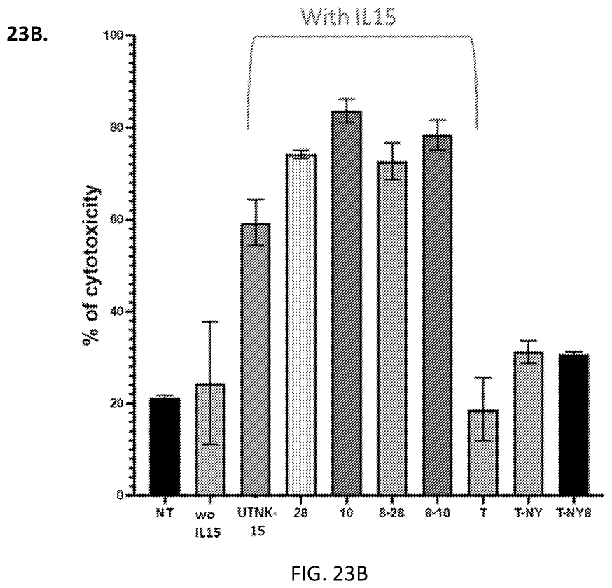

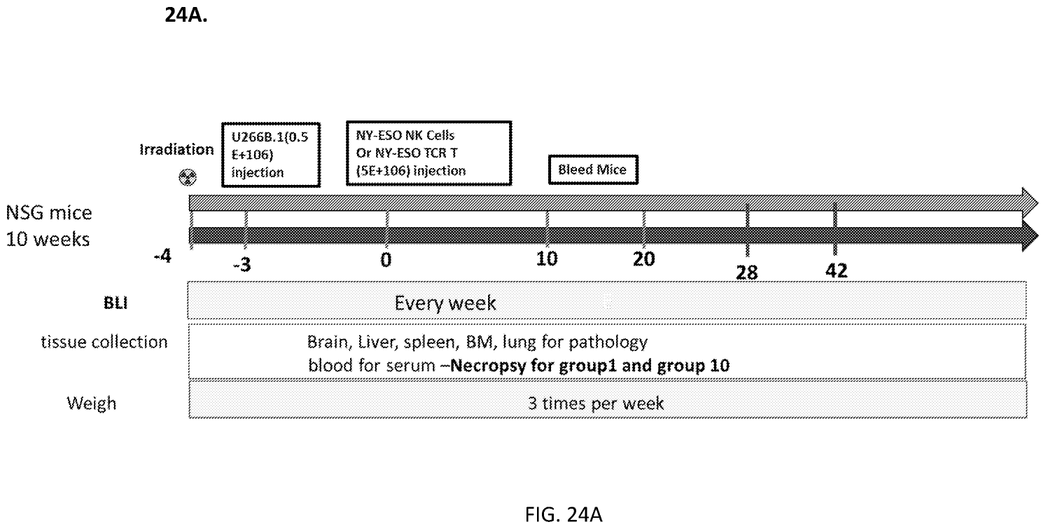

As shown in FIGS. 23A-B the in vitro activity of effector cells (e.g., NK cells or T cells) comprising NY-ESO targeted TCRs and UT-NK15 constructs was tested. FIG. 22A are images of spheroids formed by osteosarcoma tumor cell line Saos-2 that were used to test the activity of NY-ESO1-specific TCR expressing NK and T cells cytotoxicity. Saos-2 cells were stably transduced to express GFP; 10,000 of these cells were seeded per well in a 96 well plate overnight and 40,000 of NK or T cells were then added. Images of the coculture were scanned over time and analyzed by the IncuCyte® cell analysis system. Shown in FIG. 22B is a graph displaying the percentage of cytotoxicity (Y axis) for effector cells captured from representative images after 3 days of co-culture. NK cells were co-transduced with NY-ESO-TCR, and the UT-NK15 signaling complex co-expressing different co-stimulatory molecules fused to the CD3ζ signaling chain (e.g., UTNK-15-28, or UTNK-15-DAP10). T cells were only transduced with NY-ESO TCR. Abbreviations in the graph are as follows: 28=CD3ζ fused to a CD28 co-stimulatory domain; 10=CD3ζ fused to a Dap10 co-stimulatory domain; 8=CD8 alpha/beta co-receptor as part of the NY ESO TCR construct; wo IL-15=the construct only contains CD3 zeta, epsilon, gamma and delta without co-stimulation or IL-15. The best in vitro cytotoxicity was observed with TCR NK cells expression UTNK15 with CD28, or DAP10 costimulatory domains fused to CD3ζ (e.g., UTNK-15-28, or UTNK-15-DAP10; SEQ ID NO: 121 and SEQ ID NO: 119 respectively) when compared to NK cells transduced with CD3 complex only or the UT-NK15 without a co-stimulatory domain. The addition of the CD8 alpha/beta coreceptor to the TCR did not significantly improve on the cytotoxicity of NK or T cells.

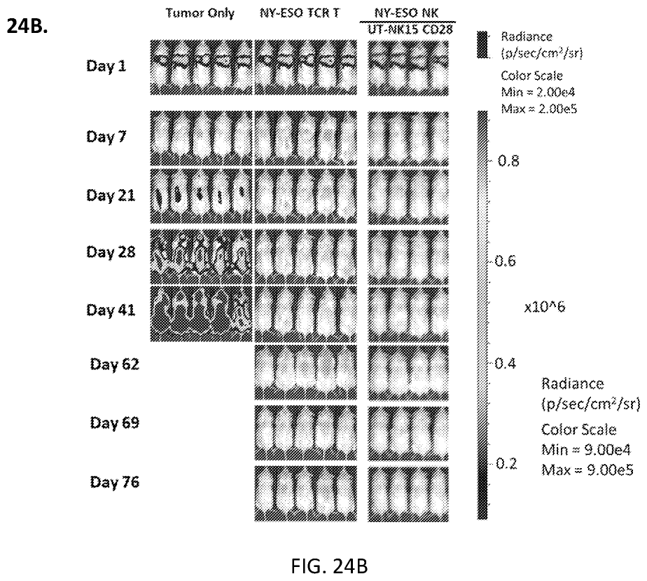

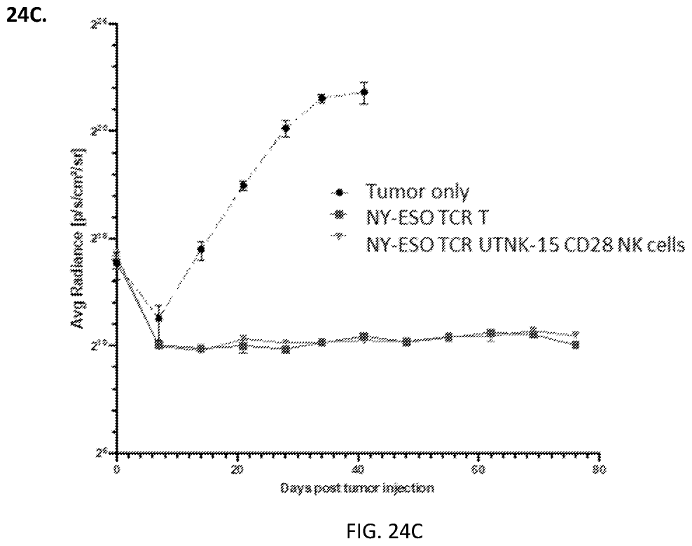

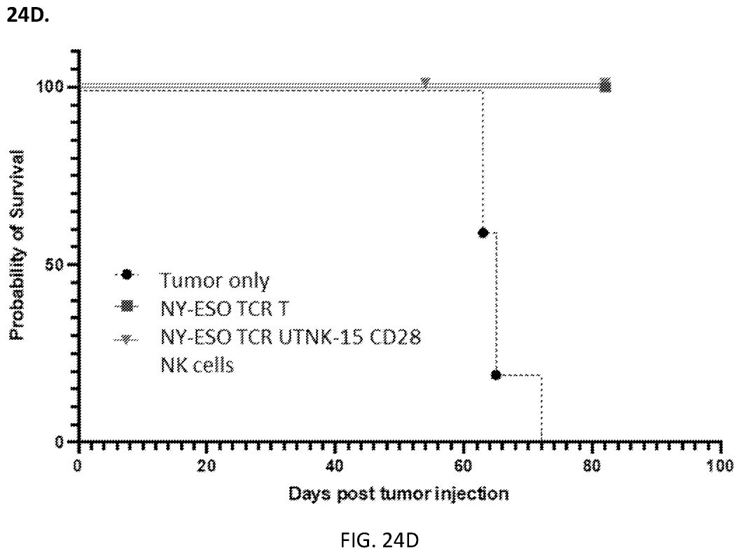

As shown in FIGS. 24A-D the in vivo activity of effector cells (e.g., NK cells or T cells) comprising NY-ESO targeted TCRs and UT-NK15 constructs was tested. FIG. 24A depicts a plan for an in vivo study to test the activity of different NY ESO TCR transduced NK and T cells. The plan was performed, wherein ten week old NSG mice were irradiated (300 cGy) and the next day they were injected with 500,000 U266 cells (HLA-A2 positive, NY-ESO-expressing myeloma cell line) via the tail vein. Three days later, the mice received 5 million TCR transduced T or TCR-transduced NK cells. Mice were then monitored for tumor control by BLI imaging. Shown in FIG. 24B are said BLI imaging results of the test outlined and performed according to FIG. 24A. Mice were injected with U266 tumor cells only, or also with T cells transduced with NY-ESO-specific TCR, or also with NK cells co-transduced with NY-ESO TCR and UT-NK15 with CD3ζ fused to CD28 (labelled as NY-ESO NK UT-NK15 CD28 or NY-ESO TCR UTNK-15 CD28 NK cells). Shown in FIG. 24C are quantifications of region of interest average radiance intensity for the animals tested according to FIG. 24A and imaged in FIG. 24B. Shown in FIG. 24D is a graph depicting the cohort survival curves for the aforementioned animals. The results showed that NY ESO TCR T and NY-ESO TCR UTNK-15-CD28 NK cells mediated strong antitumor activity in vivo.

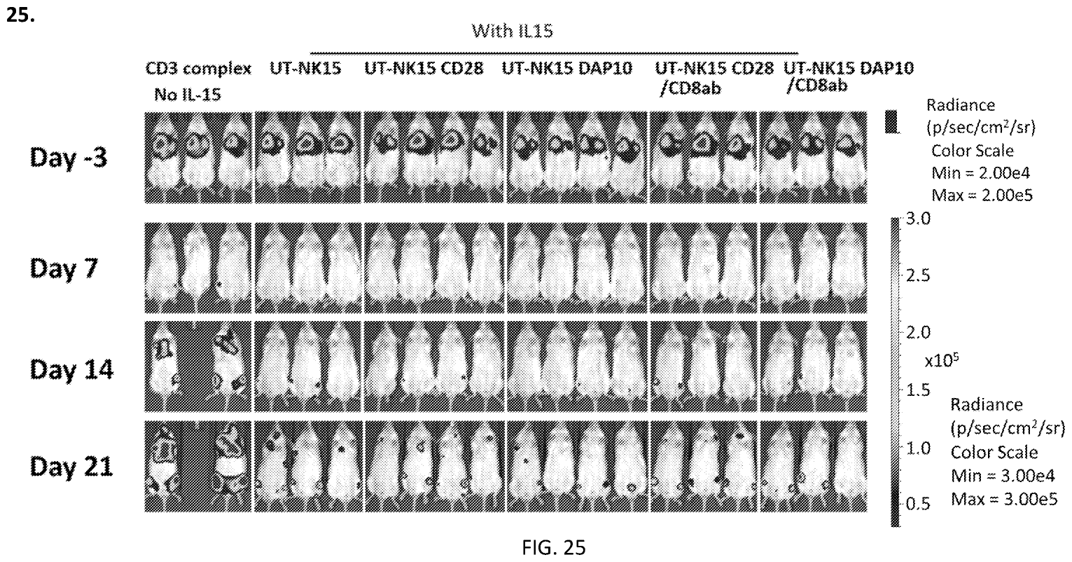

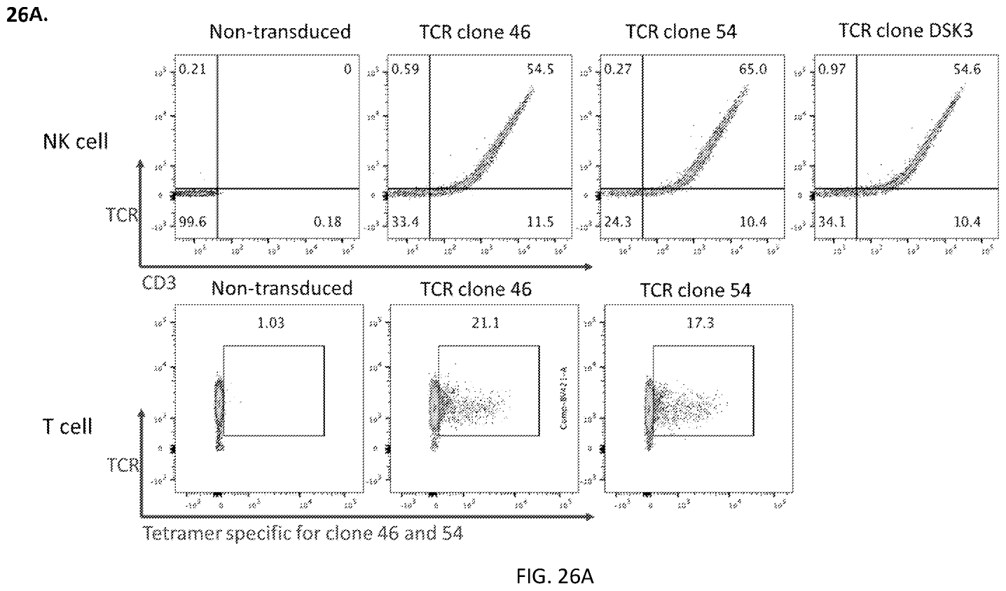

As shown in FIG. 25 the in vivo activity of effector cells (e.g., NK cells) comprising NY ESO TCR and CD3 complex with or without IL-15 was tested. NSG mice were irradiated (300 cGy) and the next day were injected with 500,000 U266 cells (HLA-A2 positive, NY-ESO-expressing myeloma cell line) via the tail vein. Three days later, mice received 5 million TCR transduced T or NK cells. Mice were monitored for tumor control by BLI imaging. NK cells were transduced with NY-ESO-specific TCR, and co-transduced with CD3 complex without IL-15 or with UT-NK15 expressing CD3ζ fused to CD28 (UT-NK15-28) or CD3ζ fused to DAP10 (UT-NK15-DAP10) co-stimulatory molecules, with or without expression of CD8 alpha/beta co-receptors. The results showed that absence of IL-15 resulted in a reduced anti-tumor activity in vivo.

Together these results showed that effector cells (e.g., NK cells) comprising constructs described herein (e.g., NY-ESO TCR constructs and/or CD3 constructs such as UT-NK15 or modified versions thereof, e.g., UT-NK-15-28 or UT-NK15-DAP10) were sufficient to robustly inhibit tumor growth in vivo.

### Example 4

### PRAME TCRs in CD3 Expressing NK Cells In Vitro

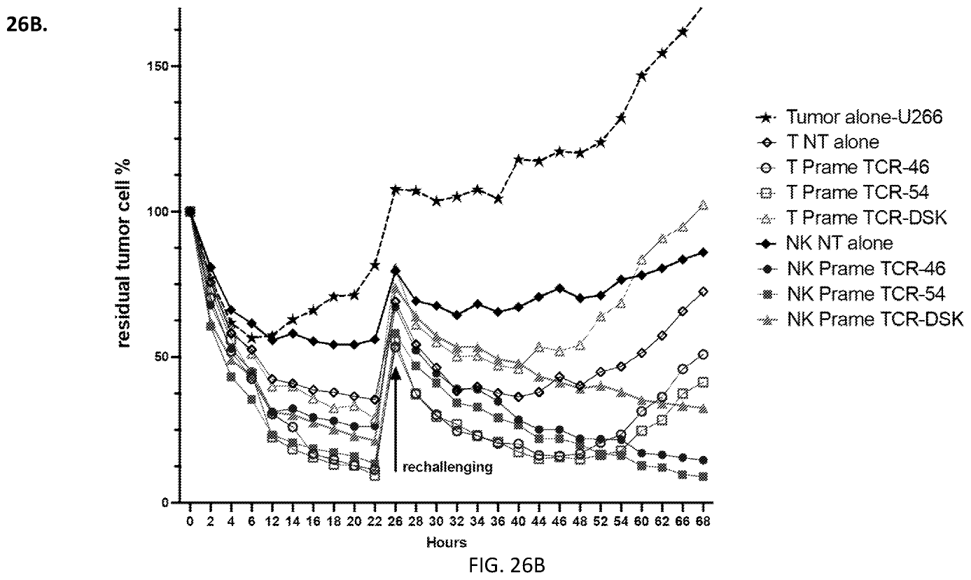

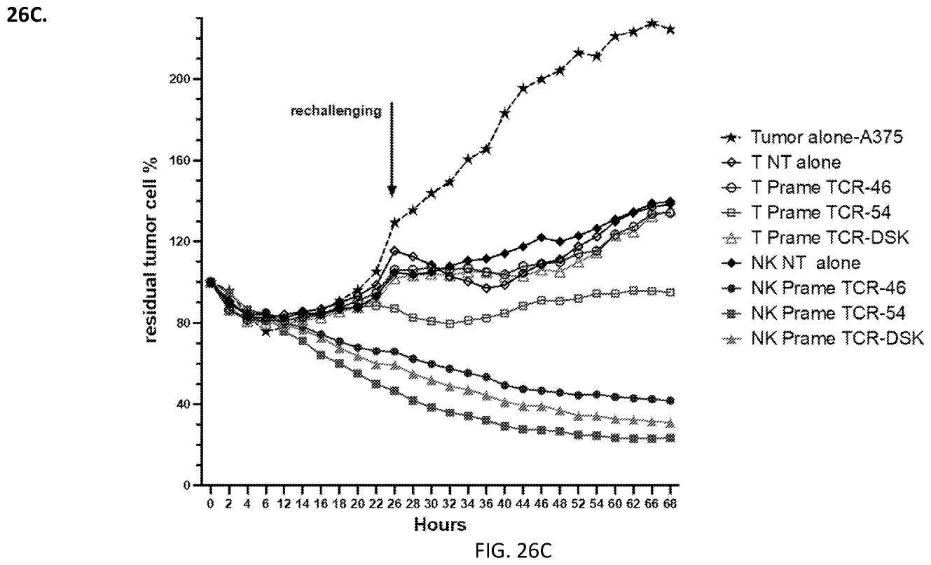

As shown in FIGS. 26A-C, NK cells comprising constructs targeting Preferentially Expressed Antigen In Melanoma (PRAME) antigen described herein were tested in-vitro and found to robustly inhibit tumor cell growth. FIG. 26A shows the expression of both UT-NK15 (x-axis, CD3) and PRAME-specific TCRs (y-axis, TCR) in NK cells (TCR clones 46, 54, or DSK3 respectively), or the expression of PRAME-specific TCRs in T cells transduced with the same (TCR clones 46 or 54). PRAME-specific TCR expression on NK cells was confirmed using antibodies against the TCR and against CD3. Expression of PRAME-specific TCR in T cells was confirmed by tetramer staining using the 46/54 peptide/MHC-specific tetramer. FIG. 26B shows the in vitro cytotoxicity of NK cells expressing a PRAME-specific TCR against the U266 myeloma cell line. IncuCyte® live cell imaging was used to measure the cytotoxicity of T cells transduced with PRAME-specific TCR and NK cells transduced with UT-NK15 and PRAME-specific TCR against U266 myeloma cells. GFP-expressing U266 cells were co-cultured with PRAME-specific TCR expressing T cell or NK cells at 1:1 effector:target ratio (50,000 effector and 50,000 target cells were seeded in each well of a 96 well plate). A reduction in GFP expression indicated cell death. After 26 hours, a second round of 50,000 tumor cells was added (noted as “rechallenging”) to each well for the tumor rechallenge assay. NK cells expressing UT-NK15 and PRAME-specific TCR clone 46 or PRAME-specific TCR clone 54 exerted the best anti-tumor activity upon rechallenge with U266 cells and displayed superior cytotoxicity when compared to control T cells transduced with PRAME-specific TCR clones 46 or 54 respectively. FIG. 26C shows the in vitro cytotoxicity of NK cells expressing a PRAME-specific TCR against the UA375 melanoma cell line. IncuCyte® live cell imaging was used to measure the cytotoxicity of T cells transduced with PRAME-specific TCR and NK cells transduced with UT-NK15 and PRAME-specific TCR against UA375 melanoma cells. GFP-expressing UA375 cells were co-cultured with PRAME-expressing T cell or NK cells at 1:1 effector:target ratio (50,000 effector and 50,000 target cells were seeded in each well of a 96 well plate). A reduction in GFP expression indicated cell death. After 26 hours, a second round of 50,000 tumor cells was added to each well for the tumor rechallenge assay. Open symbols represent T cells, while closed symbols represent NK cells. NT=non-transduced. NK cells expressing UT-NK15 and PRAME-specific TCR clone 46 (TCR-46), PRAME-specific TCR clone 54 (TCR-54), or PRAME-specific TCR clone DSK3 (DSK) exerted strong anti-tumor activity upon rechallenge with UA375 cells, and displayed superior cytotoxicity when compared to control T cells transduced with PRAME-specific TCR clones 46, 54, or DSK3 respectively.

Together these results show that effector cells (e.g., NK cells) comprising constructs described herein (e.g., PRAME-specific TCR constructs) were sufficient to robustly inhibit tumor growth in vivo. Furthermore, NK cells comprising CD3 constructs described herein coupled with PRAME-specific TCR constructs displayed increased cytotoxicity when compared to T cell control cells comprising the same TCR constructs, particularly in cases of continuous and/or rechallenge by tumor cells.

### Example 5

### TCRs in CD3 Expressing NK Cells In Vivo

NK cells comprising constructs described herein are tested in-vivo and robustly inhibit tumor growth. Experiments are performed according to schematics and experimental procedures described herein. In brief, NSG mice are irradiated (e.g., with about 300 cGy) on day −1, then on day 0 individual mice receive tail vein injections of cancer cells (e.g., 0.5×10⁶ cells e.g., cells expressing (naturally and/or transduced with) an antigen described herein) that are transduced with an appropriate marker (e.g., FireFlyluciferase (FFluc)), on day 3 mice are infused with effector cells transduced with a transgenic TCR (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, KRAS, CD19, CD20, etc.) and with or without other constructs described herein (e.g., with about 5×10⁶ TCR NK cells with a UT-NK15 construct with or without IL15, with or without CD3 fusion to a costimulatory molecule, and/or with or without additional control constructs). Animals are then monitored over time and sacrificed as appropriate. Results of the monitoring of the experiment described above are recorded, e.g., as a function of bioluminescent imaging over time (e.g., on day 1, day 7, day 14, day 21, etc).

The in vitro activity of effector cells (e.g., NK cells or T cells) comprising TCR(s) (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, KRAS, CD19, CD20, etc.) and UT-NK15 constructs are tested. Spheroids formed by an appropriate tumor cell line(s) comprising an antigen of interest (e.g., 0.5×10⁶ cells e.g., cells expressing (naturally and/or transduced with) an antigen described herein) are used to test the activity of specific TCR expressing NK and/or T cells cytotoxicity. Cancer cells are stably transduced to express an appropriate marker (e.g., GFP, FFluc, etc.); a number of these cells (e.g., about 10,000) are seeded per well in a 96 well plate overnight and a number of effector cells (e.g., about 40,000) are then added. Images of the coculture are scanned over time and analyzed by an appropriate system (e.g., an IncuCyte® cell analysis system). The percentage of cytotoxicity for effector cells are captured from representative images after a number of days (e.g., 1 day, 3 days, 7 days, etc.) of co-culture. NK cells are co-transduced with antigen targeting TCRs, and UT-NK15 signaling complex co-expressing different co-stimulatory molecules fused to the CD3ζ signaling chain (e.g., UTNK-15-28, or UTNK-15-DAP10). Appropriate control cells are transduced with appropriate constructs described herein. Superior in vitro cytotoxicity is observed with TCR NK cells expression UTNK15 with CD28, or DAP10 costimulatory domains fused to CD3ζ (e.g., UTNK-15-28, or UTNK-15-DAP10; e.g., SEQ ID NO: 121 and SEQ ID NO: 119 respectively) when compared to NK cells transduced with CD3 complex only or UT-NK15 without a co-stimulatory domain.

The in vivo activity of effector cells (e.g., NK cells or T cells) comprising antigen specific TCRs (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, KRAS, CD19, CD20, etc.) and UT-NK15 constructs are tested. Assays for in vivo analysis of effector cells (e.g., NK cells or T cells) comprising engineered constructs are performed similar to experimental plans described in FIG. 24. In brief, appropriately aged NSG mice (e.g., ten week old NSG mice) are irradiated (e.g., with about 300 cGy) and the next day they are injected with tumor cells comprising the target antigen of interest (e.g., about 500,000 cells; e.g., naturally expressing and/or transduced with an antigen described herein) via the tail vein. Three days later, the mice receive an effector cell bolus (e.g., about 5 million TCR transduced T and/or TCR-transduced NK cells). Mice are then monitored for tumor control (e.g., by BLI imaging). Average radiance for regions of interest are measured and quantified, animals comprising test constructs comprising TCRs targeting an antigen of interest and UT-NK15 constructs with or without CD3 fusions and/or IL-15 expression display improved survival relative to control animals and/or a reduction in average radiance. The results show that TCR UTNK-15 NK cells mediate strong antitumor activity in vivo.

The in vivo activity of effector cells (e.g., NK cells) comprising TCR (e.g., TCR constructs comprising gamma/delta TCR chains and/or alpha/beta TCR chains, e.g., targeting antigens described herein, e.g., NY-ESO, Tyrosinase, MAGEA3, MAGEA4, HPV E7, WT1, PRAME, gp100, MART-1, KRAS, CD19, CD20, etc.) and CD3 complex with or without IL-15 are tested. NSG mice are irradiated (e.g., with about 300 cGy) and the next day are injected with tumor cells expressing an antigen of (e.g., about 500,000 cells; e.g., naturally expressing and/or transduced with an antigen described herein) via the tail vein. Three days later, mice receive an effector cell bolus (e.g., about 5 million TCR transduced T and/or TCR transduced NK cells). Mice are monitored for tumor control (e.g., by BLI imaging). NK cells are transduced with antigen-specific TCR, and co-transduced with CD3 complex without TL-15 or with UT-NK15 expressing CD3ζ fused to CD28 (UT-NK15-28) or CD3ζ fused to DAP10 (UT-NK15-DAP10) co-stimulatory molecules, with or without expression of CD8 alpha/beta co-receptors. The results show that absence of IL-15 results in a reduced anti-tumor activity in vivo.

Together these results show that effector cells (e.g., NK cells) comprising constructs described herein (e.g., TCR constructs and/or CD3 constructs such as UT-NK15 or modified versions thereof, e.g., UT-NK-15-28 or UT-NK15-DAP10) are sufficient to robustly inhibit tumor growth in vivo.

### Example 6

### KRAS TCRs in CD3 Expressing NK Cells In Vitro

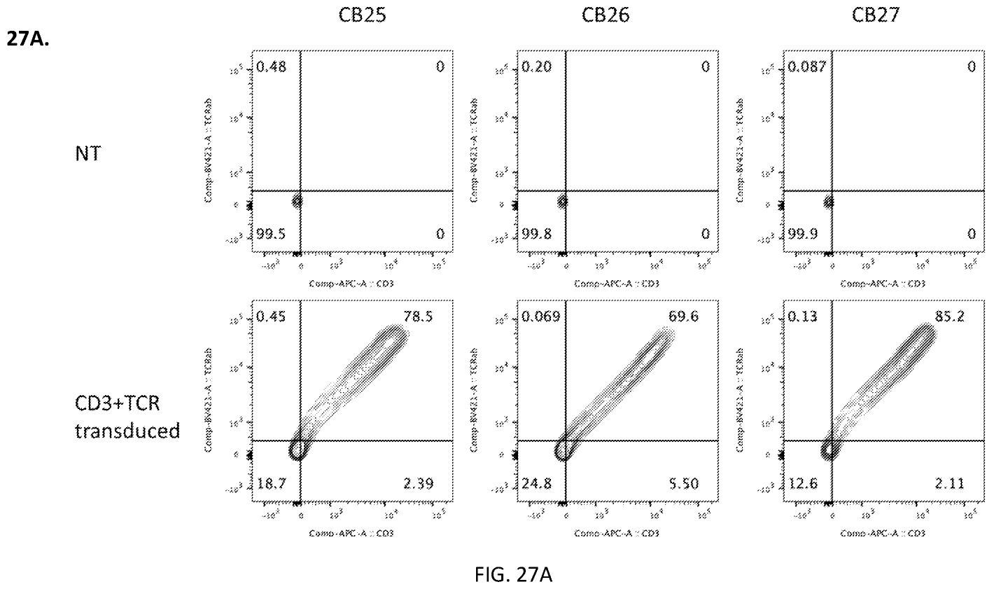

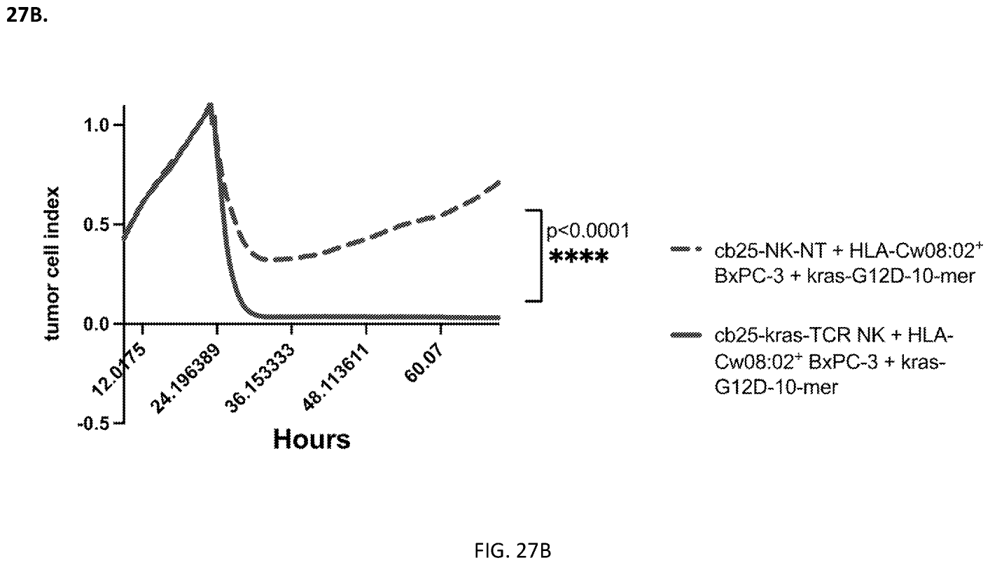

As shown in FIGS. 27A-27B, NK cells comprising TCR constructs targeting KRAS antigen described herein were tested in-vitro and found to robustly inhibit tumor cell growth. Constructs comprising KRAS-specific TCRs (e.g., SEQ ID NOs: 300 and 302) and UTNK15 constructs (e.g., SEQ ID NO: 120) were transduced into cord blood derived NK cells, and the TCR and CD3 expression profile was determined. NK cells were derived from cord blood (three donors, CD25, CD26, and CD27 respectively), and were expanded with irradiated (100 Gy) universal antigen presenting cells (uAPC) feeder cells (2:1 feeder cell:NK ratio) and recombinant human TL-2 (200 U/ml) in complete media. FIG. 27A shows the expression of both UT-NK15 (x-axis, CD3, e.g., expression of construct comprising SEQ ID NO: 120) and KRAS-specific TCRs (y-axis, TCRab) in NK cells (3 cord blood donors, CB25, CB26, and CB27 respectively) (bottom) compared to non-transduced (NT) NK cell controls (top).

As shown in FIG. 27B, the aforementioned cells (e.g., NK cells expressing KRAS-specific TCR and UT-NK15) in vitro cytotoxicity against KRAS antigen expressing tumor cell lines was tested. NK cells expressing a KRAS-specific TCR and UT-NK15 were challenged with a BcPC3 cell line (e.g., a pancreatic cancer cell line BxPC-3 transduced with a construct encoding HLA-Cw08:02 and beta-2-M (SEQ ID NO: 320) loaded with 1 μg/ml KRAS-G12D-10mer (GADGVGKSAL) peptide (SEQ ID NO: 292; noted as “G12D-10-mer”). Cytotoxicity was measured in real-time using the Xcelligence cell analysis system, the cytotoxicity's of control non-transduced NK cells (cb25-NK-NT; dashed line) or NK cells transduced with KRAS-specific TCR and UT-NK15 (cb25-kras-TCR NK; solid line) against BcPC3 cells with KRAS-G12D-10-mer (HLA-Cw08:02+ BxPC-3+kras-G12D-10-mer) were measured and compared. Cells were co-cultured at a 5:1 effector:target ratio. Assays were performed for NK cells from donors CB25, CB26, and CB27, the data presented in FIG. 27B shows data from the representative KRAS-specific TCR expressing NK cell line from CB25, with triplicate replicates for each treatment. Statistical significance was assessed by two-tailed t-test (****=P<0.0001). The tumor cell index used as a measure of cytotoxicity, and showed the significant superiority of KRAS-specific TCR UT-NK15 expressing NK cells in killing KRAS+HLA-Cw08:02+ BxPC-3 cells relative to non-transduced control NK cells

Together these results show that effector cells (e.g., NK cells) comprising constructs described herein (e.g., KRAS-specific TCR constructs and CD3 comprising constructs) were sufficient to robustly inhibit tumor cell growth and/or kill tumor cells in vitro. Furthermore, NK cells comprising KRAS-specific TCR constructs and CD3 constructs described herein displayed significantly increased cytotoxicity against cells comprising a KRAS antigen when compared to non-transduced NK cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A composition comprising engineered NK cells modified to express one or more transgenic polynucleotides encoding
a) a CD3 protein complex comprising CD3ζ, CD3δ, CD3ε, and CD3γ,
b) at least one cytokine, and
c) at least one engineered T cell receptor (TCR) comprising (i) TCR αβ, (ii) TCR γδ, or (iii) TCR αβ and TCR γδ,
wherein the engineered TCR targets a KRAS antigen.

2.-4. (canceled)

5. The composition of claim 1, wherein any one or more of the CD3ζ, CD3ε, CD3δ, and CD3γ are linked to one or more heterologous intracellular signaling domains comprising CD28, DAP10, CD16, NKG2D, DAP12, 2B4, 4-1BB, CD2, and a combination thereof.

6.-7. (canceled)

8. The composition of claim 5, wherein the intracellular signaling domain comprises an amino acid sequence at least about 85% identical to SEQ ID NO: 115, an amino acid sequence at least about 85% identical to SEQ ID NO: 116, or an amino acid sequence at least about 85% identical to SEQ ID NO: 117.

9.-15. (canceled)

16. The composition of claim 1, wherein the composition further comprises one or more monospecific, bispecific, or multi-specific antibodies.

17. The composition of claim 16, wherein the one or more bispecific or multi-specific antibodies comprise an anti-CD3 antibody.

18. (canceled)

19. The composition of claim 16, wherein the antibody is Imgatuzumab, Amivantamab, and/or Cetuximab.

20. The composition of claim 1, wherein the at least one cytokine comprises IL-15, IL-21, IL-12, IL-2, IL-18, IL-23, IL-7, GMCSF, or a combination thereof.

21. The composition of claim 1, wherein the NK cells are pre-cultured in the presence of a CD3-CD19 bispecific antibody.

22.-41. (canceled)

42. A composition comprising a complex, comprising:
a) NK cells modified to express
(i) a CD3 receptor complex comprising CD3ζ, CD3ε, CD3δ, and CD3γ; and
(ii) a T-cell receptor (TCR) complex comprising TCR αβ chains, TCR γδ chains, or both TCR αβ and γδ chains; and
b) a monospecific, bispecific, or multi-specific antibody,
wherein the bispecific or multi-specific antibody comprises an anti-CD3 antibody that is bound to CD3 on the NK cells,
wherein the TCR targets a KRAS antigen.

43. The composition of claim 42, wherein the TCR α chain is at least 85% identical to SEQ ID NO: 299, the TCR (chain is at least 85% identical to SEQ ID NO: 301, and the antibody is Imgatuzumab, Amivantamab, and/or Cetuximab.

44. (canceled)

45. The composition of claim 42, wherein any one or more of CD3ζ, CD3ε, CD3δ, and CD3γ comprise one or more heterologous intracellular signaling domains,
wherein the heterologous intracellular signaling domain is selected from the group consisting of CD28, DAP10, CD16, NKG2D, DAP12, 2B4, 4-1BB, CD2, DNAM, and a combination thereof.

46.-47. (canceled)

48. The composition of claim 45, wherein the intracellular signaling domain comprises an amino acid sequence at least about 85% identical to SEQ ID NO: 115, an amino acid sequence at least about 85% identical to SEQ ID NO: 116, or an amino acid sequence at least about 85% identical to SEQ ID NO: 117.

49.-57. (canceled)

58. A method of treating cancer in an individual, comprising administering to the individual a therapeutically effective amount of the composition of claim 42.

59. A method of treating cancer in an individual, comprising administering to the individual a therapeutically effective amount of the composition of claim 1 and one or more monospecific, bispecific, or multi-specific antibodies,
wherein the bispecific or multi-specific antibodies comprises an anti-CD3 antibody,
wherein the composition and the antibody are administered to the individual at the same time, or
wherein the composition and the antibody are administered in the same formulation.

60. The method of claim 58, wherein the NK cells and the antibody are administered to the individual at different times.

61.-63. (canceled)

64. A method of redirecting the specificity of NK cells against a cancer antigen for treatment of an individual with an anti-CD3 monospecific, bispecific or multi-specific antibody,
comprising administering to the individual the antibody and NK cells that express (i) a CD3 receptor complex comprising CD3ζ, CD3ε, CD3δ, and CD3γ.
and (ii) a TCR complex comprising αβ chains or a TCR complex comprising TCR γδ chains,
wherein the TCR targets a KRAS antigen.

65. (canceled)

66. The method of claim 64, wherein any one or more of CD3, CD3ε, CD3δ, and CD3γ are linked to one or more heterologous intracellular signaling domains comprising CD28, DAP10, CD16, NKG2D, DAP12, 2B4, 4-1BB, CD2, and a combination thereof.

67.-68. (canceled)

69. The method of claim 66, wherein the intracellular signaling domain comprises an amino acid sequence at least about 85% identical to SEQ ID NO: 115, an amino acid sequence at least about 85% identical to SEQ ID NO: 116, or an amino acid sequence at least about 85% identical to SEQ ID NO: 117.

70.-83. (canceled)
